SEQUENCE LISTING (10 sequences)

SEQ ID NO: 1 - DNA, 22 bp, Artificial Sequence
  Description of Artificial Sequence Synthetic primer
gagaaatccaaagagcaaatgg

SEQ ID NO: 2 - DNA, 21 bp, Artificial Sequence
  Description of Artificial Sequence Synthetic primer
agaatggaaaactggaatagg

SEQ ID NO: 3 - DNA, 20 bp, Artificial Sequence
  Description of Artificial Sequence Synthetic primer
tcttcgatgcttcggattcc

SEQ ID NO: 4 - DNA, 21 bp, Artificial Sequence
  Description of Artificial Sequence Synthetic primer
gaattctcggaatctctgttg

SEQ ID NO: 5 - DNA, 21 bp, Artificial Sequence
  Description of Artificial Sequence Synthetic primer
ttacaagcagtgcagaaaacc

SEQ ID NO: 6 - DNA, 22 bp, Artificial Sequence
  Description of Artificial Sequence Synthetic primer
agtaaacattgaaaccacagcc

SEQ ID NO: 7 - DNA, 20 bp, Artificial Sequence
  Description of Artificial Sequence Synthetic primer
tctgcagctctgtgtgaagg

SEQ ID NO: 8 - DNA, 22 bp, Artificial Sequence
  Description of Artificial Sequence Synthetic primer
cttcaaaaacttctccacaacc

SEQ ID NO: 9 - DNA, 17 bp, Artificial Sequence
  Description of Artificial Sequence Synthetic primer
cccacgccacgctctcc

SEQ ID NO: 10 - DNA, 19 bp, Artificial Sequence
  Description of Artificial Sequence Synthetic primer
tcctgtcagttggtgctcc

(19) United States
(12) Patent Application Publication
(10) Pub. No.: US 2016/0166619 A1
(43) Pub. Date: Jun. 16, 2016

(54) Treatment of Retinal Degeneration Using Progenitor Cells

(71) Applicant: Janssen Biotech, Inc., Horsham, PA (US)

(72) Inventors: Ian R. Harris, Spring House, PA (US); Jing Cao, Spring House, PA (US); Nadine Dejneka, Spring House, PA (US)

(73) Assignee: Janssen Biotech, Inc., Horsham, PA (US)

(21) Appl. No.: 14/960,006

(22) Filed: Dec. 4, 2015

Related U.S. Application Data
(60) Provisional application No. 62/092,658, filed on Dec. 16, 2014.
(60) Provisional application No. 62/236,732, filed on Oct. 2, 2015.

(51) Int. Cl. A61K 35/51; A61K 9/00

(52) U.S. Cl. CPC A61K 35/51; A61K 9/0048

(57) ABSTRACT

Methods and compositions for treating and reducing retinal degeneration using progenitor cells and conditioned media from progenitor cells, such as postpartum-derived cells are disclosed. Trophic factors and other agents secreted by the progenitor cells that protect retinal cells and inhibit apoptosis of retinal cells such as photoreceptor cells are also disclosed.

### CROSS REFERENCE TO RELATED APPLICATION

This application claims benefit of U.S. Provisional Application Ser. No. 62/092,658, filed Dec. 16, 2014, and U.S. Provisional Application Ser. No. 62/236,732, filed Oct. 2, 2015, the entire contents of each is incorporated by reference herein.

### FIELD OF THE INVENTION

This invention relates to the field of cell-based or regenerative therapy for ophthalmic diseases and disorders. In particular, the invention provides methods and compositions for the regeneration or repair of ocular cells and tissue using progenitor cells, such as umbilical cord tissue-derived cells and placenta tissue-derived cells, and conditioned media prepared from those cells.

### BACKGROUND

As a complex and sensitive organ of the body, the eye can experience numerous diseases and other deleterious conditions that affect its ability to function normally. Many of these conditions are associated with damage or degeneration of specific ocular cells, and tissues made up of those cells. As one example, diseases and degenerative conditions of the optic nerve and retina are the leading causes of blindness throughout the world. Damage or degeneration of the cornea, lens and associated ocular tissues represent another significant cause of vision loss worldwide.

The retina contains seven layers of alternating cells and processes that convert a light signal into a neural signal. The retinal photoreceptors and adjacent retinal pigment epithelium (RPE) form a functional unit that, in many disorders, becomes unbalanced due to genetic mutations or environmental conditions (including age). This results in loss of photoreceptors through apoptosis or secondary degeneration, which leads to progressive deterioration of vision and, in some instances, to blindness (for a review, see, e.g., Lund, R. D. et al., Progress in Retinal and Eye Research, 2001; 20: 415-449). Two classes of ocular disorders that fall into this pattern are age-related macular degeneration (AMD) and retinitis pigmentosa (RP).

AMD is the most common cause of vision loss in the United States in those people whose ages are 50 or older, and its prevalence increases with age. The primary disorder in AMD appears to be due to RPE dysfunction and changes in Bruch's membranes, characterized by, among other things, lipid deposition, protein cross-linking and decreased permeability to nutrients (see Lund et al., 2001 supra). A variety of elements may contribute to macular degeneration, including genetic makeup, age, nutrition, smoking, and exposure to sunlight or other oxidative stress. The nonexudative, or “dry” form of AMD accounts for 90% of AMD cases; the other 10% being the exudative-neovascular form (“wet” AMD). In dry-AMD patients, there is a gradual disappearance of the retinal pigment epithelium (RPE), resulting in circumscribed areas of atrophy. Since photoreceptor loss follows the disappearance of RPE, the affected retinal areas have little or no visual function.

Current therapies for AMD involve procedures, such as, for example, laser therapy and pharmacological intervention. By transferring thermal energy, the laser beam destroys the leaky blood vessels under the macula, slowing the rate of vision loss. A disadvantage of laser therapy is that the high thermal energy delivered by the beam also destroys healthy tissue nearby. Neuroscience 4^{th} edition, (Purves, D, et al. 2008) states “[c]urrently there is no treatment for dry AMD.”

RPE transplantation has been unsuccessful in humans. For example, Zarbin, M, 2003 states, “[w]ith normal aging, human Bruch's membrane, especially in the submacular region, undergoes numerous changes (e.g., increased thickness, deposition of ECM and lipids, cross-linking of protein, non-enzymatic formation of advanced glycation end products). These changes and additional changes due to AMD could decrease the bioavailability of ECM ligands (e. g., laminin, fibronectin, and collagen IV) and cause the extremely poor survival of RPE cells in eyes with AMD. Thus, although human RPE cells express the integrins needed to attach to these ECM molecules, RPE cell survival on aged submacular human Bruch's membrane is impaired.” (Zarbin, M A, Trans Am Ophthalmol Soc, 2003; 101:493-514).

Retinitis pigmentosa is mainly considered an inherited disease, with over 100 mutations being associated with photoreceptor loss (see Lund et al., 2001, supra). Though the majority of mutations target photoreceptors, some affect RPE cells directly. Together, these mutations affect such processes as molecular trafficking between photoreceptors and RPE cells and phototransduction.

Other less common, but nonetheless debilitating retinopathies can also involve progressive cellular degeneration leading to vision loss and blindness. These include, for example, diabetic retinopathy and choroidal neovascular membrane (CNVM).

The advent of stem cell-based therapy for tissue repair and regeneration provides potential treatments for a number of aforementioned cell-degenerative pathologies and other ocular disorders. Stem cells are capable of self-renewal and differentiation to generate a variety of mature cell lineages. Transplantation of such cells can be utilized as a clinical tool for reconstituting a target tissue, thereby restoring physiologic and anatomic functionality. The application of stem cell technology is wide-ranging, including tissue engineering, gene therapy delivery, and cell therapeutics, i.e., delivery of biotherapeutic agents to a target location via exogenously supplied living cells or cellular components that produce or contain those agents. (For a review, see, for example, Tresco, P. A. et al., Advanced Drug Delivery Reviews, 2000, 42: 2-37).

Recently, it has been shown that postpartum-derived cells ameliorate retinal degeneration (US 2010/0272803). The Royal College of Surgeons (RCS) rat presents with a tyrosine receptor kinase (Mertk) defect affecting outer segment phagocytosis, leading to photoreceptor cell death. (Feng W. et al., J Biol Chem., 2002, 10: 277 (19): 17016-17022). Transplantation of retinal pigment epithelial (RPE) cells into the subretinal space of RCS rats was found to limit the progress of photoreceptor loss and preserve visual function. (US 2010/0272803). It also has been demonstrated that postpartum-derived cells can be used to promote photoreceptor rescue and thus preserve photoreceptors in the RCS model. (US 2010/0272803). Injection of human umbilical cord tissue-derived cells (hUTCs) subretinally into RCS rat eye improved visual acuity and ameliorated retinal degeneration (US 2010/0272803; Lund R D, et al., Stem Cells. 2007; 25(3):602-611). Moreover, treatment with conditioned medium (CM) derived from hUTC restored phagocytosis of ROS in dystrophic RPE cells in vitro. (US 2010/0272803).

The clearance of apoptotic cells by phagocytes is an integral component of normal life, and defects in this process can have significant implications for self-tolerance and autoimmunity (Ravichandran et al., Cold Spring Harb Perspect Biol., 2013, 5(1): a008748. doi: 10.1101/cshperspect.a008748. Review). The recognition and removal of apoptotic cells are mainly mediated by professional phagocytes (receptors bind pathogen for phagocytosis), such as macrophages, monocytes, and other white blood cells, and by non-professional phagocytes (phagocytosis is not the principal function), such as epithelial cells, RPE cells, endothelial cells. Numerous “eat me” signals have been identified to date including changes in glycosylation of surface proteins or changes in surface charge (Ravichandran et al., Cold Spring Harb Perspect Biol., 2013). Externalization of phosphatidylserine (PS) is a hallmark of apoptosis, and is the best studied “eat me” signal (Wu et al., Trends. Cell Biol., 2006, 16 (4): 189-197). “Eat me” signals are recognized by phagocytic engulfment receptors either directly (as with PS receptors) or indirectly via bridge molecules and accessory receptors (Erwig et al., Cell Death. Differ., 2008; 15:243-250). The bridge molecules milk-fat-globule-EGF-factor 8 (MFG-E8), growth arrest-specific 6 (Gas6), protein S, thrombospondins (TSPs), apolipoprotein H (previously known as β2-glycoprotein I, β2-GPI) all bind to PS on the apoptotic cell surface. MFG-E8 can then be recognized by αvβ3 and αvβ5 integrins through its RGD motif (Hanayama et al., Science, 2004, 304: 1147-1150; Borisenko et al., Cell Death Differ., 2004; 11:943-945), Gas6 by receptor tyrosine kinases of the Ax1, Tyro3 and Mer family (Scott et al., Nature, 2001; 411:207-211) and apolipoprotein H to the β2-GPI receptor (Balasubramanian et al., J Bio Chem, 1997; 272:31113-31117). Other bridge molecules are linked to the recognition of altered sugars and/or lipids on the apoptotic cell surface, such as the members of the collectin family surfactant protein A and D (Vandivier et al., J Immunol, 2002; 169:3978-398).

The collectin family of molecules are then recognized through their interactions of their collagenous tails with calreticulin (CRT), which in turn signals for uptake by the phagocyte through the low-density lipoprotein (LDL)-receptor-related protein (LRP-1/CD91) (Gardai et al., Cell, 2003; 115:13-23). As another example, the first bridge molecule identified was thrombospondin (TSP)-1 (Savill et al., J Clin Invest, 1992; 90: 1513-1522), an extracellular matrix glycoprotein and thought to bind to TSP-1 binding sites on apoptotic cells and then bind to a receptor complex on the phagocyte comprising the αvβ3 and αvβ5 integrins and the scavenger receptor CD36. Annexin I belongs to the annexin family of Ca2+-dependent phospholipid-binding proteins and are preferentially located on the cytosolic face of the plasma membrane. Annexin I was shown to co-localize with PS.

Phagocytosis of ROS by RPE is essential for retinal function (Finnemann et al., PNAS, 1997; 94:12932-937). Receptors reported to participate in RPE phagocytosis of ROS include a scavenger receptor CD36, integrin receptor αvβ5, a receptor tyrosine kinase known as Mertk, and the mannose receptor (MR) (CD206) (Kevany et al., Physiology, 2009; 25:8-15). Finnemann found that isolated ROS possess externalized PS, whose blockade or removal reduces their binding and engulfment by RPE in culture (Finnemann et al., PNAS, 2012; 109 (21): 8145-8148). However, RPE phagocytosis is still poorly understood.

### SUMMARY

This invention provides compositions and methods applicable to cell-based or regenerative therapy for ophthalmic diseases and disorders. In particular, the invention features methods and compositions for treating ophthalmic disease or condition, including the regeneration or repair of ocular tissue using progenitor cells, such as postpartum-derived cells, and conditioned media generated from those cells. The postpartum-derived cells may be umbilical cord tissue-derived cells (UTCs) or placental tissue-derived cells (PDCs).

One aspect of the invention is a method of treating ophthalmic disease comprising administering to a subject progenitor cells, or a conditioned media prepared from a population of progenitor cells, wherein the cells secrete bridge molecules. In an embodiment of the invention, the bridge molecules are secreted by the cell population in the conditioned media. In a further embodiment, the bridge molecules are selected from MFG-E8, Gas6, thrombospondin (TSP)-1 and TSP-2. In embodiments of the invention, the cells are progenitor cells. In particular embodiments of the invention, the cells are postpartum-derived cells. In embodiments of the invention, the postpartum-derived cells are isolated from human umbilical cord tissue or placental tissue substantially free of blood.

In embodiments, a population of progenitor cells, for example postpartum-derived cells, secretes bridge molecules. In an embodiment, conditioned media prepared from a population of progenitor cells, for example postpartum-derived cells, contains bridge molecules secreted by the cell population. Such bridge molecules secreted by the cells and secreted in the conditioned media are selected from MFG-E8, Gas6, TSP-1 and TSP-2. The postpartum-derived cells are umbilical cord tissue-derived cells (UTCs) or placental tissue-derived cells (PDCs).

In one embodiment, the bridge molecules inhibit the apoptosis of photoreceptor cells. In another embodiment, bridge molecules secreted by progenitor cells, and secreted in conditioned media, reduce the loss of photoreceptor cells. In an embodiment, the loss of photoreceptor cells is reduced by the bridge molecules stimulating phagocytosis of photoreceptor fragments.

In another embodiment, the population of cells described above or conditioned media prepared from the population of cells described above modifies rod outer segment (ROS) to facilitate phagocytosis. In a further embodiment, the bridge molecules enhance binding and internalization of ROS by retinal pigment epithelial (RPE) cells.

In another embodiment, the population of cells described above or conditioned media prepared from the population of cells described above contains receptor tyrosine kinase (RTK) trophic factors secreted by the cell population. In a specific embodiment, the trophic factors are BDNF, NT3, HGF, PDGF-CC, PDGF-DD, and GDNF. In embodiments, the RTK trophic factors mediate phagocytosis in retinal pigment epithelial (RPE) cells.

In certain embodiments, the RTK trophic factors mediate phagocytosis by RPE cells to phagocytose shed photoreceptor fragments (photoreceptor fragments shed by the cells). In a further embodiment, the RTK trophic factors activate receptors on RCE cells to stimulate phagocytosis.

Another aspect of the invention features a method for reducing the loss of photoreceptor cells in retinal degeneration, the method comprising administering to the eye a population of progenitor cells or a conditioned media prepared from the population of progenitor cells in an amount effective to reduce the loss of photoreceptor cells. In an embodiment of the invention, the progenitor cells are postpartum-derived cells. In a particular embodiment, the postpartum-derived cells are isolated from human umbilical cord tissue or placental tissue substantially free of blood. As in other embodiments, the postpartum-derived cells secrete bridge molecules. In embodiments, the conditioned media contains bridge molecules secreted by the cell population, such as a population of postpartum-derived cells. Such bridge molecules secreted by the postpartum-derived cells are selected from MFG-E8, Gas6, TSP-1 and TSP-2.

In another embodiment, the conditioned media is generated from an isolated postpartum-derived cell or a population of postpartum-derived cells, derived from human umbilical cord tissue or placental tissue substantially free of blood. In embodiments, the postpartum-derived cell is capable of expansion in culture and has the potential to differentiate into a cell of a neural phenotype; wherein the cell requires L-valine for growth and is capable of growth in at least about 5% oxygen. This cell further comprises one or more of the following characteristics: (a) potential for at least about 40 doublings in culture; (b) attachment and expansion on a coated or uncoated tissue culture vessel, wherein the coated tissue culture vessel comprises a coating of gelatin, laminin, collagen, polyomithine, vitronectin, or fibronectin; (c) production of at least one of tissue factor, vimentin, and alpha-smooth muscle actin; (d) production of at least one of CD10, CD13, CD44, CD73, CD90, PDGFr-alpha, PD-L2 and HLA-A,B,C; (e) lack of production of at least one of CD31, CD34, CD45, CD80, CD86, CD117, CD141, CD178, B7-H2, HLA-G, and HLA-DR,DP,DQ, as detected by flow cytometry; (f) expression of a gene, which relative to a human cell that is a fibroblast, a mesenchymal stem cell, or an iliac crest bone marrow cell, is increased for at least one of a gene encoding: interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; tumor necrosis factor, alpha-induced protein 3; C-type lectin superfamily member 2; Wilms tumor 1; aldehyde dehydrogenase 1 family member A2; renin; oxidized low density lipoprotein receptor 1; Homo sapiens clone IMAGE:4179671; protein kinase C zeta; hypothetical protein DKFZp564F013; downregulated in ovarian cancer 1; and Homo sapiens gene from clone DKFZp547k1113; (g) expression of a gene, which relative to a human cell that is a fibroblast, a mesenchymal stem cell, or an iliac crest bone marrow cell, is reduced for at least one of a gene encoding: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeo box 2 (growth arrest-specific homeo box); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (T AZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeo box 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; cytochrome c oxidase subunit VIIa polypeptide 1 (muscle); similar to neuralin 1; B cell translocation gene 1; hypothetical protein FLJ23191; and DKFZp586L151; and (h) lack expression of hTERT or telomerase. In one embodiment, the umbilical cord tissue-derived cell further has the characteristics of (i) secretion of at least one of MCP-1, IL-6, IL-8, GCP-2, HGF, KGF, FGF, HB-EGF, BDNF, TPO, MIP1b, 1309, MDC, RANTES, and TIMP1; (j) lack of secretion of at least one of TGF-beta2, MIP1a, ANG2, PDGFbb, and VEGF, as detected by ELISA. In another embodiment, the placenta tissue-derived cell further has the characteristics of (i) secretion of at least one of MCP-1, IL-6, IL-8, GCP-2, HGF, KGF, HB-EGF, BDNF, TPO, MIP1a, RANTES, and TIMP1; (j) lack of secretion of at least one of TGF-beta2, MIP1b, ANG2, PDGFbb, FGF, and VEGF, as detected by ELISA.

In specific embodiments, the postpartum-derived cell has all the identifying features of cell type UMB 022803 (P7) (ATCC Accession No. PTA-6067); cell type UMB 022803 (P17) (ATCC Accession No. PTA-6068), cell type PLA 071003 (P8) (ATCC Accession No. PTA-6074); cell type PLA 071003 (P11) (ATCC Accession No. PTA-6075); or cell type PLA 071003 (P16) (ATCC Accession No. PTA-6079. In an embodiment, the postpartum-derived cell derived from umbilicus tissue has all the identifying features of cell type UMB 022803 (P7) (ATCC Accession No. PTA-6067) or cell type UMB 022803 (P17) (ATCC Accession No. PTA-6068). In another embodiment, the postpartum-derived cell derived from placenta tissue has all the identifying features of cell type PLA 071003 (P8) (ATCC Accession No. PTA-6074); cell type PLA 071003 (P11) (ATCC Accession No. PTA-6075); or cell type PLA 071003 (P16) (ATCC Accession No. PTA-6079).

In certain embodiments, postpartum-derived cells are isolated in the presence of one or more enzyme activities comprising metalloprotease activity, mucolytic activity and neutral protease activity. Preferably, the postpartum-derived cells have a normal karyotype, which is maintained as the cells are passaged in culture. In preferred embodiments, the postpartum-derived cells express each of CD10, CD13, CD44, CD73, CD90. In embodiments, the postpartum-derived cells express each of CD10, CD13, CD44, CD73, CD90, PDGFr-alpha, and HLA-A,B,C. In preferred embodiments, the postpartum-derived cells do not express CD31, CD34, CD45, CD117. In embodiments, the postpartum-derived cells do not express CD31, CD34, CD45, CD117, CD141, or HLA-DR,DP,DQ, as detected by flow cytometry. In embodiments, the cells lack expression of hTERT or telomerase.

In embodiments, as above, the cell population is a substantially homogeneous population of postpartum-derived cells. In a specific embodiment, the population is a homogeneous population of postpartum-derived cells. In embodiments of the invention, the postpartum-derived cells are derived from human umbilical cord tissue or placental tissue substantially free of blood.

In certain embodiments, the population of postpartum-derived cells or the conditioned medium prepared from the cell population as described above is administered with at least one other cell type, such as an astrocyte, oligodendrocyte, neuron, neural progenitor, neural stem cell, retinal epithelial stem cell, corneal epithelial stem cell, or other multipotent or pluripotent stem cell. In these embodiments, the other cell type can be administered simultaneously with, before, or after the population of postpartum-derived cells or the conditioned medium prepared from the population of postpartum-derived cells.

Likewise, in these and other embodiments, the population of postpartum-derived cells or the conditioned medium prepared from the population of postpartum-derived cells as described above is administered with at least one other agent, such as a drug for ocular therapy, or another beneficial adjunctive agent such as an anti-inflammatory agent, anti-apoptotic agents, antioxidants or growth factors. In these embodiments, the other agent can be administered simultaneously with, before, or after, the population of postpartum-derived cells or the conditioned medium prepared from the population of postpartum-derived cells.

In various embodiments described herein, the population of postpartum-derived cells (umbilical or placental) or the conditioned medium generated from postpartum-derived cells is administered to the eye, for example the surface of an eye, or to the interior of an eye or to a location in proximity to the eye, e.g., behind the eye. The population of postpartum-derived cells or the conditioned medium prepared from the population of postpartum-derived cells can be administered through a cannula or from a device implanted in the patient's body within or in proximity to the eye, or may be administered by implantation of a matrix or scaffold with the population of cells or the conditioned media.

Another aspect of the invention features a composition for reducing the loss of photoreceptor cells in a retinal degenerative condition, comprising a population of progenitor cells or a conditioned media prepared from a population of progenitor cells in an amount effective to reducing the loss of photoreceptor cells. Preferably, the progenitor cells are postpartum-derived cells as described above. More preferably, the postpartum-derived cells are isolated from a postpartum umbilical cord or placenta substantially free of blood as described above. The degenerative condition may be an acute, chronic or progressive condition.

In certain embodiments, the composition above comprises at least one other cell type, such as an astrocyte, oligodendrocyte, neuron, neural progenitor, neural stem cell, retinal epithelial stem cell, corneal epithelial stem cell, or other multipotent or pluripotent stem cell. In these or other embodiments, the composition comprises at least one other agent, such as a drug for treating the ocular degenerative disorder or other beneficial adjunctive agents, e.g., anti-inflammatory agents, anti-apoptotic agents, antioxidants or growth factors.

In embodiments as described above, the composition is a pharmaceutical composition further comprising a pharmaceutically-acceptable carrier. In certain embodiments, the pharmaceutical compositions are formulated for administration to the surface of an eye. Alternatively, they can be formulated for administration to the interior of an eye or in proximity to the eye (e.g., behind the eye). The pharmaceutical compositions also can be formulated as a matrix or scaffold containing the progenitor cells or conditioned media prepared from the progenitor cells as described above.

According to yet another aspect of the invention, a kit is provided for treating a patient having an ocular degenerative condition. The kit comprises a pharmaceutically acceptable carrier, progenitor cells or a conditioned media generated from progenitor cells such as cells isolated from postpartum tissue, preferably the postpartum-derived cells described above, and instructions for using the kit in a method of treating the patient. The kit may also contain one or more additional components, such as reagents and instructions for generating the conditioned medium, or a population of at least one other cell type, or one or more agents useful in the treatment of an ocular degenerative condition.

Other aspects of the invention include a method of reducing the loss of photoreceptor cells in retinal degeneration, the method comprising administering a composition comprising a population of progenitor cells or a conditioned media prepared from a population of progenitor cells, in an amount effective to reduce the loss of photoreceptor cells. Preferably the progenitor cells are postpartum-derived cells or the conditioned media is prepared from a population of postpartum-derived cells as described herein. In embodiments of the invention, the postpartum-derived cells are isolated from umbilical cord tissue or placental tissue substantially free of blood. In a specific embodiment, the postpartum-derived cells or the conditioned media prepared from a population of postpartum-derived cells contains bridge molecules secreted by the cell population. Such bridge molecules secreted by the postpartum-derived cells or secreted in the conditioned media are selected from MFG-E8, Gas6, TSP-1 and TSP-2.

In some embodiments, the present invention provides a method for reducing the loss of photoreceptor cells in retinal degeneration, the method comprising administering to the eye postpartum-derived cells or a conditioned media prepared from a population of postpartum-derived cells in an amount effective to reduce or prevent the loss of photoreceptor cells. The postpartum-derived cells are derived from umbilical cord tissue or placental tissue substantially free of blood. In some embodiments, the population of postpartum-derived cells is a substantially homogeneous population. In particular embodiments, the population of cells is homogeneous.

In further aspects of the invention described herein, the population of postpartum-derived cells (umbilical or placental) or the conditioned medium generated from postpartum-derived cells protects retinal cells or improves retinal damage from oxidative stress or oxidative damage. In an embodiment, the present invention is a method of reducing retinal degeneration, the method comprising administering a population of postpartum-derived cells or conditioned media generated from a population of postpartum-derived cells to the eye in an amount effective to reduce or protect from oxidative stress or damage. In embodiments of the invention, the postpartum-derived cells are isolated from umbilical cord tissue or placental tissue substantially free of blood. In embodiments, retinal cells and tissue are exposed to oxidative stress or oxidative damage. In embodiments herein, retinal cells and tissue are photoreceptor cells or retinal epithelium, including retinal pigment epithelial (RPE) cells. In embodiments herein, oxidative stress or oxidative damage is high oxygen tension, sunlight exposure, including chronic sunlight exposure.

An embodiment is a method of reducing the loss of photoreceptor cells in retinal degeneration, the method comprising administering a population of postpartum-derived cells or conditioned media generated from a population of postpartum-derived cells to the eye in an amount effective to reduce or protect from oxidative stress or damage. In embodiments, the postpartum-derived cells are isolated from umbilical cord tissue or placental tissue substantially free of blood. In embodiments, retinal cells and tissue are exposed to oxidative stress or oxidative damage. In embodiments herein, retinal cells and tissue are photoreceptor cells or retinal epithelium, including retinal pigment epithelial (RPE) cells. In embodiments herein, oxidative stress or oxidative damage is selected from high oxygen tension, sunlight exposure, including chronic sunlight exposure, free radical stress, photoxidation, and light-induced damage.

In one embodiment, the present invention is a method for reducing the loss of photoreceptor cells in retinal degeneration, the method comprising administering a population of postpartum-derived cells or conditioned media generated from a population of postpartum-derived cells in an amount effective to reduce or prevent the loss of photoreceptor cells, wherein the cell population is isolated from postpartum tissue substantially free of blood, and wherein the cell population is capable of expansion in culture, has the potential to differentiate into cells of at least a neural phenotype, maintains a normal karyotype upon passaging, and has the following characteristics:
a) potential for 40 population doublings in culture;
b) production of CD10, CD13, CD44, CD73, and CD90; and
c) lack of production of CD31, CD34, CD45, CD117, and CD141, and
   wherein the population of postpartum-derived cells secretes bridge molecules, wherein conditioned media prepared from a population of postpartum-derived cells contains bridge molecules secreted by the cell population. In some embodiments, the bridge molecules secreted by the postpartum-derived cells are selected from MFG-E8, Gas6, TSP-1 and TSP-2. In some embodiments, the population of cells is a substantially homogeneous population. In particular embodiments, the population of cells is homogeneous. The postpartum-derived cells are umbilical cord tissue-derived cells or placental tissue-derived cells. In an embodiment, the umbilical cord tissue-derived cell population secretes MCP-1, IL-6, IL-8, GCP-2, HGF, KGF, FGF, HB-EGF, BDNF, TPO, MIP1b, 1309, MDC, RANTES, and TIMP1. In embodiments, the umbilical cord tissue-derived cell population lacks secretion of TGF-beta2, MIP1a, ANG2, PDGFbb, and VEGF, as detected by ELISA. In another embodiment, the placental tissue-derived cell population secretes MCP-1, IL-6, IL-8, GCP-2, HGF, KGF, HB-EGF, BDNF, TPO, MIP1a, RANTES, and TIMP1. In embodiments, the placental tissue-derived cell population lacks secretion of TGF-beta2, MIP1b, ANG2, PDGFbb, FGF, and VEGF, as detected by ELISA. In embodiments, the umbilicus-derived cells or placental-derived cells are positive for HLA-A,B,C, and negative for HLA-DR,DP,DQ. In further embodiments, the umbilicus-derived cells lack expression of hTERT or telomerase.

In one embodiment, the present invention is a method for reducing the loss of photoreceptor cells in retinal degeneration, the method comprising administering a population of postpartum-derived cells, or a conditioned media prepared from a population of postpartum-derived cells, in an amount effective to reduce the loss of photoreceptor cells, wherein the cell population is isolated from human umbilical cord tissue substantially free of blood, and wherein the cell population is capable of expansion in culture, has the potential to differentiate into cells of at least a neural phenotype, maintains a normal karyotype upon passaging, and has the following characteristics:
a) potential for 40 population doublings in culture;
b) production of CD10, CD13, CD44, CD73, and CD90;
c) lack of production of CD31, CD34, CD45, CD117, and CD141, and
d) increased expression of genes encoding interleukin 8 and reticulon 1 relative to a human cell that is a fibroblast, a mesenchymal stem cell, or an iliac crest bone marrow cell, and
   wherein the population of postpartum-derived cells secretes bridge molecules, or wherein the conditioned media prepared from a population of postpartum-derived cells contains bridge molecules secreted by the cell population. In embodiments, the bridge molecules secreted by the population of postpartum-derived cells, or bridge molecules in the conditioned media secreted by the population of postpartum-derived cells are selected from MFG-E8, Gas6, TSP-1 and TSP-2. In embodiments, the cell population secretes MCP-1, IL-6, IL-8, GCP-2, HGF, KGF, FGF, HB-EGF, BDNF, TPO, MIP1b, 1309, MDC, RANTES, and TIMP1. In some embodiments, the cell population lacks secretion of TGF-beta2, MIP1a, ANG2, PDGFbb, and VEGF, as detected by ELISA. In embodiments, the cell population is positive for HLA-A,B,C, and negative for HLA-DR,DP,DQ. In some embodiments, the population of cells is a substantially homogeneous population. In particular embodiments, the population of cells is homogeneous. Further, the cell population lacks expression of hTERT or telomerase.

In certain embodiments, the present invention provides a method for reducing the loss of photoreceptor cells in retinal degeneration, the method comprising administering a population of umbilicus-derived cells, or conditioned media prepared from a population of umbilicus-derived cells, in an amount effective to reduce or prevent the loss of photoreceptor cells, wherein the cell population is isolated from human umbilical cord tissue substantially free of blood, and wherein the cell population is capable of expansion in culture, has the potential to differentiate into cells of at least a neural phenotype, and has the following characteristics:
a. potential for 40 population doublings in culture;
b. production of CD10, CD13, CD44, CD73, and CD90; and
c. increased expression of genes, encoding interleukin 8 and reticulon 1 relative to a human cell that is a fibroblast, a mesenchymal stem cell, or an iliac crest bone marrow cell, and
   the population of postpartum-derived cells secretes bridge molecules, wherein the conditioned media prepared from a population of postpartum-derived cells contains bridge molecules secreted by the cell population. In embodiments, the bridge molecules secreted in the population of postpartum-derived cells secretes bridge molecules, or bridge molecules secreted in conditioned media are selected from MFG-E8, Gas6, TSP-1 and TSP-2. In embodiments, the cells lack production of, or are negative for CD31, CD34, CD45, CD117, and CD141. In an embodiment, the umbilical cord tissue-derived cell population secretes MCP-1, IL-6, IL-8, GCP-2, HGF, KGF, FGF, HB-EGF, BDNF, TPO, MIP1b, 1309, MDC, RANTES, and TIMP1, and lacks secretion of TGF-beta2, MIP1a, ANG2, PDGFbb, and VEGF, as detected by ELISA. In embodiments, the population of umbilicus-derived cells are positive for HLA-A,B,C, and negative for HLA-DR,DP,DQ. Further, the cell population lacks expression of hTERT or telomerase. In some embodiments, the population of cells is a substantially homogeneous population. In particular embodiments, the population of cells is homogeneous.

Another aspect of the invention is a method of making a conditioned media comprising culturing a population of cells, wherein the conditioned media contains bridge molecules secreted by the cell population. In an embodiment of the invention, the bridge molecules are secreted by the cell population in the conditioned media. In a further embodiment, the bridge molecules are selected from MFG-E8, Gas6, thrombospondin (TSP)-1 and TSP-2. In embodiments of the invention, the cells are progenitor cells. In particular embodiments of the invention, the cells are postpartum-derived cells. In embodiments of the invention, the postpartum-derived cells are isolated from human umbilical cord tissue or placental tissue substantially free of blood.

In the embodiments of the invention as described above, the population of postpartum-derived cells have the following characteristics: attachment and expansion on a coated or uncoated tissue culture vessel, wherein the coated tissue culture vessel comprises a coating of gelatin, laminin, collagen, polyornithine, vitronectin, or fibronectin; production of vimentin and alpha-smooth muscle actin; and positive for HLA-A,B,C, and negative for HLA-DR,DP,DQ.

In embodiments of the invention as described above, the retinal degeneration, retinopathy or retinal/macular disorder is age-related macular degeneration. In an alternate embodiment, the retinal degeneration, retinopathy or retinal/macular disorder is dry age-related macular degeneration.

In embodiments of the invention as described above, the loss of photoreceptor cells is reduced or prevented by inhibiting the apoptosis of the photoreceptor cells. In embodiments, the loss of photoreceptor cells is reduced or prevented by stimulating the phagocytosis of shed photoreceptor fragments.

### BRIEF DESCRIPTION OF THE DRAWINGS

FIGS. 1A-1B show the effect on dystrophic RPE phagocytosis with pre-incubation in a preparation of hUTC CM1 with serum. FIG. 1A. Pigmented dystrophic RPE was preincubated with the CM1 preparation (with serum). For controls, tan hooded normal and pigmented dystrophic RPE were preincubated with control medium (DMEM:F12 medium+10% FBS+Pen (50 U/ml)/Strep (50 μg/ml)). Incubation time was 16 hours. (tan N: tan hooded normal RPE; pig D: pigmented dystrophic RPE; con: control; M: medium; CM: conditioned medium; w: with). Values are mean±SD of number of phagocytized ROS in the counted fields in one sample (n=11 or 12 per sample; n: number of fields counted); p<0.05 versus pig D control. FIG. 1B. Dystrophic RPE was preincubated with CM1 (with serum). For controls, normal and dystrophic RPE were preincubated in control media with serum. Incubation time was 24 hours. (N: normal RPE; D: dystrophic RPE; D (1): triplicate 1; D (2): triplicate 2; D (3): triplicate 3; con: control; M: medium; CM: conditioned medium; w: with). Values are mean±SD of number of phagocytized ROS in the counted fields in one sample (n=11, 12 or 13 per sample; n: number of fields counted). p<0.05 versus D control.

FIG. 2 shows the effect on dystrophic RPE phagocytosis with pre-incubation in a preparation of hUTC CM1 without serum. Pigmented dystrophic RPE was preincubated with CM1 (without serum). For controls, tan hooded normal and pigmented dystrophic RPE were preincubated in control media without serum. Incubation time was 24 hours. (tan N: tan hooded normal RPE; pig D: pigmented dystrophic RPE; con: control. M: medium; CM: conditioned medium; wo: without. Values are mean±SD of number of phagocytized ROS in the counted fields in one sample (n=9, 10, 11 or 12 per sample; n: number of fields counted); p<0.05 versus pig D control.

FIGS. 3A-3D show the effect on phagocytosis with pre-incubation in hUTC CM. (FIG. 3A) Effect on phagocytosis with pre-incubation in hUTC CM2. Pigmented dystrophic RPE was preincubated with CM2. For controls, tan hooded normal and pigmented dystrophic RPE were preincubated in control media. Incubation time was 24 hours. (tan N: tan hooded normal RPE; pig D: pigmented dystrophic RPE; con: control. M: medium; CM: conditioned medium). Values are mean±SD of number of phagocytized ROS in the counted fields in one sample (n=8 or 10 per sample; n: number of fields counted). (FIGS. 3B-3D) Effect on phagocytosis with pre-incubation in hUTC CM3. Dystrophic RPE was preincubated with the CM3. For controls, normal and dystrophic RPE were preincubated in control media. Incubation time was 24 hours. FIG. 3B. (N: normal RPE; D: dystrophic RPE; con: control; M: medium; CM: conditioned medium). Values are mean±SD of number of phagocytized ROS in the counted fields in triplicate samples (n=5-12 per sample; n: number of fields counted); p<0.05 versus D+con M control. FIG. 3C. Normal RPE control was from CM3 test 1. (N: normal RPE; D: dystrophic RPE; con: control; M: medium; CM: conditioned medium). Values are mean±SD of number of phagocytized ROS in the counted fields in one sample (n=11 or 14 per sample; n: number of fields counted); p<0.05 versus D+con M control. FIG. 3D. (N: normal RPE; N1: normal RPE from culture; N2: normal RPE from N2 culture; tan N: tan hooded normal RPE; D: dystrophic RPE; con: control; M: medium; CM: conditioned medium). Values are mean±SD of number of phagocytized ROS in the counted fields in one sample (n=12 or 13 per sample; n: number of fields counted); p<0.05 versus D+con M control.

FIGS. 4A-4C show the effect of hUTC on phagocytosis in RCS RPE cells in vitro. RCS RPE cells co-cultured with hUTC plated in transwells (FIG. 4A) or incubated with hUTC CM (FIG. 4B) for 24 hours and then subjected to phagocytosis assay. N, normal RPE; D: dystrophic RCS RPE; CM, conditioned medium. Increased phagocytosis was observed in RCS RPE co-cultured with hUTC or incubated with hUTC CM. (FIG. 4C) Photoreceptor OS incubated with hUTC CM for 24 hours and then fed to RCS RPE cells for phagocytosis assay in the absence of hUTC CM. Phagocytosis of hUTC CM-treated OS by RCS RPE was restored. Data represent the mean±SEM (n=3). ****p<0.0001.

FIGS. 5A-5B show the results of RTK ligand assays for BDNF or HB-EGF. Dystrophic RPE cells were incubated with BDNF (200 ng/ml) (FIG. 5A) or HB-EGF (200 ng/ml) (FIG. 5B) in MEM+5% FBS (MEM5) for 24 h. For positive control, dystrophic RPE cells were incubated in CM3 for 24 h. For other controls, normal and dystrophic RPE were incubated in MEM5 for 24 h and subjected to phagocytosis assay. (N: normal RPE; D: dystrophic RPE; con: control; M: medium; CM: conditioned medium). Values are mean±SD of number of phagocytized ROS in the counted fields in duplicate or triplicate samples (n=10, 11 or 12 per sample; n: number of fields counted); p<0.05 versus D control.

FIGS. 6A-6E show the results of RTK ligand assays for PDGF-DD, Ephrin A4, and HGF. Dystrophic RPE cells were incubated with PDGF-DD (FIGS. 6A, 6B), Ephrin A4 (FIG. 6C) or HGF (200 ng/ml) (FIGS. 6D, 6E) in MEM5 for 24 h and then subjected to phagocytosis assay with the addition of ROS in MEM5 containing PDGF-DD, Ephrin A4 or HGF (medium was not changed when adding ROS). For positive control, dystrophic RPE cells were incubated in CM3 for 24 h. For other controls, normal and dystrophic RPE were incubated in MEM5 for 24 h and subjected to phagocytosis assay. (N: normal RPE; D: dystrophic RPE; con: control; M: medium; CM: conditioned medium). Values are mean±SD of number of phagocytized ROS in the counted fields in duplicate or triplicate samples (n=10, 11 or 12 per sample; n: number of fields counted); p<0.05 versus D control.

FIGS. 7A-7B show the results of RTK ligand assays for Ephrin B2. Dystrophic RPE cells were incubated with Ephrin B2 (200 ng/ml). For positive control, dystrophic RPE cells were incubated in CM3. For other controls, normal and dystrophic RPE were incubated in MEM5 for 24 h and subjected to phagocytosis assay. (N: normal RPE; D: dystrophic RPE; con: control; M: medium; CM: conditioned medium). Values are mean±SD of number of phagocytized ROS in the counted fields in duplicate or triplicate samples (n=10, 11 or 12 per sample; n: number of fields counted); p<0.05 versus D control.

FIGS. 8A-8C show dystropic RPE cells treated with endothelin-1, TGF-β1, or IL-6. (FIG. 8A) Endothelin-1 or TGF-β1 (at 200 ng/mL) assayed for phagocytosis compared to normal controls. (FIGS. 8B, 8C) Dystrophic RPE cells incubated with recombinant human endothelin-1, TGF-β1 or IL-6 at 200 ng/mL and assayed for phagocytosis. hUTC CM3 was used as a positive control. For other controls, normal and dystrophic RPE were incubated in MEM5 for 24 h and subject to phagocytosis assay. (N: normal RPE; D: dystrophic RPE). Values are mean±SD of number of phagocytized ROS in the counted fields per sample (n=10 per sample; n: number of fields counted).

FIG. 9 shows dystrophic RPE cells fed with ROS preincubated with various concentrations of vitronectin and assayed for phagocytosis, along with normal controls. ROS was preincubated with control medium (DMEM+10% FBS) or CM3. In parallel, ROS was preincubated in MEM20 with various concentrations of human recombinant vitronectin (4, 2, 1, 0.5 ug/ml) respectively. For controls, normal RPE alone or dystrophic RPE alone was cultured in MEM20, then changed to MEM5 in the presence of untreated ROS (resuspended in MEM20 and fed to RPE cells) for phagocytosis assay. (N: normal RPE; D: dystrophic RPE; Con M: control medium; V: vitronectin). Values are mean±SD of number of phagocytized ROS in the counted fields per sample (n=10 per sample; n: number of fields counted).

FIG. 10 shows gene expression level of RTK ligands identified in hUTC. Total mRNAs were extracted from hUTC and RNA-Seq was performed to identify and quantify the RTK ligands gene expression in hUTC. The identified RTK ligands were sorted based on the corresponding RTK subfamilies and ranked according to their FPKM value. Gene expression of multiple RTK ligands for 15 RTK subfamilies were detected in hUTC.

FIGS. 11A-11G show levels of selected RTK ligands measured in hUTC CM. (FIGS. 11A-11F) Levels of RTK ligands compared to those from NHDF and ARPE-19 conditioned medium. BDNF is secreted in high level in hUTC conditioned medium compared to NHDF and ARPE-19 conditioned medium (FIG. 11A). NT3 level is high in hUTC CM compared to NHDF CM, whereas the amount of NT3 in ARPE-19 conditioned medium and control medium was undetectable (FIG. 11B). HGF is secreted in high level in hUTC CM compared to NHDF and ARPE-19 conditioned medium (FIG. 11C). PDGF-CC and PDGF-DD levels in hUTC conditioned medium are low compared to NHDF and ARPE-19 conditioned medium (FIG. 11D and FIG. 11E, respectively). GDNF is secreted in hUTC and NHDF conditioned medium, with trace amount in ARPE-19 conditioned medium (FIG. 11F). All values are mean±SD of triplicate samples, except NT3 is mean±SD of duplicate samples. (FIG. 11G) Levels of RTK ligands measured by ELISA. (Data shown are the mean±SEM; n=3).

FIGS. 12A-12 E show levels of bridge molecules measured in hUTC CM. (FIGS. 12A-12E). Levels of bridge molecules compared to NHDF and ARPE-19 conditioned medium. FIG. 12A shows the MFG-E8 level in hUTC, ARPE-19 and NHDF conditioned medium. Values are mean±SD of duplicate or triplicate samples. FIG. 12B shows the Gas6 level in hUTC, ARPE-19 and NHDF conditioned medium. Values are mean±SD of duplicate samples. FIG. 12C shows TSP-1 level in hUTC, ARPE-19 and NHDF conditioned medium. Values are mean±SD of duplicate samples. FIG. 12D shows the TSP-2 level in hUTC, ARPE-19 and NHDF conditioned medium. Values are mean±SD of duplicate samples. (FIG. 12E) Levels of bridge molecules measured by ELISA. Data shown are the mean±SEM (n=2 for TSP-1 and TSP-2; n=3 for all MFG-E8).

FIGS. 13A-13D demonstrate the effect on phagocytosis with preincubation of dystrophic RPE cells with hUTC conditioned medium (CM). For controls, normal RPE alone or dystrophic RPE alone was preincubated in its regular growth medium MEM20 (MEM+20% FBS). In parallel, dystrophic RPE was also preincubated with CM3. In addition, ROS was tested to be preincubated with hUTC CM3. Values are mean±SD of number of phagocytized ROS in the counted fields per sample. FIG. 13A, n=10-20 per sample; FIG. 13B is raw data. FIG. 13C, n=12 per sample and each sample is in duplicate. (n: number of fields counted); FIG. 13D is raw data.

FIGS. 14A-14K show the effect of bridge molecules and RTK ligands on rod outer segment (ROS) phagocytosis by RCS RPE cells. ROS was preincubated with control medium (DMEM+10% FBS) or hUTC conditioned media. In parallel, ROS was preincubated in control medium with various concentrations of human recombinant MFG-E8 (FIG. 14A), Gas6 (FIG. 14B), TSP-1 (FIG. 14C), or TSP-2 (FIG. 14D). For controls, normal RPE alone or dystrophic RPE alone was cultured for phagocytosis assay. (N: normal RPE; N+ROS: normal RPE cells fed with untreated ROS during phagocytosis assay; D: dystrophic RPE cells; D+ROS: dystrophic RPE cells fed with untreated ROS during phagocytosis assay; Con M: control medium; D+ROS+Con: dystrophic RPE cells fed with control medium pre-incubated ROS; CM4: the 4th batch of hUTC conditioned medium). D+ROS+CM4: dystrophic RPE cells fed with CM4 pre-incubated ROS; D+ROS+MFG-E8: dystrophic RPE cells fed with MFG-E8 pre-incubated ROS. Values are mean±SD of number of phagocytized ROS in the counted fields per sample (n=10 per sample; n: number of fields counted). *P<0.001, D+ROS pretreated with MFG-E8, Gas6, TSP-1 or TSP-2 vs. D+ROS+ConM, and D+ROS; **P<0.0001, D+ROS+CM4 vs. D+ROS, and D+ROS+ConM. (FIGS. 14E-14H) Photoreceptor OS were incubated with recombinant human MFG-E8 (FIG. 14E), Gas6 (FIG. 14F), TSP-1 (FIG. 14G), or TSP-2 (FIG. 14H) for 24 hours and then fed to RCS RPE cells for phagocytosis assay in the absence of hUTC CM. OS pre-incubated with hUTC CM was used as a positive control for the assay. Phagocytosis of OS by RCS RPE cells was rescued in a dose-dependent manner by MFG-E8, Gas6, TSP-1 or TSP-2. (FIGS. 14I-14K) RCS RPE cells were incubated with recombinant human BDNF (FIG. 14I), HGF (FIG. 14J) or GDNF (FIG. 14K) for 24 hours, and then subject to phagocytosis assay. RCS RPE incubated with hUTC CM was used as a positive control for the assay. BDNF, HGF or GDNF dose-dependently increased the phagocytosis in RCS RPE cells. Data represent the mean±SEM (n=3). ****p<0.0001, ***p<0.001, **p<0.01, *p<0.05, n.s. not significant.

FIGS. 15A-15C. RTK ligands and bridge molecules for hUTC-induced phagocytosis rescue in RCS RPE. (FIG. 15A) ELISA of cell culture supernatants collected from untransfected hUTC and hUTC transfected with siRNA. (FIG. 15B) Expression of RTK ligands BDNF, HGF and GDNF were silenced by siRNA transfection of hUTC. Knockdown (KD) hUTC CM were harvested. RCS RPE were incubated with KD hUTC CM for 24 hours and then subject to phagocytosis assay. The effect of hUTC on RCS RPE phagocytosis was abolished when BDNF, HGF or GDNF was knocked down. (FIG. 15C) Expression of bridge molecules MFG-E8, TSP-1 and TSP-2 were silenced in hUTC by siRNA transfection. KD hUTC CM were harvested. RCS RPE were fed with OS pre-incubated with KD hUTC CM for 24 hours and subject to phagocytosis assay. Knocking-down of MFG-E8, TSP-1 or TSP-2 reduced the hUTC-mediated OS phagocytosis rescue in RCS RPE. CM prepared from untransfected and scrambled siRNA transfected hUTC were used as controls. Data represent the mean±SEM, n=3 for (B) and (C), n=6 for untransfected, mock and scrambled siRNA transfected hUTC CM ELISA (A). ****p<0.0001, ***p<0.001, **p<0.01, *p<0.05, n. s. not significant.

FIGS. 16A-16D. hUTC-secreted bridge molecules bind to photoreceptor OS. Immunofluorescence (IF) staining of OS incubated with individual recombinant human MFG-E8 (124 ng/mL), Gas6 (8.75 ng/mL), TSP-1 (1.2 [tg/mL) or TSP-2 (238 ng/mL) (FIG. 16A), or hUTC CM (FIG. 16B), or control medium (FIG. 16C) for 24 hours with subsequent double IF staining of rhodopsin with Alexa Fluor 568 conjugated anti-rhodopsin antibody and of bridge molecules with Alexa Fluor 488 conjugated anti-MFG-E8, anti-Gas6, anti-TSP-1, anti-TSP-2, mouse IgG2A or mouse IgG2B isotype control antibody. The rhodopsin-stained OS particles also stained positively with each of the recombinant bridge molecule proteins or the secreted bridge molecules present in hUTC CM. (FIG. 16D) The specificity of anti-rhodopsin antibody was confirmed by double IF staining of OS with Alexa Fluor 568 conjugated anti-rhodopsin antibody and Alexa Fluor 488 conjugated mouse IgG2b, x isotype control antibody. Upper panel (FIGS. 16A-16D), bridge molecule and isotype antibody staining; lower panel (FIGS. 16A-16D), rhodopsin antibody staining.

FIGS. 17A-17F show hUTC and hUTC conditioned media protection from oxidative stress or damage. FIGS. 17A-17B illustrate hUTC conditioned media protected A2E-containing RPE cells from non-viability after 430 nm irradiation. (FIG. 17A). Cell death was assayed using a two-color fluorescence assay. hUTC conditioned media and unconditioned control media (250 μL/well) were incubated with A2E-laden ARPE-19 cells for 7 days. The percent of nonviable cells was determined by a two-color fluorescence assay; 5 replicates. FIG. 17B shows pooled data from the 5% and 10% FBS treatments. Values are mean+/−SEM. p<0.05; one-way ANOVA and Newman Keuls multiple comparison test. FIGS. 17C-17D illustrate hUTC conditioned media protected ARPE-19 cells against A2E photooxidation-associated reduced cell viability. (FIG. 17C). Cell viability was assayed by MTT. hUTC conditioned media and unconditioned control media (250 μL/well) were incubated with A2E-laden ARPE-19 cells (7 days, 37° C., 5% CO2, 5% FBS). Bar height is indicative of MTT absorbance and reflects cell viability. FIG. 17D shows pooled data from the 5% and 10% FBS treatments. Values are mean+/−SEM; 4 replicates/2 experiments. * p>0.05; ** p<0.05; one-way ANOVA and Newman Keuls multiple comparison test. FIGS. 17E-17F illustrate hUTC conditioned media protected ARPE-19 cells against acute H₂O₂-associated reduced cell viability. Cell viability was assayed by MTT (FIG. 17E) and crystal violet (FIG. 17F). The y axis represents the corrected OD reading at 550 nm. Data is presented as the mean±standard deviation. p<0.05 by two-way ANOVA.

Other features and advantages of the invention will be apparent from the detailed description and examples that follow.

### DETAILED DESCRIPTION

Various patents and other publications are referred to throughout the specification. Each of these publications is incorporated by reference herein, in its entirety. In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments are defined by the appended claims.

### DEFINITIONS

Various terms used throughout the specification and claims are defined as set forth below and are intended to clarify the invention.

Stem cells are undifferentiated cells defined by the ability of a single cell both to self-renew, and to differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation, and to contribute substantially to most, if not all, tissues following injection into blastocysts.

Stem cells are classified according to their developmental potential as: (1) totipotent; (2) pluripotent; (3) multipotent; (4) oligopotent; and (5) unipotent. Totipotent cells are able to give rise to all embryonic and extraembryonic cell types. Pluripotent cells are able to give rise to all embryonic cell types. Multipotent cells include those able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood). Cells that are oligopotent can give rise to a more restricted subset of cell lineages than multipotent stem cells; and cells that are unipotent are able to give rise to a single cell lineage (e.g., spermatogenic stem cells).

Stem cells are also categorized on the basis of the source from which they may be obtained. An adult stem cell is generally a multipotent undifferentiated cell found in tissue comprising multiple differentiated cell types. The adult stem cell can renew itself. Under normal circumstances, it can also differentiate to yield the specialized cell types of the tissue from which it originated, and possibly other tissue types. Induced pluripotent stem cells (iPS cells) are adult cells that are converted into pluripotent stem cells. (Takahashi et al., Cell, 2006; 126(4):663-676; Takahashi et al., Cell, 2007; 131:1-12). An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the placenta and the umbilical cord. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta).

Embryonic tissue is typically defined as tissue originating from the embryo (which in humans refers to the period from fertilization to about six weeks of development). Fetal tissue refers to tissue originating from the fetus, which in humans refers to the period from about six weeks of development to parturition. Extraembryonic tissue is tissue associated with, but not originating from, the embryo or fetus. Extraembryonic tissues include extraembryonic membranes (chorion, amnion, yolk sac and allantois), umbilical cord and placenta (which itself forms from the chorion and the maternal decidua basalis).

Differentiation is the process by which an unspecialized (“uncommitted”) or less specialized cell acquires the features of a specialized cell, such as a nerve cell or a muscle cell, for example. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e. which cells it came from and what cells it can give rise to. The lineage of a cell places the cell within a hereditary scheme of development and differentiation.

In a broad sense, a progenitor cell is a cell that has the capacity to create progeny that are more differentiated than itself, and yet retains the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

As used herein, the phrase “differentiates into an ocular lineage or phenotype” refers to a cell that becomes partially or fully committed to a specific ocular phenotype, including without limitation, retinal and corneal stem cells, pigment epithelial cells of the retina and iris, photoreceptors, retinal ganglia and other optic neural lineages (e.g., retinal glia, microglia, astrocytes, Mueller cells), cells forming the crystalline lens, and epithelial cells of the sclera, cornea, limbus and conjunctiva. The phrase “differentiates into a neural lineage or phenotype” refers to a cell that becomes partially or fully committed to a specific neural phenotype of the CNS or PNS, i.e., a neuron or a glial cell, the latter category including without limitation astrocytes, oligodendrocytes, Schwann cells and microglia.

The cells exemplified herein and preferred for use in the present invention are generally referred to as postpartum-derived cells (or PPDCs). They also may sometimes be referred to more specifically as umbilicus-derived cells or placenta-derived cells (UDCs or PDCs). In addition, the cells may be described as being stem or progenitor cells, the latter term being used in the broad sense. The term derived is used to indicate that the cells have been obtained from their biological source and grown or otherwise manipulated in vitro (e.g., cultured in a Growth Medium to expand the population and/or to produce a cell line). The in vitro manipulations of umbilical stem cells and placental stem cells and the unique features of the umbilicus-derived cells and placental-derived cells of the present invention are described in detail below. Cells isolated from postpartum placenta and umbilicus by other means is also considered suitable for use in the present invention. These other cells are referred to herein as postpartum cells (rather than postpartum-derived cells).

Various terms are used to describe cells in culture. Cell culture refers generally to cells taken from a living organism and grown under controlled conditions (“in culture” or “cultured”). A primary cell culture is a culture of cells, tissues, or organs taken directly from an organism(s) before the first subculture. Cells are expanded in culture when they are placed in a Growth Medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, growth conditions, and time between passaging.

The term Growth Medium generally refers to a medium sufficient for the culturing of PPDCs. In particular, one presently preferred medium for the culturing of the cells of the invention comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics ((preferably 50-100 Units/milliliter penicillin, 50-100 microgram/milliliter streptomycin, and 0-0.25 microgram/milliliter amphotericin B; Invitrogen, Carlsbad, Calif.)), and 0.001% (v/v) 2-mercaptoethanol (Sigma, St. Louis Mo.). As used in the Examples below, Growth Medium refers to DMEM-low glucose with 15% fetal bovine serum and antibiotics/antimycotics (when penicillin/streptomycin are included, it is preferably at 50 Um′ and 50 microgram/ml respectively; when penicillin/streptomycin/amphotericin are used, it is preferably at 100 U/ml, 100 microgram/ml and 0.25 microgram/ml, respectively). In some cases different growth media are used, or different supplementations are provided, and these are normally indicated in the text as supplementations to Growth Medium.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

Generally, a trophic factor is defined as a substance that promotes survival, growth, differentiation, proliferation and/or maturation of a cell, or stimulates increased activity of a cell. The interaction between cells via trophic factors may occur between cells of different types. Cell interaction by way of trophic factors is found in essentially all cell types, and is a particularly significant means of communication among neural cell types. Trophic factors also can function in an autocrine fashion, i.e., a cell may produce trophic factors that affect its own survival, growth, differentiation, proliferation and/or maturation.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone.

The terms ocular, ophthalmic and optic are used interchangeably herein to define “of, or about, or related to the eye.” The term ocular degenerative condition (or disorder) is an inclusive term encompassing acute and chronic conditions, disorders or diseases of the eye, inclusive of the neural connection between the eye and the brain, involving cell damage, degeneration or loss. An ocular degenerative condition may be age-related, or it may result from injury or trauma, or it may be related to a specific disease or disorder. Acute ocular degenerative conditions include, but are not limited to, conditions associated with cell death or compromise affecting the eye including conditions arising from cerebrovascular insufficiency, focal or diffuse brain trauma, diffuse brain damage, infection or inflammatory conditions of the eye, retinal tearing or detachment, intra-ocular lesions (contusion penetration, compression, laceration) or other physical injury (e.g., physical or chemical burns). Chronic ocular degenerative conditions (including progressive conditions) include, but are not limited to, retinopathies and other retinal/macular disorders such as retinitis pigmentosa (RP), age-related macular degeneration (AMD), choroidal neovascular membrane (CNVM); retinopathies such as diabetic retinopathy, occlusive retinopathy, sickle cell retinopathy and hypertensive retinopathy, central retinal vein occlusion, stenosis of the carotid artery, optic neuropathies such as glaucoma and related syndromes; disorders of the lens and outer eye, e.g., limbal stem cell deficiency (LSCD), also referred to as limbal epithelial cell deficiency (LECD), such as occurs in chemical or thermal injury, Steven-Johnson syndrome, contact lens-induced keratopathy, ocular cicatricial pemphigoid, congenital diseases of aniridia or ectodermal dysplasia, and multiple endocrine deficiency-associated keratitis.

The term treating (or treatment of) an ocular degenerative condition refers to ameliorating the effects of, or delaying, halting or reversing the progress of, or delaying or preventing the onset of, an ocular degenerative condition as defined herein.

The term effective amount refers to a concentration or amount of a reagent or pharmaceutical composition, such as a growth factor, differentiation agent, trophic factor, cell population or other agent, that is effective for producing an intended result, including cell growth and/or differentiation in vitro or in vivo, or treatment of ocular degenerative conditions, as described herein. With respect to growth factors, an effective amount may range from about 1 nanogram/milliliter to about 1 microgram/milliliter. With respect to PPDCs as administered to a patient in vivo, an effective amount may range from as few as several hundred or fewer, to as many as several million or more. In specific embodiments, an effective amount may range from 10³ to 11¹¹, more specifically at least about 10⁴ cells. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or pharmaceutical composition to achieve its intended result.

The term patient or subject refers to animals, including mammals, preferably humans, who are treated with the pharmaceutical compositions or in accordance with the methods described herein.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

Several terms are used herein with respect to cell replacement therapy. The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy. Transplantation as used herein refers to the introduction of autologous, or allogeneic donor cell replacement therapy into a recipient.

As used herein, the term “about” when referring to a measurable value such as an amount, a temporal duration, and the like, is meant to encompass variations of between ±20% and ±0.1%, preferably ±20% or ±10%, more preferably ±5%, even more preferably ±1%, and still more preferably ±0.1% from the specified value, as such variations are appropriate to perform the disclosed methods.

### DESCRIPTION

Ocular degenerative conditions, which encompass acute, chronic and progressive disorders and diseases having divergent causes, have as a common feature the dysfunction or loss of a specific or vulnerable group of ocular cells. This commonality enables development of similar therapeutic approaches for the repair or regeneration of vulnerable, damaged or lost ocular tissue, one of which is cell-based therapy. Development of cell therapy for ocular degenerative conditions has been limited to a comparatively few types of stem or progenitor cells, including ocular-derived stem cells themselves (e.g., retinal and corneal stem cells), embryonic stem cells and a few types of adult stem or progenitor cells (e.g., neural, mucosal epithelial and bone marrow stem cells). Cells isolated from the postpartum umbilical cord and placenta have been identified as a significant new source of progenitor cells for this purpose. (US 2005-0037491 and US 2010-0272803) Moreover, conditioned media generated from cells isolated from the postpartum placenta and umbilical cord tissue provides another new source for treating ocular degenerative conditions. Accordingly, in its various embodiments described herein, the present invention features methods and compositions (including pharmaceutical compositions) for repair and regeneration of ocular tissues, which use conditioned media from progenitor cells and cell populations isolated from postpartum tissues. The invention is applicable to ocular degenerative conditions, but is expected to be particularly suitable for a number of ocular disorders for which treatment or cure has been difficult or unavailable. These include, without limitation, age-related macular degeneration, retinitis pigmentosa, diabetic and other retinopathies.

Conditioned media derived from progenitor cells, such as cells isolated from postpartum umbilical cord or placenta in accordance with any method known in the art is expected to be suitable for use in the present invention. In one embodiment, however, the invention uses conditioned media derived from umbilical cord tissue-derived cells (hUTCs) or placental-tissue derived cells (PDCs) as defined above, which are derived from umbilical cord tissue or placenta that has been rendered substantially free of blood, preferably in accordance with the method set forth below. The hUTCs or PDCs are capable of expansion in culture and have the potential to differentiate into cells of other phenotypes. Certain embodiments feature conditioned media prepared from such progenitor cells, compositions comprising the conditioned media, and methods of using compositions such as pharmaceutical compositions for treatment of patients with acute or chronic ocular degenerative conditions. The postpartum-derived cells of the present invention have been characterized by their growth properties in culture, by their cell surface markers, by their gene expression, by their ability to produce certain biochemical trophic factors, and by their immunological properties. The conditioned media derived from the postpartum-derived cells have been characterized by the trophic factors and bridge molecules secreted by the cells.

### Preparation of Progenitor Cells

The cells, cell populations and preparations comprising cell lysates, conditioned media and the like, used in the compositions and methods of the present invention are described herein, and in detail in U.S. Pat. Nos. 7,524,489, and 7,510,873, and U.S. Pub. App. No. 2005/0058634, both incorporated by reference herein. According to the methods, a mammalian umbilical cord and placenta are recovered upon or shortly after termination of either a full-term or pre-term pregnancy, for example, after expulsion of after-birth. The postpartum tissue may be transported from the birth site to a laboratory in a sterile container such as a flask, beaker, culture dish, or bag. The container may have a solution or medium, including but not limited to a salt solution, such as, for example, Dulbecco's Modified Eagle's Medium (DMEM) or phosphate buffered saline (PBS), or any solution used for transportation of organs used for transplantation, such as University of Wisconsin solution or perfluorochemical solution. One or more antibiotic and/or antimycotic agents, such as but not limited to penicillin, streptomycin, amphotericin B, gentamicin, and nystatin, may be added to the medium or buffer. The postpartum tissue may be rinsed with an anticoagulant solution such as heparin-containing solution. It is preferable to keep the tissue at about 4-10° C. prior to extraction of PPDCs. It is even more preferable that the tissue not be frozen prior to extraction of PPDCs.

Isolation of PPDCs preferably occurs in an aseptic environment. The umbilical cord may be separated from the placenta by means known in the art. Alternatively, the umbilical cord and placenta are used without separation. Blood and debris are preferably removed from the postpartum tissue prior to isolation of PPDCs. For example, the postpartum tissue may be washed with buffer solution, such as but not limited to phosphate buffered saline. The wash buffer also may comprise one or more antimycotic and/or antibiotic agents, such as but not limited to penicillin, streptomycin, amphotericin B, gentamicin, and nystatin.

Postpartum tissue comprising a whole placenta or umbilical cord, or a fragment or section thereof is disaggregated by mechanical force (mincing or shear forces). In a presently preferred embodiment, the isolation procedure also utilizes an enzymatic digestion process. Many enzymes are known in the art to be useful for the isolation of individual cells from complex tissue matrices to facilitate growth in culture. Ranging from weakly digestive (e.g. deoxyribonucleases and the neutral protease, dispase) to strongly digestive (e.g. papain and trypsin), such enzymes are available commercially. A nonexhaustive list of enzymes compatible herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest singlestranded DNA and can minimize cell clumping during isolation. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods that employ digestion in the presence of at least one collagenase from Clostridium histolyticum, and either of the protease activities, dispase and thermo lysin. Still more preferred are methods employing digestion with both collagenase and dispase enzyme activities. Also preferred are methods that include digestion with a hyaluronidase activity in addition to collagenase and dispase activities. The skilled artisan will appreciate that many such enzyme treatments are known in the art for isolating cells from various tissue sources. For example, the LIBERASE™ Blendzyme 3 (Roche) series of enzyme combinations are suitable for use in the instant methods. Other sources of enzymes are known, and the skilled artisan may also obtain such enzymes directly from their natural sources. The skilled artisan is also well equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37° C. during the enzyme treatment of the dissociation step.

In some embodiments of the invention, postpartum tissue is separated into sections comprising various aspects of the tissue, such as neonatal, neonatal/maternal, and maternal aspects of the placenta, for instance. The separated sections then are dissociated by mechanical and/or enzymatic dissociation according to the methods described herein. Cells of neonatal or maternal lineage may be identified by any means known in the art, for example, by karyotype analysis or in situ hybridization for a Y chromosome.

Isolated cells or postpartum tissue from which PPDCs grow out may be used to initiate, or seed, cell cultures. Isolated cells are transferred to sterile tissue culture vessels either uncoated or coated with extracellular matrix or ligands such as laminin, collagen (native, denatured or crosslinked), gelatin, fibronectin, and other extracellular matrix proteins. PPDCs are cultured in any culture medium capable of sustaining growth of the cells such as, but not limited to, DMEM (high or low glucose), advanced DMEM, DMEM/MCDB 201, Eagle's basal medium, Ham's F10 medium (F10), Ham's F-12 medium (F12), Iscove's modified Dulbecco's medium, Mesenchymal Stem Cell Growth Medium (MSCGM), DMEM/F12, RPMI 1640, and cellgro FREE™. The culture medium may be supplemented with one or more components including, for example, fetal bovine serum (FBS), preferably about 2-15% (v/v); equine serum (ES); human serum (HS); beta-mercaptoethanol (BME or 2-ME), preferably about 0.001% (v/v); one or more growth factors, for example, platelet-derived growth factor (PDGF), epidermal growth factor (EGF), fibroblast growth factor (FGF), vascular endothelial growth factor (VEGF), insulin-like growth factor-1 (IGF-1), leukocyte inhibitory factor (LIF) and erythropoietin; amino acids, including L-valine; and one or more antibiotic and/or antimycotic agents to control microbial contamination, such as, for example, penicillin G, streptomycin sulfate, amphotericin B, gentamicin, and nystatin, either alone or in combination. The culture medium preferably comprises Growth Medium (DMEM-low glucose, serum, BME, and an antibiotic agent).

The cells are seeded in culture vessels at a density to allow cell growth. In a preferred embodiment, the cells are cultured at about 0 to about 5 percent by volume CO₂ in air. In some preferred embodiments, the cells are cultured at about 2 to about 25 percent O₂ in air, preferably about 5 to about 20 percent ₂ in air. The cells preferably are cultured at about 25 to about 40° C. and more preferably are cultured at 37° C. The cells are preferably cultured in an incubator. The medium in the culture vessel can be static or agitated, for example, using a bioreactor. PPDCs preferably are grown under low oxidative stress (e.g., with addition of glutathione, Vitamin C, Catalase, Vitamin E, N-Acetylcysteine). “Low oxidative stress”, as used herein, refers to conditions of no or minimal free radical damage to the cultured cells.

Methods for the selection of the most appropriate culture medium, medium preparation, and cell culture techniques are well known in the art and are described in a variety of sources, including Doyle et al., (eds.), 1995, CELL & TISSUE CULTURE: LABORATORY PROCEDURES, John Wiley & Sons, Chichester; and Ho and Wang (eds.), 1991, ANIMAL CELL BIOREACTORS, Butterworth-Heinemann, Boston, which are incorporated herein by reference.

After culturing the isolated cells or tissue fragments for a sufficient period of time, PPDCs will have grown out, either as a result of migration from the postpartum tissue or cell division, or both. In some embodiments of the invention, PPDCs are passaged, or removed to a separate culture vessel containing fresh medium of the same or a different type as that used initially, where the population of cells can be mitotically expanded. The cells of the invention may be used at any point between passage 0 and senescence. The cells preferably are passaged between about 3 and about 25 times, more preferably are passaged about 4 to about 12 times, and preferably are passaged 10 or 11 times. Cloning and/or subcloning may be performed to confirm that a clonal population of cells has been isolated.

In some aspects of the invention, the different cell types present in postpartum tissue are fractionated into subpopulations from which the PPDCs can be isolated. This may be accomplished using standard techniques for cell separation including, but not limited to, enzymatic treatment to dissociate postpartum tissue into its component cells, followed by cloning and selection of specific cell types, for example but not limited to selection based on morphological and/or biochemical markers; selective growth of desired cells (positive selection), selective destruction of unwanted cells (negative selection); separation based upon differential cell agglutinability in the mixed population as, for example, with soybean agglutinin; freeze-thaw procedures; differential adherence properties of the cells in the mixed population; filtration; conventional and zonal centrifugation; centrifugal elutriation (counter-streaming centrifugation); unit gravity separation; countercurrent distribution; electrophoresis; and fluorescence activated cell sorting (FACS). For a review of clonal selection and cell separation techniques, see Freshney, 1994, CULTURE OF ANIMAL CELLS: A MANUAL OF BASIC TECHNIQUES, 3rd Ed., Wiley-Liss, Inc., New York, which is incorporated herein by reference.

The culture medium is changed as necessary, for example, by carefully aspirating the medium from the dish, for example, with a pipette, and replenishing with fresh medium. Incubation is continued until a sufficient number or density of cells accumulates in the dish. The original explanted tissue sections may be removed and the remaining cells trypsinized using standard techniques or using a cell scraper. After trypsinization, the cells are collected, removed to fresh medium and incubated as above. In some embodiments, the medium is changed at least once at approximately 24 hours post-trypsinization to remove any floating cells. The cells remaining in culture are considered to be PPDCs.

PPDCs may be cryopreserved. Accordingly, in a preferred embodiment described in greater detail below, PPDCs for autologous transfer (for either the mother or child) may be derived from appropriate postpartum tissues following the birth of a child, then cryopreserved so as to be available in the event they are later needed for transplantation.

### Characteristics of Progenitor Cells

The progenitor cells of the invention, such as PPDCs, may be characterized, for example, by growth characteristics (e.g., population doubling capability, doubling time, passages to senescence), karyotype analysis (e.g., normal karyotype; maternal or neonatal lineage), flow cytometry (e.g., FACS analysis), immunohistochemistry and/or immunocytochemistry (e.g., for detection of epitopes), gene expression profiling (e.g., gene chip arrays; polymerase chain reaction (for example, reverse transcriptase PCR, real time PCR, and conventional PCR), protein arrays, protein secretion (e.g., by plasma clotting assay or analysis of PDC-conditioned medium, for example, by Enzyme Linked ImmunoSorbent Assay (ELISA)), mixed lymphocyte reaction (e.g., as measure of stimulation of PBMCs), and/or other methods known in the art.

Examples of PPDCs derived from umbilicus tissue were deposited with the American Type Culture Collection on (ATCC, 10801 University Boulevard, Manassas, Va., 20110) Jun. 10, 2004, and assigned ATCC Accession Numbers as follows: (1) strain designation UMB 022803 (P7) was assigned Accession No. PTA-6067; and (2) strain designation UMB 022803 (P17) was assigned Accession No. PTA-6068. Examples of PPDCs derived from placental tissue were deposited with the American Type Culture Collection (ATCC, Manassas, Va.) and assigned ATCC Accession Numbers as follows: (1) strain designation PLA 071003 (P8) was deposited Jun. 15, 2004 and assigned Accession No. PTA-6074; (2) strain designation PLA 071003 (P11) was deposited Jun. 15, 2004 and assigned Accession No. PTA-6075; and (3) strain designation PLA 071003 (P16) was deposited Jun. 16, 2004 and assigned Accession No. PTA-6079.

In various embodiments, the PPDCs possess one or more of the following growth features: (1) they require L-valine for growth in culture; (2) they are capable of growth in atmospheres containing oxygen from about 5% to at least about 20%; (3) they have the potential for at least about 40 doublings in culture before reaching senescence; and (4) they attach and expand on a coated or uncoated tissue culture vessel, wherein the coated tissue culture vessel comprises a coating of gelatin, laminin, collagen, polyomithine, vitronectin or fibronectin.

In certain embodiments the PPDCs possess a normal karyotype, which is maintained as the cells are passaged. Karyotyping is particularly useful for identifying and distinguishing neonatal from maternal cells derived from placenta. Methods for karyotyping are available and known to those of skill in the art.

In other embodiments, the PPDCs may be characterized by production of certain proteins, including: (1) production of at least one of vimentin and alpha-smooth muscle actin; and (2) production of at least one of CD10, CD13, CD44, CD73, CD90, PDGFr-alpha, PD-L2 and HLA-A,B,C cell surface markers, as detected by flow cytometry. In other embodiments, the PPDCs may be characterized by lack of production of at least one of CD31, CD34, CD45, CD80, CD86, CD117, CD141, CD178, B7-H2, HLA-G, and HLA-DR,DP,DQ cell surface markers, as detected by flow cytometry. Particularly preferred are cells that produce vimentin and alpha-smooth muscle actin.

In other embodiments, the PPDCs may be characterized by gene expression, which relative to a human cell that is a fibroblast, a mesenchymal stem cell, or an iliac crest bone marrow cell, is increased for a gene encoding at least one of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; tumor necrosis factor, alpha-induced protein 3; C-type lectin superfamily member 2; Wilms tumor 1; aldehyde dehydrogenase 1 family member A2; renin; oxidized low density lipoprotein receptor 1; Homo sapiens clone IMAGE:4179671; protein kinase C zeta; hypothetical protein DKFZp564F013; downregulated in ovarian cancer 1; and Homo sapiens gene from clone DKFZp547k1113. In an embodiment, the PPDCs derived from umbilical cord tissue may be characterized by gene expression, which relative to a human cell that is a fibroblast, a mesenchymal stem cell, or an iliac crest bone marrow cell, is increased for a gene encoding at least one of interleukin 8; reticulon 1; or chemokine (C-X-C motif) ligand 3. In another embodiment, the PPDCs derived from placental tissue may be characterized by gene expression, which relative to a human cell that is a fibroblast, a mesenchymal stem cell, or an iliac crest bone marrow cell, is increased for a gene encoding at least one of renin or oxidized low density lipoprotein receptor 1.

In yet other embodiments, the PPDCs may be characterized by gene expression, which relative to a human cell that is a fibroblast, a mesenchymal stem cell, or an iliac crest bone marrow cell, is reduced for a gene encoding at least one of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeo box 2 (growth arrest-specific homeo box); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FU12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAAI034 protein; vesicle-associated membrane protein 5 (myobrevin); EGF-containing fibulin-like extracellular matrix protein 1; early growth response 3; distal-less homeo box 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle).

In other embodiments, the PPDCs may be characterized by secretion of bridge molecules selected from MFG-E8, Gas6, TSP-1 and TSP-2. Further, the PPDCs derived from umbilical cord tissue may be characterized by secretion of at least one of MCP-1, IL-6, IL-8, GCP-2, HGF, KGF, FGF, HB-EGF, BDNF, TPO, MIP1b, 1309, RANTES, MDC, and TIMP1. In some embodiments, the PPDCs derived from umbilical cord tissue may be characterized by lack of secretion of at least one of TGF-beta2, ANG2, PDGFbb, MIP1a and VEGF, as detected by ELISA. In alternative embodiments, PPDCs derived from placenta tissue may be characteristized by secretion of at least one of MCP-1, IL-6, IL-8, GCP-2, HGF, KGF, HB-EGF, BDNF, TPO, MIP1a, RANTES, and TIMP1, and lack of secretion of at least one of TGF-beta2, MIP1b, ANG2, PDGFbb, FGF, and VEGF, as detected by ELISA. In further embodiments, the PPDCs lack expression of hTERT or telomerase.

In preferred embodiments, the cell comprises two or more of the above-listed growth, protein/surface marker production, gene expression or substance-secretion characteristics. More preferred are those cells comprising, three, four, or five or more of the characteristics. Still more preferred are PPDCs comprising six, seven, or eight or more of the characteristics. Still more preferred presently are those cells comprising all of above characteristics.

In particularly preferred embodiments, the cells isolated from human umbilical cord tissue substantially free of blood, which are capable of expansion in culture, lack the production of CD117 or CD45, and do not express hTERT or telomerase. In one embodiment, the cells lack production of CD117 and CD45 and, optionally, also do not express hTERT and telomerase. In another embodiment, the cells do not express hTERT and telomerase. In yet another embodiment, the cells are isolated from human umbilical cord tissue substantially free of blood, are capable of expansion in culture, lack the production of CD117 or CD45, and do not express hTERT or telomerase, and have one or more of the following characteristics: express CD10, CD13, CD44, CD73, and CD90; do not express CD31 or CD34; express, relative to a human fibroblast, mesenchymal stem cell, or iliac crest bone marrow cell, increased levels of interleukin 8 or reticulon 1; and have the potential to differentiate.

Among cells that are presently preferred for use with the invention in several of its aspects are postpartum cells having the characteristics described above and more particularly those wherein the cells have normal karyotypes and maintain normal karyotypes with passaging, and further wherein the cells express each of the markers CD10, CD13, CD44, CD73, CD90, PDGFr-alpha, and HLA-A,B,C, wherein the cells produce the immunologically-detectable proteins which correspond to the listed markers. Still more preferred are those cells which in addition to the foregoing do not produce proteins corresponding to any of the markers CD31, CD34, CD45, CD117, CD141, or HLA-DR,DP,DQ, as detected by flow cytometry. In further preferred embodiments, the cells lack expression of hTERT or telomerase.

Certain cells having the potential to differentiate along lines leading to various phenotypes are unstable and thus can spontaneously differentiate. Presently preferred for use with the invention are cells that do not spontaneously differentiate, for example along neural lines. Preferred cells, when grown in Growth Medium, are substantially stable with respect to the cell markers produced on their surface, and with respect to the expression pattern of various genes, for example as determined using an Affymetrix GENECHIP. The cells remain substantially constant, for example in their surface marker characteristics over passaging, through multiple population doublings.

However, one feature of PPDCs is that they may be deliberately induced to differentiate into various lineage phenotypes by subjecting them to differentiation-inducing cell culture conditions. Of use in treatment of certain ocular degenerative conditions, the PPDCs may be induced to differentiate into neural phenotypes using one or more methods known in the art. For instance, as exemplified herein, PPDCs may be plated on flasks coated with laminin in Neurobasal-A medium (Invitrogen, Carlsbad, Calif.) containing B27 (B27 supplement, Invitrogen), L-glutamine and Penicillin/Streptomycin, the combination of which is referred to herein as Neural Progenitor Expansion (NPE) medium. NPE media may be further supplemented with bFGF and/or EGF. Alternatively, PPDCs may be induced to differentiate in vitro by: (1) co-culturing the PPDCs with neural progenitor cells; or (2) growing the PPDCs in neural progenitor cell-conditioned medium.

Differentiation of the PPDCs into neural phenotypes may be demonstrated by a bipolar cell morphology with extended processes. The induced cell populations may stain positive for the presence of nestin. Differentiated PPDCs may be assessed by detection of nest in, TuJ1 (BIII tubulin), GFAP, tyrosine hydroxylase, GABA, 04 and/or MBP. In some embodiments, PPDCs have exhibited the ability to form three-dimensional bodies characteristic of neuronal stem cell formation of neurospheres.

### Cell Populations

Another aspect of the invention features populations of progenitor cells, such as postpartum-derived cells. The postpartum-derived cells may be isolated from placental or umbilical tissue. In a preferred embodiment, the cell populations comprise the PPDCs described above, and these cell populations are described in the section below.

In some embodiments, the cell population is heterogeneous. A heterogeneous cell population of the invention may comprise at least about 5%, 10%, 20%, 30%, 40%, 50%, 60%, 70%, 80%, 90%, or 95% of the cell. The heterogeneous cell populations of the invention may further comprise the progenitor cells (postpartum-derived cells), or other progenitor cells, such as epithelial or neural progenitor cells, or it may further comprise fully differentiated cells.

In some embodiments, the population is substantially homogeneous, i.e., comprises substantially only PPDCs (preferably at least about 96%, 97%, 98%, 99% or more of the cells). In some embodiments, the cell population is homogeneous. In embodiments, the homogeneous cell population of the invention may comprise umbilicus- or placenta-derived cells. Homogeneous populations of umbilicus-derived cells are preferably free of cells of maternal lineage. Homogeneous populations of placenta-derived cells may be of neonatal or maternal lineage. Homogeneity of a cell population may be achieved by any method known in the art, for example, by cell sorting (e.g., flow cytometry) or by clonal expansion in accordance with known methods. Thus, preferred homogeneous PPDC populations may comprise a clonal cell line of postpartum-derived cells. Such populations are particularly useful when a cell clone with highly desirable functionality has been isolated.

Also provided herein are populations of cells incubated in the presence of one or more factors, or under conditions, that stimulate stem cell differentiation along a desired pathway (e.g., neural, epithelial). Such factors are known in the art and the skilled artisan will appreciate that determination of suitable conditions for differentiation can be accomplished with routine experimentation. Optimization of such conditions can be accomplished by statistical experimental design and analysis, for example response surface methodology allows simultaneous optimization of multiple variables, for example in a biological culture. Presently preferred factors include, but are not limited to factors, such as growth or trophic factors, demethylating agents, co-culture with neural or epithelial lineage cells or culture in neural or epithelial lineage cell-conditioned medium, as well other conditions known in the art to stimulate stem cell differentiation along these pathways (for factors useful in neural differentiation, see, e.g., Lang, K. J. D. et al., 2004, J. Neurosci. Res. 76: 184-192; Johe, K. K. et al., 1996, Genes Devel. 10: 3129-3140; Gottleib, D., 2002, Ann. Rev. Neurosci. 25: 381-407).

### Conditioned Medium

In one aspect, the invention provides conditioned medium from cultured progenitor cells, such as postpartum-derived cells, or other progenitor cells, for use in vitro and in vivo as described below. Use of such conditioned medium allows the beneficial trophic factors secreted by the cells to be used allogeneically in a patient without introducing intact cells that could trigger rejection, or other adverse immunological responses. Conditioned medium is prepared by culturing cells (such as a population of cells) in a culture medium, then removing the cells from the medium. In certain embodiments, the postpartum cells are UTCs or PDCs, more preferably hUTCs.

Conditioned medium prepared from populations of cells as described above may be used as is, further concentrated, by for example, ultrafiltration or lyophilization, or even dried, partially purified, combined with pharmaceutically-acceptable carriers or diluents as are known in the art, or combined with other compounds such as biologicals, for example pharmaceutically useful protein compositions. Conditioned medium may be used in vitro or in vivo, alone or for example, with autologous or syngeneic live cells. The conditioned medium, if introduced in vivo, may be introduced locally at a site of treatment, or remotely to provide, for example needed cellular growth or trophic factors to a patient.

Previously, it has been demonstrated that human umbilical cord tissue-derived cells improved visual function and ameliorated retinal degeneration (See US 2010/0272803. It also has been demonstrated that postpartum-derived cells can be used to promote photoreceptor rescue and thus preserve photoreceptors in the RCS model. (See US 2010/0272803). Injection of hUTC subretinally into RCS rat eye improved visual acuity and ameliorated retinal degeneration.

As provided herein, various preparations of hUTC conditioned medium were prepared and evaluated for phagocytosis rescue activities. Seeding density and culture conditions were found to affect activity level for conditioned media. For hUTC in serum (CM1), hUTCs were seeded at 5,000 viable cells/cm² in T75 cell culture flask in hUTC growth medium (DMEM low glucose+15% FBS+4 mM L-glutamine), and cultured for 24 hours. Medium was replaced with 21 mL of DMEM/F12 complete medium (DMEM:F12 medium+10% FBS+Pen (50 U/ml)/Strep (50 μg/ml)), cells were cultured for another 54 hours, and the culture supernatant was collected and frozen at −70° C. (cryopreserved).

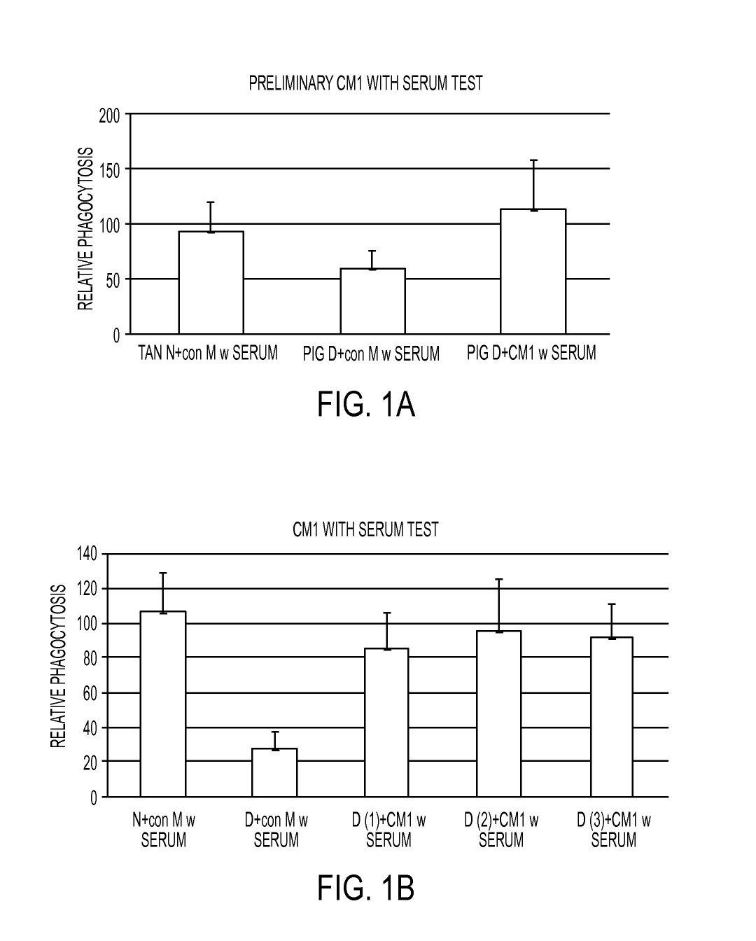

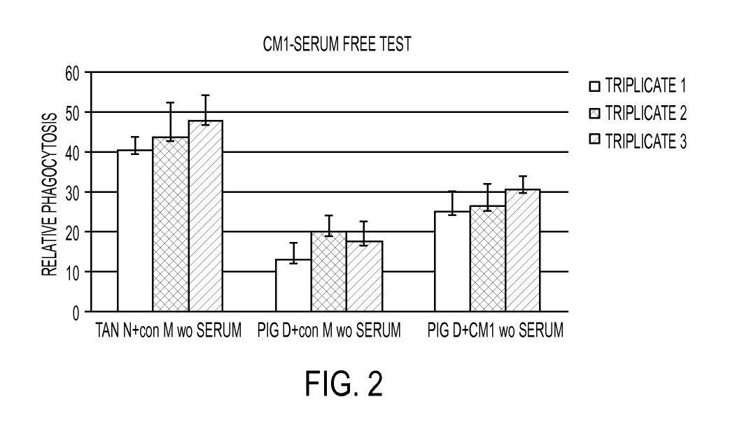

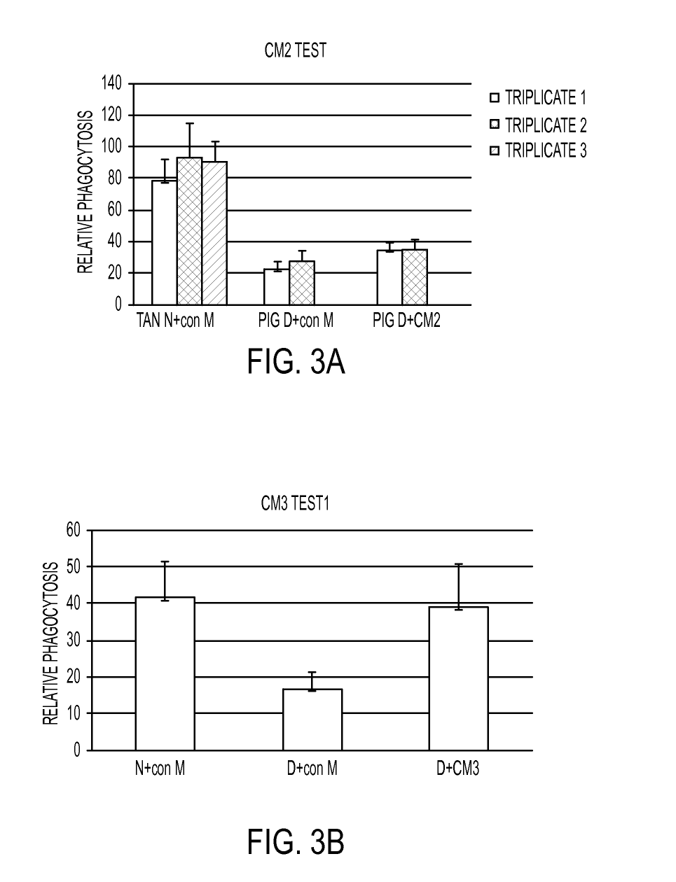

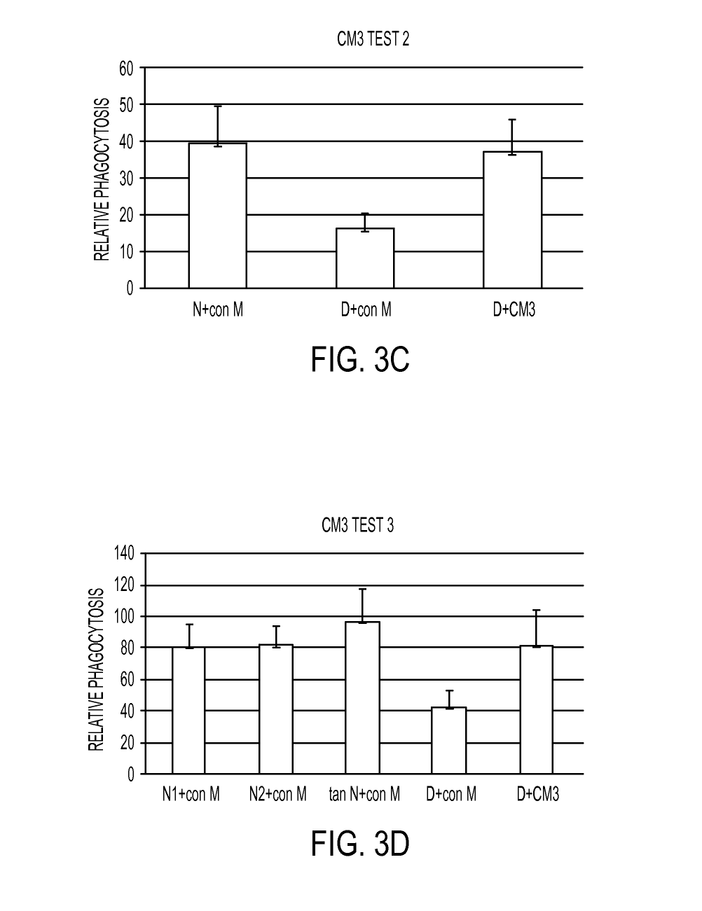

For serum-free medium (CM1 serum-free), medium was replenished at day 2 with 21 mL of DMEM/F12 serum-free medium (DMEM:F12 medium+Pen (50 U/ml)/Strep (50 μg/ml)). CM1 with or without serum restored phagocytosis activity (FIGS. 1A-1B and 2). Another conditioned medium (CM2) was prepared under the same procedure as CM1 with serum, except the cells were cultured T225 flasks with 63 mL of medium per flask, and the incubation time after medium change was 48 hours. This media, however, had no activity. (FIG. 3A). A CM3 was prepared with the same conditions as CM2 but with 10,000 viable cells/cm², and stimulated phagocytosis in dystrophic RPE. (FIGS. 3B-3D).

Of the conditions tested, CM2 was found to lack activity (FIG. 3A). CM2 had a shortened incubation time of 48 h, compared to the incubation time for CM1 of 54 hours. CM3 was prepared by doubling the cell seeding density with the same incubation time after medium change, compared to CM2, and found to be active. Therefore, to obtain an active CM, initial cell seeding density and cell incubation time after medium change are two important conditions.

Retinal pigment epithelium (RPE) cells from Royal College of Surgeons (RCS) rat have defective phagocytosis of rod outer segment (ROS) due to mutation in the Mertk gene. Mertk is a member of receptor tyrosine kinase (RTK) family and is thought to play a role in RPE phagocytosis. Basic fibroblast growth factor (bFGF), a ligand of FGF RTK, was shown to induce phagocytic competence in cultured RPE cells from RCS rats (McLaren, et al., FEBS Letters, 1997; 412:21-29). In an embodiment of the invention, hUTC rescue of dystrophic RPE phagocytosis is through secretion of RTK ligands, activating RTK signaling and enhancing signaling of other phagocytosis-related receptors.

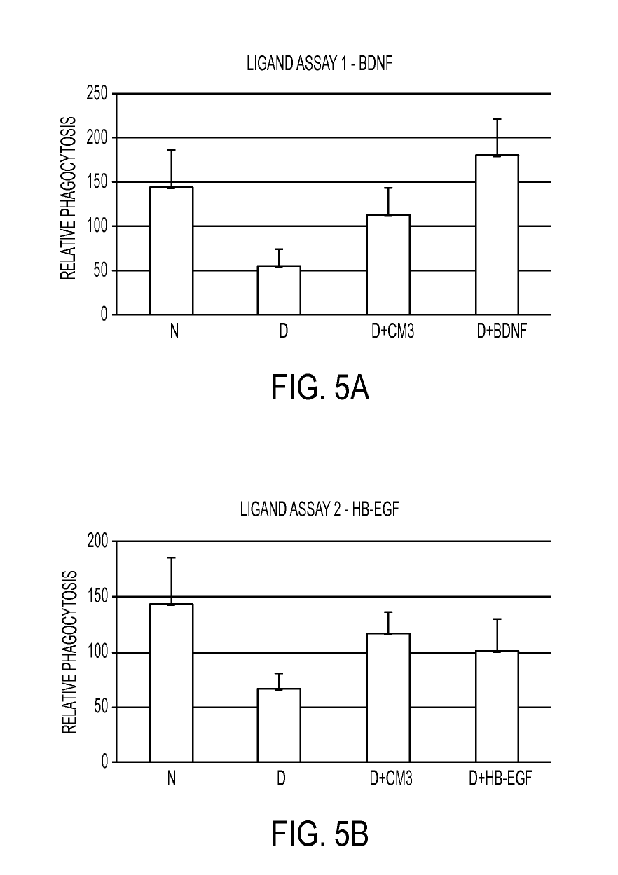

In an embodiment of the invention, RTK ligands BDNF, HB-EGF, PDGF-DD, Ephrin A4, HGF, and Ephrin B2, have rescue effect on phagocytosis by the RCS dystrophic RPE cells. In a particular embodiment, BDNF, PDGF-DD, and Ephrin B2 have positive rescue effects. (See FIGS. 5A, 6A-6B and 7A-7B).

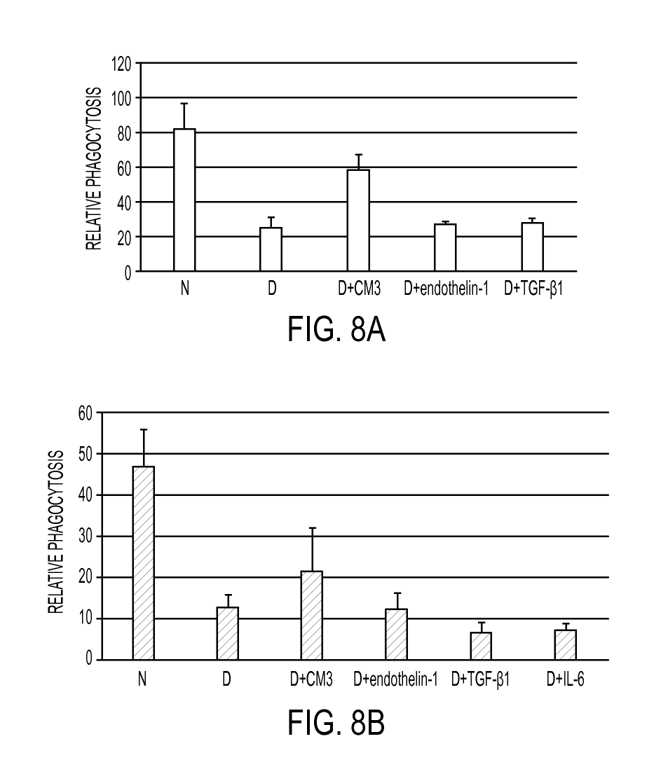

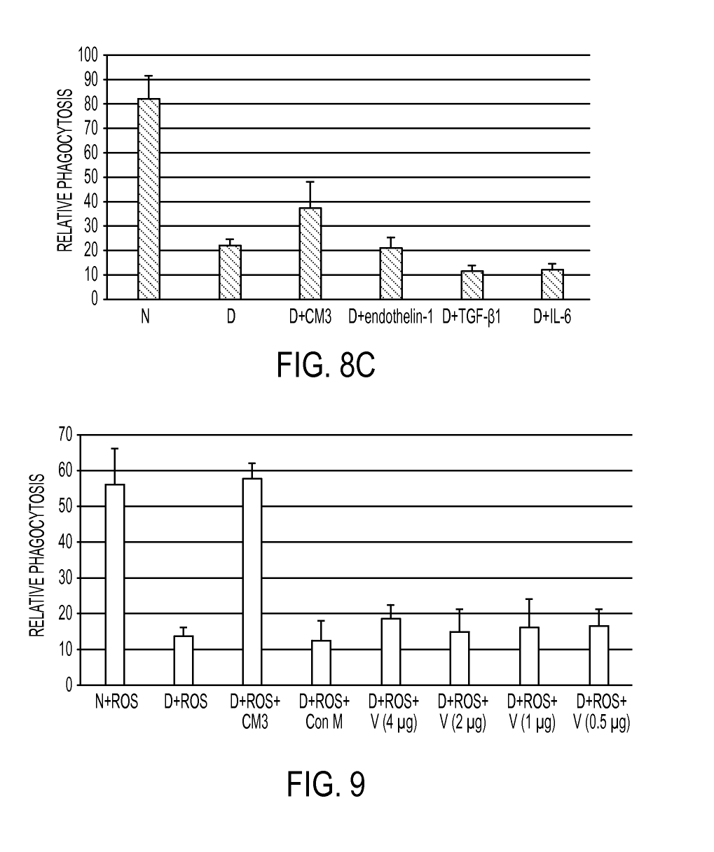

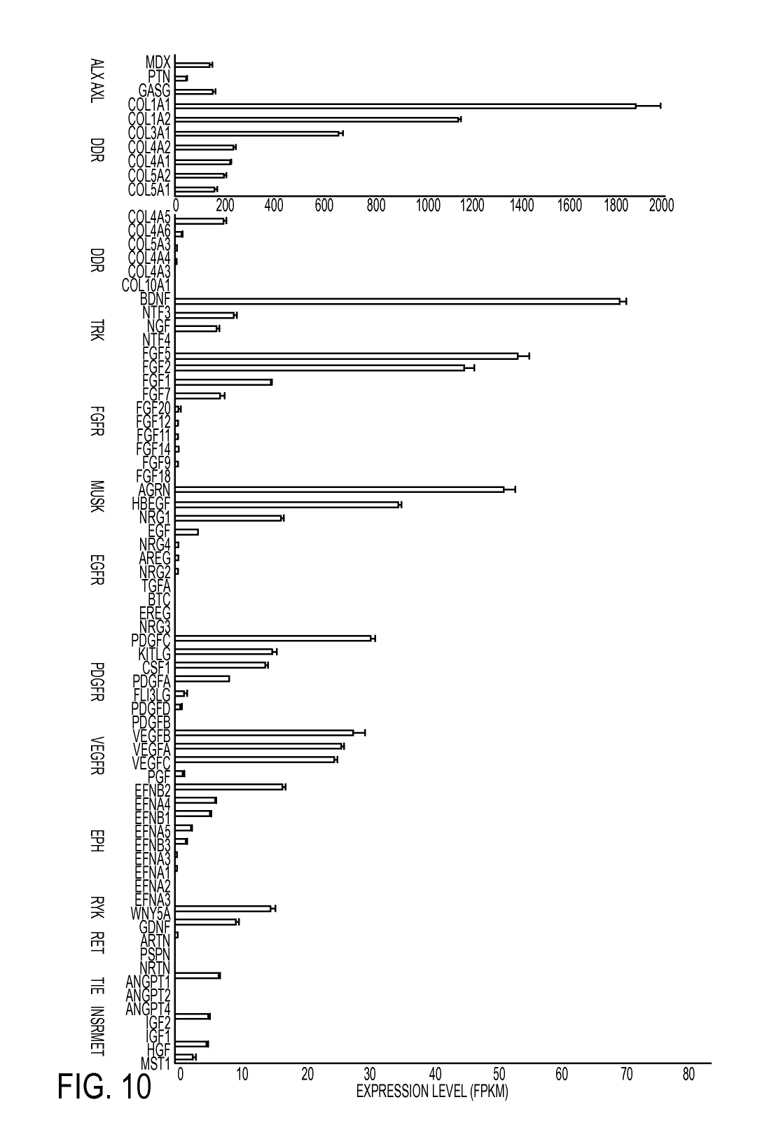

Non-RTK ligands activate different receptors from RTK, and do not have a similar effect on phagocytosis as RTK ligands (FIGS. 8A-8C and FIG. 9). hUTC has been shown to secrete vitronectin, endothelin-1, TGF-β1, and IL-6. Receptors for vitronectin include αvβ3 and αvβ5 integrins. Finnemann et al. reported that phagocytosis of ROS by RPE cells requires αvβ5 integrin (Finnemann et al., 1997, supra). While hUTC CM increased phagocytosis in dystrophic RPE cells, endothelin-1, TGF-β1 or IL-6 (concentration (200 ng/mL), and vitronectin (various concentrations) had no effect on RCS RPE phagocytosis (FIGS. 8A-8C, FIG. 9).

RNA analysis from conditioned media-treated and untreated dystrophic RPE for gene expression profiling show that hUTC express multiple genes of RTK ligands within 15 RTK subfamilies (FIG. 10, and Table 1-1). hUTC also express genes of bridge molecules, including MFG-E8, Gas6, protein S, TSP-1 and TSP-2 (Table 1-3). RCS RPE express genes in 18 RTK subfamilies (Table 1-2). Among the 18 are the 15 RTK subfamilies corresponding to the RTK ligand genes expressed in hUTC. RCS RPE also express receptor genes for bridge molecule binding, including integrin αvβ3, αvβ5, Ax1, Tyro3, MerTK, and CD36 (Table 1-4).

The transcriptomic profile of both RCS RPE cells and hUTC using RNA-Seq followed by informatics data analysis shows RCS RPE cells express multiple RTK genes, while hUTC expresses genes for multiple RTK ligands (Tables 1-1 to 1-4 and Table 2-1). Specifically, RTK ligands of seven RTK subfamilies have relatively high gene expression levels. These ligands include BDNF (brain-derived neurotrophic factor) and NT3 (neurotrophin 3)—ligands of Trk family, HGF (hepatocyte growth factor)—a ligand of Met family, PDGF-DD (platelet-derived growth factor type D) and PDGF-CC (platelet-derived growth factor type C)—ligands of PDGF family, ephrin-B2—a ligand of Eph family, HB-EGF (heparin-binding epidermal growth factor)—a ligand of ErbB family, GDNF (glial cell-derived neurotrophic factor)—a ligand of Ret family, as well as agrin—a ligand of Musk family.

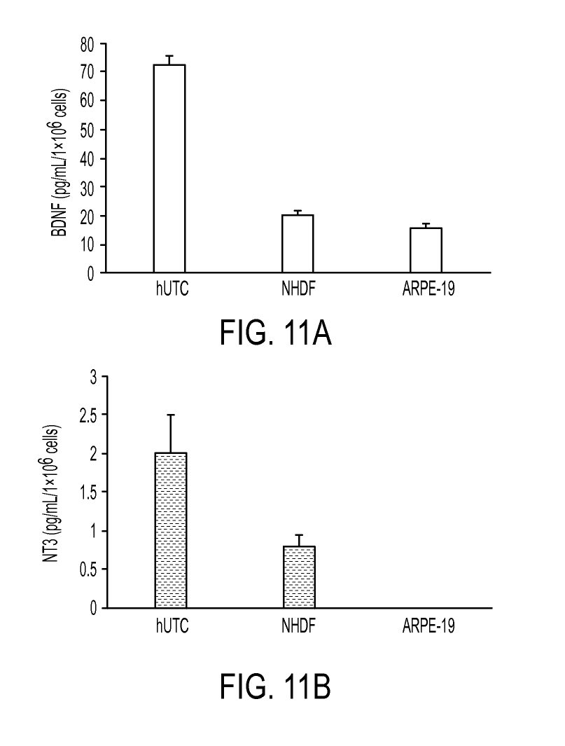

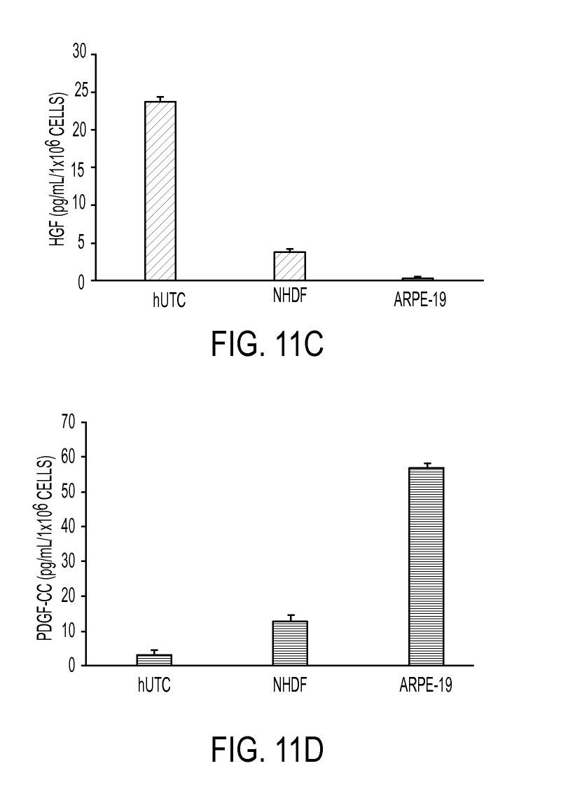

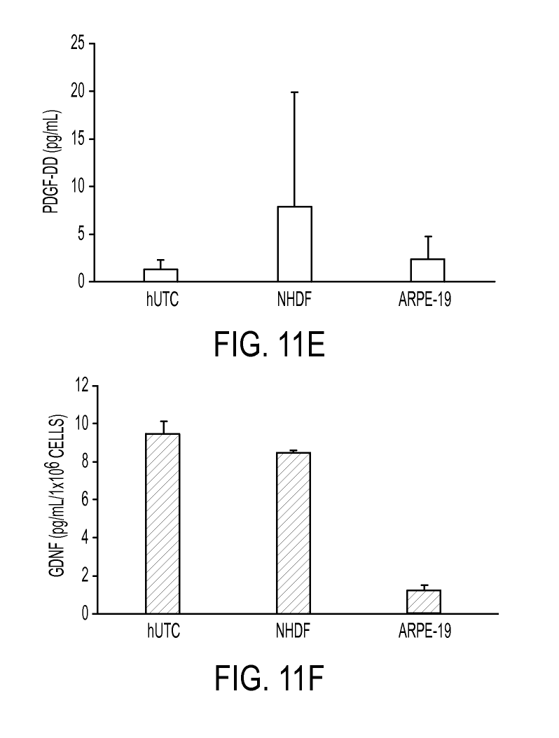

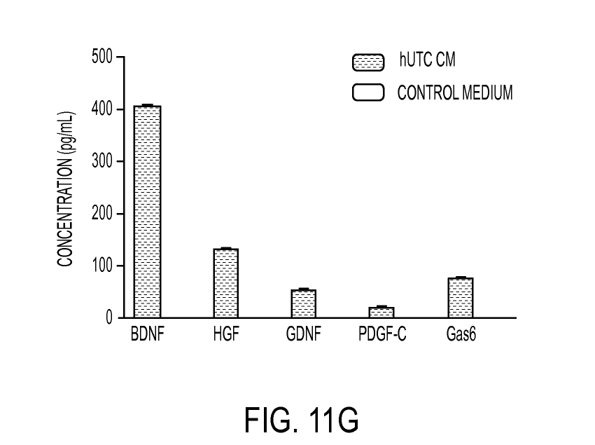

In an embodiment of the invention, BDNF, NT3, HGF, and GDNF are secreted in hUTC conditioned media, and at higher levels in comparison with those from normal human dermal fibroblast (NHDF) and ARPE-19 cells, as measured in ELISA assays. (FIGS. 11A-11C and 11F). In another embodiment, hUTC secrete low levels of PDGF-CC and PDGF-DD compared to NHDF or ARPE-19 (FIGS. 11D, 11E). In a further embodiment, ephrin-B2 and HB-EGF are not detected in conditioned medium of hUTC, NHDF, and ARPE-19, as measured in ELISA assays. These cells either do not secrete the two proteins, or the levels are below the limit of detection of the ELISA assay. The levels of agrin in hUTC, NHDF and ARPE-19 conditioned medium are similar to that in control medium; agrin detected in all the conditioned medium samples may be from the medium.

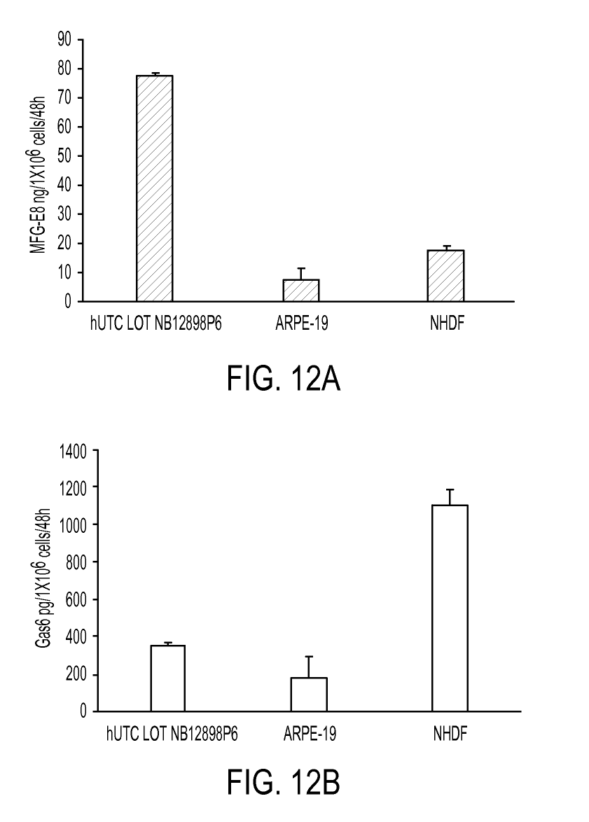

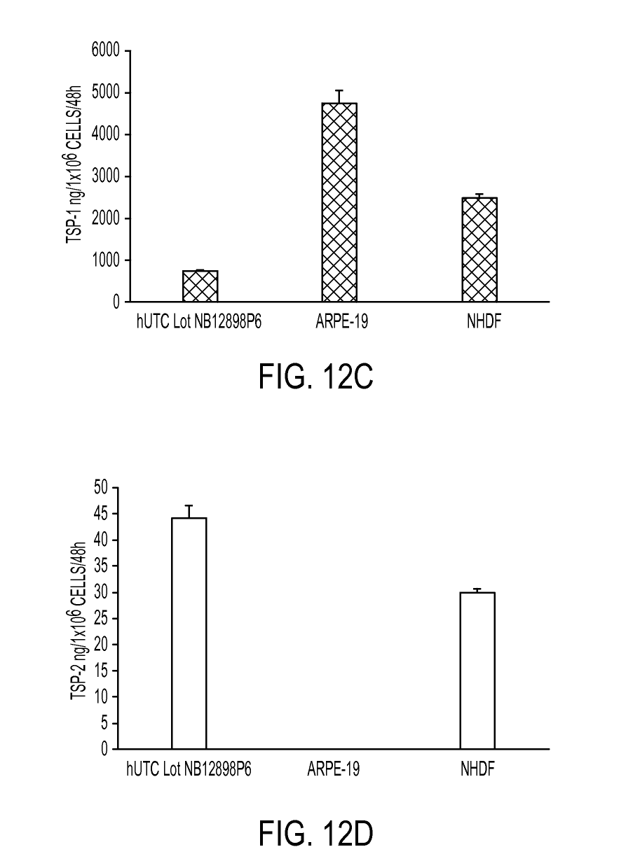

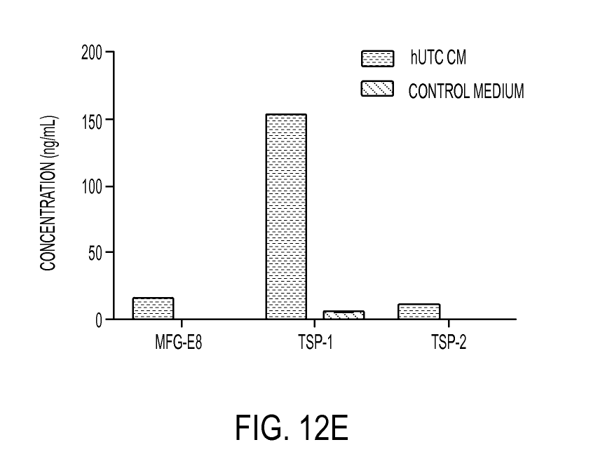

Based on the RNA-Seq-based transcriptome profile analysis of RCS RPE cells, the level of bridge molecules and other factors secreted in hUTC conditioned medium demonstrates further the effect on phagocytosis, and consequently, apoptosis. As shown, RCS RPE cells express genes of many receptors identified to date that recognize “eat me” signals on apoptotic cells. These receptors include scavenger receptors (SR-A, LOX-1, CD68, CD36, CD14), integrins (αvβ3 and αvβ5), receptor tyrosine kinases of the Ax1 and Tyro3, LRP-1/CD91, and PS receptor Stabilin 1 (Table 3-1; adapted from Erwig L-P and Henson P M, Cell Death and Differentiation 2008; 15: 243-250). Moreover, hUTC expresses a number of bridge molecule genes including TSP-1, TSP-2, surfactant protein D (SP-D), MFG-E8, Gas6, apolipoprotein H, and annexin 1. In an embodiment of the invention, hUTC secrete MFG-E8, Gas6, TSP-1, TSP-2 in hUTC conditioned media. (FIGS. 12A-12E and Table 3-2). In another embodiment, hUTC do not secrete apolipoprotein H, SP-D or annexin I (Table 3-2). In certain embodiments, hUTC secrete MFG-E8 and TSP-2 at significantly higher levels than NHDF and ARPE-19. (FIGS. 12A and 12D).

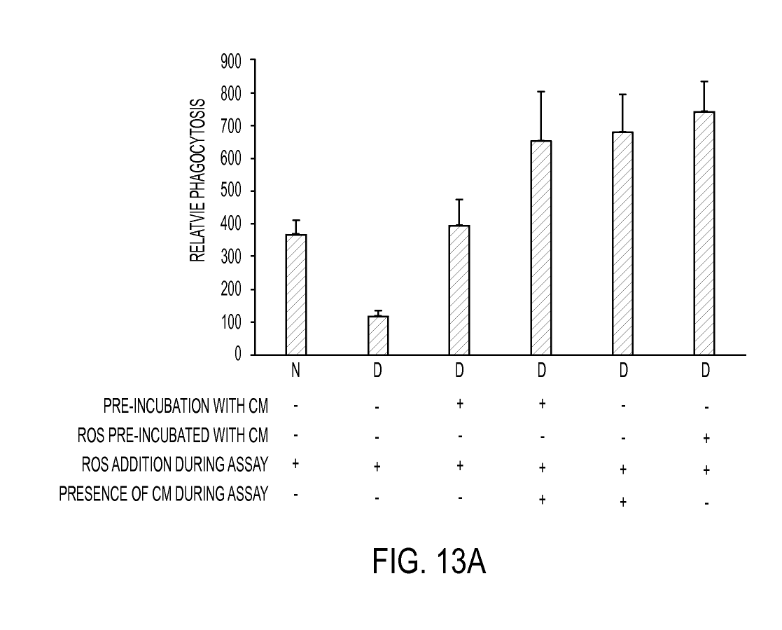

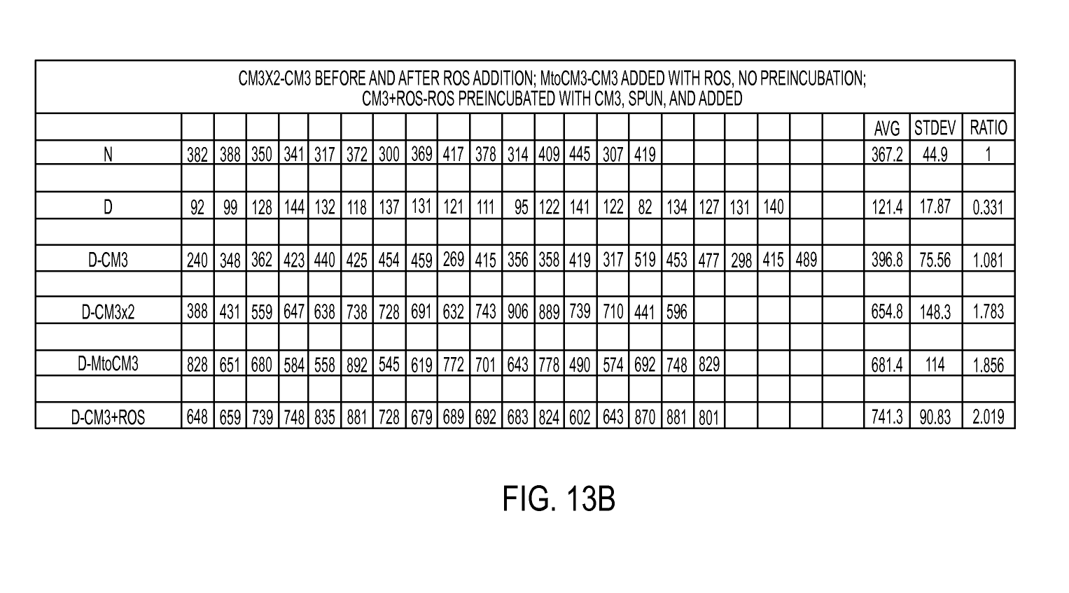

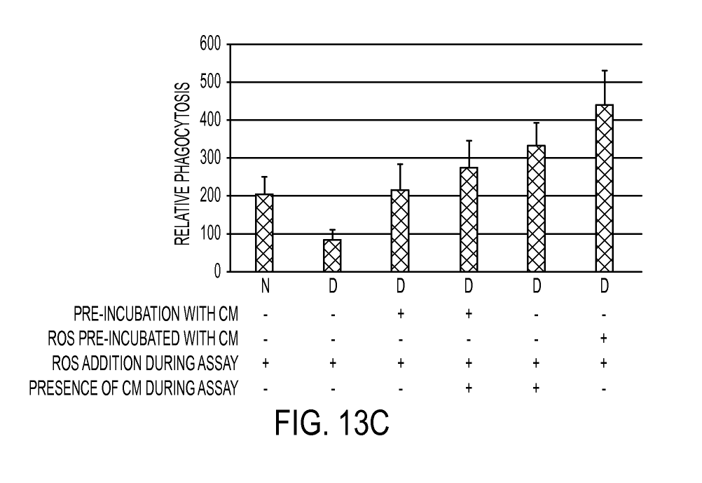

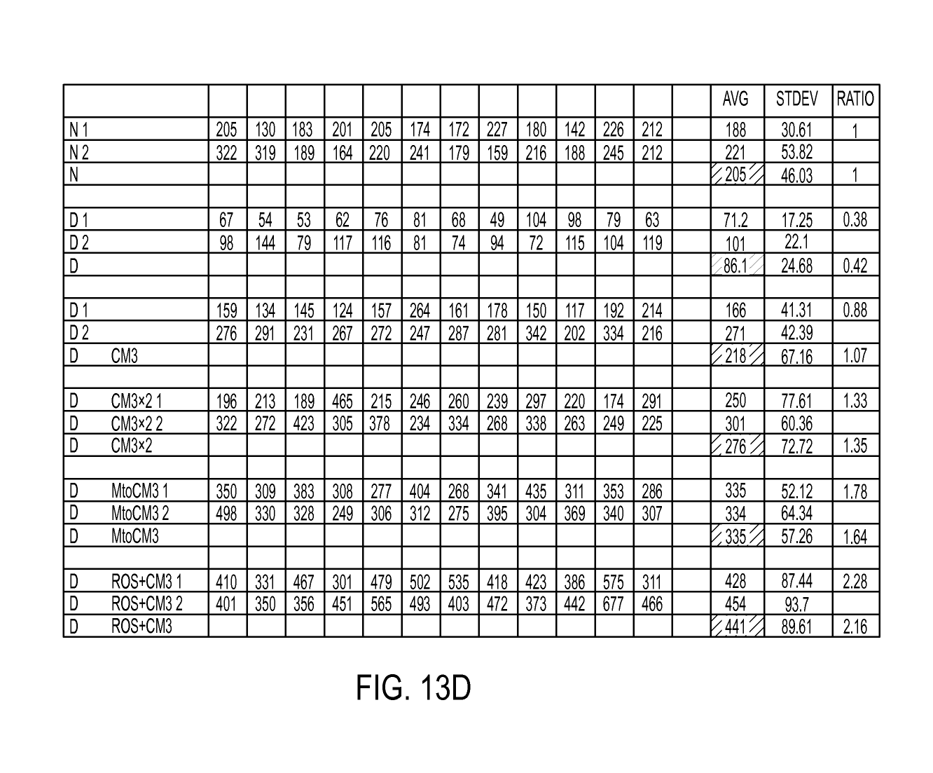

In an aspect of the invention, hUTC conditioned media stimulates ROS phagocytosis when feeding RCS RPE cells with ROS preincubated with hUTC CM. Phagocytosis of the dystrophic RPE cells was completely rescued. As shown in FIGS. 13A-13D, untreated dystrophic RPE cells have reduced phagocytosis compared to normal RPE cells. In an embodiment of the invention, preincubation of dystrophic RPE cells with hUTC CM rescues phagocytosis. This occurs even without hUTC CM being present during the assay. In embodiments of the invention, phagocytic-related receptors and their signaling pathways are up-regulated during the preincubation period. Robust enhancement of phagocytosis was observed when hUTC CM was present throughout the phagocytosis assay whether dystrophic RPE cells were pretreated with hUTC CM or not. In an embodiment, dystrophic RPE cells, fed with ROS pretreated with hUTC CM, restores or rescues phagocytosis. This occurs even in the absence of hUTC CM during the phagocytosis assay. In particular embodiments of the invention, hUTC CM may prime or modify ROS that enhances ROS binding and internalization, through for example, bridge molecules/opsonins that favorably facilitate phagocytosis.

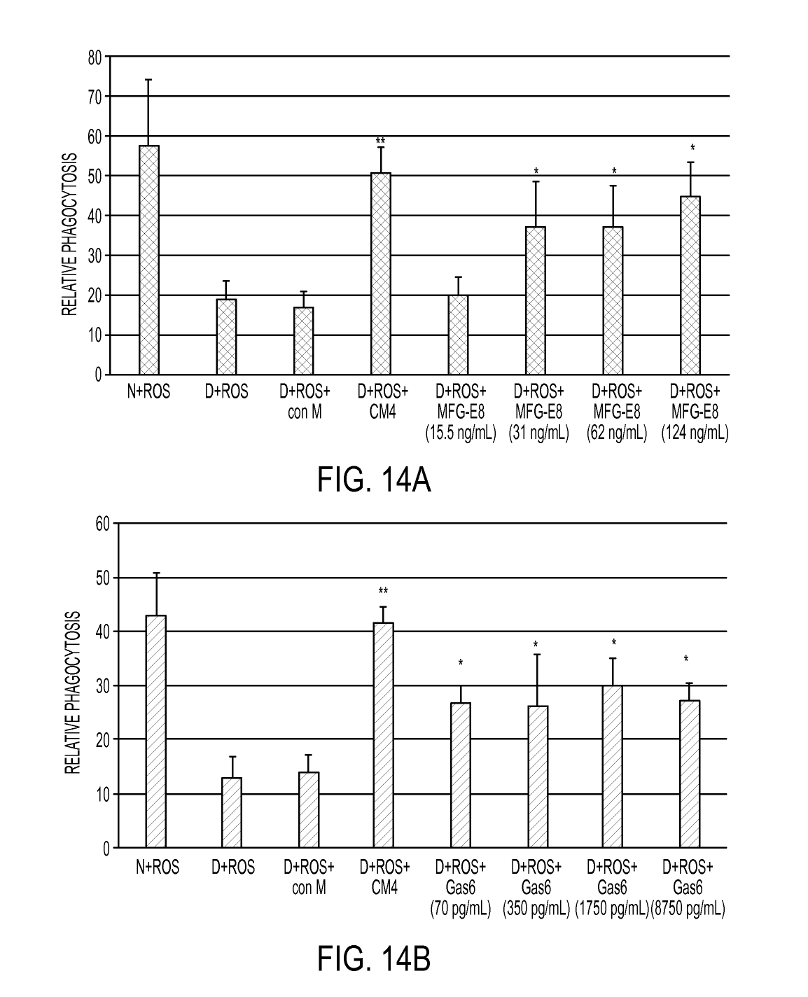

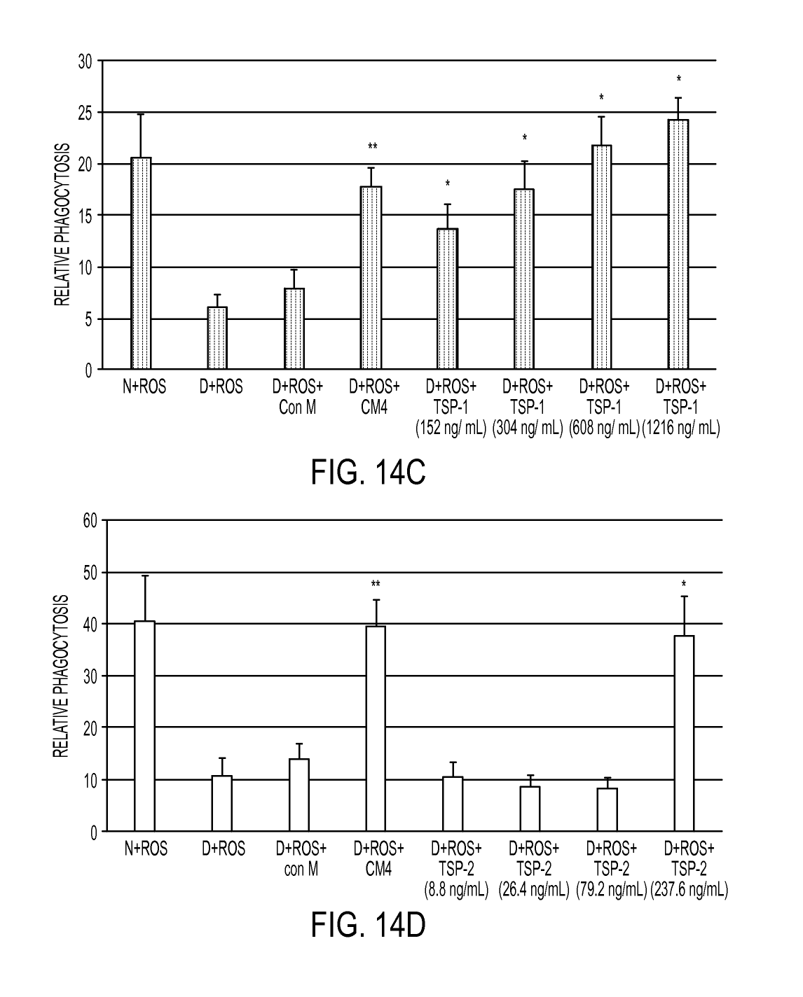

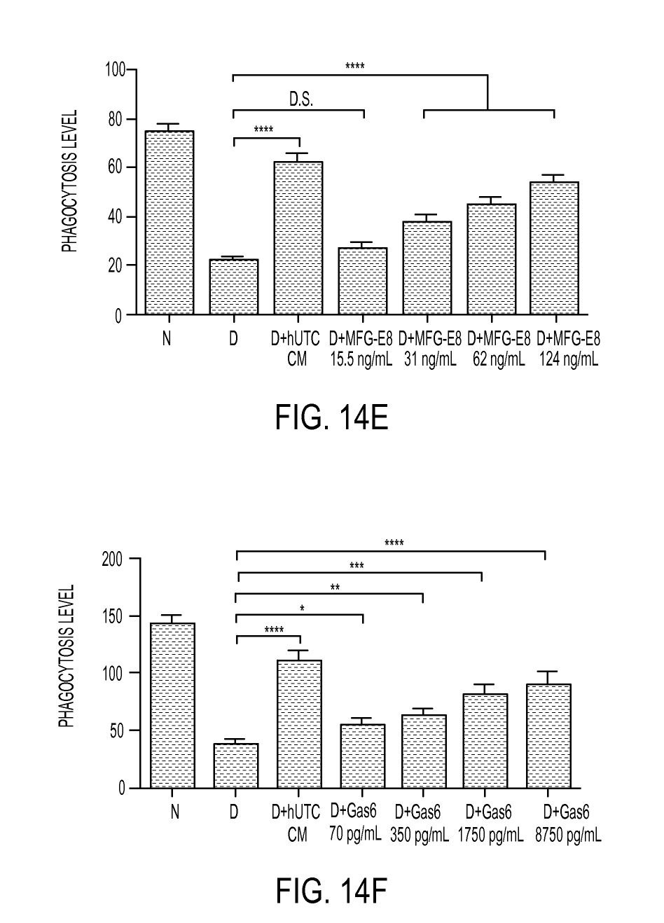

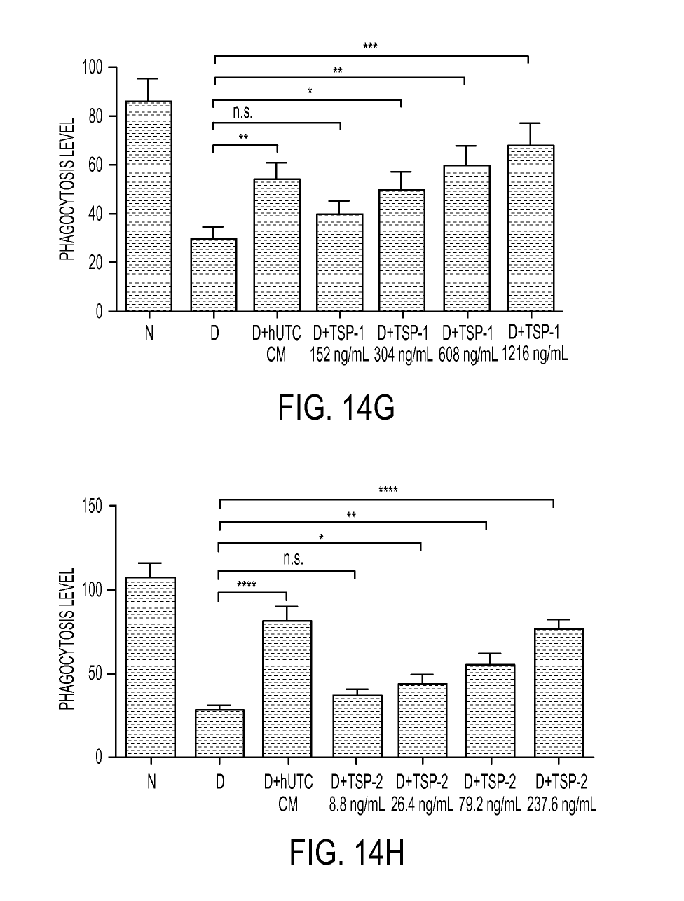

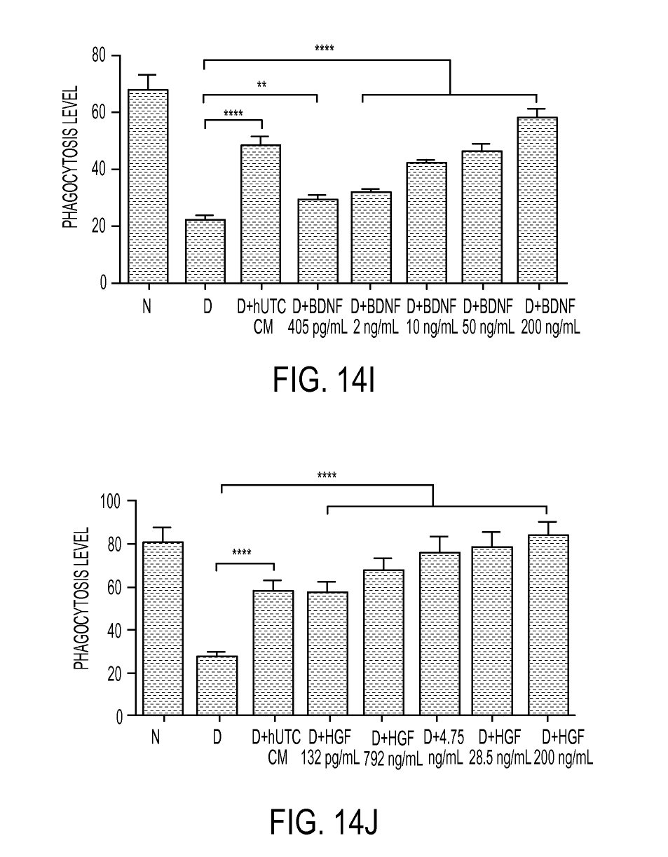

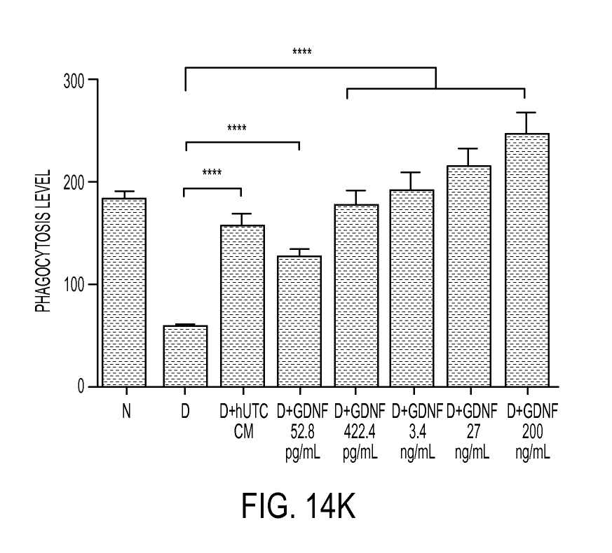

In embodiments of the invention, bridge molecules MFG-E8, Gas6, TSP-1 and TSP-2 mediate ROS phagocytosis by RCS RPE cells. Dystrophic RPE cells fed with ROS preincubated with various concentrations of MFG-E8, Gas6, TSP-1 or TSP-2 and assayed for phagocytosis showed rescue of ROS phagocytosis (FIGS. 14A-14H). In a particular embodiment, hUTC conditioned media mediates RCS RPE phagocytosis rescue through secretion of bridge molecules, for example, MFG-E8, Gas6, TSP-1 and TSP-2.

In an embodiment, RTK ligands, such as BDNF, HGF and GDNF stimulate hUTC-mediated phagocytosis rescue in RCS RPE. RTK ligands BDNF, HGF and GDNF rescued phagocytosis in RCS RPE (FIGS. 14I-J). Recombinant RTK ligand and bridge molecule proteins can mimic the effect of hUTC CM and restore RCS RPE phagocytosis, and are involved in hUTC-mediated phagocytosis rescue in RCS RPE.

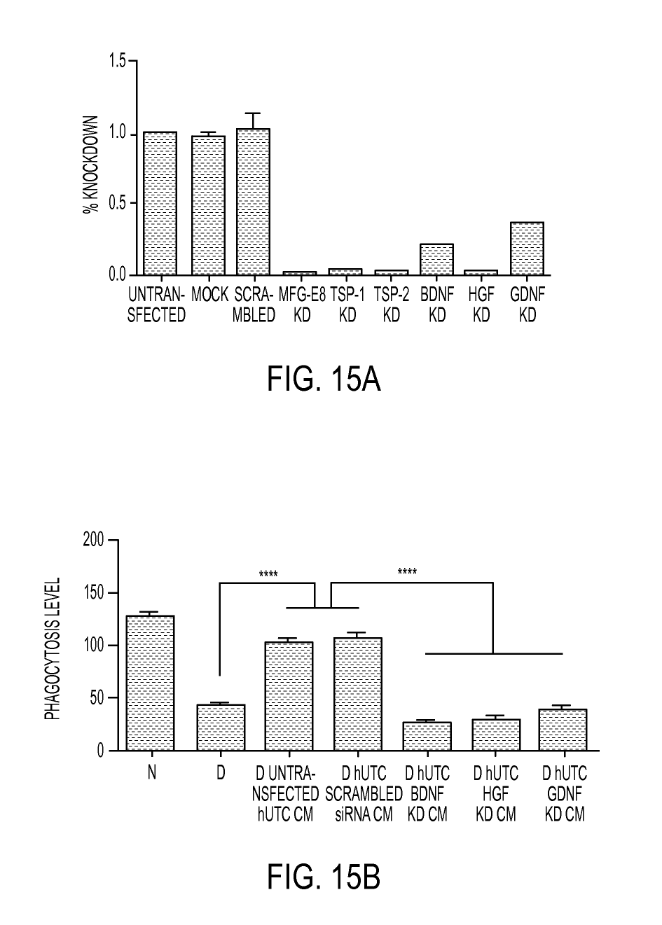

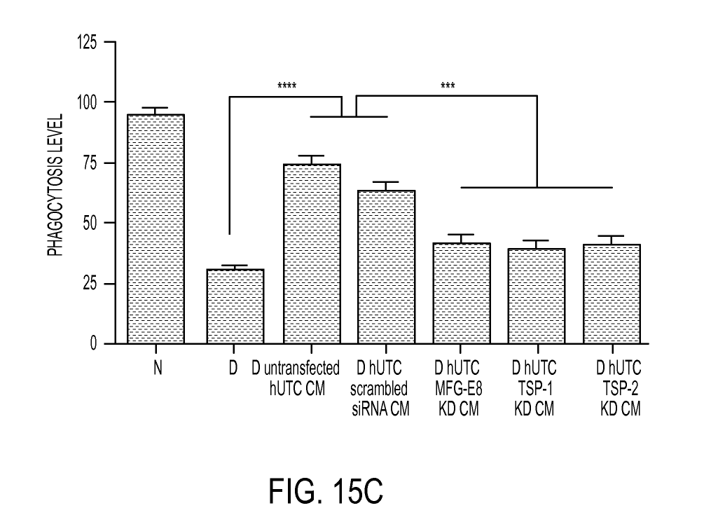

siRNA mediated gene silencing demonstrated BDNF, HGF, GDNF, MFG-E8, Gas6, TSP-1 and TSP-2 knocked down (silenced) in hUTC. Mock or scrambled siRNA transfection had no effect on hUTC secretion of these factors. siRNA targeting MFG-E8, TSP-1, TSP-2 and HGF yielded almost 100% knockdown efficiency; 80% and 65% knockdown were observed for BDNF and GDNF, respectively (FIGS. 15A-15B). Knocking-down each of the bridge molecules MFG-E8, TSP-1, TSP-2 decreased the phagocytosis of OS by RCS RPE (FIG. 15C). In a particular embodiment, RTK ligands BDNF, HGF and GDNF, are required for hUTC-mediated phagocytosis rescue in RCS RPE. In another embodiment, bridge molecules such as MFG-E8, Gas6, TSP-1 and TSP-2 are required for hUTC-mediated phagocytosis rescue in RCS RPE.

### Cell Modifications, Components and Products

Progenitor cells, such as postpartum cells, may also be genetically modified to produce therapeutically useful gene products, or to produce antineoplastic agents for treatment of tumors. Genetic modification may be accomplished using any of a variety of vectors including, but not limited to, integrating viral vectors, e.g., retrovirus vector or adeno-associated viral vectors; non-integrating replicating vectors, e.g., papilloma virus vectors, SV40 vectors, adenoviral vectors; or replication-defective viral vectors. Other methods of introducing DNA into cells include the use of liposomes, electroporation, a particle gun, or by direct DNA injection.

Hosts cells are preferably transformed or transfected with DNA controlled by or in operative association with, one or more appropriate expression control elements such as promoter or enhancer sequences, transcription terminators, polyadenylation sites, among others, and a selectable marker. Any promoter may be used to drive the expression of the inserted gene. For example, viral promoters include, but are not limited to, the CMV promoter/enhancer, SV40, papillomavirus, Epstein-Barr virus or elastin gene promoter. In some embodiments, the control elements used to control expression of the gene of interest can allow for the regulated expression of the gene so that the product is synthesized only when needed in vivo. If transient expression is desired, constitutive promoters are preferably used in a non-integrating and/or replication-defective vector. Alternatively, inducible promoters could be used to drive the expression of the inserted gene when necessary. Inducible promoters include, but are not limited to those associated with metallothionein and heat shock proteins.

Following the introduction of the foreign DNA, engineered cells may be allowed to grow in enriched media and then switched to selective media. The selectable marker in the foreign DNA confers resistance to the selection and allows cells to stably integrate the foreign DNA as, for example, on a plasmid, into their chromosomes and grow to form foci which, in turn, can be cloned and expanded into cell lines. This method can be advantageously used to engineer cell lines that express the gene product.

Cells may be genetically engineered to “knock out” or “knock down” expression of factors that promote inflammation or rejection at the implant site. Negative modulatory techniques for the reduction of target gene expression levels or target gene product activity levels are discussed below. “Negative modulation,” as used herein, refers to a reduction in the level and/or activity of target gene product relative to the level and/or activity of the target gene product in the absence of the modulatory treatment. The expression of a gene native to a neuron or glial cell can be reduced or knocked out using a number of techniques including, for example, inhibition of expression by inactivating the gene using the homologous recombination technique. Typically, an exon encoding an important region of the protein (or an exon 5′ to that region) is interrupted by a positive selectable marker, e.g., neo, preventing the production of normal mRNA from the target gene and resulting in inactivation of the gene. A gene may also be inactivated by creating a deletion in part of a gene, or by deleting the entire gene. By using a construct with two regions of homology to the target gene that are far apart in the genome, the sequences intervening the two regions can be deleted (Mombaerts et al., 1991, Proc. Nat. Acad. Sci. U.S.A. 88:3084-3087). Antisense, DNAzymes, ribozymes, small interfering RNA (siRNA) and other such molecules that inhibit expression of the target gene can also be used to reduce the level of target gene activity. For example, antisense RNA molecules that inhibit the expression of major histocompatibility gene complexes (HLA) have been shown to be most versatile with respect to immune responses. Still further, triple helix molecules can be utilized in reducing the level of target gene activity. These techniques are described in detail by L. G. Davis et al. (eds), 1994, BASIC METHODS IN MOLECULAR BIOLOGY, 2nd ed., Appleton & Lange, Norwalk, Conn.

In other aspects, the invention provides cell lysates and cell soluble fractions prepared from postpartum cells, preferably PPDCs, or heterogeneous or homogeneous cell populations comprising PPDCs cells, as well as PPDCs or populations thereof that have been genetically modified or that have been stimulated to differentiate along a neurogenic pathway. Such lysates and fractions thereof have many utilities. Use of the cell lysate soluble fraction (i.e., substantially free of membranes) in vivo, for example, allows the beneficial intracellular milieu to be used allogeneically in a patient without introducing an appreciable amount of the cell surface proteins most likely to trigger rejection, or other adverse immunological responses. Methods of lysing cells are well known in the art and include various means of mechanical disruption, enzymatic disruption, or chemical disruption, or combinations thereof. Such cell lysates may be prepared from cells directly in their growth medium and thus containing secreted growth factors and the like, or may be prepared from cells washed free of medium in, for example, PBS or other solution. Washed cells may be resuspended at concentrations greater than the original population density if preferred.

In one embodiment, whole cell lysates are prepared, e.g., by disrupting cells without subsequent separation of cell fractions. In another embodiment, a cell membrane fraction is separated from a soluble fraction of the cells by routine methods known in the art, e.g., centrifugation, filtration, or similar methods.

Cell lysates or cell soluble fractions prepared from populations of progenitor cells, such as postpartum-derived cells, may be used as is, further concentrated, by for example, ultrafiltration or lyophilization, or even dried, partially purified, combined with pharmaceutically-acceptable carriers or diluents as are known in the art, or combined with other compounds such as biologicals, for example pharmaceutically useful protein compositions. Cell lysates or fractions thereof may be used in vitro or in vivo, alone or for example, with autologous or syngeneic live cells. The lysates, if introduced in vivo, may be introduced locally at a site of treatment, or remotely to provide, for example needed cellular growth factors to a patient.

In a further embodiment, postpartum cells, preferably PPDCs, can be cultured in vitro to produce biological products in high yield. For example, such cells, which either naturally produce a particular biological product of interest (e.g., a trophic factor), or have been genetically engineered to produce a biological product, can be clonally expanded using the culture techniques described herein. Alternatively, cells may be expanded in a medium that induces differentiation to a desired lineage. In either case, biological products produced by the cell and secreted into the medium can be readily isolated from the conditioned medium using standard separation techniques, e.g., such as differential protein precipitation, ion-exchange chromatography, gel filtration chromatography, electrophoresis, and HPLC, to name a few. A “bioreactor” may be used to take advantage of the flow method for feeding, for example, a three-dimensional culture in vitro. Essentially, as fresh media is passed through the three-dimensional culture, the biological product is washed out of the culture and may then be isolated from the outflow, as above.

Alternatively, a biological product of interest may remain within the cell and, thus, its collection may require that the cells be lysed, as described above. The biological product may then be purified using anyone or more of the above-listed techniques.

In another embodiment, an extracellular matrix (ECM) produced by culturing postpartum cells (preferably PPDCs) on liquid, solid or semi-solid substrates is prepared, collected and utilized as an alternative to implanting live cells into a subject in need of tissue repair or replacement. The cells are cultured in vitro, on a three dimensional framework as described elsewhere herein, under conditions such that a desired amount of ECM is secreted onto the framework. The cells and the framework are removed, and the ECM processed for further use, for example, as an injectable preparation. To accomplish this, cells on the framework are killed and any cellular debris removed from the framework. This process may be carried out in a number of different ways. For example, the living tissue can be flash-frozen in liquid nitrogen without a cryopreservative, or the tissue can be immersed in sterile distilled water so that the cells burst in response to osmotic pressure.

Once the cells have been killed, the cellular membranes may be disrupted and cellular debris removed by treatment with a mild detergent rinse, such as EDTA, CHAPS or a zwitterionic detergent. Alternatively, the tissue can be enzymatically digested and/or extracted with reagents that break down cellular membranes and allow removal of cell contents. Example of such enzymes include, but are not limited to, hyaluronidase, dispase, proteases, and nucleases. Examples of detergents include non-ionic detergents such as, for example, alkylaryl polyether alcohol (TRITON X-100), octylphenoxy polyethoxy-ethanol (Rohm and Haas Philadelphia, Pa.), BRIJ-35, a polyethoxyethanollauryl ether (Atlas Chemical Co., San Diego, Calif.), polysorbate 20 (TWEEN 20), a polyethoxyethanol sorbitan mono laureate (Rohm and Haas), polyethylene lauryl ether (Rohm and Haas); and ionic detergents such as, for example, sodium dodecyl sulphate, sulfated higher aliphatic alcohols, sulfonated alkanes and sulfonated alkylarenes containing 7 to 22 carbon atoms in a branched or unbranched chain.

The collection of the ECM can be accomplished in a variety of ways, depending, for example, on whether the new tissue has been formed on a three-dimensional framework that is biodegradable or non-biodegradable. For example, if the framework is non-biodegradable, the ECM can be removed by subjecting the framework to sonication, high-pressure water jets, mechanical scraping, or mild treatment with detergents or enzymes, or any combination of the above.

If the framework is biodegradable, the ECM can be collected, for example, by allowing the framework to degrade or dissolve in solution. Alternatively, if the biodegradable framework is composed of a material that can itself be injected along with the ECM, the framework and the ECM can be processed in toto for subsequent injection. Alternatively, the ECM can be removed from the biodegradable framework by any of the methods described above for collection of ECM from a non-biodegradable framework. All collection processes are preferably designed so as not to denature the ECM.

After it has been collected, the ECM may be processed further. For example, the ECM can be homogenized to fine particles using techniques well known in the art such as by sonication, so that it can pass through a surgical needle. The components of the ECM can be crosslinked, if desired, by gamma irradiation. Preferably, the ECM can be irradiated between 0.25 to 2 mega rads to sterilize and cross link the ECM. Chemical crosslinking using agents that are toxic, such as glutaraldehyde, is possible but not generally preferred.

The amounts and/or ratios of proteins, such as the various types of collagen present in the ECM, may be adjusted by mixing the ECM produced by the cells of the invention with ECM of one or more other cell types. In addition, biologically active substances such as proteins, growth factors and/or drugs, can be incorporated into the ECM. Exemplary biologically active substances include tissue growth factors, such as TGF-beta, and the like, which promote healing and tissue repair at the site of the injection. Such additional agents may be utilized in any of the embodiments described herein above, e.g., with whole cell lysates, soluble cell fractions, or further purified components and products produced by the cells.

### Pharmaceutical Compositions

In another aspect, the invention provides pharmaceutical compositions that use progenitor cells such as postpartum cells (preferably PPDCs), cell populations thereof, conditioned media produced by such cells, and cell components and products produced by such cells in various methods for treatment of ocular degenerative conditions. Certain embodiments encompass pharmaceutical compositions comprising live cells (e.g., PPDCs alone or admixed with other cell types). Other embodiments encompass pharmaceutical compositions comprising PPDC conditioned medium. Additional embodiments may use cellular components of PPDC (e.g., cell lysates, soluble cell fractions, ECM, or components of any of the foregoing) or products (e.g., trophic and other biological factors produced naturally by the cells or through genetic modification, conditioned medium from culturing the cells). In either case, the pharmaceutical composition may further comprise other active agents, such as anti-inflammatory agents, anti-apoptotic agents, antioxidants, growth factors, neurotrophic factors or neuroregenerative, neuroprotective or ophthalmic drugs as known in the art.

Examples of other components that may be added to the pharmaceutical compositions include, but are not limited to: (1) other neuroprotective or neurobeneficial drugs; (2) selected extracellular matrix components, such as one or more types of collagen known in the art, and/or growth factors, platelet-rich plasma, and drugs (alternatively, PPDCs may be genetically engineered to express and produce growth factors); (3) anti-apoptotic agents (e.g., erythropoietin (EPO), EPO mimetibody, thrombopoietin, insulin-like growth factor (IGF)-I, IGF-II, hepatocyte growth factor, caspase inhibitors); (4) anti-inflammatory compounds (e.g., p38 MAP kinase inhibitors, TGF-beta inhibitors, statins, IL-6 and IL-1 inhibitors, PEMIROLAST, TRANILAST, REMICADE, SIROLIMUS, and non-steroidal anti-inflammatory drugs (NSAIDS) (such as TEPDXALIN, TOLMETIN, and SUPROFEN); (5) immunosuppressive or immunomodulatory agents, such as calcineurin inhibitors, mTOR inhibitors, antiproliferatives, corticosteroids and various antibodies; (6) antioxidants such as probucol, vitamins C and E, conenzyme Q-10, glutathione, L-cysteine and N-acetylcysteine; and (6) local anesthetics, to name a few.

Pharmaceutical compositions of the invention comprise progenitor cells, such as postpartum cells (preferably PPDCs), conditioned media generated from those cells, or components or products thereof, formulated with a pharmaceutically acceptable carrier or medium. Suitable pharmaceutically acceptable carriers include water, salt solution (such as Ringer's solution), alcohols, oils, gelatins, and carbohydrates, such as lactose, amylose, or starch, fatty acid esters, hydroxymethylcellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Typically, but not exclusively, pharmaceutical compositions comprising cellular components or products, but not live cells, are formulated as liquids. Pharmaceutical compositions comprising PPDC live cells are typically formulated as liquids, semisolids (e.g., gels) or solids (e.g., matrices, scaffolds and the like, as appropriate for ophthalmic tissue engineering).

Pharmaceutical compositions may comprise auxiliary components as would be familiar to medicinal chemists or biologists. For example, they may contain antioxidants in ranges that vary depending on the kind of antioxidant used. Reasonable ranges for commonly used antioxidants are about 0.01% to about 0.15% weight by volume of EDTA, about 0.01% to about 2.0% weight volume of sodium sulfite, and about 0.01% to about 2.0% weight by volume of sodium metabisulfite. One skilled in the art may use a concentration of about 0.1% weight by volume for each of the above. Other representative compounds include mercaptopropionyl glycine, N-acetyl cysteine, beta-mercaptoethylamine, glutathione and similar species, although other antioxidant agents suitable for ocular administration, e.g. ascorbic acid and its salts or sulfite or sodium metabisulfite may also be employed.

A buffering agent may be used to maintain the pH of eye drop formulations in the range of about 4.0 to about 8.0; so as to minimize irritation of the eye. For direct intravitreal or intraocular injection, formulations should be at pH 7.2 to 7.5, preferably at pH 7.3-7.4. The ophthalmologic compositions may also include tonicity agents suitable for administration to the eye. Among those suitable is sodium chloride to make formulations approximately isotonic with 0.9% saline solution.

In certain embodiments, pharmaceutical compositions are formulated with viscosity enhancing agents. Exemplary agents are hydroxyethylcellulose, hydroxypropylcellulose, methylcellulose, and polyvinylpyrrolidone. The pharmaceutical compositions may have cosolvents added if needed. Suitable cosolvents may include glycerin, polyethylene glycol (PEG), polysorbate, propylene glycol, and polyvinyl alcohol. Preservatives may also be included, e.g., benzalkonium chloride, benzethonium chloride, chlorobutanol, phenylmercuric acetate or nitrate, thimerosal, or methyl or propylparabens.

Formulations for injection are preferably designed for single-use administration and do not contain preservatives. Injectable solutions should have isotonicity equivalent to 0.9% sodium chloride solution (osmolality of 290-300 milliosmoles). This may be attained by addition of sodium chloride or other co-solvents as listed above, or excipients such as buffering agents and antioxidants, as listed above.

The tissues of the anterior chamber of the eye are bathed by the aqueous humor, while the retina is under continuous exposure to the vitreous. These fluids/gels exist in a highly reducing redox state because they contain antioxidant compounds and enzymes. Therefore, it may be advantageous to include a reducing agent in the ophthalmologic compositions. Suitable reducing agents include N-acetylcysteine, ascorbic acid or a salt form, and sodium sulfite or metabisulfite, with ascorbic acid and/or N-acetylcysteine or glutathione being particularly suitable for injectable solutions.

Pharmaceutical compositions comprising cells or conditioned medium, or cell components or cell products may be delivered to the eye of a patient in one or more of several delivery modes known in the art. In one embodiment that may be suitable for use in some instances, the compositions are topically delivered to the eye in eye drops or washes. In another embodiment, the compositions may be delivered to various locations within the eye via periodic intraocular injection or by infusion in an irrigating solution such as BSS or BSS PLUS (Alcon USA, Fort Worth, Tex.). Alternatively, the compositions may be applied in other ophthalmologic dosage forms known to those skilled in the art, such as pre-formed or in situ-formed gels or liposomes, for example as disclosed in U.S. Pat. No. 5,718,922 to Herrero-Vanrell. In another embodiment, the composition may be delivered to or through the lens of an eye in need of treatment via a contact lens (e.g. Lidofilcon B, Bausch & Lomb CW79 or DELTACON (Deltafilcon A) or other object temporarily resident upon the surface of the eye. In other embodiments, supports such as a collagen corneal shield (e.g. BIO-COR dissolvable corneal shields, Summit Technology, Watertown, Mass.) can be employed. The compositions can also be administered by infusion into the eyeball, either through a cannula from an osmotic pump (ALZET, Alza Corp., Palo Alto, Calif.) or by implantation of timed-release capsules (OCCUSENT) or biodegradable disks (OCULEX, OCUSERT). These routes of administration have the advantage of providing a continuous supply of the pharmaceutical composition to the eye. This may be an advantage for local delivery to the cornea.

Pharmaceutical compositions comprising live cells in a semi-solid or solid carrier are typically formulated for surgical implantation at the site of ocular damage or distress. It will be appreciated that liquid compositions also may be administered by surgical procedures, for example conditioned media. In particular embodiments, semi-solid or solid pharmaceutical compositions may comprise semi-permeable gels, lattices, cellular scaffolds and the like, which may be non-biodegradable or biodegradable. For example, in certain embodiments, it may be desirable or appropriate to sequester the exogenous cells from their surroundings, yet enable the cells to secrete and deliver biological molecules to surrounding cells. In these embodiments, cells may be formulated as autonomous implants comprising living PPDCs or cell population comprising PPDCs surrounded by a non-degradable, selectively permeable barrier that physically separates the transplanted cells from host tissue. Such implants are sometimes referred to as “immunoprotective,” as they have the capacity to prevent immune cells and macromolecules from killing the transplanted cells in the absence of pharmacologically induced immunosuppression (for a review of such devices and methods, see, e.g., P. A. Tresco et al., 2000, Adv. Drug Delivery Rev. 42: 3-27).

In other embodiments, different varieties of degradable gels and networks are utilized for the pharmaceutical compositions of the invention. For example, degradable materials particularly suitable for sustained release formulations include biocompatible polymers, such as poly (lactic acid), poly (lactic-co-glycolic acid), methylcellulose, hyaluronic acid, collagen, and the like. The structure, selection and use of degradable polymers in drug delivery vehicles have been reviewed in several publications, including, A. Domb et al., 1992, Polymers for Advanced Technologies 3:279-291. U.S. Pat. No. 5,869,079 to Wong et al. discloses combinations of hydrophilic and hydrophobic entities in a biodegradable sustained release ocular implant. In addition, U.S. Pat. No. 6,375,972 to Guo et al., U.S. Pat. No. 5,902,598 to Chen et al., U.S. Pat. No. 6,331,313 to Wong et al., U.S. Pat. No. 5,707,643 to Ogura et al., U.S. Pat. No. 5,466,233 to Weiner et al. and U.S. Pat. No. 6,251,090 to Avery et al. each describes intraocular implant devices and systems that may be used to deliver pharmaceutical compositions.

In other embodiments, e.g., for repair of neural lesions, such as a damaged or severed optic nerve, it may be desirable or appropriate to deliver the cells on or in a biodegradable, preferably bioresorbable or bioabsorbable, scaffold or matrix. These typically three-dimensional biomaterials contain the living cells attached to the scaffold, dispersed within the scaffold, or incorporated in an extracellular matrix entrapped in the scaffold. Once implanted into the target region of the body, these implants become integrated with the host tissue, wherein the transplanted cells gradually become established (see, e.g., P. A. Tresco et al., 2000, supra; see also D. W. Hutmacher, 2001, J. Biomater. Sci. Polymer Edn. 12: 107-174).

Examples of scaffold or matrix (sometimes referred to collectively as “framework”) material that may be used in the present invention include nonwoven mats, porous foams, or self-assembling peptides. Nonwoven mats may, for example, be formed using fibers comprised of a synthetic absorbable copolymer of glycolic and lactic acids (PGA/PLA), sold under the trade name VICRYL (Ethicon, Inc., Somerville, N.J). Foams, composed of, for example, poly (epsilon-caprolactone)/poly (glycolic acid) (PCL/PGA) copolymer, formed by processes such as freeze-drying, or lyophilized, as discussed in U.S. Pat. No. 6,355,699 also may be utilized. Hydrogels such as self-assembling peptides (e.g., RAD16) may also be used. In situ-forming degradable networks are also suitable for use in the invention (see, e.g., Anseth, K. S. et al., 2002, J. Controlled Release 78: 199-209; Wang, D. et al., 2003, Biomaterials 24: 3969-3980; U.S. Patent Publication 2002/0022676 to He et al.). These materials are formulated as fluids suitable for injection, and then may be induced by a variety of means (e.g., change in temperature, pH, exposure to light) to form degradable hydrogel networks in situ or in vivo.

In another embodiment, the framework is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another embodiment, cells are seeded onto foam scaffolds that may be composite structures.

In many of the abovementioned embodiments, the framework may be molded into a useful shape. Furthermore, it will be appreciated that PPDCs may be cultured on pre-formed, non-degradable surgical or implantable devices, e.g., in a manner corresponding to that used for preparing fibroblast-containing GDC endovascular coils, for instance (Marx, W. F. et al., 2001, Am. J. Neuroradiol. 22: 323-333).

The matrix, scaffold or device may be treated prior to inoculation of cells in order to enhance cell attachment. For example, prior to inoculation, nylon matrices can be treated with 0.1 molar acetic acid and incubated in polylysine, PBS, and/or collagen to coat the nylon. Polystyrene can be similarly treated using sulfuric acid. The external surfaces of a framework may also be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, and plant gums, among others.

Frameworks containing living cells are prepared according to methods known in the art. For example, cells can be grown freely in a culture vessel to sub-confluency or confluency, lifted from the culture and inoculated onto the framework. Growth factors may be added to the culture medium prior to, during, or subsequent to inoculation of the cells to trigger differentiation and tissue formation, if desired. Alternatively, the frameworks themselves may be modified so that the growth of cells thereon is enhanced, or so that the risk of rejection of the implant is reduced. Thus, one or more biologically active compounds, including, but not limited to, anti-inflammatory agents, immunosuppressants or growth factors, may be added to the framework for local release.

### Methods of Use

Progenitor cells, such as postpartum cells (preferably hUTCs or PDCs), or cell populations thereof, or conditioned medium or other components of or products produced by such cells, may be used in a variety of ways to support and facilitate repair and regeneration of ocular cells and tissues. Such utilities encompass in vitro, ex vivo and in vivo methods. The methods set forth below are directed to PPDCs, but other postpartum cells may also be suitable for use in those methods.

### In Vitro and Ex Vivo Methods

In one embodiment, progenitor cells, such as postpartum cells (preferably hUTCs or PDCs), and conditioned media generated therefrom may be used in vitro to screen a wide variety of compounds for effectiveness and cytotoxicity of pharmaceutical agents, growth factors, regulatory factors, and the like. For example, such screening may be performed on substantially homogeneous populations of PPDCs to assess the efficacy or toxicity of candidate compounds to be formulated with, or co-administered with, the PPDCs, for treatment of a an ocular condition. Alternatively, such screening may be performed on PPDCs that have been stimulated to differentiate into a cell type found in the eye, or progenitor thereof, for the purpose of evaluating the efficacy of new pharmaceutical drug candidates. In this embodiment, the PPDCs are maintained in vitro and exposed to the compound to be tested. The activity of a potentially cytotoxic compound can be measured by its ability to damage or kill cells in culture. This may readily be assessed by vital staining techniques.

As discussed above, PPDCs can be cultured in vitro to produce biological products that are either naturally produced by the cells, or produced by the cells when induced to differentiate into other lineages, or produced by the cells via genetic modification. For instance, TIMP1, TPO, KGF, HGF, FGF, HBEGF, BDNF, MIP1b, MCP1, RANTES, 1309, TARC, MDC, and IL-8 were found to be secreted from umbilicus-derived cells grown in Growth Medium. TIMP1, TPO, KGF, HGF, HBEGF, BDNF, MIP1a, MCP-1, RANTES, TARC, Eotaxin, and IL-8 were found to be secreted from placenta-derived PPDCs cultured in Growth Medium (see Examples).

In this regard, an embodiment of the invention features use of PPDCs for production of conditioned medium. Production of conditioned media from PPDCs may either be from undifferentiated PPDCs or from PPDCs incubated under conditions that stimulate differentiation. Such conditioned media are contemplated for use in in vitro or ex vivo culture of epithelial or neural precursor cells, for example, or in vivo to support transplanted cells comprising homogeneous populations of PPDCs or heterogeneous populations comprising PPDCs and other progenitors.

Cell lysates, soluble cell fractions or components from PPDCs, or ECM or components thereof, may be used for a variety of purposes. As mentioned above, some of these components may be used in pharmaceutical compositions. In other embodiments, a cell lysate or ECM is used to coat or otherwise treat substances or devices to be used surgically, or for implantation, or for ex vivo purposes, to promote healing or survival of cells or tissues contacted in the course of such treatments.

As described in Examples 14 and 16, PPDCs have demonstrated the ability to support survival, growth and differentiation of adult neural progenitor cells when grown in co-culture with those cells. Likewise, previous studies indicate that PPDCs may function to support cells of the retina via trophic mechanisms. (US 2010-0272803). Accordingly, PPDCs are used advantageously in co-cultures in vitro to provide trophic support to other cells, in particular neural cells and neural and ocular progenitors (e.g., neural stem cells and retinal or corneal epithelial stem cells). For co-culture, it may be desirable for the PPDCs and the desired other cells to be co-cultured under conditions in which the two cell types are in contact. This can be achieved, for example, by seeding the cells as a heterogeneous population of cells in culture medium or onto a suitable culture substrate. Alternatively, the PPDCs can first be grown to confluence, and then will serve as a substrate for the second desired cell type in culture. In this latter embodiment, the cells may further be physically separated, e.g., by a membrane or similar device, such that the other cell type may be removed and used separately, following the co-culture period. Use of PPDCs in co-culture to promote expansion and differentiation of neural or ocular cell types may find applicability in research and in clinical/therapeutic areas. For instance, PPDC co-culture may be utilized to facilitate growth and differentiation of such cells in culture, for basic research purposes or for use in drug screening assays, for example. PPDC co-culture may also be utilized for ex vivo expansion of neural or ocular progenitors for later administration for therapeutic purposes. For example, neural or ocular progenitor cells may be harvested from an individual, expanded ex vivo in co-culture with PPDCs, then returned to that individual (autologous transfer) or another individual (syngeneic or allogeneic transfer). In these embodiments, it will be appreciated that, following ex vivo expansion, the mixed population of cells comprising the PPDCs and progenitors could be administered to a patient in need of treatment. Alternatively, in situations where autologous transfer is appropriate or desirable, the co-cultured cell populations may be physically separated in culture, enabling removal of the autologous progenitors for administration to the patient.

### In Vivo Methods

As set forth in the Examples, conditioned media may effectively be used for treating an ocular degenerative condition. Once transplanted into a target location in the eye, conditioned media from progenitor cells, such as PPDCs provides trophic support for ocular cells in situ.

The conditioned media from progenitor cells, such as PPDCs may be administered with other beneficial drugs, biological molecules, such as growth factors, trophic factors, conditioned medium (from progenitor or differentiated cell cultures), or other active agents, such as anti-inflammatory agents, anti-apoptotic agents, antioxidants, growth factors, neurotrophic factors or neuroregenerative or neuroprotective drugs as known in the art. When conditioned media is administered with other agents, they may be administered together in a single pharmaceutical composition, or in separate pharmaceutical compositions, simultaneously or sequentially with the other agents (either before or after administration of the other agents).

Examples of other components that may be administered with progenitor cells, such as PPDCs, and conditioned media products include, but are not limited to: (1) other neuroprotective or neurobeneficial drugs; (2) selected extracellular matrix components, such as one or more types of collagen known in the art, and/or growth factors, platelet-rich plasma, and drugs (alternatively, the cells may be genetically engineered to express and produce growth factors); (3) anti-apoptotic agents (e.g., erythropoietin (EPO), EPO mimetibody, thrombopoietin, insulin-like growth factor (IGF)-I, IGF-II, hepatocyte growth factor, caspase inhibitors); (4) anti-inflammatory compounds (e.g., p38 MAP kinase inhibitors, TGF-beta inhibitors, statins, IL-6 and IL-I inhibitors, PEMIROLAST, TRANILAST, REMICADE, SIROLIMUS, and non-steroidal anti-inflammatory drugs (NSAIDS) (such as TEPDXALIN, TOLMETIN, and SUPROFEN); (5) immunosuppressive or immunomodulatory agents, such as calcineurin inhibitors, mTOR inhibitors, antiproliferatives, corticosteroids and various antibodies; (6) antioxidants such as probucol, vitamins C and E, conenzyme Q-10, glutathione, L-cysteine and N-acetylcysteine; and (6) local anesthetics, to name a few.

Liquid or fluid pharmaceutical compositions may be administered to a more general location in the eye (e.g., topically or intra-ocularly).

Other embodiments encompass methods of treating ocular degenerative conditions by administering pharmaceutical compositions comprising conditioned medium from progenitor cells, such as PPDCs, or trophic and other biological factors produced naturally by those cells or through genetic modification of the cells. Again, these methods may further comprise administering other active agents, such as growth factors, neurotrophic factors or neuroregenerative or neuroprotective drugs as known in the art.

Dosage forms and regimes for administering conditioned media from progenitor cells, such as PPDCs, or any of the other pharmaceutical compositions described herein are developed in accordance with good medical practice, taking into account the condition of the individual patient, e.g., nature and extent of the ocular degenerative condition, age, sex, body weight and general medical condition, and other factors known to medical practitioners. Thus, the effective amount of a pharmaceutical composition to be administered to a patient is determined by these considerations as known in the art.

It may be desirable or appropriate to pharmacologically immunosuppress a patient prior to initiating cell therapy. This may be accomplished through the use of systemic or local immunosuppressive agents, or it may be accomplished by delivering the cells in an encapsulated device, as described above. These and other means for reducing or eliminating an immune response to the transplanted cells are known in the art. As an alternative, conditioned media may be prepared from PPDCs genetically modified to reduce their immunogenicity, as mentioned above.

Survival of transplanted cells in a living patient can be determined through the use of a variety of scanning techniques, e.g., computerized axial tomography (CAT or CT) scan, magnetic resonance imaging (MRI) or positron emission tomography (PET) scans. Determination of transplant survival can also be done post mortem by removing the tissue and examining it visually or through a microscope. Alternatively, cells can be treated with stains that are specific for neural or ocular cells or products thereof, e.g., neurotransmitters. Transplanted cells can also be identified by prior incorporation of tracer dyes such as rhodamine- or fluorescein-labeled microspheres, fast blue, ferric microparticles, bisbenzamide or genetically introduced reporter gene products, such as beta-galactosidase or beta-glucuronidase.

Functional integration of transplanted cells or conditioned medium into ocular tissue of a subject can be assessed by examining restoration of the ocular function that was damaged or diseased. For example, effectiveness in the treatment of macular degeneration or other retinopathies may be determined by improvement of visual acuity and evaluation for abnormalities and grading of stereoscopic color fundus photographs. (Age-Related Eye Disease Study Research Group, NEI, NIH, AREDS Report No. 8, 2001, Arch. Ophthalmol. 119: 1417-1436).

### Kits and Banks

In another aspect, the invention provides kits that utilize progenitor cells, such as PPDCs, and cell populations, conditioned medium prepared from the cells, preferably from PPDCs, and components and products thereof in various methods for ocular regeneration and repair as described above. Where used for treatment of ocular degenerative conditions, or other scheduled treatment, the kits may include one or more cell populations or conditioned medium, including at least postpartum cells or conditioned medium derived from postpartum cells, and a pharmaceutically acceptable carrier (liquid, semi-solid or solid). The kits also optionally may include a means of administering the cells and conditioned medium, for example by injection. The kits further may include instructions for use of the cells and conditioned medium. Kits prepared for field hospital use, such as for military use may include full-procedure supplies including tissue scaffolds, surgical sutures, and the like, where the cells or conditioned medium are to be used in conjunction with repair of acute injuries. Kits for assays and in vitro methods as described herein may contain, for example, one or more of: (1) PPDCs or components thereof, or conditioned medium or other products of PPDCs; (2) reagents for practicing the in vitro method; (3) other cells or cell populations, as appropriate; and (4) instructions for conducting the in vitro method.

In yet another aspect, the invention also provides for banking of tissues, cells, cell populations, conditioned medium, and cellular components of the invention. As discussed above, the cells and and conditioned medium are readily cryopreserved. The invention therefore provides methods of cryopreserving the cells in a bank, wherein the cells are stored frozen and associated with a complete characterization of the cells based on immunological, biochemical and genetic properties of the cells. The frozen cells can be thawed and expanded or used directly for autologous, syngeneic, or allogeneic therapy, depending on the requirements of the procedure and the needs of the patient. Preferably, the information on each cryopreserved sample is stored in a computer, which is searchable based on the requirements of the surgeon, procedure and patient with suitable matches being made based on the characterization of the cells or populations. Preferably, the cells of the invention are grown and expanded to the desired quantity of cells and therapeutic cell compositions are prepared either separately or as co-cultures, in the presence or absence of a matrix or support. While for some applications it may be preferable to use cells freshly prepared, the remainder can be cryopreserved and banked by freezing the cells and entering the information in the computer to associate the computer entry with the samples. Even where it is not necessary to match a source or donor with a recipient of such cells, for immunological purposes, the bank system makes it easy to match, for example, desirable biochemical or genetic properties of the banked cells to the therapeutic needs. Upon matching of the desired properties with a banked sample, the sample is retrieved and prepared for therapeutic use. Cell lysates, ECM or cellular components prepared as described herein may also be cryopreserved or otherwise preserved (e.g., by lyophilization) and banked in accordance with the present invention.

The following examples are provided to describe the invention in greater detail. They are intended to illustrate, not to limit, the invention.

The following abbreviations may appear in the examples and elsewhere in the specification and claims: ANG2 (or Ang2) for angiopoietin 2; APC for antigen-presenting cells; BDNF for brain-derived neurotrophic factor; bFGF for basic fibroblast growth factor; bid (BID) for “bis in die” (twice per day); CK18 for cytokeratin 18; CNS for central nervous system; CXC ligand 3 for chemokine receptor ligand 3; DMEM for Dulbecco's Minimal Essential Medium; DMEM:Ig (or DMEM:Lg, DMEM:LG) for DMEM with low glucose; EDTA for ethylene diamine tetraacetic acid; EGF (or E) for epidermal growth factor; FACS for fluorescent activated cell sorting; FBS for fetal bovine serum; FGF (or F) for fibroblast growth factor; GCP-2 for granulocyte chemotactic protein-2; GDNF for glial cell-derived neurotrophic factor; GF AP for glial fibrillary acidic protein; HB-EGF for heparin-binding epidermal growth factor; HCAEC for Human coronary artery endothelial cells; HGF for hepatocyte growth factor; hMSC for Human mesenchymal stem cells; HNF-1alpha for hepatocyte-specific transcription factor; HVVEC for Human umbilical vein endothelial cells; 1309 for a chemokine and the ligand for the CCR8 receptor; IGF-1 for insulin-like growth factor 1; IL-6 for interleukin-6; IL-8 for interleukin 8; K19 for keratin 19; K8 for keratin 8; KGF for keratinocyte growth factor; LIF for leukemia inhibitory factor; MBP for myelin basic protein; MCP-1 for monocyte chemotactic protein 1; MDC for macrophage-derived chemokine; MIP1alpha for macrophage inflammatory protein 1 alpha; MIP1beta for macrophage inflammatory protein 1 beta; MMP for matrix metalloprotease (MMP); MSC for mesenchymal stem cells; NHDF for Normal Human Dermal Fibroblasts; NPE for Neural Progenitor Expansion media; NT3 for neurotrophin 3; 04 for oligodendrocyte or glial differentiation marker 04; PBMC for Peripheral blood mononuclear cell; PBS for phosphate buffered saline; PDGF-CC for platelet derived growth factor C; PDGF-DD for platelet derived growth factor D; PDGFbb for platelet derived growth factor bb; P0 for “per os” (by mouth); PNS for peripheral nervous system; Rantes (or RANTES) for regulated on activation, normal T cell expressed and secreted; rhGDF-5 for recombinant human growth and differentiation factor 5; SC for subcutaneously; SDF-1alpha for stromal-derived factor 1 alpha; SHH for sonic hedgehog; SOP for standard operating procedure; TARC for thymus and activation-regulated chemokine; TCP for Tissue culture plastic; TCPS for tissue culture polystyrene; TGFbeta2 for transforming growth factor beta2; TGF beta-3 for transforming growth factor beta-3; TIMP1 for tissue inhibitor of matrix metalloproteinase 1; TPO for thrombopoietin; TUJ1 for BIII Tubulin; VEGF for vascular endothelial growth factor; vWF for von Willebrand factor; and alphaFP for alpha-fetoprotein.

The present invention is further illustrated, but not limited by, the following examples.

### Example 1

### Effect of Umbilicus-Derived Cell Conditioned Media to Rescue Phagocytosis Activity In Vitro with Dystrophic RPE Cells

It is well established that retinal pigment epithelium (RPE) cells from Royal College of Surgeons (RCS) rat exhibit impaired rod outer segment (ROS) phagocytosis due to a null mutation in the Mertk gene. (Feng W. et al., J Biol Chem. 2002, 10: 277 (19): 17016-17022). It has been shown that injection of human umbilical tissue derived cell (hUTC) subretinally into RCS rat eye improved visual acuity and appear to ameliorate retinal degeneration. (US 2010/0272803). In this example, treatment with conditioned medium (CM) derived from hUTC restored phagocytosis of ROS in dystrophic RPE cells in vitro.

hUTC conditioned medium was examined: 1) to evaluate the effect on dystrophic RPE phagocytosis using new preparations of hUTC CM; 2) to isolate RNA of acceptable quality from CM-treated and untreated dystrophic RPE for use in gene expression profiling by RNA-Seq; 3) to examine whether selected RTK ligands can increase the level of phagocytosis in RPE from the RCS rat that cannot use the Mertk signaling pathway; and 4) to test whether other non-RTK ligands, which activate different receptors from RTK, could exhibit similar function.

### Materials and Methods

Human umbilical tissue derived cell (hUTC) were obtained from the methods described in Examples 6-18 following, and in detail in U.S. Pat. Nos. 7,524,489, and 7,510,873, and U.S. Pub. App. No. 2005/0058634, each incorporated by reference herein. Briefly, human umbilical cords were obtained with donor consent following live births from the National Disease Research Interchange (Philadelphia, Pa.). Tissues were minced and enzymatically digested. After almost complete digestion with a Dulbecco's modified Eagle's medium (DMEM)-low glucose (Lg) (Invitrogen, Carlsbad, Calif.) medium containing a mixture of 50 U/mL collagenase (Sigma, St. Louis), the cell suspension was filtered through a 70 [tm filter, and the supernatant was centrifuged at 350 g. Isolated cells were washed in DMEM-Lg a few times and seeded at a density of 5,000 cells/cm² in DMEM-Lg medium containing 15% (v/v) FBS (Hyclone, Logan, Utah) and 4 mM L-glutamine (Gibco, Grand Island, N.Y.). When cells reached approximately 70% confluence, they were passaged using TrypLE (Gibco, Grand Island, N.Y.). Cells were harvested after several passages and banked.

Primary Culture of RPE Cells:

RPE cells were obtained from 6-11 day old pigmented normal (RCS rdy+/p+) (congenic control) or dystrophic (RCS rdy−/p+) rats. The anterior part of the eye was removed anterior to the limbus. The retina was gently removed and the eye cup was incubated in 4% (w/v) dispase (>0.8 U/mg, Roche Diagnostics, Mannheim, Germany) for 20-30 minutes. The RPE sheets were removed, suspended in growth medium (DMEM+10% FBS [new paper 20%]+Pen (200 U/ml)/Strep (200 μg/ml)), triturated with trypsin treatment, and plated in either a 8-well chamber slide well or on a circular glass cover slip placed in a well of a 24-well dish. The cells were incubated at 37° C. in 5% v/v CO2.

Sulforhodamine Staining of RPE Cells:

RPE culture were maintained for 24 h to 72 h in growth medium containing sulforhodamine (40 mg/ml final concentration). The cells were stained 36 h to 48 h before the addition of ROS. The sulforhodamine-containing medium was removed 6 h to 18 h before the addition of ROS, and the culture was maintained in several changes of fresh growth medium.

Isolation of Rat Photoreceptor OS:

Eyes were obtained from 2-4 or 6-8-week old Long Evans rats several hours after light onset. Retinas were isolated, homogenized with Polytron (8 mm generator) or a Dounce glass homogenizer, layered on top of 27%-50% linear sucrose gradient, and centrifuged at 38,000 rpm in SW41 rotor (240,000×g) for 1 hour at 4° C. The top two ROS bands were collected, diluted with HBSS, and centrifuged at 7000 rpm in HB-4 rotor (8000×g) for 10 minutes to pellet the ROS.

FITC Staining of ROS:

The ROS pellet was resuspended in serum-free culture medium (MEM basal medium only) at about 1 ml per pellet. The FITC stock solution (2 mg/ml in 0.1M sodium bicarbonate, pH 9.0-9.5) was added to a final concentration of 10 mg/ml and incubated at room temperature for 1 h. The FITC-stained ROS is pelleted by centrifugation in a microfuge at 8000 rpm, 10 minutes, resuspended in growth medium (MEM20), counted, and diluted to a final concentration of 107/ml.

hUTC Conditioned Medium (CM):

Three sets of hUTC conditioned medium (CM) and a control medium were prepared and used for testing in the phagocytosis assay.

1. CM1 with Serum Preparation

On day 1, hUTC was seeded at 5,000 viable cells/cm² in T75 cell culture flask in hUTC growth medium (DMEM low glucose+15% FBS+4 mM L-glutamine). Cells were cultured for 24 hours in 37° C., 5% CO2 incubator. On day 2, medium was aspirated, washed twice with DPBS, and replenished with 21 mL of DMEM/F12 complete medium (DMEM:F12 medium+10% FBS+Pen (50 U/ml)/Strep (50 μg/ml)). Cells were cultured for another 54 hours. Control medium (DMEM:F12 medium+10% FBS+Pen (50 U/ml)/Strep (50 μg/ml)) alone was also cultured for 54 h. On day 4, cell culture supernatant and control medium were collected and centrifuged at 250 g, 5 min at RT, aliquoted in cryotube at 3 mL/tube, and frozen immediately at −70° C. freezer.

2. CM1-Serum Free Preparation

On day 1, hUTC was seeded at 5,000 viable cells/cm² in T75 cell culture flask in hUTC growth medium (DMEM low glucose+15% FBS+4 mM L-glutamine). Cells were cultured for 24 hours in 37° C., 5% CO2 incubator. On day 2, medium was aspirated, washed twice with DPBS, and replenished with 21 mL of DMEM/F12 serum-free medium (DMEM:F12 medium+Pen (50 U/ml)/Strep (50 μg/ml)). Cells were cultured for another 54 h. Serum-free control medium (DMEM:F12 medium+Pen (50 U/ml)/Strep (50 μg/ml)) alone was also cultured for 54 h. On day 4, cell culture supernatant and control medium were collected and centrifuged at 250 g, 5 min at RT, aliquoted in cryotube at 3 mL/tube, and frozen immediately at −70° C. freezer.

3. CM2 Preparation

The same preparation as CM1 with serum preparation and hUTC seeded at 5,000 viable cells/cm², except the cell culture flasks are T225 flasks with 63 mL of medium per flask, and the incubation time after medium change was 48 hours.

4. CM3 Preparation

The same preparation as CM2, except the hUTC seeding density was increased to 10,000 viable cells/cm², and the incubation time after medium change was 48 hours.

Phagocytosis Assay:

5×10⁴ sulforhodamine-stained RPE cells were plated in multi-well plate, maintained in MEM+20% (v/v) FBS for 6 days, then in MEM+5% (v/v) FBS 24 h before the assay (2 or more replicates per sample). Fresh medium was added 3 h before the addition of ROS, and the assay was started by overlaying the culture with FITC-ROS (10⁷/ml in MEM+20% (v/v) FBS) and incubating at 37° C. for 3 to 19 h (8 h typically). At the end of the incubation, the cells were vigorously washed to remove unbound ROS and fixed with 2% (w/v) paraformaldehyde (Sigma, St. Louis, Mo.).

RPE phagocytosis of ROS was optimized with respect to the protocols for preparation and culture of primary RPE, preparation of ROS, and the phagocytosis assay itself.

RTK ligands:

The RTK ligands used were: Recombinant Human Ephrin-B2 (Cat # pro-937, Lot #1112PEFNB2, ProSpec-Tany TechnoGene Ltd., Israel), Recombinant Human BDNF (Cat #248-BD-025/CF, Lot # NG4012051, R&D Systems, Inc., Minneapolis, Minn.), Recominant Human HB-EGF (Cat #259-HE-050/CF, Lot # JI3012021, R&D Systems, Inc., Minneapolis, Minn.), Recombinant Human HGF (Cat # PHG0254, Lot #73197181A, Life Technologies, Carlsbad, Calif.), Recombinant Human Ephrin A4 (Cat # E199, Lot #1112R245, Leinco Technologies, Inc., St. Louis, Mo.), and Recombinant Human PDGF-DD (Cat #1159-SB-025/CF, Lot # OTH0412071, R&D Systems, Inc., Minneapolis, Minn.). Reconstitution of individual RTK ligand stock solution followed the vendors' data sheets: Recombinant Human BDNF and HB-EGF were reconstituted at 100 μg/mL and 250 μg/mL in sterile PBS, respectively. Recombinant Human HGF was reconstituted at 500 μg/mL in sterile, distilled water. Recombinant Human Ephrin A4 was reconstituted at 100 μg/mL in sterile PBS. Recombinant Human PDGF-DD was reconstituted at 100 μg/mL in sterile 4 mM HCl. The reconstituted stocks were aliquoted and frozen at −70° C. freezer.

The culture media was changed to MEM+5% (v/v) FBS (MEM5), and ligands were added to the dystrophic cells at 200 ng/ml, incubated for 24 h followed by the addition of ROS in the presence of the ligands, and the cells were subjected to phagocytosis assay. Normal RPE replicates were preincubated in MEM5 and assayed for phagocytosis as controls.

Non-RTK Ligands:

Non-RTK ligands used were: Recombinant Human Vitronectin (Cat #2308-VN-050, Lot # NBH0713021, R&D Systems, Inc., Minneapolis, Minn.), Recombinant Human TGF-β1 (Cat #240-B-010/CF, Lot # AV5412113, R&D Systems, Inc., Minneapolis, Minn.), Recombinant Human IL-6 (Cat #206-IL-010/CF, Lot # OJZ0712041, R&D Systems, Inc., Minneapolis, Minn.), and Human Endothelin-1 (Cat # hor-307, Lot #1211PEDN112, ProSpec-Tany TechnoGene Ltd., Israel). Reconstitution of individual non-RTK ligand stock solution followed the vendors' data sheets: Recombinant Human Vitronectin and IL-6 were reconstituted at 100 μg/mL in sterile PBS, respectively. Recombinant Human TGF-β1 was reconstituted at 100 μg/mL in sterile 4 mM HCl. Recombinant Human Endothelin-1 was reconstituted at 100 μg/mL in sterile 18MΩ-cm H₂O. The reconstituted stocks were aliquoted and frozen at −70° C. freezer.

Assay of Conditioned Media for Phagocytosis Rescue Activity:

Replicates of dystrophic (D) RPE were incubated with 1 ml each of conditioned media for about 24 h. For control, replicates of congenic control (N) RPE were incubated in Control medium (DMEM:F12 medium+10% FBS+Pen (50 U/ml)/Strep (50 μg/ml)) for the same time. After the incubation, the conditioned media and control media were removed, replaced with fresh MEM5, and the RPE were subjected to phagocytosis assay.

Assays to Examine Effects of Non-RTK Ligands on RCS RPE Cell Phagocytosis:

hUTC CM3 was used as a positive control for the assays. For tests of endothelin-1, TGF-β1 and IL-6, dystrophic (D) RPE were incubated with 1 ml each of conditioned media for 24 h. Conditioned media was then removed, replaced with fresh MEM+5% (v/v) FBS (MEM5) and subject to phagocytosis assay (fed with ROS for 8 h in MEM5). For other controls, normal and dystrophic RPE were incubated in MEM5 for 24 h and subject to phagocytosis assay. Dystrophic RPE cells were incubated with recombinant human endothelin-1, TGF-β1 or IL-6 at 200 ng/mL in MEM5 for 24 h and then subjected to phagocytosis assay with the addition of ROS in MEM5 containing recombinant human endothelin-1, TGF-β1 or IL-6 (medium was not changed when adding ROS).

For tests of vitronectin, ROS was preincubated with control medium (DMEM+10% FBS) or conditioned media for 24 h in CO2 cell culture incubator at 37° C. In parallel, ROS was preincubated in MEM+20% (v/v) FBS (MEM20) with various concentrations of human recombinant vitronectin (4, 2, 1, 0.5 μg/ml) respectively for 24 h in CO2 cell culture incubator at 37° C. After the incubation, the ROS was spun down without wash, resuspended in MEM20 and fed to the dystrophic RPE cells in the presence of MEM5 for phagocytosis assay. For controls, normal RPE alone or dystrophic RPE alone was cultured in MEM20, then changed to MEM5 in the presence of untreated ROS (resuspended in MEM20 and fed to RPE cells) for phagocytosis assay.

Imaging and Quantitation:

The living RPE cells were examined by phase contrast and fluorescence microscopy using an inverted microscope equipped with epifluorescence optics, a fluorescence microscope, and a digital camera. FITC-ROS bound to the cell surface, ingested FITC-ROS, and phagolysosomes were identified as defined in McLaren et al. (Invest Ophthalmol Vis Sci., 1993; 34(2):317-326.). Quantitation of bound and ingested ROS was performed on fixed cells on cover slips. Counts were made at 250× magnification with the appropriate filters and a grid (field size, 40×40 μm). Representative fields (10 to 15 per culture) were counted for various types of cells, and the data were expressed as the mean of pooled values obtained per time point from 2-3 separate experiments. Statistical significance was assessed by Student's t-test for paired data, and was considered at p<0.05.

Assay Acceptance Criteria:

The absolute level of phagocytosis varies in experiments depending on a multitude of factors, including the quality of isolated RPE and prepared ROS. Effort was made to use RPE of the same lineage, i.e., time of harvest and preparation, for comparison of the effects of different treatments on the cells. The assay was judged to be legitimate if the relationship of the phagocytosis level in the normal compared to the dystrophic is approximately 1:0.3.

Relative Phagocytosis:

Relative phagocytosis is the level of phagocytosis shown by dystrophic RPE compared to the congenic control (normal) as the reference point. The level of phagocytosis could be expressed as a mean number of ROS/field, or as a mean number of ROS/cell.

Isolation of RNA.

hUTC was seeded at 5,000 cells/cm² and grown in DMEM-Lg medium containing 15% (v/v) FBS (Hyclone, Logan, Utah) and 4 mM L-glutamine (Gibco, Grand Island, N.Y.) for 24 hours followed by medium change to DMEM:F12 medium containing 10% (v/v) FBS and grown for another 48 hours. Cells were then collected for total RNA extraction and DNA removal using the Qiagen RNAeasy extraction and on-column DNAse kit (Qiagen, Valencia, Calif.). The integrity and quantity of RNA in the samples was determined using NanoDrop 1000 spectrophotometer (Thermo Fisher Scientific, ‘Waltham, Mass.) and Agilent 2100 Bioanalyzer (Agilent Technologies, Santa Clara, Calif.). Library preparation and sequencing were performed by Expression Analysis Inc., Durham, N.C. RNA libraries were prepared using Illumina's TruSeq RNA-Seq Sample Prep kit following manufacturer's instructions, and sequenced with Illumina's HiSeq 2000. Sequencing reads were mapped to the reference human genome (GRCh37 patch8) using the software ArrayStudio version 6.1. Fragments Per Kilobase of transcript per Million mapped reads (FPKM) was used to calculate gene expression.

After isolation, RCS RPE cells were first seeded in 96-well plate in triplicates for about a week, the time point when the medium was changed to either hUTC conditioned media or control medium was considered as 0 h. RNA was extracted at 2, 4, 8 and 24 h, respectively, using Trizol (Life Technologies, Carlsbad, Calif.) according to the manufacturer's protocol. RNA extracted from triplicate at each time point was pooled as one sample. The concentrations of the samples were determined spectrophotometrically, along with the 260/280 absorbance ratio.

Replicates of dystrophic RPE (˜2.4×10⁴ each) were treated with or without conditioned media as shown in the scheme below:

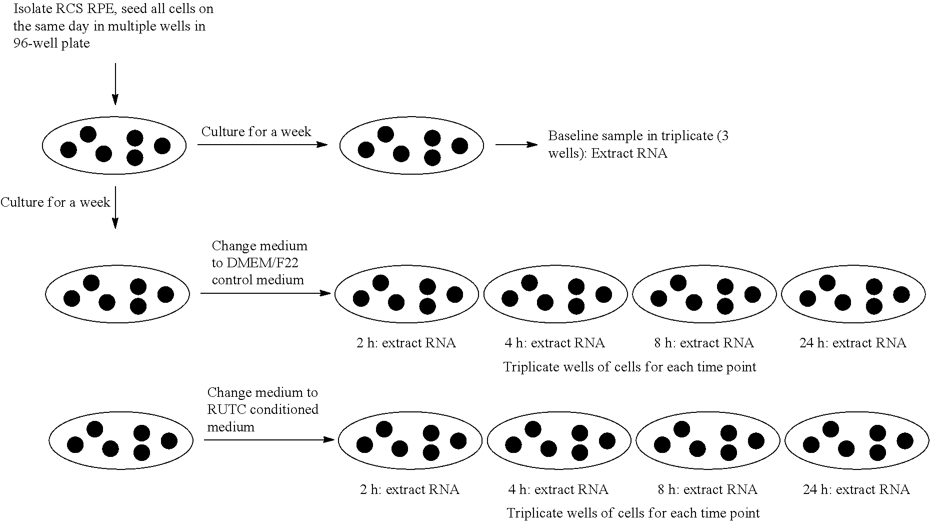

### Results

### Conditioned Media Tests

Three conditioned media preps (CM1, CM2, CM3) were tested for their phagocytosis rescue activities with dystrophic RPE and comparison with normal RPE as described in Methods.

CM1: CM1 was tested twice (FIGS. 1A and 1B), once using tan normal RPE, and again using pigmented RPE. Phagocytosis rescue activity was observed. It should be noted that in FIG. 1A, the basal level of phagocytosis in pigmented dystrophic RPE was almost 50% of the level of tan hooded normal RPE, which falls beyond the acceptance range (<30% of normal phagocytosis level). Effect of CM1-serum free medium was also tested (FIG. 2). The difference between dystrophic cells with and without CM1-serum free was statistically significant.

CM2: CM2 was tested and found to lack activity (FIG. 3).

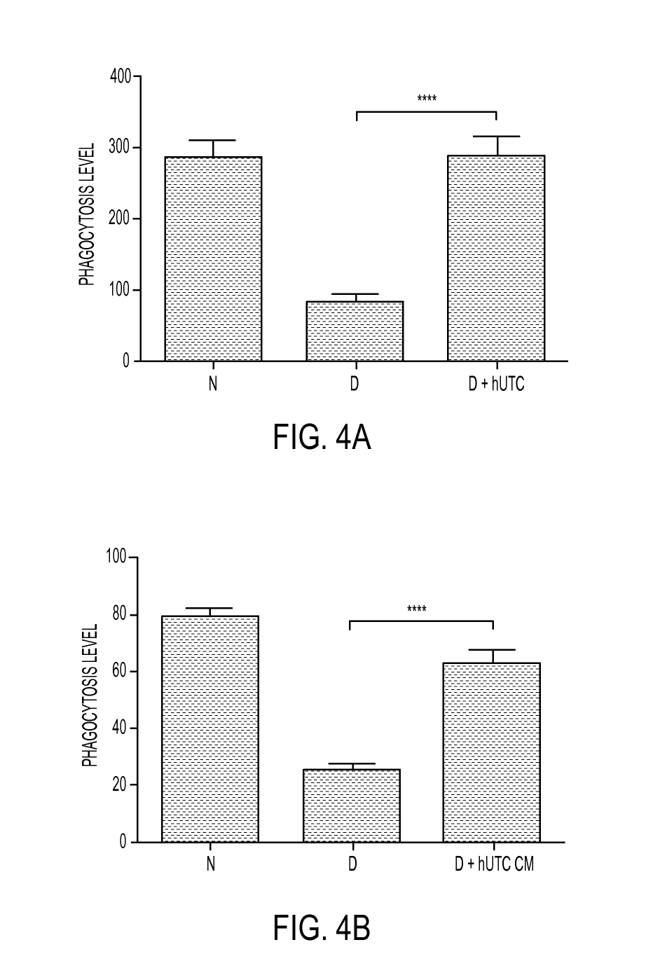

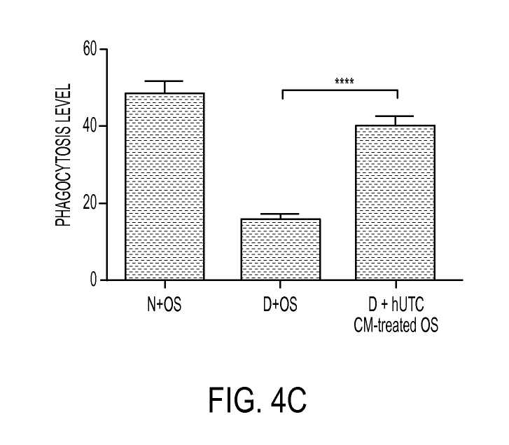

CM3: After the medium change on day 1, the incubation time for CM2 (not active) was 48 h whereas the incubation time for CM1 (active) was 54 h. To obtain an active conditioned media, initial cell seeding density and cell incubation time after medium change are two aspects to be considered. CM3 was prepared by doubling the cell seeding density with the same incubation time after medium change, compared to CM2. CM3 was assayed multiple times to confirm the presence of activity (FIGS. 4A, 4B, 4C), and showed a phagocytosis rescue activity of up to 100%.

### Phagocytosis in RCS RPE

Isolated RPE cells from RCS rat eyes were cultured in vitro for phagocytosis assay. Untreated RPE cells from normal congenic control rat eye were used as control to show normal level of phagocytosis. The defective phagocytosis in RCS RPE was restored to the level of normal RPE when the RCS RPE are co-cultured with hUTC (FIG. 4A) or treated with hUTC conditioned medium (CM) (FIG. 4B). Phagocytosis of OS by RCS RPE was rescued when the cells were fed with OS pre-incubated with hUTC CM and subjected to phagocytosis in the absence of hUTC CM (FIG. 4C). hUTC as demonstrated secrete specific factors to promote RPE phagocytosis.

### RTK Ligand Assays

BDNF and HB-EGF Assays:

Duplicates of dystrophic RPE were treated with BDNF and HB-EGF and assayed for phagocytosis as described in Methods, along with normal controls (FIGS. 5A, 5B). Ten to twelve observations were made per sample. The dystrophic cells tended to show a higher rate of phagocytosis than usual compared to normal cells, but it did not prevent the interpretation of the results. BDNF showed phagocytosis rescue activity, higher than that of CM3.

PDGF-DD, Ephrin A4, and HGF Assays:

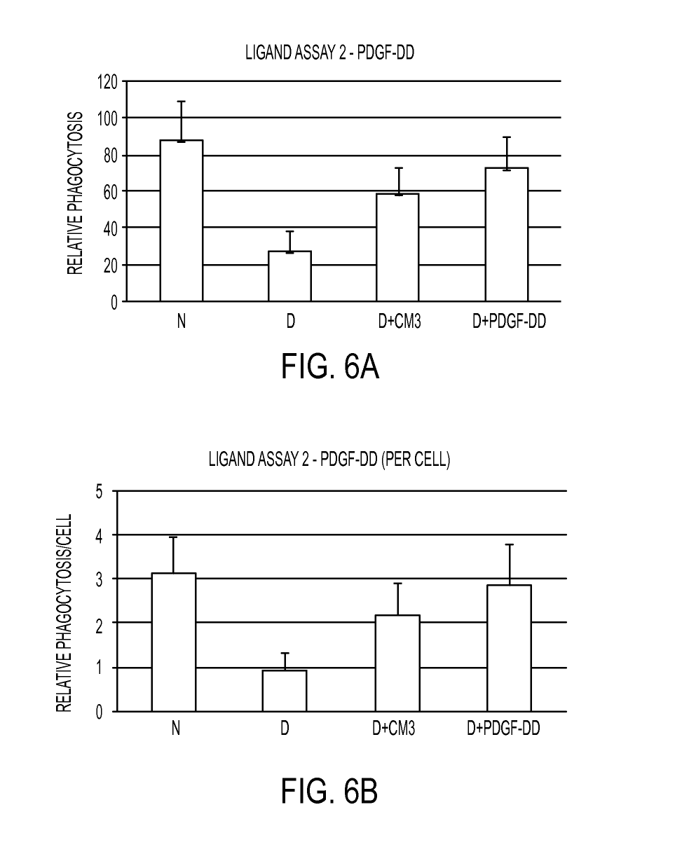

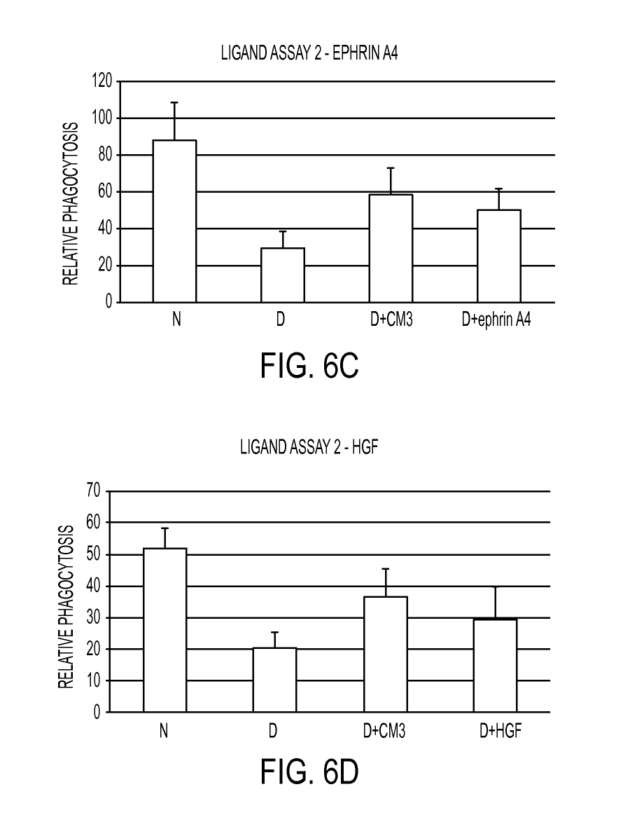

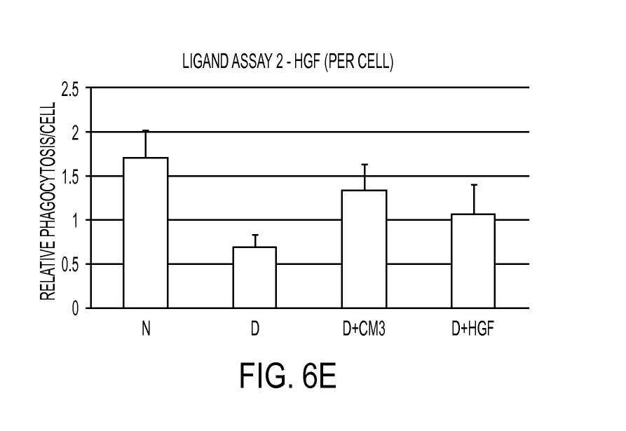

The number of cells being counted for every sampling were expressed as both per field (FIGS. 6A, 6C, 6D) and per cell (FIGS. 6B, 6E). The results were not affected, since the number of cells counted for every frame was constant. PDGF-DD (FIG. 6A), Ephrin A4 (FIG. 6C) and HGF (FIG. 6D) all upregulated phagocytosis in dystrophic RPE cells compared to untreated control. PDGF-DD showed the most rescue effect, greater than that of CM3.

Ephrin B2 Assay:

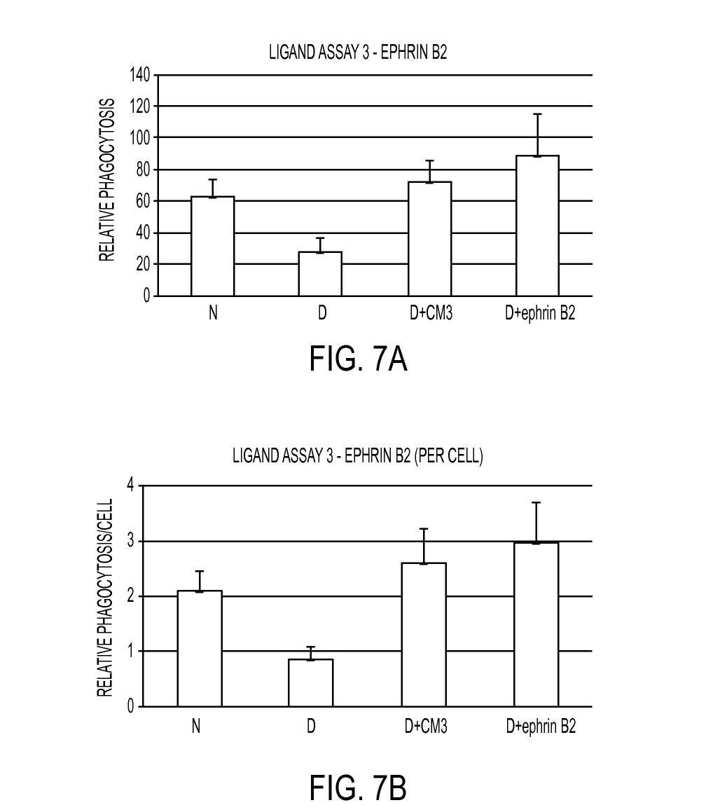

Ephrin B2 showed a very high phagocytosis rescue activity, higher than that of CM3. Both per field (FIG. 7A) and per cell (FIG. 7B) results were determined.

### Non-RTK Ligand Assays

Effect of Endothelin-1, TGF-β1 or IL-6 on RCS RPE Phagocytosis:

Dystropic RPE cells were treated with endothelin-1, TGF-β1 or IL-6 and assayed for phagocytosis as described in Materials and Methods, along with normal controls (FIGS. 8A-8C). Ten observations were made per sample. FIGS. 8A and 8B show two separate assays. The basal phagocytosis level of normal and dystrophic RPE cells in FIG. 8B is lower than that in FIG. 8A due to the variation of cells isolated from rats and different preparation of ROS; the assay is considered valid as the relationship of the phagocytosis level in the normal compared to the dystrophic is approximately 1:0.3. To compare with the results in FIG. 8A, the data in FIG. 8C was normalized so that the phagocytosis level of the normal and dystrophic RPE cells were the same as those from FIG. 8A. hUTC CM3 increased phagocytosis in dystrophic RPE cells, while endothelin-1, TGF-β1 or IL-6, at the concentration (200 ng/mL) tested in the assays, had no effect on RCS RPE phagocytosis.

Effect of Vitronectin on RCS RPE Phagocytosis:

Dystrophic RPE cells were fed with ROS preincubated with various concentrations of vitronectin and assayed for phagocytosis as described in Materials and Methods, along with normal controls (FIG. 9). Ten observations were made per sample.

As for the control medium and hUTC conditioned media used for the study, control medium contained 10% FBS, the amount of vitronectin in the control medium was 500 ng/ml. hUTC conditioned media may contain more vitronectin compared to control medium as hUTC constitutively secretes vitronectin. In contrast to ROS pretreated with hUTC CM3, ROS pretreated with control medium appears to have had no effect on dystrophic RPE phagocytosis. Vitronectin, at all the concentrations tested, had a similar effect to that of the control medium. These results are consistent with results reported in previous publications (Edwards et al., J Cell Physiol. 1986, 127: 293-296; Miceli et al., Invest Ophthalmol Vis Sci., 1997; 38(8): 1588-1597). Vitronectin is an active component of serum and is responsible for serum-stimulated uptake of ROS by cultured RPE cells isolated from human donor eyes (Miceli et al., 1997). However, for rat RPE cells, the effect of serum on phagocytosis in normal RPE compared to dystrophic RCS RPE is different. Edwards et al. showed that cultured RCS rat RPE cells and normal congenic control RPE cells phagocytized comparable low amounts of ROS in serum-free medium. The presence of 20% of serum in medium dramatically increased (6-fold) phagocytosis in normal RPE cells, but not in RCS RPE cells (Edwards et al., J Cell Physiol. 1986, 127: 293-296)

The current results indicate that vitronectin is not involved in hUTC CM-mediated enhancement of phagocytosis in RCS RPE cells.

Isolation of RNA from Conditioned Media-Treated Dystrophic RPE

Several rounds of this experiment were performed as described in Methods to obtain the necessary RNA samples. The RNA samples were used for RNA sequencing by Expression Analysis, Inc. For experiment 1, the RN scores of sample 8 and 9 did not meet the RNA sequencing criteria. Both samples were removed from the sequencing list. For experiment 2, the RIN score of sample 1 did not meet the RNA sequencing criteria and was removed from the sequencing list. (Sequencing results below).

### Experiment 1

| Tube | 260/280 | conc | vol | amt | for RIN | RIN |
|---|---|---|---|---|---|---|
| 1. control | 2.12 | 93 ng/ul | 57 ul | 5.2 ug | 93 ng/5 ul | 8.6 |
| 2. 2 h con med | 1.91 | 64 ng/ul | 57 ul | 3.6 ug | 64 ng/5 ul | 8.6 |
| 3. 2 h CM | 1.69 | 34 ng/ul | 57 ul | 1.9 ug | 34 ng/5 ul | 8.6 |
| 4. 4 h con med | 1.75 | 44 ng/5 ul | 57 ul | 2.5 ug | 44 ng/5 ul | 6.9 |
| 5. 4 h CM | 1.90 | 34 ng/5 ul | 57 ul | 1.9 ug | 34 ng/5 ul | 8.3 |
| 6. 8 h con med | 1.94 | 46 ng/5 ul | 57 ul | 2.6 ug | 46 ng/5 ul | 8.5 |
| 7. 8 h CM | 1.90 | 45 ng/5 ul | 57 ul | 2.6 ug | 45 ng/5 ul | 7.7 |
| 8. 24 h con | 1.94 | 53 ng/5 ul | 57 ul | 3.0 ug | 53 ng/5 ul | N/A |
| med | | | | | | |
| 9. 24 h CM | 1.84 | 55 ng/5 ul | 57 ul | 3.1 ug | 55 ng/5 ul | N/A |

### Experiment 2

| Tube | 260/280 | concentration | volume | amount | RIN |
|---|---|---|---|---|---|
| Untreated | 2.11 | 145 ng/ul | 19 ul | 2.8 ug | 1.0 |
| control | | | | | |
| 2h control | 2.00 | 153 ng/ul | 19 ul | 2.9 ug | 9.1 |
| 2h CM3 | 2.04 | 237 ng/ul | 19 ul | 4.5 ug | 8.7 |
| 4h control | 2.00 | 120 ng/ul | 19 ul | 2.3 ug | 8.8 |
| 4h CM3 | 2.05 | 179 ng/ul | 19 ul | 3.4 ug | 9.1 |
| 8h control | 2.13 | 158 ng/ul | 19 ul | 3.0 ug | 9.0 |
| 8h CM3 | 2.20 | 181 ng/ul | 19 ul | 3.4 ug | 9.4 |
| 24h control | 2.07 | 124 ng/ul | 19 ul | 2.4 ug | 8.8 |
| 24h CM3 | 2.20 | 154 ng/ul | 19 ul | 2.9 ug | 8.1 |

hUTC expression of the genes of RTK ligands and bridge molecules was shown by RNA-Seq-based transcriptome profiling of hUTC. Gene expression of multiple RTK ligands for 15 RTK subfamilies were detected (Table 1-1). The expression level of the RTK ligand genes for each RTK subfamily were sorted and graphed based on the values of Fragments Per Kilobase of transcript per Million mapped reads (FPKM) (FIG. 10; Table 1-1). Gene expression of bridge molecules, including MFG-E8, Gash, protein S, TSP-1 and TSP-2, were also detected (Table 1-3).

Gene expression for the corresponding RTK subfamilies and receptors for the bridge molecules in RCS RPE show RTK superfamily can be grouped into 20 subfamilies based on kinase domain sequences (Robinson D R, et al., Oncogene. 2000; 19(49):5548-5557). Gene expression of 18 out of 20 RTK subfamilies were detected in RCS RPE (Table 1-2). Among the 18 are the 15 RTK subfamilies corresponding to the RTK ligand genes expressed in hUTC. Gene expression of receptors reported for bridge molecule binding (Kevany B M, et al., Physiology, 2010; 25(1):8-15) were also detected in RCS RPE (Table 1-4), including integrin avP3, avP5, Ax1, Tyro3, MerTK, and CD36.

TABLE 1-1
Identification of RTK ligand gene expression in hUTC by RNA-Seq

| Ligands for | | RTK ligand gene | | |
|---|---|---|---|---|
| RTK | Gene | expression level | | |
| subfamilies | symbol | (FPKM) in hUTC | Gene synonyms | Gene full name |
| Ligands for DDR | COL1A1 | 1877.890 | 014 | collagen, type I, alpha 1 |
| family | COL1A2 | 1157.386 | 014 | collagen, type I, alpha 2 |
| | COL3A1 | 669.952 | EDS4A | collagen, type III, alpha 1 |
| | COL4A2 | 235.031 | ICH,POREN2 | collagen, type IV, alpha 2 |
| | COL4A1 | 220.303 | HANAC,ICH,POREN1, | collagen, type IV, alpha 1 |
| | | | arresten | |
| | COL5A2 | 201.936 | | collagen, type V, alpha 2 |
| | COL5A1 | 156.091 | | collagen, type V, alpha 1 |
| | COL4A5 | 7.592 | ASLN,ATS,CA54 | collagen, type IV, alpha 5 |
| | COL4A6 | 1.075 | CXDELq22.3,DELXq22.3 | collagen, type IV, alpha 6 |
| | COL5A3 | 0.204 | | collagen, type V, alpha 3 |
| | COL4A4 | 0.119 | CA44 | collagen, type IV, alpha 4 |
| | COL4A3 | 0.037 | | collagen, type IV, alpha 3 |
| | | | | (Goodpasture antigen) |
| | COLIOAI | 0.003 | | collagen, type X, alpha 1 |
| Ligands for AXL | GAS6 | 151.146 | AXLLG,AXSF | growth arrest-specific 6 |
| family | | | | |
| Ligands for ALK | MDK | 144.837 | ARAP,MK,NEGF2 | midkine (neurite growth- |
| family | | | | promoting factor 2) |
| | PTN | 46.156 | HARP,HBGF8,HBNF,NEG | pleiotrophin |
| | | | F1 | |
| Ligands for TRK | BDNF | 69.197 | ANON2,BULN2 | brain-derived neu o rophic |
| family | | | | factor |
| | NTF3 | 9.066 | HDNF,NGF-2,NGF2,NT3 | neurotrophin 3 |
| | NGF | 6.654 | Beta-NGF,HSAN5,NGFB | nerve growth factor (beta |
| | | | | polypeptide) |
| | NTF4 | 0.025 | | neurotrophin 4 |
| Ligands for | FGF5 | 53.267 | HBGF-5,Smag-82 | fibroblast growth factor 5 |
| FGFR family | FGF2 | 45.392 | BFGF,FGF-2,FGFB,HBGF- | fibroblast growth factor 2 |
| | | | 2 | (basic) |
| | FGF1 | 14.544 | AFGF,ECGF,ECGF- | fibroblast growth factor 1 |
| | | | beta,ECGFA,ECGFB,FGF- | (acidic) |
| | | | 1,FGF- | |
| | | | alpha,FGFA,GLI0703,HBG | |
| | FGF7 | 6.721 | HBGF-7,KGF | fibroblast growth factor 7 |
| | FGF20 | 0.404 | FGF-20 | fibroblast growth factor 20 |
| | FGF12 | 0.140 | FGF12B,FHF1 | fibroblast growth factor 12 |
| | FGF11 | 0.127 | FHF3 | fibroblast growth factor 11 |
| | FGF14 | 0.109 | FGF-14,FHF- | fibroblast growth factor 14 |
| | | | 4,FHF4,SCA27 | |
| | FGF9 | 0.060 | GAF,FIBFG-9,SYNS3 | fibroblast growth factor 9 |
| | | | | (glia-activating factor) |
| | FGF18 | 0.027 | FGF-18,ZFGF5 | fibroblast growth factor 18 |
| Ligands for | AGRN | 51.041 | | agrin |
| MUSK family | | | | |
| Ligands for | HBEGF | 35.111 | DTR,DTS,DTSF,HEGFL | heparin-binding EGF-like |
| EGFR Family | | | | growth factor |
| | NRG1 | 16.230 | ARIA,GGF,GGF2,HGL, | neuregulin 1 |
| | | | HRG,HR | |
| | EGF | 3.240 | HOMG4,URG | epidermal growth factor |
| | NRG4 | 0.266 | HRG4 | neuregulin 4 |
| | AREG | 0.233 | AR,CRDGF,SDGF | amphiregulin |
| | NRG2 | 0.055 | DON I ,HRG2,NTAK | neuregulin 2 |
| | EREG | 0.027 | ER | epiregulin |
| | TGFA | 0.049 | TFGA | transforming growth |
| | | | | factor, alpha |
| | BTC | 0.039 | | betacellulin |
| | NRG3 | 0.003 | HRG3,pro-NRG3 | neuregulin 3 |
| Ligands for | PDGFC | 30.229 | FALLOTEIN,SCDGF | platelet derived |
| | | | growth factor C | |
| | KITLG | 15.059 | FPH2,KL- | KIT ligand |
| | | | 1,Kit1,MGF,SCF,SF,SHEP7 | |
| | CSF1 | 13.916 | CSF-1,MCSF | colony stimulating factor |
| | | | | 1 (macrophage) |
| | PDGFA | 7.748 | PDGF-A,PDGF1 | platelet-derived |
| | | | | growth factor alpha |
| | FLT3LG | 1.335 | FL,FLT3L | fms-related tyrosine |
| | | | | kinase 3 ligand |
| | PDGFD | 0.815 | IEGF,SCDGF-B,SCDGFB | platelet derived |
| | | | | growth factor D |
| PDGFR family | PDGFB | 0.009 | PDGF2,SIS,SSV,c-sis | platelet-derived |
| | | | | growth factor beta |
| Ligands for | VEGFB | 27.744 | VEGFL,VRF | vascular endothelial |
| VEGFR family | | | | growth factor B |
| | VEGFA | 26.080 | MVCD1,VEGF,VPF | vascular endothelial |
| | | | | growth factor A |
| | VEGFC | 24.438 | Flt4-L,VRP | vascular endothelial |
| | | | | growth factor C |
| | PGF | 0.828 | D12 | placental growth factor |
| | | | S1900,PGFL,PLGF,PIGF- | |
| Ligands for EPH | EFNB2 | 16.895 | EPLG5,HTKL,Htk- | ephrin-B2 |
| family | | | L,LERK5 | |
| | EFNA4 | 5.884 | EFL4,EPLG4,LERK4 | ephrin-A4 |
| | EFNB1 | 4.779 | CFND,CFNS,EFL3,EPLG2, | ephrin-B1 |
| | | | Elk- L,LERK2 | |
| | EFNA5 | 1.923 | AF1,EFL5,EPLG7,GLCIM, | ephrin-A5 |
| | | | LERK 7,RAGS | |
| | EFNB3 | 1.301 | EFL6,EPLG8,LERK8 | ephrin-B3 |
| | EFNA3 | 0.416 | EFL2,EPLG3,Ehkl- | ephrin-A3 |
| | | | L,LERK3 | |
| | EFNA1 | 0.137 | B61,ECKLG,EFL1,EPLG1, | ephrin-Ai |
| | | | LERK-I,LERKLTNFAIP4 | |
| | EFNA2 | 0.043 | ELF-1,EPLG6,HEK7- | ephrin-A2 |
| | | | L,LERK-6,LERK6 | |
| Ligands for RYK | WNT5A | 14.539 | hWNT5A | wingless-type MMTV |
| family | | | | integration site family, |
| | | | | member 5A |
| Ligands for RET | GDNF | 9.169 | ATFI,ATF2,HFB1- | glial cell derived |
| family | | | GDNF,HS CR3 | neurotrophic factor |
| | ARTN | 0.482 | ENOVIN,EVN,NBN | artemin |
| | PSPN | 0.020 | PSP | persephin |
| | NRTN | 0.018 | NTN | neurturin |
| Ligands for TIE | ANGPT1 | 6.251 | AGP1,AGPT,ANG I | angiopoietin 1 |
| family | ANGPT2 | 0.060 | AGPT2,ANG2 | angiopoietin 2 |
| | ANGPT4 | 0.007 | AGP4,ANG-3,ANG4 | angiopoietin 4 |
| Ligands for | IGF2 | 4.455 | C11 orf43,IGF-II,PP9974 | insulin-like growth factor |
| 1NSR Family | | | | 2 (somatomedin A) |
| | IGF1 | 0.003 | IGF-I,IGF1A,IGFI | insulin-like growth factor |
| | | | | 1 (somatomedin C) |
| Ligands for MET | HGF | 4.175 | DFNB39,F- | hepatocyte growth |
| family | | | TCF,HGFB,HPTA,SF | factor (hepapoietin A; |
| | | | | scatter factor) |
| | MST1 | 2.179 | D3F15S2,DNF15S2,HGFL, | macrophage stimulating 1 |
| | | | MSP,NF15S2 | (hepatocyte growth |
| | | | | factor-like) |

TABLE 1-2
Identification of RTK ligand gene expression in RCS RPE cells by RNA-Seq

| | | RTK gene | | |
|---|---|---|---|---|
| | | expression | | |
| RTK | Gene | level (FPKM) in | | |
| subfamilies | symbol | RCS RPE | Gene synonyms | Gene full name |
| DDR family | Ddr1 | 138.385 | Cak,Drd1,PTK3D | discoidin domain receptor tyrosine |
| | | | | kinase |
| | Ddr2 | 7.314 | Tyro 10 | discoidin domain receptor tyrosine |
| | | | | kinase |
| FGFR family | Fgfr2 | 95.112 | | fibroblast growth factor receptor 2 |
| | Fgfr1 | 43.382 | | Fibroblast growth factor receptor 1 |
| | Fgfr3 | 24.743 | | fibroblast growth factor receptor 3 |
| | Fgfr4 | 0.084 | | fibroblast growth factor receptor 4 |
| PDGFR family | Pdgfrb | 76.095 | PDGFR-1 | platelet derived growth factor |
| | | | | receptor, beta polypeptide |
| | Pdgfra | 9.439 | APDGFR,PDG | platelet derived growth factor |
| | | | FAC E | receptor, alpha polypeptide |
| | Csflr | 5.491 | CSF-1-R,CSF- | colony stimulating factor 1 receptor |
| | | | 1R,M-CSF-R,c- | |
| | Flt3 | 1.095 | Flk2,CD135 | fins-related tyrosine kinase 3 |
| | Kit | 0.429 | SCFR,CD117 | v-kit Hardy-uckerman 4 feline |
| | | | | sarcoma viral oncogene homolog |
| AXL family | Ax1 | 62.618 | | Ax1 receptor tyrosine kinase |
| | Mertk | 29.044 | rdy | c-mer proto-oncogene tyrosine |
| | | | | kinase |
| | Tyro3 | 8.929 | Brt | TYRO3 protein tyrosine kinase |
| INSR Family | Igt2r | 41.997 | | insulin-like growth factor 2 receptor |
| | Igflr | 11.571 | IGFIRC,JTK13 | insulin-like growth factor 1 receptor |
| | Insr | 6.479 | | insulin receptor |
| PTK7 family | Ptk7 | 40.073 | | tyrosine-protein kinase-like 7 |
| | | | | [Source: RefSeq |
| | | | | peptide;Acc: NP_001100359] |
| RYK family | Ryk | 29.103 | | receptor-like tyrosine kinase |
| EPH family | Ephb4 | 21.284 | | Eph _{receptor:} B4 [Source: MGI |
| | | | | Symbol;AMGI: 104757] |
| | Epha2 | 13.613 | | Eph receptor A2 |
| | Epha4 | 11.658 | RGD 15 60587 | Eph receptor A4 |
| | Ephb3 | 4.508 | | Eph receptor 133 |
| | Ephb6 | 3.519 | | Eph receptor B6 |
| | Epha7 | 3.493 | | Eph receptor A7 |
| | Ephb2 | 2.191 | RGD15 64232 | Eph receptor B2 |
| | Epha3 | 1.871 | | Eph receptor A3 |
| | Epha1 | 1.364 | | Eph receptor A1 |
| | Epha5 | 0.421 | EHK- | EPH receptor AS |
| | | | 1,E1s1,E1s1. | |
| | Ephb1 | 0.162 | Ephb2,Erk,elk | Eph receptor 131 |
| | Epha8 | 0.015 | | Ephrin type-A receptor 8 |
| | | | | [Source:UniProtKB/Swiss- |
| | | | | Prot;Acc: P29321] |
| MET family | Met | 17.608 | Hgfr | met proto-cmcogene |
| | Mst 1 r | 1.054 | Cdw136,Ptk8, | macrophage stimulating 1 receptor met- |
| | | | Ron,Stk | relate (c-ci tosine kinase |
| ROR family | Ror1 | 7.241 | RGD1559469 | receptor tyrosine kinase-like |
| | | | | orphan receptor 1 |
| | Ror2 | 4.023 | | receptor tyrosine kinase-like |
| | | | | orphan receptor 2 |
| EGFR Family | Erbb2 | 6.467 | | v-erb-b2 erythroblastic leukemia |
| | | | | viral oncogene homolog 2, |
| | | | | neuro/glioblastoma derived |
| | | | | oncogene homolo (avian) |
| | Egfr | 1.353 | ERBB1,ErbB- | epidermal growth factor receptor |
| | | | 1,Errp | |
| | Erbb4 | 0.174 | | v-erb-a erythroblastic leukemia |
| | | | | viral oncogene homolog 4 |
| | | | | (avian) |
| | Erbb3 | 0.042 | nuc-ErbB3 | v-erb-b2 erythroblastic leukemia |
| | | | | viral oncogene homolog 3 (avian) |
| MUSK family | Musk | 3.251 | Nsk1 | muscle, skeletal, receptor tyrosine |
| | | | | kinase |
| TRK family | Ntrk3 | 1.883 | trkC | neurotrophic tyrosine kinase, |
| | | | | receptor, type 3 |
| | Ntrk2 | 0.755 | RATTRKB1,TR | neurotrophic tyrosine kinase, |
| | | | KB1,Tkrb,t rk- | receptor, type 2 |
| | | | B,trkB | |
| | Ntrk1 | 0.051 | Trk | neurotrophic tyrosine kinase, |
| | | | | receptor, e 1 |
| VEGFR family | F1t4 | 1.799 | Vegfr3 | fins-related tyrosine kinase 4 |
| | Kdr | 1.629 | | vascular endothelial growth |
| | | | | factor receptor 2 precursor |
| | | | | [Source: RefSeq e tide;Acc: NP |
| | | | | 037194 |
| | Flt1 | 0.280 | VEGFR-1 | FMS-related tyrosine kinase 1 |
| ALK family | Ltk | 1.427 | | leukocyte receptor tyrosine kinase |
| | ALK | 0.027 | | anaplastic lymphoma receptor |
| | | | | tyrosine kinase |
| STYK1 family | Styk1 | 1.300 | RGD1564211 | serine/threonine/tyrosine kinase 1 |
| RET family | Ret | 0.337 | ret proto-oncogene | |
| TIE family | Tek | 0.116 | Tie-2,Tie2 | TEK tyrosine kinase, endothelial |

TABLE 1-3
Identification of bridge molecule gene expression in hUTC by RNA-Seq

| | bridge molecule | | |
|---|---|---|---|
| | gene expression | | |
| Gene | level (FPKNI) | | |
| symbol | in hUTC | Gene synonyms | Gene full name |
| MFGE8 | 217.889 | BA46,EDIL1,HMFG,HsT19888,MFG- | milk fat |
| | | E8,MFGM,OAcGD3 S,SED1,SPAG I 0,hP47 | globule-EGF |
| | | | factor 8 |
| GAS6 | 151.146 | AXLLG,AXSF | growth arrest- |
| | | | specific 6 |
| THBS1 | 123.018 | THBS,THBS-1,TSP,TSP-1,TSP1 | thrombospondin 1 |
| THBS2 | 114.245 | TSP2 | thrombospondin 2 |
| PROS1 | 1.2068 | PROS,PS21,PS22,PS23,PS24,PS25,PSA,THPH5,THP | protein S (alpha) |
| | | H6 | |

TABLE 1-4
Identification of bridge molecule receptor gene expression in RCS RPE by
RNA-Seq

| | bridge molecule | | |
|---|---|---|---|
| | receptor | | |
| Gene | gene expression | | |
| symbol | level | Gene synonyms | Gene full name |
| Itgav | 47.430 | | integrin alpha V |
| Itgb5 | 119.220 | RGD1563276 | integrin, beta 5 |
| Itgb3 | 3.185 | | integrin beta 3 |
| Ax1 | 62.618 | | Axl receptor tyrosine |
| | | | kinase |
| Mertk | 29.044 | rdy | c-mer proto-oncogene |
| | | | tyrosine kinase |
| Tyro3 | 8.929 | Brt | TYRO3 protein tyrosine |
| | | | kinase |
| Cd36 | 0.028 | | CD36 molecule |
| | | | (thrombospondin receptor) |

### Example 2

### Receptor Tyrosine Kinase (RTK) Ligands Measurement in hUTC Conditioned Medium

The transcriptomic profile of both RCS RPE cells and hUTC using RNA-Seq and informatics data analysis showed that RCS RPE cells express multiple RTK genes, while hUTC expresses genes for multiple RTK ligands (Table 2-1). RTK ligands of seven RTK subfamilies, having relatively high gene expression levels in hUTC, were measured in hUTC conditioned media in comparison with those from normal human dermal fibroblast (NHDF) and ARPE-19 cells. These ligands include BDNF and NT3—ligands of Trk family, HGF—a ligand of Met family, PDGF-DD and PDGF-CC—ligands of PDGF family, ephrin-B2—a ligand of Eph family, HB-EGF—a ligand of ErbB family, GDNF—a ligand of Ret family, as well as agrin—a ligand of Musk family.

TABLE 2-1
Summary of RTK gene expression classification in
RCS RPE cells and RTK ligands gene expression
classification in hUTC from transcriptome profile analysis.

| | RTK subfamily | RTK ligand |
|---|---|---|
| | genes expressed | genes expressed |
| | by RCS RPE | by hUTC |
| RTK subfamilies | (RNA-Seq) | (RNA-Seq) |
| EGF receptor (ErbB) family | + | + |
| Insulin receptor family | + | + |
| PDGF receptor family | + | + |
| FGF receptor family | + | + |
| VEGF receptor family | + | + |
| HGF receptor/Met family | + | + |
| Trk family | + | + |
| Eph family | + | + |
| AXL (TAM) family | + | + |
| Tie family | + | + |
| DDR family | + | + |
| Ret family | + | + |
| Musk family | + | + |
| ALK family | − | − |

### Materials and Methods

hUTC (Lot NB12898P7 prepared from PDL20 Research Bank, page 7), ARPE-19 cells (passage 3) and NHDF (passage 10) were used for the study.

Human BDNF ELISA kit (catalog #DBD00, lot #311655, standard detection range: 62.5-4000 pg/mL; sensitivity: 20 pg/mL), human HGF ELISA kit (catalog #DHG00, lot #307319, standard detection range: 125-8000 pg/mL; sensitivity: <40 pg/mL), human PDGF-CC ELISA kit (catalog #DCC00, lot #309376, standard detection range: 62.5-4000 pg/mL; sensitivity: 4.08 pg/mL), human PDGF-DD ELISA kit (catalog #DDD00, lot #310518, standard detection range: 31.3-2000 pg/mL; sensitivity: 1.67 pg/mL) were from R&D Systems, Inc., Minneapolis, Minn. HB-EGF ELISA kit (catalog #ab100531, lot #GR135979-1, standard detection range: 16.4-4000 pg/mL; sensitivity: <20 pg/mL) and NT3 ELISA kit (catalog #ab100615, lot #GR141281-1, standard detection range: 4.12-3000 pg/mL; sensitivity: <4 pg/mL) were from abcam, Cambridge, Mass. Human GDNF ELISA kit (catalog #RAB0205, lot #0919130270, standard detection range: 2.74-2000 pg/mL; sensitivity: 2.74 pg/mL) was from Sigma, St. Louis, Mich. Human ephrin-B2 ELISA kit (catalog #MBS916324, lot #R21199424, standard detection range: 15.6-1000 pg/mL; sensitivity: 15.6 pg/mL) and agrin ELISA kit (catalog #MBS454684, lot #EDL201310110, standard detection range: 31.2-2000 pg/mL; sensitivity: <13.6 pg/mL) were from MyBioSource, Inc., San Diego, Calif.

Preparation of hUTC, ARPE-19 and NHDF Conditioned Media:

On day 1, hUTC, ARPE-19 and NHDF were seeded, respectively, at 10,000 viable cells/cm² in T75 cell culture flasks in 15 mL of hUTC growth medium (DMEM low glucose+15% v/v FBS+4 mM L-glutamine). Cells were cultured for 24 h in 37° C. 5% CO2 incubator. On day 2, media were aspirated and replenished with 21 mL of DMEM/F12 complete medium (DMEM/F12 medium+10% v/v FBS+50 U/ml Pen/50 μg/ml Strep). Cells were cultured for another 48 h. Control medium (DMEM/F12 complete medium) alone was also cultured for 48 h. On day 4, cell culture supernatants and control medium were collected and centrifuged at 250 g, 5 min at 4° C., aliquoted in cryotube at 0.5 mL/tube, and frozen immediately at −70° C. freezer. The frozen samples were thawed and used for ELISA.

### Results

Levels of selected RTK ligands measured in hUTC conditioned medium are summarized in Table 2-2.

TABLE 2-2
Concentrations of BDNF, NT3, HGF, PDGF-CC, PDGF-DD, GDNF, ephrin-B2,
HB-EGF, and Agrin in hUTC conditioned medium.

| RTK ligands | | | | | | | | | |
|---|---|---|---|---|---|---|---|---|---|
| (pg/mL) | BDNF | NT3 | HGF | PDGF-CC | PDGF-DD | GDNF | Ephrin-B2 | HB-EGF | Agrin |
| hUTC | 405 | 11 | 131.7 | 17.1 | 4.4 | 52.8 | Undetectable | Undetectable | 5S, similar to that |
| conditioned | | | | | | | | | in control medium |
| medium | | | | | | | | | |

Levels of selected RTK ligands measured in hUTC conditioned medium were also compared with those from NHDF and ARPE-19 conditioned medium, as normalized at pg/mL/1×10⁶ cells. hUTC secreted 72.7 pg/mL of BDNF per million cells per 48 h, compared to 20.4 pg/mL and 16.2 pg/mL of BDNF per million cells per 48 h from NHDF and ARPE-19, respectively (FIG. 11A). The amount of BDNF in control medium was undetectable. (FIG. 11G).

hUTC and NHDF secreted a low amount of NT3 (FIG. 11B). The amount of NT3 in ARPE-19 conditioned medium and control medium was undetectable.

hUTC secreted 23.7 pg/mL of HGF per million cells per 48 h, compared to 3.9 pg/mL and 0.4 pg/mL from NHDF and ARPE-19, respectively (FIG. 11C). The amount of HGF in the control medium was undetectable. (FIG. 11G).

The amounts of PDGF-CC and PDGF-DD in hUTC CM compared to those in NHDF and ARPE-19 CM are shown in FIG. 11D and FIG. 11E. 0.3 pg/mL of PDGF-CC and 3.1 pg/mL of PDGF-DD were detected in control medium, respectively.

hUTC secreted 9.5 pg/mL of GDNF per million cells per 48 h compared to 8.5 pg/mL of GDNF from NHDF. ARPE-19 only released trace amount of 1.3 pg/mL of GDNF per million cells per 48 h (FIG. 11F). The amount of GDNF in control medium was undetectable. (FIG. 11G).

The levels of ephrin-B2 and HB-EGF in conditioned medium of hUTC, NHDF, and ARPE-19 were under the detection limit of the ELISAs (15.6 pg/mL and 20 pg/mL, respectively). The levels of agrin in hUTC, NHDF and ARPE-19 conditioned medium were similar to that in control medium.

Effects of BDNF, HGF, PDGF-DD, ephrin-B2 and HB-EGF tested in RCS RPE phagocytosis assay using the doses at 200 ng/mL all showed positive effect on rescuing phagocytosis in RCS RPE cells. (Example 1). However, the actual concentrations of these ligands secreted from hUTC in conditioned medium appear lower than the level tested.

### Example 3

### Bridge Molecules in hUTC Conditioned Medium

The transcriptomic profile of both RCS RPE cells and hUTC showed that RCS RPE cells express genes of receptors that recognize “eat me” signals on apoptotic cells. (Erwig L-P, Cell Death and Differentiation 2008; 15: 243-250). These receptors include scavenger receptors (SR-A, LOX-1, CD68, CD36, CD14), integrins (αvβ3 and αvβ5), receptor tyrosine kinases of the Ax1 and Tyro3, LRP-1/CD91, and PS receptor Stabilin 1. Moreover, hUTC expresses a number of bridge molecule genes including TSP-1, TSP-2, surfactant protein D (SP-D), MFG-E8, Gas6, apolipoprotein H, and annexin 1. Secretion of bridge molecules in hUTC conditioned medium was examined and the levels compared with those from ARPE-19 and normal human dermal fibroblast (NHDF).

### Materials and Methods

hUTC (PDL20, master cell bank number 25126057), ARPE-19 cells (passage 3) (ATCC, Manassas, Va.) and NHDF (passage 11) (Lonza, South Plainfield, N.J.) were used for the study.

Human Gas6 ELISA kit (catalog #SK00098-01, lot #20111218) and human SP-D ELISA kit (catalog #SK00457-01, lot #20111135) from Aviscera Bioscience, Santa Clara, Calif. The standard detection range of human Gas6 ELISA kit is 62.5-8000 pg/mL with the sensitivity of 31 pg/ml. The standard detection range of human SP-D ELISA kit is 78-5000 pg/mL with the sensitivity of 30 pg/ml. Human MFG-E8 ELISA kit (catalog #DFGE80, lot #307254, standard detection range: 62.5-4000 pg/mL; sensitivity: 4.04 pg/mL), human TSP-1 ELISA kit (catalog #DTSP10, lot #307182, standard detection range: 7.81-500 ng/mL; sensitivity: 0.355 ng/mL), and human TSP-2 ELISA kit (catalog #DTSP10, lot #307266, standard detection range: 0.31-20 ng/mL; sensitivity: 0.025 ng/mL) were from R&D Systems, Inc., Minneapolis, Minn. Apolipoprotein H human ELISA kit (catalog #ab108814, lot #GR126938, standard detection range: 0.625-40 ng/mL, sensitivity: 0.6 ng/mL) was from Abcam, Cambridge, Mass. Human Annexin I (ANX-I) ELISA kit (catalog #MBS704042, lot #N10140947, standard detection range: 0.312-20 ng/mL; sensitivity: 0.078 ng/mL) was from MyBioSource, Inc., San Diego, Calif.

TABLE 3-1
Summary of phagocyte receptors, bridge molecules and
phagocyte binding sites on apoptotic cells.

| Recognition | | Binding site |
|---|---|---|
| receptor | Bridge | on apoptotic |
| on phagocyte | molecule | cell |
| Scavenger receptors | Thrombospondin | OxLDL-like sites |
| SR-A | | OxLDL-like sites |
| LDX-1 | | OxLDL-like sites |
| CD88 | | TSP-1 binding sites |
| CD36 | | ICAM-3 |
| CD 14 | | Unidentified |
| Unidentified lectins | | glycoproteins |
| CD91 calreticulin | MBL | Collectin-binding sites |
| | SP-A | Collectin-binding sites |
| | SP-D | Collectin-binding sites |
| | Ctq | Ctq-binding sites |
| | SP-A, SP-D, MBL | Nucleic acid |
| Integrin receptors | | |
| Vitronectin receptor αvβ3 | Thrombospondin | TSP-1-binding sites |
| αvβ5 | Thrombospondin | TSP-1-binding sites |
| Complement receptor 3 umβ2 | C3b/bi | C3b/bi binding sites |
| Complement receptor 4 umβ2 | C3b/bi | C3b/bi binding sites |
| PS-bridge molecule receptors | | |
| Mer | Gas7 | Phosphatidylserine |
| β2-GPI-receptor | β2-GPI | Phosphatidylserine |
| Vitronectin receptor αvβ3 | MFG-E8 | Phosphatidylserine |
| Not yet identified | Annexin 1 | Phosphatidylserine |
| Mer | Protein S | Phosphatidylserine |
| PS receptor | | |
| Not yet identified | | Phosphatidylserine |
| ATP-binding cassette | | ABCA? |
| transporter | | |

Note
that receptor tyrosine kinases Tyro3 and Ax1 are also PS-bridge molecule receptors and bind to Gas6.

Preparation of hUTC, ARPE-19 and NHDF Conditioned Media:

On day 1, hUTC, ARPE-19 and NHDF were seeded, respectively, at 10,000 viable cells/cm² in T75 cell culture flasks in 15 mL of hUTC growth medium (DMEM low glucose+15% FBS+4 mM L-glutamine). Culture for 24 h in 37° C. 5% CO2 incubator. On day 2, media was aspirated and replenished with 18 mL of DMEM/F12 complete medium (DMEM:F12 medium+10% FBS+Pen (50 U/ml)/Strep (50 μg/ml)). Cells were cultured for another 48 h. Control medium (DMEM:F12 medium+10% FBS+Pen (50 U/ml)/Strep (50 μg/ml)) alone was also cultured for 48 h. On day 4, cell culture supernatants and control medium were collected and centrifuged at 250 g, 5 min at 4° C., aliquoted in cryotube at 0.5 mL/tube, and frozen immediately at −70° C. freezer. The frozen samples were thawed and used for ELISA.

### Results

hUTC secreted 77.2 ng of MFG-E8 per million cells per 48 h, compared to 7.6 ng and 17.5 ng of MFG-E8 per million cells per 48 h from ARPE-19 and NHDF, respectively (FIG. 12A). The concentration of MFG-E8 in hUTC conditioned medium was 15.5 ng/mL. The amount of MFG-E8 in control medium was undetectable. (FIG. 12E).

hUTC secreted 352.8 pg of Gas6 per million cells per 48 h, compared to 183.9 pg from ARPE-19 and 1101 pg from NHDF (FIG. 12B). The concentration of Gas6 in hUTC conditioned medium was 70.6 pg/mL. The amount of Gas6 in control medium was undetectable. (FIG. 11G).

hUTC secreted 759.2 ng of TSP-1 per million cells per 48 h, compared to 4744.68 ng of TSP-1 per million cells per 48 h from ARPE-19 and 2487.55 ng from NHDF (FIG. 12C). The concentration of TSP-1 in hUTC conditioned medium was 151.8 ng/mL. 4.0 ng/mL of TSP-1 was detected in control medium. (FIG. 12E).

hUTC secreted 44.2 ng of TSP-2 per million cells per 48 h compared to 30 ng of TSP-2 from NHDF. ARPE-19 released negligible amount of 0.08 ng of TSP-2 per million cells per 48 h (FIG. 12D). The concentration of TSP-2 in hUTC conditioned medium was 8.8 ng/mL. Trace amount of TSP-2 (0.02 ng/mL) was detected in control medium. (FIG. 12E).

The level of Apolipoprotein H in the conditioned medium of hUTC, ARPE-19 and NHDF was similar to that in control medium (6.8 ng/mL). Levels of SP-D and Annexin I in hUTC, ARPE-19 and NHDF conditioned media as well as control medium were under the detection limit of the ELISAs (<30 pg/mL and <78 pg/mL, respectively). The cells either do not secrete the two proteins, or the levels are below the limit of detection.

A summary of bridge molecules examined in hUTC conditioned medium and their concentrations is listed in Table 3-2.

TABLE 3-2
Summary of bridge molecules examined in hUTC conditioned medium

| Bridge | | | | | | | |
|---|---|---|---|---|---|---|---|
| molecules | MFG-E8 | Gas6 | TSP-1 | TSP-2 | Apolipoprotein H | SP-D | Annexin I |
| hUTC | 15.5 ng/mL | 70.6 pg/mL | 151.8 ng/mL | 8.8 ng/mL | 6.7 ng/mL, similar to | undetectable | undetectable |
| conditioned | | | | | the level in control | | |
| medium | | | | | medium | | |

Among the bridge molecules measured, MFG-E8, Gas6, TSP-1 and TSP-2 are bridging molecule candidates involved in hUTC-mediated phagocytosis rescue in RCS RPE cells.

Binding of bridge molecules opsonized ROS would activate the integrin and RTK signaling pathways, which would compensate for the absence of Mertk signaling, and lead to rescue of phagocytosis.

### Example 4

### Effect of hUTC Conditioned Medium and Bridge Molecules on Rod Outer Segment (ROS) Phagocytosis by RCS RPE Cells

The direct effect of hUTC conditioned media on ROS phagocytosis was examined by feeding RCS RPE cells with ROS preincubated with hUTC conditioned media. (US 2010/0272803). The phagocytosis of the dystrophic RPE cells was completely rescued. Here, the effect of bridge molecules present or secreted in hUTC conditioned media was investigated. Currently, the only “eat-me” signal identified on ROS is phosphatidylserine (PS) (Finnemann et al., PNAS, 2012; 109 (21):8145-8148).

### Materials and Methods

Procedures for RPE isolations, primary culture of RPE cells, sulforhodamine staining of RPE cells, isolation of rat ROS, FITC staining of ROS, phagocytosis assay, imaging and quantitation, assay acceptance criteria, and relative phagocytosis level are described in Example 1.

hUTC Conditioned Medium (CM):

hUTC CM3 was used for the study. On day 1, hUTC was seeded at 10,000 viable cells/cm² in T75 cell culture flask in hUTC growth medium (DMEM low glucose+15% FBS+4 mM L-glutamine). Culture for 24 hours in 37° C. 5% CO2 incubator. On day 2, medium was aspirated and replenished with 21 mL of DMEM/F12 complete medium (DMEM:F12 medium+10% FBS+Pen (50 U/ml)/Strep (50 μg/ml)). Cells were cultured for another 48 hours. Control medium (DMEM:F12 medium+10% FBS+Pen (50 U/ml)/Strep (50 μg/ml)) alone was also cultured for 48 h. On day 4, cell culture supernatant and control medium were collected and centrifuged at 250 g, 5 min at RT, aliquoted in cryotube at 3 mL/tube, and frozen immediately at −70° C. freezer.

Recombinant Human Bridge Molecules:

Recombinant Human MFG-E8 (Cat #2767-MF-050, Lot # MPP2012061), recombinant Human Gas6 (Cat #885-GS-050, Lot # GNT5013011), recombinant Human TSP-1 (Cat #3074-TH-050, Lot # MVF3613041), recombinant Human TSP-2 (Cat #1635-T2-050, Lot # HUZ1713021), were all obtained from R&D Systems, Inc., Minneapolis, Minn. Reconstitution of individual protein stock solution was to follow the vendor's data sheets: Recombinant Human MFG-E8, TSP-1 and TSP-2 were reconstituted at 100 μg/mL in sterile PBS, respectively. Recombinant Human Gas6 was reconstituted at 100 μg/mL in sterile water. The reconstituted stocks were aliquoted and frozen at −70° C. freezer.

Effects of Bridge Molecules on RCS RPE Cell Phagocytosis:

ROS was preincubated with control medium (DMEM+10% FBS) or CM3 for 24 h in CO2 cell culture incubator at 37° C. In parallel, ROS was preincubated in control medium with various concentrations of human recombinant MFG-E8, Gas6, TSP-1 or TSP-2 for 24 h in CO2 cell culture incubator at 37° C. After the incubation, the ROS was spun down without wash, resuspended in MEM5 and fed to the dystrophic RPE cells in the presence of MEM5 for phagocytosis assay. For controls, normal RPE alone or dystrophic RPE alone was cultured in MEM20, then changed to MEM5 in the presence of untreated ROS (resuspended in MEM20 and fed to RPE cells) for phagocytosis assay.

Effects of RTK Ligands on RCS PRE Cell Phagocytosis:

RCS RPE were incubated with recombinant human BDNF, HGF and GDNF individually for 24 hours, and then OS was added for phagocytosis assay. RCS RPE incubated with hUTC CM was used as a positive control.

siRNA Knockdown:

The On-TARGETplus human siRNA-SMARTpools directed against human BDNF, HGF, GDNF, MFG-E8, Gas6, TSP-1 and TSP-2, as well as ON-TARGETplus Non-targeting pool (scrambled siRNA pool) were purchased from GE Dharmacon (Lafayette, Colo.). 25 nM of each siRNA pool was incorporated into hUTC respectively, using DharmaFECT transfection reagent (GE Dharmacon).

Antibodies for Immunofluorescence Staining:

Unconjugated monoclonal antibodies for the bridge molecules (human MFG-E8, Gas6, TSP-1 and TSP-2), as well as mouse IgG2A and IgG2B isotype control antibodies were obtained from R&D Systems, Inc., Minneapolis, Minn. These antibodies were conjugated with Alexa Fluor 488 fluorophore by Life Technologies (Eugene, Oreg.). An unconjugated monoclonal anti-rhodopsin antibody (EMD Millipore Corp., Temecula Calif.) was conjugated with Alexa Fluor 568 by Life Technologies (Eugene, Oreg.). An unconjugated mouse IgG2b, x isotype control antibody was obtained from Biolegend Inc. (San Diego, Calif.) and conjugated with Alexa Fluor 488 fluorophore by Life Technologies (Eugene, Oreg.). It was used as an isotype control antibody for Alexa Fluor 568 conjugated anti-rhodopsin antibody. Alexa Fluor 488 conjugated mouse IgG2A was used as an isotype control antibody for Alexa Fluor 488 conjugated anti-human MFG-E8, Gas6, or TSP-2 antibodies. Alexa Fluor 488 conjugated mouse IgG2B was used as an isotype control antibody for Alexa Fluor 488 conjugated human TSP-1 antibody.

Immunofluorescence:

10×10⁶ OS were incubated for 24 hours at 37° C. in 1 mL of hUTC CM, 1 mL of control medium, or 1 mL of control medium containing 124 ng/mL MFG-E8, 8.75 ng/mL Gas6, 1.2 μg/mL TSP-1 or 238 ng/mL TSP-2. OS were pelleted, washed and embedded in Tissue-Tek O.C.T compound (Sakura Finetek USA, Inc., Torrance, Calif.). A cryostat (Leica CM1950, Leica Microsystems, Inc., Buffalo Grove, Ill.) was used to obtain 10 iAm serial sections. The sections were transferred to glass slides for the immunofluorescence staining. Circled spots with the OS pieces were treated with blocking buffer (10% (v/v) goat serum, 1% (v/v) BSA, and 0.1% (v/v) Triton x 100 in PBS) for 1 hour at room temperature and then double stained with Alexa Fluor 568 conjugated anti-rhodopsin antibody and Alexa Fluor 488 conjugated anti-MFG-E8, anti-Gas6, anti-TSP-1, anti-TSP-2, or mouse IgG2A or IgG2B isotype control antibody for 2 hours at 4° C. After washing three times with PBS, sections were mounted in Vectashield mounting media (Vector Laboratories, Inc., Burlingame, Calif.) and evaluated with a Zeiss Photomicroscope III (Carl Zeiss, Oberkochen, Germany) equipped with epifluorescence. Images were captured with a Kodak 290 digital camera and analyzed using Kodak Microscopy Documentation System 290 Photoshop image analysis software (Eastman Kodak, Rochester, N.Y.). Images were made at 250× magnification with the appropriate filters.

### Results

As shown in FIGS. 13A and 13B (experiment 1) and FIGS. 13C and 13D (experiment 2), untreated dystrophic RPE cells reduced phagocytosis compared to normal RPE cells. Preincubation of dystrophic RPE cells with hUTC conditioned media completely rescued phagocytosis without hUTC conditioned media being present during the assay. More robust enhancement of phagocytosis was observed when hUTC conditioned media was present throughout the phagocytosis assay whether dystrophic RPE cells were pretreated with hUTC conditioned media or not. Dystrophic RPE cells, fed with ROS pretreated with hUTC conditioned media, showed a restoration of phagocytosis in the absence of hUTC conditioned media during the phagocytosis assay.

Dystrophic RPE cells were fed with ROS preincubated with various concentrations of MFG-E8 (15.5 ng/mL; 31 ng/mL; 62 ng/mL; 124 ng/mL), Gas6 (70 pg/mL; 350 pg/mL; 1750 pg/mL; 8750 pg/mL), TSP-1 (152 ng/mL; 304 ng/mL; 608 ng/mL; 1216 ng/mL) or TSP-2 8.8 ng/mL; 26.4 ng/mL; 79.2 ng/mL; 237.6 ng/mL) and assayed for phagocytosis (FIGS. 14 A-14D). Ten observations were made per sample. The ROS phagocytosis was rescued by feeding RCS RPE cells with ROS preincubated with MFG-E8, Gas6, TSP-1 or TSP-2.

Each of MFG-E8, Gas6, TSP-1 and TSP-2 dose-dependently increased the phagocytosis level in RCS RPE cells (FIGS. 14E-14H). Similarly, BDNF, HGF and GDNF dose-dependently increased the phagocytosis level in RCS RPE cells, with the effect of HGF being the strongest even at the lowest dose. When applied at higher concentrations, BDNF, HGF and GDNF were able to rescue phagocytosis in RCS RPE (FIGS. 14I-J). These results show that recombinant RTK ligand and bridge molecule proteins can mimic the effect of hUTC CM and restore RCS RPE phagocytosis, and are involved in hUTC-mediated phagocytosis rescue in RCS RPE.

BDNF, HGF, GDNF, MFG-E8, Gas6, TSP-1 and TSP-2 were knocked down in hUTC by siRNA mediated gene silencing. Scrambled siRNA pool that does not target any genes was used as knockdown control. The knockdown efficiency of each factor was examined by measuring the level of each factor in the cell culture supernatants collected from hUTC transfected with siRNA (FIG. 15A). Mock or scrambled siRNA transfection had no effect on hUTC secretion of these factors. siRNA targeting MFG-E8, TSP-1, TSP-2 and HGF yielded almost 100% knockdown efficiency; 80% and 65% knockdown were observed for BDNF and GDNF, respectively (FIG. 15A). siRNA targeting Gas6 in hUTC did not work (data not shown). CM was produced from siRNA-transfected hUTC and applied to RCS RPE to identify the effects of RTK ligands and bridge molecule knockdown. RCS RPE were cultured with CM produced from hUTC transfected with siRNA targeting BDNF, HGF or GDNF (FIG. 15B), or were fed with OS pre-treated with CM produced from hUTC transfected with siRNA targeting MFG-E8, TSP-1 or TSP-2 (FIG. 15C). CM prepared from untransfected and scrambled siRNA transfected hUTC were used as knockdown control CMs. Individual knockdown of each of the RTK ligands abolished the effect of hUTC CM on phagocytosis rescue compared to that of knockdown control CMs (FIG. 15B). Knocking-down of each of the bridge molecules decreased the phagocytosis of OS by RCS RPE (FIG. 15C). These RTK ligands and bridge molecules are required for hUTC-mediated phagocytosis rescue in RCS RPE.

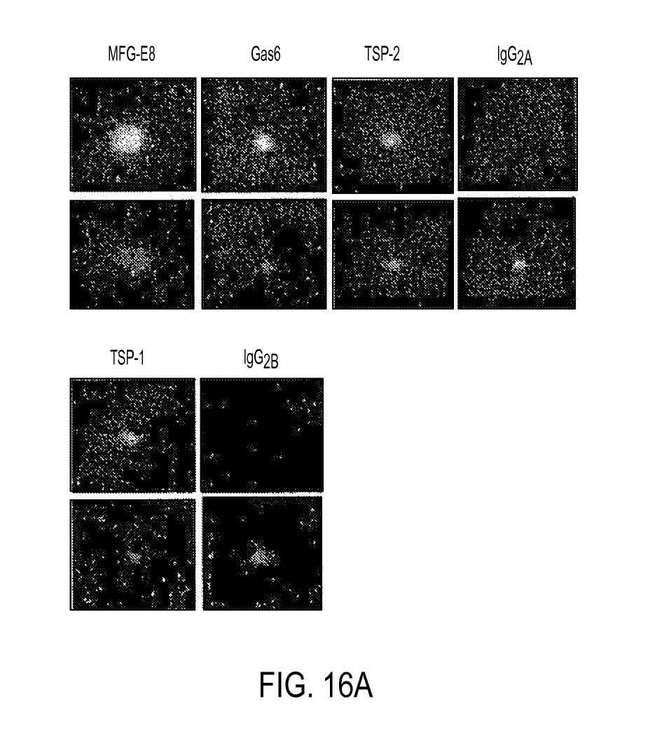

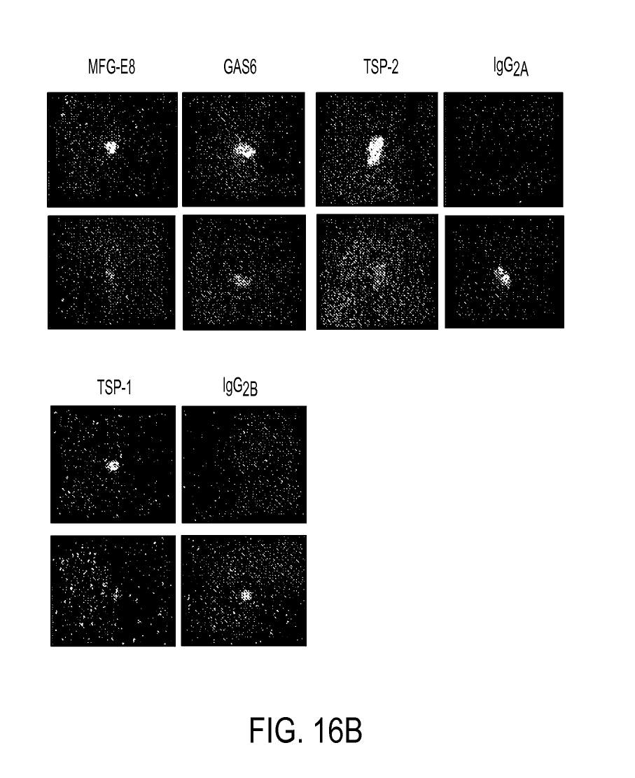

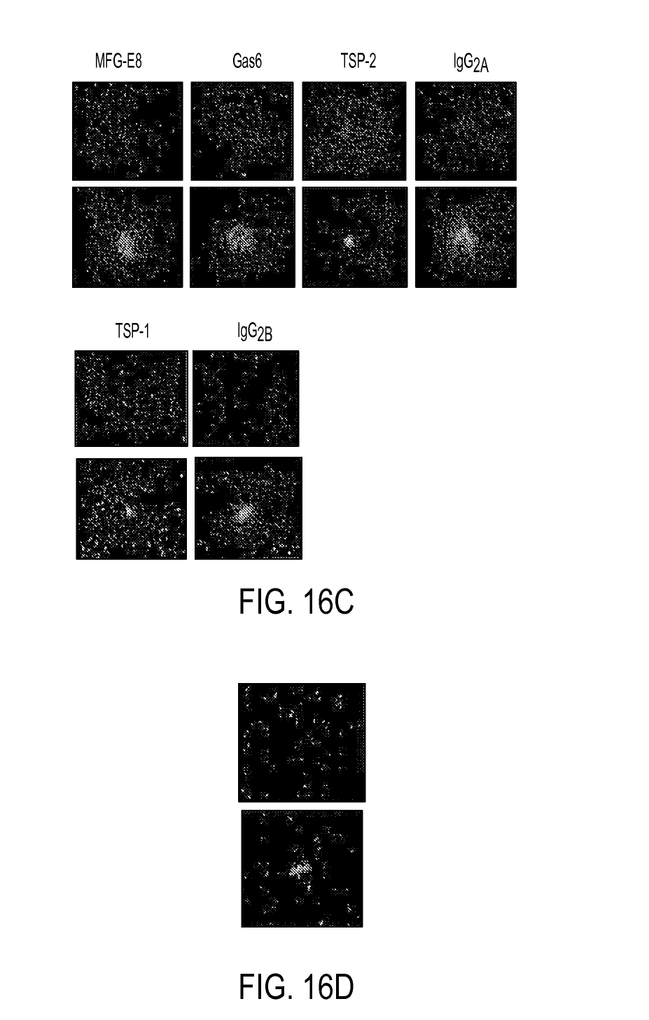

Dual staining for each individual bridge molecule and rhodopsin ensured that OS was evaluated. Rhodopsin is the visual pigments localized in photoreceptor OS and is a hallmark for OS staining (Szabo K, et al. Cell Tissue Res. 2014; 356(1):49-63). Rhodopsin-stained (Alexa Fluor 568 conjugated, red) particles, pre-incubated with individual recombinant human bridge molecule, stained positively with each of the four bridge molecule antibodies (Alexa Fluor 488 conjugated, green), but not with the Alexa Fluor 488 conjugated mouse IgG2A or IgG2B isotype control antibody (FIG. 16A). Similar results were obtained for OS incubated with hUTC CM (FIG. 16B), whereas no staining for any of the bridge molecules was observed for control medium-incubated OS (FIG. 16C). The specificity of the anti-rhodopsin antibody was confirmed by double staining of the OS particles with Alexa Fluor 568 conjugated anti-rhodopsin antibody and Alexa Fluor 488 conjugated mouse IgG2b, x isotype control antibody. The OS was stained positively only with anti-rhodopsin antibody (FIG. 16D). Bridge molecules MFG-E8, Gas6, TSP-1 and TSP-2 in hUTC CM co-localized with rhodopsin on OS demonstrated that the bridge molecules bind to OS.

### Example 5

### hUTC Protection of RPE from Oxidative Damage

Oxidative stress can compromise the health of retinal pigment epithelium. The effect of hUTC and hUTC conditioned media to improve the health of RPE cells exposed to oxidative damage was investigated.

### Materials and Methods

Hydrogen peroxide (H₂O₂), crystal violet and Thiazolyl Blue Tetrazolium Bromide (MTT) were obtained from Sigma-Aldrich (St Louis, Mo.).

Ham's F10 medium, Penicillin-Streptomycin Solution (5000 units/mL penicillin/5000 μg/mL streptomycin), Trypsin-EDTA solution (0.05%), low glucose DMEM, L-glutamine 200 mM were obtained from Life Technologies).

Hyclone FBS and formaldehyde were purchased from Thermo Scientific. Isopropanol, glacial acetic acid and hydrochloric acid were purchased from Fisher Scientific (Pittsburgh, Pa.). Ethanol was obtained Decon Labs Inc. (King of Prussia, Pa.). PBS was obtained from Lonza (South Plainfield, N.J.).

ARPE Growth Medium:

DMEM with 4.5 g/L glucose and sodium pyruvate without L-glutamine and phenol red) (Mediatech, Inc. A Corning Subsidiary, Manassas, Va.) supplemented with 5% or 10% heat-inactivated fetal bovine serum (FBS, Life Technologies, Grand Island, N.Y.), 1× Minimum Essential Medium—Non-Essential Amino Acids (MEM-NEAA, Life Technologies) and 0.01 mg/mL Gentamicin Reagent Solution (Life Technologies).

5% ARPE Growth Medium Containing 10 μM A2E:

DMEM (Mediatech, Inc.) supplemented with 5% heat-inactivated FBS (Life Technologies), 1×MEM-NEAA (Life Technologies), 0.01 mg/mL Gentamicin Reagent Solution (Life Technologies) and 10 μM A2E (prepared by the lab of Dr. Janet Sparrow).

hUTC Complete Medium:

DMEM low glucose (Life Technologies) supplemented with 15% Hyclone® FBS (Thermo Scientific, Logan, Utah) and 4 mM L-glutamine (Life Technologies).

hUTC FBS Medium:

DMEM (Mediatech, Inc.) supplemented with 5% or 10% heat-inactivated FBS (Life Technologies), 1×MEM-NEAA (Life Technologies), 0.01 mg/mL Gentamicin Reagent Solution (Life Technologies) and 4 mM L-glutamine (Mediatech, Inc.).

hUTC Conditioned Medium:

hUTC (Research Bank NB12898P6, PDL20) were seeded at 5000 cells/cm² in hUTC complete medium (15 mL) in 2 T75 culture flasks at 37° C., 5% CO2. 24 hours post-seeding, medium was removed from each flask and cells were washed 3 times with 15 mL 1×Dulbecco's phosphate-buffered saline (DPBS). Following the third wash, 15 mL of 5% or 10% FBS hUTC medium were added to each of the flasks. Fifteen mL of 5% or 10% FBS hUTC media was also added to 2 empty T75 flasks and served as controls. All flasks were returned to 37° C., 5% CO2 for 48 hours. After 48 hours, media was removed from each flask and centrifuged at 250×g for 5 minutes at 4° C. Media was placed on ice and then aliquoted and stored at −80° C.

ARPE-19 Cell Culture for A2E Study:

On Day 1 ARPE-19 cells were seeded in S-well Nunc™ Lab-Tek™ II chamber slides (Nalge Nunc International Corporation, Rochester, N.Y.) at a density of 40,000 cells/well in a final volume of 300 μL 10% ARPE growth medium. 24-hours post-seeding (Day 2) the medium on the cells was removed and replaced with 300 μL 5% ARPE growth medium. One week later (Day 9), medium was again removed and replaced with fresh 5% ARPE growth medium. On Day 14, media was removed and replaced with fresh 5% ARPE growth medium containing 10 μM A2E. On Days 17 and 21, media was removed again and replaced with fresh A2E-containing medium. On Day 24, the A2E containing medium was removed and replaced with fresh 5% ARPE growth medium, and the cells were allowed to quiesce for five days.

On Day 29, medium was removed from each well and replaced with 250 μL of 5 or 10% hUTC conditioned or control media (5% and 10% FBS hUTC media not exposed to cells). On Days 32 and 35, the conditioned and control media were removed from the cells and replaced with fresh conditioned or control media.

On Day 36, all media were removed from each well and cells were washed once with 1×DPBS. 200 μL fresh DPBS was subsequently added to each well and cells were exposed to 430 nm light delivered from a tungsten halogen source for 20 minutes. Following light exposure the DPBS was removed and cell viability assays were performed.

MTT Assay for A2E Study:

Cytotoxicity was measured by a metabolic (MTT, (3-(4,5-Dimethylthiazol-2-yl)-2,5-Diphenyltetrazolium Bromide) colorimetric microtiter assay (Roche Diagnostics Corporation, Indianapolis, Ind.). To perform the MTT assay, 20 μL of MTT labeling reagent (Roche Diagnostics Corporation) was added to 0.2 mL of 5% culture medium in each well. After 4 hours of incubation, another 200 μL of solubilization solution was added to each well for an overnight incubation. After centrifugation at 13,000 rpm for 2 minutes, supernatants were measured spectrophotometrically at 570 nm (SpectaMax MJ, Molecular Devices, Sunnyvale, Calif.). A decrease in the absorbance at 570 nm of reduced MTT is indicative of diminished cellular viability. Data were analyzed with Prism Software.

Dead Red Assay for A2E Study:

Nonviable cells were quantified after labeling by a fluorescence exclusion assay that allowed for the labeling of apoptotic nuclei because of a loss of plasma membrane integrity during the latter stages of cell death. Following light exposure cells were returned to 5% ARPE growth medium. Eight hours after blue light exposure, the nuclei of dead cells were stained with the membrane impermeable dye Dead Red (Life Technologies; 1/500 dilution, 15 min incubation) and all nuclei were stained with 4′,6′-diamino-2-phenylindole (DAPI) (Life Technologies). Briefly, cells were washed twice with prewarmed (37° C.) Hank's Balanced Salt Solution (1×) HBSS (Life Technologies). 250 μL of Dead Red working solution (8 μL Dead Red stock+4 mL HBSS) were added to each well for 15 minutes at room temperature. After 15 minutes cells were washed twice with HBSS. 300 μL of 4% formaldehyde prepared is 1×DPBS were added to each well for 30 minutes at room temperature. Cells were washed 3 times with 1×DPBS and incubated with DAPI (1:300 prepared in 1×DPBS) working solution for 5 minutes at room temperature. Cells were washed 3 times with 1×DPBS. Slides were mounted and coverslipped and replicates were assayed by counting DAPI-stained and Dead-Red stained nuclei in at least 5 microscopic fields within the area of illumination in each well. Values are presented as Dead-Red-stained nuclei/DAPI stained nuclei×100.

Cell Culture for H₂O₂ Study:

ARPE-19 cells (American Type Culture Collection; Manassas, Va.) were grown in Ham's F10 Medium containing 10% FBS and 50 units/mL penicillin/50 μg/mL streptomycin as monolayers in T75 flasks at 37° C., 5% CO2. For co-culture experiments with hUTC, the ARPE19 cells were grown to 80-90% confluency in T-75 flasks and subsequently seeded in 24-well cell culture plates. ARPE-19 cells were allowed to grow in 10% FBS growth medium until the 3rd day after seeding when the media was changed to basal medium (Ham's F10 media supplemented with 2% FBS and 50 units/mL penicillin/50 μg/mL streptomycin).

hUTC were seeded onto cell culture inserts (pore size 1 μm) at 5000 cells/cm² in hUTC complete growth (low glucose DMEM supplemented with 15% FBS and 4 mM L-glutamine) for 24 hours. Inserts were transferred to ARPE-19 cells growing on cell culture plates for 72 hours and grown in hUTC complete medium. Inserts were removed and ARPE-19 cells were treated with H₂O₂ (0-1500 μM) prepared in serum free Ham's F10 media for 9 hours.

Crystal Violet Cell Viability Assay for H₂O₂ Study:

The relative cell viability was determined by crystal violet uptake. Following treatments, cells were fixed in 4% paraformaldehyde in PBS and stained in a solution of 0.1% crystal violet, 10% ethanol. After washing with water, the remaining stain was dissolved in 10% acetic acid and the absorbance measured with a microplate reader at 550 nm.

MTT Assay for H₂O₂ Study:

Cells were incubated with 0.25 mg/mL MTT in serum-free medium at 37° C. for 3 hours. The medium was then removed and acidic isopropanol (1 μL concentrated HCL per 1 mL isopropanol) was added to solubilize the produced blue formazan (MTT metabolic product). The density of blue formazan was measured at 550 nm with a background wavelength at 630 nm using a microplate reader.

### Results

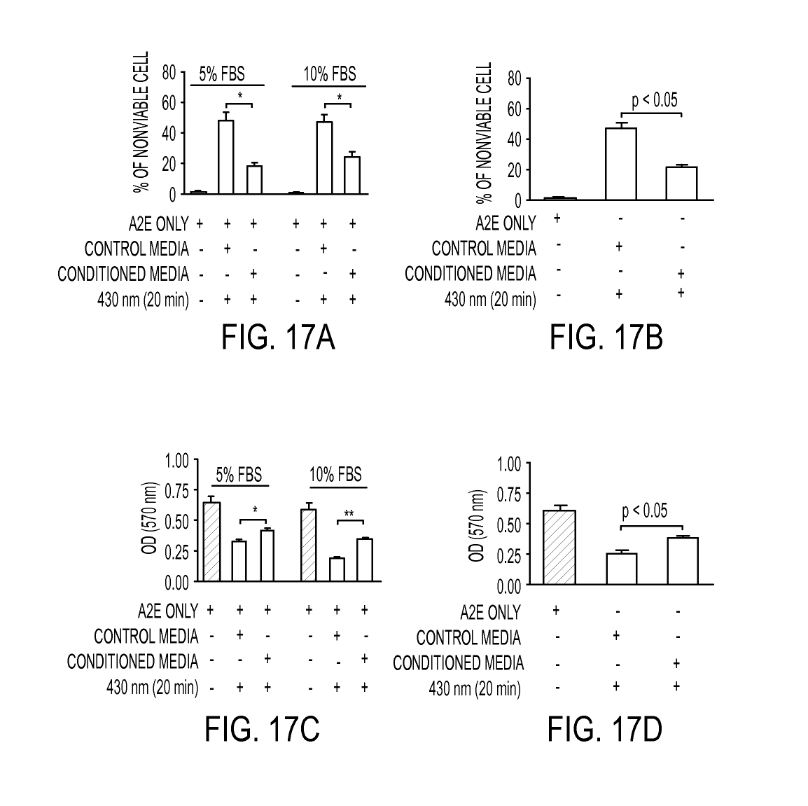

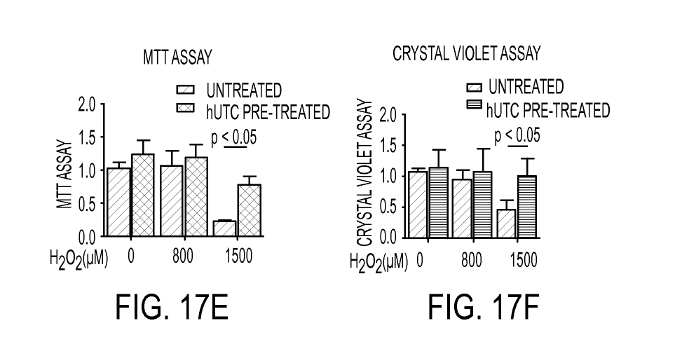

hUTC conditioned media protected A2E-laden RPE cells from blue light-induced damage. ARPE-19 viability was assessed post-irradiation by labeling cells with the membrane-impermeant dye Dead Red and all nuclei with DAPI. Nuclei counted in digital images provided the percent of viable and non-viable cells (FIGS. 17A-17B). In the absence of 430 nm illumination, 10 μM A2E had no effect on ARPE-19 viability. Cells that were incubated with control media and subjected to 430 nm illumination showed high levels of nonviable cells (˜50%). In contrast, treatment with hUTC conditioned media resulted in a reduction in the number of nonviable cells (˜20%).

Cell viability was also measured by MTT assay (FIGS. 17C-17D), which is based on the ability of healthy cells to cleave the yellow tetrazolium salt MTT to purple formazan crystals. Production of formazan is proportional to the number of viable cells in the culture. ARPE-19 cells treated with control media and exposed to light showed a reduction in viability compared to ARPE-19 cells that were loaded with 10 μM A2E and not exposed to light. Cells treated with 5 or 10% hUTC conditioned media showed higher viabilities than those exposed to control media.

Following total H₂O₂ treatment, ARPE19 cell viability was determined by the crystal violet and MTT assays (FIGS. 17E-17F). ARPE-19 cells that were co-cultured with hUTC showed improved viabilities following treatment with 1500 μM H₂O₂ compared to untreated control cells.

### Example 6

### Derivation of Cells from Postpartum Tissue

This example describes the preparation of postpartum-derived cells from placental and umbilical cord tissues. Postpartum umbilical cords and placentae were obtained upon birth of either a full term or pre-term pregnancy. Cells were harvested from five separate donors of umbilicus and placental tissue. Different methods of cell isolation were tested for their ability to yield cells with: 1) the potential to differentiate into cells with different phenotypes, a characteristic common to stem cells; or 2) the potential to provide trophic factors useful for other cells and tissues.

### Methods & Materials

Umbilical Cell Isolation:

Umbilical cords were obtained from National Disease Research Interchange (NDR1, Philadelphia, Pa.). The tissues were obtained following normal deliveries. The cell isolation protocol was performed aseptically in a laminar flow hood. To remove blood and debris, the cord was washed in phosphate buffered saline (PBS; Invitrogen, Carlsbad, Calif.) in the presence of antimycotic and antibiotic (100 units/milliliter penicillin, 100 micrograms/milliliter streptomycin, 0.25 micrograms/milliliter amphotericin B). The tissues were then mechanically dissociated in 150 cm² tissue culture plates in the presence of 50 milliliters of medium (DMEM-Low glucose or DMEM-High glucose; Invitrogen), until the tissue was minced into a fine pulp. The chopped tissues were transferred to 50 milliliter conical tubes (approximately 5 grams of tissue per tube).

The tissue was then digested in either DMEM-Low glucose medium or DMEM-High glucose medium, each containing antimycotic and antibiotic as described above. In some experiments, an enzyme mixture of collagenase and dispase was used (“C:D”) collagenase (Sigma, St Louis, Mo.), 500 Units/milliliter; and dispase (Invitrogen), 50 Units/milliliter in DMEM-Low glucose medium). In other experiments a mixture of collagenase, dispase and hyaluronidase (“C:D:H”) was used (collagenase, 500 Units/milliliter; dispase, 50 Units/milliliter; and hyaluronidase (Sigma), 5 Units/milliliter, in DMEM-Low glucose). The conical tubes containing the tissue, medium and digestion enzymes were incubated at 37° C. in an orbital shaker (Environ, Brooklyn, N.Y.) at 225 rpm for 2 hrs.

After digestion, the tissues were centrifuged at 150×g for 5 minutes, and the supernatant was aspirated. The pellet was resuspended in 20 milliliters of Growth Medium (DMEM-Low glucose (Invitrogen), 15 percent (v/v) fetal bovine serum (FBS; defined bovine serum; Lot#AND18475; Hyclone, Logan, Utah), 0.001% (v/v) 2-mercaptoethanol (Sigma), 1 milliliter per 100 milliliters of antibiotic/antimycotic as described above. The cell suspension was filtered through a 70-micrometer nylon cell strainer (BD Biosciences). An additional 5 milliliters rinse comprising Growth Medium was passed through the strainer. The cell suspension was then passed through a 40-micrometer nylon cell strainer (BD Biosciences) and chased with a rinse of an additional 5 milliliters of Growth Medium.

The filtrate was resuspended in Growth Medium (total volume 50 milliliters) and centrifuged at 150×g for 5 minutes. The supernatant was aspirated and the cells were resuspended in 50 milliliters of fresh Growth Medium. This process was repeated twice more.

Upon the final centrifugation, supernatant was aspirated and the cell pellet was resuspended in 5 milliliters of fresh Growth Medium. The number of viable cells was determined using Trypan Blue staining. Cells were then cultured under standard conditions.

The cells isolated from umbilical cords were seeded at 5,000 cells/cm² onto gelatin-coated T-75 cm² flasks (Corning Inc., Corning, N.Y.) in Growth Medium with antibiotics/antimycotics as described above. After 2 days (in various experiments, cells were incubated from 2-4 days), spent medium was aspirated from the flasks. Cells were washed with PBS three times to remove debris and blood-derived cells. Cells were then replenished with Growth Medium and allowed to grow to confluence (about 10 days from passage 0) to passage 1. On subsequent passages (from passage 1 to 2 and so on), cells reached sub-confluence (75-85 percent confluence) in 4-5 days. For these subsequent passages, cells were seeded at 5000 cells/cm². Cells were grown in a humidified incubator with 5 percent carbon dioxide and atmospheric oxygen, at 37° C.

Placental Cell Isolation: Placental tissue was obtained from NDRI (Philadelphia, Pa.). The tissues were from a pregnancy and were obtained at the time of a normal surgical delivery. Placental cells were isolated as described for umbilical cell isolation.

The following example applies to the isolation of separate populations of maternal-derived and neonatal-derived cells from placental tissue.

The cell isolation protocol was performed aseptically in a laminar flow hood. The placental tissue was washed in phosphate buffered saline (PBS; Invitrogen, Carlsbad, Calif.) in the presence of antimycotic and antibiotic (as described above) to remove blood and debris. The placental tissue was then dissected into three sections: top-line (neonatal side or aspect), mid-line (mixed cell isolation neonatal and maternal) and bottom line (maternal side or aspect).

The separated sections were individually washed several times in PBS with antibiotic/antimycotic to further remove blood and debris. Each section was then mechanically dissociated in 150 cm² tissue culture plates in the presence of 50 milliliters of DMEM-Low glucose, to a fine pulp. The pulp was transferred to 50 milliliter conical tubes. Each tube contained approximately 5 grams of tissue. The tissue was digested in either DMEM-Low glucose or DMEM-High glucose medium containing antimycotic and antibiotic (100 U/milliliter penicillin, 100 micrograms/milliliter streptomycin, 0.25 micrograms/milliliter amphotericin B) and digestion enzymes. In some experiments an enzyme mixture of collagenase and dispase (“C:D”) was used containing collagenase (Sigma, St Louis, Mo.) at 500 Units/milliliter and dispase (Invitrogen) at 50 Units/milliliter in DMEM-Low glucose medium. In other experiments a mixture of collagenase, dispase and hyaluronidase (C:D:H) was used (collagenase, 500 Units/milliliter; dispase, 50 Units/milliliter; and hyaluronidase (Sigma), 5 Units/milliliter in DMEM-Low glucose). The conical tubes containing the tissue, medium, and digestion enzymes were incubated for 2 h at 37° C. in an orbital shaker (Environ, Brooklyn, N.Y.) at 225 rpm.

After digestion, the tissues were centrifuged at 150×g for 5 minutes, the resultant supernatant was aspirated off. The pellet was resuspended in 20 milliliters of Growth Medium with penicillin/streptomycin/amphotericin B. The cell suspension was filtered through a 70 micometer nylon cell strainer (BD Biosciences), chased by a rinse with an additional 5 milliliters of Growth Medium. The total cell suspension was passed through a 40 micometer nylon cell strainer (BD Biosciences) followed with an additional 5 milliliters of Growth Medium as a rinse.

The filtrate was resuspended in Growth Medium (total volume 50 milliliters) and centrifuged at 150×g for 5 minutes. The supernatant was aspirated and the cell pellet was resuspended in 50 milliliters of fresh Growth Medium. This process was repeated twice more. After the final centrifugation, supernatant was aspirated and the cell pellet was resuspended in 5 milliliters of fresh Growth Medium. A cell count was determined using the Trypan Blue Exclusion test. Cells were then cultured at standard conditions.

LIBERASE Cell Isolation:

Cells were isolated from umbilicus tissues in DMEM-Low glucose medium with LIBERASE (Boehringer Mannheim Corp., Indianapolis, Ind.) (2.5 milligrams per milliliter, Blendzyme 3; Roche Applied Sciences, Indianapolis, Ind.) and hyaluronidase (5 Units/milliliter, Sigma). Digestion of the tissue and isolation of the cells was as described for other protease digestions above, using the LIBERASE/hyaluronidase mixture in place of the C:D or C:D:H enzyme mixture. Tissue digestion with LIBERASE resulted in the isolation of cell populations from postpartum tissues that expanded readily.

Cell Isolation Using Other Enzyme Combinations:

Procedures were compared for isolating cells from the umbilical cord using differing enzyme combinations. Enzymes compared for digestion included: i) collagenase; ii) dispase; iii) hyaluronidase; iv) collagenase: dispase mixture (C:D); v) collagenase: hyaluronidase mixture (C:H); vi) dispase: hyaluronidase mixture (D:H); and vii) collagenase: dispase: hyaluronidase mixture (C:D:H). Differences in cell isolation utilizing these different enzyme digestion conditions were observed (Table 6-1).

Isolation of Cells from Residual Blood in the Cords:

Other attempts were made to isolate pools of cells from umbilical cord by different approaches. In one instance umbilical cord was sliced and washed with Growth Medium to dislodge the blood clots and gelatinous material. The mixture of blood, gelatinous material and Growth Medium was collected and centrifuged at 150″ g. The pellet was resuspended and seeded onto gelatin-coated flasks in Growth Medium. From these experiments a cell population was isolated that readily expanded.

Isolation of Cells from Cord Blood:

Cells have also been isolated from cord blood samples attained from NDR1. The isolation protocol used here was that of International Patent Application WO 2003/025149 by Ho et al. (Ho, T. W., et al., “Cell Populations Which Co-Express CD49C and CD90,” Application No. PCT/US02/29971). Samples (50 milliliter and 10.5 milliliters, respectively) of umbilical cord blood (NDR1, Philadelphia Pa.) were mixed with lysis buffer (filter-sterilized 155 mM ammonium chloride, 10 millimolar potassium bicarbonate, 0.1 millimolar EDT A buffered to pH 7.2 (all components from Sigma, St. Louis, Mo.)). Cells were lysed at a ratio of 1:20 cord blood to lysis buffer. The resulting cell suspension was vortexed for 5 seconds, and incubated for 2 minutes at ambient temperature. The lysate was centrifuged (10 minutes at 200×g). The cell pellet was resuspended in complete minimal essential medium (Gibco, Carlsbad, Calif.) containing 10 percent fetal bovine serum (Hyclone, Logan Utah), 4 millimolar glutamine (Mediatech, Herndon, Va.), 100 Units penicillin per 100 milliliters and 100 micrograms streptomycin per 100 milliliters (Gibco, Carlsbad, Calif.). The resuspended cells were centrifuged (10 minutes at 200×g), the supernatant was aspirated, and the cell pellet was washed in complete medium. Cells were seeded directly into either T75 flasks (Corning, N.Y.), T75 laminin-coated flasks, or T175 fibronectin-coated flasks (both Becton Dickinson, Bedford, Mass.).

Isolation of Cells Using Different Enzyme Combinations and Growth Conditions:

To determine whether cell populations could be isolated under different conditions and expanded under a variety of conditions immediately after isolation, cells were digested in Growth Medium with or without 0.001 percent (v/v) 2-mercaptoethanol (Sigma, St. Louis, Mo.), using the enzyme combination of C:D:H, according to the procedures provided above. Placental-derived cells so isolated were seeded under a variety of conditions. All cells were grown in the presence of penicillin/streptomycin. (Table 6-2).

Isolation of Cells Using Different Enzyme Combinations and Growth Conditions:

In all conditions cells attached and expanded well between passage 0 and 1 (Table 6-2). Cells in conditions 5-8 and 13-16 were demonstrated to proliferate well up to 4 passages after seeding at which point they were cryopreserved and banked.

### Results

Cell Isolation Using Different Enzyme Combinations:

The combination of C:D:H, provided the best cell yield following isolation, and generated cells, which expanded for many more generations in culture than the other conditions (Table 6-1). An expandable cell population was not attained using collagenase or hyaluronidase alone. No attempt was made to determine if this result is specific to the collagen that was tested.

TABLE 6-1
Isolation of cells from umbilical cord tissue
using varying enzyme combinations

| Enzyme Digest | Cells Isolated | Cell Expansion |
|---|---|---|
| Collagenase | X | X |
| Dispase | +(>10 h) | + |
| Hyaluronidase | X | X |
| Collagenase: Dispase | ++(<3 h) | ++ |
| Collagenase: Hyaluronidase | ++(<3 h) | + |
| Dispase: Hyaluronidase | +(>10 h) | + |
| Collagenase: Dispase: Hyaluronidase | +++ (<3 h) | +++ |

Key:
+ = good,
++ = very good,
+++ = excellent,
X = no success

Isolation of Cells Using Different Enzyme Combinations and Growth Conditions:

Cells attached and expanded well between passage 0 and 1 under all conditions tested for enzyme digestion and growth (Table 6-2). Cells in experimental conditions 5-8 and 13-16 proliferated well up to 4 passages after seeding, at which point they were cryopreserved. All cells were cryopreserved for further investigation.

TABLE 6-2
Isolation and culture expansion of postpartum cells under varying conditions:

| Condition | Medium | 15% FBS | BME | Gelatin | 20% O₂ | Growth Factors |
|---|---|---|---|---|---|---|
| 1 | DMEM-Lg | Y | Y | Y | Y | N |
| 2 | DMEM-Lg | Y | Y | Y | N (5%) | N |
| 3 | DMEM-Lg | Y | Y | N | Y | N |
| 4 | DMEM-Lg | Y | Y | N | N (5%) | N |
| 5 | DMEM-Lg | N (2%) | Y | N (Laminin) | Y | EGF/FGF (20 ng/ml) |
| 6 | DMEM-Lg | N (2%) | Y | N (Laminin) | N (5%) | EGF/FGF (20 ng/ml) |
| 7 | DMEM-Lg | N (2%) | Y | N | Y | PDGF/VEGF |
| | | | | (Fibronectin) | | |
| 8 | DMEM-Lg | N (2%) | Y | N | N (5%) | PDGF/VEGF |
| | | | | (Fibronectin) | | |
| 9 | DMEM-Lg | Y | N | Y | Y | N |
| 10 | DMEM-Lg | Y | N | Y | N (5%) | N |
| 11 | DMEM-Lg | Y | N | N | Y | N |
| 12 | DMEM-Lg | Y | N | N | N (5%) | N |
| 13 | DMEM-Lg | N (2%) | N | N (Laminin) | Y | EGF/FGF (20 ng/ml) |
| 14 | DMEM-Lg | N (2%) | N | N (Laminin) | N (5%) | EGF/FGF (20 ng/ml) |
| 15 | DMEM-Lg | N (2%) | N | N | Y | PDGF/VEGF |
| | | | | (Fibronectin) | | |
| 16 | DMEM-Lg | N (2%) | N | N | N (5%) | PDGF/VEGF |
| | | | | (Fibronectin) | | |

Isolation of Cells from Residual Blood in the Cords:

Nucleated cells attached and grew rapidly. These cells were analyzed by flow cytometry and were similar to cells obtained by enzyme digestion.

Isolation of Cells from Cord Blood:

The preparations contained red blood cells and platelets. No nucleated cells attached and divided during the first 3 weeks. The medium was changed 3 weeks after seeding and no cells were observed to attach and grow.

Summary:

Populations of cells can be derived from umbilical cord and placental tissue efficiently using the enzyme combination collagenase (a matrix metalloprotease), dispase (a neutral protease) and hyaluronidase (a mucolytic enzyme that breaks down hyaluronic acid). LIBERASE, which is a Blendzyme, may also be used. Specifically, Blendzyme 3, which is collagenase (4 Wunsch units/g) and thermolysin (1714 casein Units/g) was also used together with hyaluronidase to isolate cells. These cells expanded readily over many passages when cultured in Growth Medium on gelatin-coated plastic.

Cells were also isolated from residual blood in the cords, but not cord blood. The presence of cells in blood clots washed from the tissue that adhere and grow under the conditions used may be due to cells being released during the dissection process.

### Example 7

### Karyotype Analysis of Postpartum-Derived Cells

Cell lines used in cell therapy are preferably homogeneous and free from any contaminating cell type. Cells used in cell therapy should have a normal chromosome number (46) and structure. To identify placenta- and umbilicus-derived cell lines that are homogeneous and free from cells of non-postpartum tissue origin, karyotypes of cell samples were analyzed.

### Methods & Materials

PPDCs from postpartum tissue of a male neonate were cultured in Growth Medium containing penicillin/streptomycin. Postpartum tissue from a male neonate (X,Y) was selected to allow distinction between neonatal-derived cells and maternal derived cells (X,X). Cells were seeded at 5,000 cells per square centimeter in Growth Medium in a T25 flask (Corning Inc., Corning, N.Y.) and expanded to 80% confluence. A T25 flask containing cells was filled to the neck with Growth Medium. Samples were delivered to a clinical cytogenetics laboratory by courier (estimated lab to lab transport time is one hour). Cells were analyzed during metaphase when the chromosomes are best visualized. Of twenty cells in metaphase counted, five were analyzed for normal homogeneous karyotype number (two). A cell sample was characterized as homogeneous if two karyotypes were observed. A cell sample was characterized as heterogeneous if more than two karyotypes were observed. Additional metaphase cells were counted and analyzed when a heterogeneous karyotype number (four) was identified.

### Results

All cell samples sent for chromosome analysis were interpreted as exhibiting a normal appearance. Three of the 16 cell lines analyzed exhibited a heterogeneous phenotype (XX and XY) indicating the presence of cells derived from both neonatal and maternal origins (Table 7-1). Cells derived from tissue Placenta-N were isolated from the neonatal aspect of placenta. At passage zero, this cell line appeared homogeneous XY. However, at passage nine, the cell line was heterogeneous (XX/XY), indicating a previously undetected presence of cells of maternal origin.

TABLE 7-1
Karyotype results of PPDCs.

| | | Metaphase | Metaphase | Number of | ISCN |
|---|---|---|---|---|---|
| Tissue | passage | cells counted | cells analyzed | karyotypes | Karyotype |
| Placenta | 22 | 20 | 5 | 2 | 46 , XX |
| Umbilical | 23 | 20 | 5 | 2 | 46, XX |
| Umbilical | 6 | 20 | 5 | 2 | 46, XY |
| Placenta | 2 | 20 | 5 | 2 | 46, XX |
| Umbilical | 3 | 20 | 5 | 2 | 46, XX |
| Placenta-N | 0 | 20 | 5 | 2 | 46, XY |
| Placenta-V | 0 | 20 | 5 | 2 | 46, XY |
| Placenta-M | 0 | 21 | 5 | 4 | 46, XY[18]/ |
| | | | | | 46, XX[3] |
| Placenta-M | 4 | 20 | 5 | 2 | 46,XX |
| Placenta-N | 9 | 25 | 5 | 4 | 46, XY[5]/ |
| | | | | | 46, XX[20] |
| Placenta-N | 1 | 20 | 5 | 2 | 46, XY |
| C1 | | | | | |
| Placenta-N | 1 | 20 | 6 | 4 | 46, XY[2]/ |
| C3 | | | | | 46, XX[18] |
| Placenta-N | 1 | 20 | 5 | 2 | 46, XY |
| C4 | | | | | |
| Placenta-N | 1 | 20 | 5 | 2 | 46, XY |
| C15 | | | | | |
| Placenta-N | 1 | 20 | 5 | 2 | 46, XY |
| C20 | | | | | |
| Placenta-N | 1 | 20 | 5 | 2 | 46, XY |
| C22 | | | | | |

Key:
N-Neonatal side;
V-villous region;
M-maternal side;
C-clone

Summary:

Chromosome analysis identified placenta- and umbilicus-derived cells whose karyotypes appeared normal as interpreted by a clinical cytogenetic laboratory. Karyotype analysis also identified cell lines free from maternal cells, as determined by homogeneous karyotype.

### Example 8

### Evaluation of Human Postpartum-Derived Cell Surface Markers by Flow Cytometry

Characterization of cell surface proteins or “markers” by flow cytometry can be used to determine a cell line's identity. The consistency of expression can be determined from multiple donors, and in cells exposed to different processing and culturing conditions. Postpartum-derived cell (PPDC) lines isolated from the placenta and umbilicus were characterized (by flow cytometry), providing a profile for the identification of these cell lines.

### Methods & Materials

Media and Culture Vessels:

Cells were cultured in Growth Medium (Gibco Carlsbad, Calif.) with penicillin/streptomycin. Cells were cultured in plasma-treated T75, T150, and T225 tissue culture flasks (Corning Inc., Corning, N.Y.) until confluent. The growth surfaces of the flasks were coated with gelatin by incubating 2% (w/v) gelatin (Sigma, St. Louis, Mo.) for 20 minutes at room temperature.

Antibody Staining and Flow Cytometry Analysis:

Adherent cells in flasks were washed in PBS and detached with Trypsin/EDTA. Cells were harvested, centrifuged, and resuspended in 3% (v/v) FBS in PBS at a cell concentration of 1×10⁷ per milliliter. In accordance to the manufacture's specifications, antibody to the cell surface marker of interest (see below) was added to one hundred microliters of cell suspension and the mixture was incubated in the dark for 30 minutes at 4° C. After incubation, cells were washed with PBS and centrifuged to remove unbound antibody. Cells were resuspended in 500 microliter PBS and analyzed by flow cytometry. Flow cytometry analysis was performed with a FACScalibur™ instrument (Becton Dickinson, San Jose, Calif.). Table 8-1 lists the antibodies to cell surface markers that were used.

TABLE 8-1
Antibodies used in characterizing cell surface markers.

| Antibody | Manufacture | Catalog Number |
|---|---|---|
| CD10 | BD Pharmingen (San Diego, CA) | 555375 |
| CD13 | BD Pharmingen (San Diego, CA) | 555394 |
| CD31 | BD Pharmingen (San Diego, CA) | 555446 |
| CD34 | BD Pharmingen (San Diego, CA) | 555821 |
| CD44 | BD Pharmingen (San Diego, CA) | 555478 |
| CD45RA | BD Pharmingen (San Diego, CA) | 555489 |
| CD73 | BD Pharmingen (San Diego, CA) | 550257 |
| CD90 | BD Pharmingen (San Diego, CA) | 555596 |
| CD117 | BD Biosciences (San Jose, CA) | 340529 |
| CD141 | BD Pharmingen (San Diego, CA) | 559781 |
| PDGFr-alpha | BD Pharmingen (San Diego, CA) | 556002 |
| HLA-A, B, C | BD Pharmingen (San Diego, CA) | 555553 |
| HLA-DR, DP, DQ | BD Pharmingen (San Diego, CA) | 555558 |
| IgG-FITC | Sigma (St. Louis, MO) | F-6522 |
| IgG-PE | Sigma (St. Louis, MO) | P-4685 |

Placenta and Umbilicus Comparison:

Placenta-derived cells were compared to umbilicus-derive cells at passage 8.

Passage to Passage Comparison:

Placenta- and umbilicus-derived cells were analyzed at passages 8, 15, and 20.

Donor to Donor Comparison:

To compare differences among donors, placenta-derived cells from different donors were compared to each other, and umbilicus-derived cells from different donors were compared to each other.

Surface Coating Comparison:

Placenta-derived cells cultured on gelatin-coated flasks was compared to placenta-derived cells cultured on uncoated flasks. Umbilicus-derived cells cultured on gelatin-coated flasks was compared to umbilicus-derived cells cultured on uncoated flasks.

Digestion Enzyme Comparison:

Four treatments used for isolation and preparation of cells were compared. Cells isolated from placenta by treatment with 1) collagenase; 2) collagenase/dispase; 3) collagenase/hyaluronidase; and 4) collagenase/hyaluronidase/dispase were compared.

Placental Layer Comparison:

Cells derived from the maternal aspect of placental tissue were compared to cells derived from the villous region of placental tissue and cells derived from the neonatal fetal aspect of placenta.

### Results

Placenta Vs. Umbilicus Comparison:

Placenta- and umbilicus-derived cells analyzed by flow cytometry showed positive expression of CD10, CD13, CD44, CD73, CD90, PDGFr-alpha and HLA-A, B, C, indicated by the increased values of fluorescence relative to the IgG control. These cells were negative for detectable expression of CD31, CD34, CD45, CD117, CD141, and HLA-DR, DP, DQ, indicated by fluorescence values comparable to the IgG control. Variations in fluorescence values of positive curves were accounted. The mean (i.e. CD13) and range (i.e. CD90) of the positive curves showed some variation, but the curves appeared normal, confirming a homogenous population. Both curves individually exhibited values greater than the IgG control.

Passage to Passage Comparison—Placenta-Derived Cells:

Placenta-derived cells at passages 8, 15, and 20 analyzed by flow cytometry all were positive for expression of CD10, CD13, CD44, CD73, CD90, PDGFr-alpha and HLA-A, B, C, as reflected in the increased value of fluorescence relative to the IgG control. The cells were negative for expression of CD31, CD34, CD45, CD117, CD141, and HLA-DR, DP, DQ having fluorescence values consistent with the IgG control.

Passage to Passage Comparison—Umbilicus-Derived Cells:

Umbilicus-derived cells at passage 8, 15, and 20 analyzed by flow cytometry all expressed CD10, CD13, CD44, CD73, CD90, PDGFr-alpha and HLA-A, B, C, indicated by increased fluorescence relative to the IgG control. These cells were negative for CD31, CD34, CD45, CD117, CD141, and HLA-DR, DP, DQ, indicated by fluorescence values consistent with the IgG control.

Donor to Donor Comparison—Placenta-Derived Cells:

Placenta-derived cells isolated from separate donors analyzed by flow cytometry each expressed CD10, CD13, CD44, CD73, CD90, PDGFr-alpha and HLA-A, B, C, with increased values of fluorescence relative to the IgG control. The cells were negative for expression of CD31, CD34, CD45, CD117, CD141, and HLA-DR, DP, DQ as indicated by fluorescence value consistent with the IgG control.

Donor to Donor Comparison—Umbilicus Derived Cells:

Umbilicus-derived cells isolated from separate donors analyzed by flow cytometry each showed positive expression of CD10, CD13, CD44, CD73, CD90, PDGFr-alpha and HLA-A, B, C, reflected in the increased values of fluorescence relative to the IgG control. These cells were negative for expression of CD31, CD34, CD45, CD117, CD141, and HLA-DR, DP, DQ with fluorescence values consistent with the IgG control.

The Effect of Surface Coating with Gelatin on Placenta-Derived Cells:

Placenta-derived cells expanded on either gelatin-coated or uncoated flasks analyzed by flow cytometry all expressed of CD10, CD13, CD44, CD73, CD90, PDGFr-alpha and HLA-A, B, C, reflected in the increased values of fluorescence relative to the IgG control. These cells were negative for expression of CD31, CD34, CD45, CD117, CD141, and HLA-DR, DP, DQ indicated by fluorescence values consistent with the IgG control.

The Effect of Surface Coating with Gelatin on Umbilicus-Derived Cells:

Umbilicus-derived cells expanded on gelatin and uncoated flasks analyzed by flow cytometry all were positive for expression of CD10, CD13, CD44, CD73, CD90, PDGFr-alpha and HLA-A, B, C, with increased values of fluorescence relative to the IgG control. These cells were negative for expression of CD31, CD34, CD45, CD117, CD141, and HLA-DR, DP, DQ, with fluorescence values consistent with the IgG control.

Effect of Enzyme Digestion Procedure Used for Preparation of the Cells on the Cell Surface Marker Profile:

Placenta-derived cells isolated using various digestion enzymes analyzed by flow cytometry all expressed CD10, CD13, CD44, CD73, CD90, PDGFr-alpha and HLA-A, B, C, as indicated by the increased values of fluorescence relative to the IgG control. These cells were negative for expression of CD31, CD34, CD45, CD117, CD141, and HLADR, DP, DQ as indicated by fluorescence values consistent with the IgG control.

Placental Layer Comparison:

Cells isolated from the maternal, villous, and neonatal layers of the placenta, respectively, analyzed by flow cytometry showed positive expression of CD10, CD13, CD44, CD73, CD90, PDGFr-alpha and HLA-A, B, C, as indicated by the increased value of fluorescence relative to the IgG control. These cells were negative for expression of CD31, CD34, CD45, CD117, CD141, and HLA-DR, DP, DQ as indicated by fluorescence values consistent with the IgG control.

Summary:

Analysis of placenta- and umbilicus-derived cells by flow cytometry has established of an identity of these cell lines. Placenta- and umbilicus-derived cells are positive for CD10, CD13, CD44, CD73, CD90, PDGFr-alpha, HLA-A,B,C and negative for CD31, CD34, CD45, CD117, CD141 and HLA-DR, DP, DQ. This identity was consistent between variations in variables including the donor, passage, culture vessel surface coating, digestion enzymes, and placental layer. Some variation in individual fluorescence value histogram curve means and ranges was observed, but all positive curves under all conditions tested were normal and expressed fluorescence values greater than the IgG control, thus confirming that the cells comprise a homogenous population that has positive expression of the markers.

### Example 9

### Immunohistochemical Characterization of Postpartum Tissue Phenotypes

The phenotypes of cells found within human postpartum tissues, namely umbilical cord and placenta, was analyzed by immunohistochemistry.

### Methods & Materials

Tissue Preparation:

Human umbilical cord and placenta tissue was harvested and immersion fixed in 4% (w/v) paraformaldehyde overnight at 4° C. Immunohistochemistry was performed using antibodies directed against the following epitopes: vimentin (1:500; Sigma, St. Louis, Mo.), desmin (1:150, raised against rabbit; Sigma; or 1:300, raised against mouse; Chemic on, Temecula, Calif.), alpha-smooth muscle actin (SMA; 1:400; Sigma), cytokeratin 18 (CK18; 1:400; Sigma), von Willebrand Factor (vWF; 1:200; Sigma), and CD34 (human CD34 Class III; 1:100; DAKOCytomation, Carpinteria, Calif.). In addition, the following markers were tested: antihuman GROalpha—PE (1: 100; Becton Dickinson, Franklin Lakes, N.J), antihuman GCP-2 (1:100; Santa Cruz Biotech, Santa Cruz, Calif.), anti-human oxidized LDL receptor 1 (ox-LDL R1; 1:100; Santa Cruz Biotech), and anti-human NOGO-A (1:100; Santa Cruz Biotech). Fixed specimens were trimmed with a scalpel and placed within OCT embedding compound (Tissue-Tek OCT; Sakura, Torrance, Calif.) on a dry ice bath containing ethanol. Frozen blocks were then sectioned (10 μm thick) using a standard cryostat (Leica Microsystems) and mounted onto glass slides for staining.

Immunohistochemistry:

Immunohistochemistry was performed similar to previous studies (e.g., Messina, et al., 2003, Exper. Neurol. 184: 816-829). Tissue sections were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma) for 1 hour to access intracellular antigens. In instances where the epitope of interest would be located on the cell surface (CD34, ox-LDL R1), Triton was omitted in all steps of the procedure in order to prevent epitope loss. Furthermore, in instances where the primary antibody was raised against goat (GCP-2, ox-LDL R1, NOGO-A), 3% (v/v) donkey serum was used in place of goat serum throughout the procedure. Primary antibodies, diluted in blocking solution, were then applied to the sections for a period of 4 hours at room temperature. Primary antibody solutions were removed, and cultures washed with PBS prior to application of secondary antibody solutions (1 hour at room temperature) containing block along with goat anti-mouse IgG-Texas Red (1:250; Molecular Probes, Eugene, Oreg.) and/or goat anti-rabbit IgG-Alexa 488 (1:250; Molecular Probes) or donkey anti-goat IgG-FITC (1:150; Santa Cruz Biotech). Cultures were washed, and 10 micromolar DAPI (Molecular Probes) was applied for 10 minutes to visualize cell nuclei.

Following immunostaining, fluorescence was visualized using the appropriate fluorescence filter on an Olympus inverted epi-fluorescent microscope (Olympus, Melville, N.Y.). Positive staining was represented by fluorescence signal above control staining. Representative images were captured using a digital color video camera and ImagePro software (Media Cybernetics, Carlsbad, Calif.). For triple-stained samples, each image was taken using only one emission filter at a time. Layered montages were then prepared using Adobe Photoshop software (Adobe, San Jose, Calif.).

### Results

Umbilical Cord Characterization:

Vimentin, desmin, SMA, CKI8, vWF, and CD34 markers were expressed in a subset of the cells found within umbilical cord. In particular, vWF and CD34 expression were restricted to blood vessels contained within the cord. CD34+ cells were on the innermost layer (lumen side). Vimentin expression was found throughout the matrix and blood vessels of the cord. SMA was limited to the matrix and outer walls of the artery & vein, but not contained with the vessels themselves. CK18 and desmin were observed within the vessels only, desmin being restricted to the middle and outer layers.

Placenta Characterization:

Vimentin, desmin, SMA, CKI8, vWF, and CD34 were all observed within the placenta and regionally specific.

GROalpha, GCP-2, Ox-LDL RI, and NOGO-A Tissue Expression:

None of these markers were observed within umbilical cord or placental tissue.

Summary:

Vimentin, desmin, alpha-smooth muscle actin, cytokeratin 18, von Willebrand Factor, and CD34 are expressed in cells within human umbilical cord and placenta.

### Example 10

### Analysis of Postpartum Tissue-Derived Cells Using Oligonucleotide Arrays

Affymetrix GENECHIP arrays were used to compare gene expression profiles of umbilicus- and placenta-derived cells with fibroblasts, human mesenchymal stem cells, and another cell line derived from human bone marrow. This analysis provided a characterization of the postpartum-derived cells and identified unique molecular markers for these cells.

### Methods & Materials

Isolation and Culture of Cells:

Human umbilical cords and placenta were obtained from National Disease Research Interchange (NDRI, Philadelphia, Pa.) from normal full term deliveries with patient consent. The tissues were received and cells were isolated as described in Example 6. Cells were cultured in Growth Medium (using DMEM-LG) on gelatin-coated tissue culture plastic flasks. The cultures were incubated at 37° C. with 5% CO2.

Human dermal fibroblasts were purchased from Cambrex Incorporated (Walkersville, Md.; Lot number 9F0844) and ATCC CRL-1501 (CCD39SK). Both lines were cultured in DMEM/F12 medium (Invitrogen, Carlsbad, Calif.) with 10% (v/v) fetal bovine serum (Hyclone) and penicillin/streptomycin (Invitrogen). The cells were grown on standard tissue-treated plastic.

Human mesenchymal stem cells (hMSC) were purchased from Cambrex Incorporated (Walkersville, Md.; Lot numbers 2F1655, 2F1656 and 2F1657) and cultured according to the manufacturer's specifications in MSCGM Media (Cambrex). The cells were grown on standard tissue cultured plastic at 37° C. with 5% CO2.

Human iliac crest bone marrow was received from the NDRI with patient consent. The marrow was processed according to the method outlined by Ho, et al. (WO03/025149). The marrow was mixed with lysis buffer (155 mM NH 4Cl, 10 mM KHCO₃, and 0.1 mM EDTA, pH 7.2) at a ratio of 1 part bone marrow to 20 parts lysis buffer. The cell suspension was vortexed, incubated for 2 minutes at ambient temperature, and centrifuged for 10 minutes at 500×g. The supernatant was discarded and the cell pellet was resuspended in Minimal Essential Medium-alpha (Invitrogen) supplemented with 10% (v/v) fetal bovine serum and 4 mM glutamine. The cells were centrifuged again and the cell pellet was resuspended in fresh medium. The viable mononuclear cells were counted using trypan-blue exclusion (Sigma, St. Louis, Mo.). The mononuclear cells were seeded in tissue-cultured plastic flasks at 5×10⁴ cells/cm². The cells were incubated at 37° C. with 5% CO2 at either standard atmospheric O₂ or at 5% O₂. Cells were cultured for 5 days without a media change. Media and non-adherent cells were removed after 5 days of culture. The adherent cells were maintained in culture.

Isolation of mRNA and GENECHIP Analysis:

Actively growing cultures of cells were removed from the flasks with a cell scraper in cold PBS. The cells were centrifuged for 5 minutes at 300×g. The supernatant was removed and the cells were resuspended in fresh PBS and centrifuged again. The supernatant was removed and the cell pellet was immediately frozen and stored at −80° C. Cellular mRNA was extracted and transcribed into cDNA, which was then transcribed into cRNA and biotin-labeled. The biotin-labeled cRNA was hybridized with HG-U133A GENECHIP oligonucleotide array (Affymetrix, Santa Clara Calif.). The hybridization and data collection was performed according to the manufacturer's specifications. Analyses were performed using “Significance Analysis of Microarrays” (SAM) version 1.21 computer software (Stanford University; Tusher, V. G. et al., 2001, Proc. Natl. Acad. Sci. USA 98: 5116-5121).

### Results

Fourteen different populations of cells were analyzed. The cells along with passage information, culture substrate, and culture media are listed in Table 10-1.

TABLE 10-1
Cells analyzed by the microarray study. The cells lines are
listed by their identification code along with passage at the time
of analysis, cell growth substrate, and growth media.

| Cell Population | Passage | Substrate | Medium |
|---|---|---|---|
| Umbilical (022803) | 2 | Gelatin | DMEM, 15% FBS, 2-ME |
| Umbilical (042103) | 3 | Gelatin | DMEM, 15% FBS, 2-ME |
| Umbilical (071003) | 4 | Gelatin | DMEM, 15% FBS, 2-ME |
| Placenta (042203) | 12 | Gelatin | DMEM, 15% FBS, 2-ME |
| Placenta (042903) | 4 | Gelatin | DMEM, 15% FBS, 2-ME |
| Placenta (071003) | 3 | Gelatin | DMEM, 15% FBS, 2-ME |
| ICBM (070203) (5% O₂) | 3 | Plastic | MEM 10% FBS |
| ICBM (062703) (std O₂) | 5 | Plastic | MEM 10% FBS |
| ICBM (062703 )(5% O₂) | 5 | Plastic | MEM 10% FBS |
| hMSC (Lot 2F1655) | 3 | Plastic | MSCGM |
| hMSC (Lot 2F1656) | 3 | Plastic | MSCGM |
| hMSC (Lot 2F1657) | 3 | Plastic | MSCGM |
| hFibroblast (9F0844) | 9 | Plastic | DMEM-F12, 10% FBS |
| hFibroblast (CCD39SK) | 4 | Plastic | DMEM-F12, 10% FBS |

The data were evaluated by a Principle Component Analysis, analyzing the 290 genes that were differentially expressed in the cells. This analysis allows for a relative comparison for the similarities between the populations.

Table 10-2 shows the Euclidean distances that were calculated for the comparison of the cell pairs. The Euclidean distances were based on the comparison of the cells based on the 290 genes that were differentially expressed among the cell types. The Euclidean distance is inversely proportional to similarity between the expression of the 290 genes (i.e., the greater the distance, the less similarity exists).

TABLE 10-2
The Euclidean Distances for the Cell Pairs.

| Cell Pair | Euclidean Distance |
|---|---|
| ICBM-hMSC | 24.71 |
| Placenta-umbilical | 25.52 |
| ICBM-Fibroblast | 36.44 |
| ICBM-placenta | 37.09 |
| Fibroblast-MSC | 39.63 |
| ICBM-Umbilical | 40.15 |
| Fibroblast-Umbilical | 41.59 |
| MSC-Placenta | 42.84 |
| MSC-Umbilical | 46.86 |
| ICBM-placenta | 48.41 |

Tables 10-3, 10-4, and 10-5 show the expression of genes increased in placenta-derived cells (Table 10-3), increased in umbilicus-derived cells (Table 10-4), and reduced in umbilicus- and placenta-derived cells (Table 10-5). The column entitled “Probe Set ID” refers to the manufacturer's identification code for the sets of several oligonucleotide probes located on a particular site on the chip, which hybridize to the named gene (column “Gene Name”), comprising a sequence that can be found within the NCBI (GenBank) database at the specified accession number (column “NCBI Accession Number”).

TABLE 10-3
Genes shown to have specifically increased expression in the placenta-derived
cells as compared to other cell lines assayed
Genes Increased in Placenta-Derived Cells

| | | NCBI Accession |
|---|---|---|
| Probe Set ID | Gene Name | Number |
| 209732_at | C-type (calcium dependent, carbohydrate-recognition domain) | AF070642 |
| | lectin, superfamily member 2 (activation-induced) | |
| 206067_s_at | Wilms tumor 1 | NM_024426 |
| 207016_s_at | aldehyde dehydrogenase 1 family, member A2 | AB015228 |
| 206367_at | renin | NM_000537 |
| 210004_at | oxidized low density lipoprotein (lectin-like) receptor 1 | AF035776 |
| 214993_at | Homo sapiens, clone IMAGE:4179671, mRNA, partial cds | AF070642 |
| 202178_at | protein kinase C, zeta | NM_002744 |
| 209780_at | hypothetical protein DKFZp564F013 | AL136883 |
| 204135_at | downregulated in ovarian cancer 1 | NM_014890 |
| 213542_at | Homo sapiens mRNA; cDNA DKFZp547K1113 (from clone | AI246730 |
| | DKFZp547K1113) | |

TABLE 10-4
Genes shown to have specifically increased expression in
the umbilicus-derived cells as compared
to other cell lines assayed
Genes Increased in Umbilicus-Derived Cells

| | | NCBI |
|---|---|---|
| | | Accession |
| Probe Set ID | Gene Name | Number |
| 202859_x_at | interleukin 8 | NM_000584 |
| 211506_s_at | interleukin 8 | AF043337 |
| 210222_s_at | reticulon 1 | BC000314 |
| 204470_at | chemokine (C-X-C motif) ligand 1 | NM_001511 |
| | (melanoma growth stimulating activity | |
| 206336_at | chemokine (C-X-C motif) ligand 6 | NM_002993 |
| | (granulocyte chemotactic protein 2) | |
| 207850_at | chemokine (C-X-C motif) ligand 3 | NM_002090 |
| 203485_at | reticulon 1 | NM_021136 |
| 202644_s_at | tumor necrosis factor, alpha- | NM_006290 |
| | induced protein 3 | |

TABLE 10-5
Genes shown to have decreased expression in umbilicus- and placenta-derived
cells as compared to other cell lines assayed
Genes Decreased in Umbilicus- and Placenta-Derived Cells

| | | NCBI Accession |
|---|---|---|
| Probe Set ID | Gene name | Number |
| 210135_s_at | short stature homeobox 2 | AF022654.1 |
| 205824_at | heat shock 27 kDa protein 2 | NM_001541.1 |
| 209687_at | chemokine (C-X-C motif) ligand 12 (stromal cell-derived factor 1) | U19495.1 |
| 203666_at | chemokine (C-X-C motif) ligand 12 (stromal cell-derived factor 1) | NM_000609.1 |
| 212670_at | elastin (supravalvular aortic stenosis, Williams-Beuren | AA479278 |
| | syndrome) | |
| 213381_at | Homo sapiens mRNA; cDNA DKFZp586M2022 (from clone | N91149 |
| | DKFZp586M2022) | |
| 206201_s_at | mesenchyme homeo box 2 (growth arrest-specific homeo box) | NM_005924.1 |
| 205817_at | sine oculis homeobox homolog 1 (Drosophila) | NM_005982.1 |
| 209283_at | crystallin, alpha B | AF007162.1 |
| 212793_at | dishevelled associated activator of morphogenesis 2 | BF513244 |
| 213488_at | DKFZP586B2420 protein | AL050143.1 |
| 209763_at | similar to neuralin 1 | AL049176 |
| 205200_at | tetranectin (plasminogen binding protein) | NM_003278.1 |
| 205743_at | src homology three (SH3) and cysteine rich domain | NM_003149.1 |
| 200921_s_at | B-cell translocation gene 1, anti-proliferative | NM_001731.1 |
| 206932_at | cholesterol 25-hydroxylase | NM_003956.1 |
| 204198_s_at | runt-related transcription factor 3 | AA541630 |
| 219747_at | hypothetical protein FLJ23191 | NM_024574.1 |
| 204773_at | interleukin 11 receptor, alpha | NM_004512.1 |
| 202465_at | procollagen C-endopeptidase enhancer | NM_002593.2 |
| 203706_s_at | frizzled homolog 7 (Drosophila) | NM_003507.1 |
| 212736_at | hypothetical gene BC008967 | BE299456 |
| 214587_at | collagen, type VIII, alpha 1 | BE877796 |
| 201645_at | tenascin C (hexabrachion) | NM_002160.1 |
| 210239_at | iroquois homeobox protein 5 | U90304.1 |
| 203903_s_at | Hephaestin | NM_014799.1 |
| 205816_at | integrin, beta 8 | NM_002214.1 |
| 203069_at | synaptic vesicle glycoprotein 2 | NM_014849.1 |
| 213909_at | Homo sapiens cDNA FLJ12280 fis, clone MAMMA1001744 | AU147799 |
| 206315_at | cytokine receptor-like factor 1 | NM_004750.1 |
| 204401_at | potassium intermediate/small conductance calcium-activated | NM_002250.1 |
| | channel, subfamily N, member 4 | |
| 216331_at | integrin, alpha 7 | AK022548.1 |
| 209663_s_at | integrin, alpha 7 | AF072132.1 |
| 213125_at | DKFZP586L151 protein | AW007573 |
| 202133_at | transcriptional co-activator with PDZ-binding motif (TAZ) | AA081084 |
| 206511_s_at | sine oculis homeobox homolog 2 (Drosophila) | NM_016932.1 |
| 213435_at | KIAA1034 protein | AB028957.1 |
| 206115_at | early growth response 3 | NM_004430.1 |
| 213707_s_at | distal-less homeo box 5 | NM_005221.3 |
| 218181_s_at | hypothetical protein FLJ20373 | NM_017792.1 |
| 209160_at | aldo-keto reductase family 1, member C3 (3-alpha | AB018580.1 |
| | hydroxysteroid dehydrogenase, type II) | |
| 213905_x_at | Biglycan | AA845258 |
| 201261_x_at | Biglycan | BC002416.1 |
| 202132_at | transcriptional co-activator with PDZ-binding motif (TAZ) | AA081084 |
| 214701_s_at | fibronectin 1 | AJ276395.1 |
| 213791_at | Proenkephalin | NM_006211.1 |
| 205422_s_at | integrin, beta-like 1 (with EGF-like repeat domains) | NM_004791.1 |
| 214927_at | Homo sapiens mRNA full length insert cDNA clone | AL359052.1 |
| | EUROIMAGE 1968422 | |
| 206070_s_at | EphA3 | AF213459.1 |
| 212805_at | KIAA0367 protein | AB002365.1 |
| 219789_at | natriuretic peptide receptor C/guanylate cyclase C | Al628360 |
| | (atrionatriuretic peptide receptor C) | |
| 219054_at | hypothetical protein FLJ14054 | NM_024563.1 |
| 213429_at | Homo sapiens mRNA; cDNA DKFZp564B222 (from clone | AW025579 |
| | DKFZp564B222) | |
| 204929_s_at | vesicle-associated membrane protein 5 (myobrevin) | NM_006634.1 |
| 201843_s_at | EGF-containing fibulin-like extracellular matrix protein 1 | NM_004105.2 |
| 221478_at | BCL2/adenovirus E1B 19 kDa interacting protein 3-like | AL132665.1 |
| 201792_at | AE binding protein 1 | NM_001129.2 |
| 204570_at | cytochrome c oxidase subunit Vila polypeptide 1 (muscle) | NM_001864.1 |
| 201621_at | neuroblastoma, suppression of tumorigenicity 1 | NM_005380.1 |
| 202718_at | insulin-like growth factor binding protein 2, 36 kDa | NM_000597.1 |

Tables 10-6, 10-7, and 10-8 show the expression of genes increased in human fibroblasts (Table 10-6), ICBM cells (Table 10-7), and MSCs (Table 10-8).

TABLE 10-6
Genes that were shown to have increased
expression in fibroblasts as compared to the other cell lines
assayed.
Genes increased in fibroblasts
dual specificity phosphatase 2
KIAA0527 protein
Homo sapiens cDNA: FLJ23224 fis, clone ADSU02206
dynein, cytoplasmic, intermediate polypeptide 1
ankyrin 3, node of Ranvier (ankyrin G)
inhibin, beta A (activin A, activin AB alpha polypeptide)
ectonucleotide pyrophosphatase/phosphodiesterase 4 (putative
function)
KIAA1053 protein
microtubule-associated protein 1A
zinc finger protein 41
HSPC019 protein
Homo sapiens cDNA: FLJ23564 fis, clone LNG10773
Homo sapiens mRNA; cDNA DKFZp564A072 (from clone
DKFZp564A072)
LIM protein (similar to rat protein kinase C-binding enigma)
inhibitor of kappa light polypeptide gene enhancer in B-cells,
kinase complex-associated protein
hypothetical protein FLJ22004
Human (clone CTG-A4) mRNA sequence
ESTs, Moderately similar to cytokine receptor-like factor 2; cytokine
receptor CRL2 precursor [Homo sapiens]
transforming growth factor, beta 2
hypothetical protein MGC29643
antigen identified by monoclonal antibody MRC OX-2
putative X-linked retinopathy protein

TABLE 10-7
Genes that were shown to have increased expression in the
ICBM-derived cells as compared to the other cell lines assayed.
Genes Increased In ICBM Cells
cardiac ankyrin repeat protein
MHC class I region ORF
integrin, alpha 10
hypothetical protein FLJ22362
UDP-N-acetyl-alpha-D-galactosamine:polypeptide N-
acetylgalactosaminyltransferase 3 (GalNAc-T3)
interferon-induced protein 44
SRY (sex determining region Y)-box 9
(campomelic dysplasia, autosomal sex-reversal)
keratin associated protein 1-1
hippocalcin-like 1
jagged 1 (Alagille syndrome)
roteoglycan 1, secretory granule

TABLE 10-8
Genes that were shown to have increased
expression in the MSC cells as compared to the other cell lines
assayed.
Genes Increased In MSC Cells
interleukin 26
maltase-glucoamylase (alpha-glucosidase)
nuclear receptor subfamily 4, group A, member 2
v-fos FBJ murine osteosarcoma viral oncogene homolog
hypothetical protein DC42
nuclear receptor subfamily 4, group A, member 2
FBJ murine osteosarcoma viral oncogene homolog B
WNT1 inducible signaling pathway protein 1
MCF.2 cell line derived transforming sequence
otassium channel, subfamily K, member 15
cartilage paired-class homeoprotein 1
Homo sapiens cDNA FLJ12232 fis, clone MAMMA1001206
Homo sapiens cDNA FLJ34668 fis, clone LIVER2000775
jun B proto-oncogene
B-cell CLL/lymphoma 6 (zinc finger protein 51)
zinc finger protein 36, C3H type, homolog (mouse)

Summary:

The present examination was performed to provide a molecular characterization of the postpartum cells derived from umbilical cord and placenta. This analysis included cells derived from three different umbilical cords and three different placentas. The examination also included two different lines of dermal fibroblasts, three lines of mesenchymal stem cells, and three lines of iliac crest bone marrow cells. The mRNA that was expressed by these cells was analyzed using an oligonucleotide array that contained probes for 22,000 genes. Results showed that 290 genes are differentially expressed in these five different cell types. These genes include ten genes that are specifically increased in the placenta-derived cells and seven genes specifically increased in the umbilical cord-derived cells. Fifty-four genes were found to have specifically lower expression levels in placenta and umbilical cord, as compared with the other cell types. The expression of selected genes has been confirmed by PCR (see the example that follows). These results demonstrate that the postpartum-derived cells have a distinct gene expression profile, for example, as compared to bone marrow-derived cells and fibroblasts.

### Example 11

### Cell Markers in Postpartum-Derived Cells

In the preceding example, similarities and differences in cells derived from the human placenta and the human umbilical cord were assessed by comparing their gene expression profiles with those of cells derived from other sources (using an oligonucleotide array). Six “signature” genes were identified: oxidized LDL receptor 1, interleukin-8, rennin, reticulon, chemokine receptor ligand 3 (CXC ligand 3), and granulocyte chemotactic protein 2 (GCP-2). These “signature” genes were expressed at relatively high levels in postpartum-derived cells.

The procedures described in this example were conducted to verify the microarray data and find concordance/discordance between gene and protein expression, as well as to establish a series of reliable assay for detection of unique identifiers for placenta- and umbilicus-derived cells.

### Methods & Materials

Cells:

Placenta-derived cells (three isolates, including one isolate predominately neonatal as identified by karyotyping analysis), umbilicus-derived cells (four isolates), and Normal Human Dermal Fibroblasts (NHDF; neonatal and adult) grown in Growth Medium with penicillin/streptomycin in a gelatin-coated T75 flask. Mesechymal Stem Cells (MSCS) were grown in Mesenchymal Stem Cell Growth Medium Bullet kit (MSCGM; Cambrex, Walkerville, Md.).

For the IL-8 protocol, cells were thawed from liquid nitrogen and plated in gelatin-coated flasks at 5,000 cells/cm′, grown for 48 hours in Growth Medium and then grown for further 8 hours in 10 milliliters of serum starvation medium [DMEM—low glucose (Gibco, Carlsbad, Calif.), penicillin/streptomycin (Gibco, Carlsbad, Calif.) and 0.1% (w/v) Bovine Serum Albumin (BSA; Sigma, St. Louis, Mo.)]. After this treatment RNA was extracted and the supernatants were centrifuged at 150×g for 5 minutes to remove cellular debris. Supernatants were then frozen at −80° C. for ELISA analysis.

Cell Culture for ELISA Assay:

Postpartum cells derived from placenta and umbilicus, as well as human fibroblasts derived from human neonatal foreskin were cultured in Growth Medium in gelatin-coated T75 flasks. Cells were frozen at passage 11 in liquid nitrogen. Cells were thawed and transferred to 15-milliliter centrifuge tubes. After centrifugation at 150×g for 5 minutes, the supernatant was discarded. Cells were resuspended in 4 milliliters culture medium and counted. Cells were grown in a 75 cm² flask containing 15 milliliters of Growth Medium at 375,000 cells/flask for 24 hours. The medium was changed to a serum starvation medium for 8 hours. Serum starvation medium was collected at the end of incubation, centrifuged at 14,000×g for 5 minutes (and stored at −20° C.).

To estimate the number of cells in each flask, 2 milliliters of tyrpsin/EDTA (Gibco, Carlsbad, Calif.) was added each flask. After cells detached from the flask, trypsin activity was neutralized with 8 milliliters of Growth Medium. Cells were transferred to a 15 milliliters centrifuge tube and centrifuged at 150×g for 5 minutes. Supernatant was removed and 1 milliliter Growth Medium was added to each tube to resuspend the cells. Cell number was estimated using a hemocytometer.

ELISA Assay:

The amount of IL-8 secreted by the cells into serum starvation medium was analyzed using ELISA assays (R&D Systems, Minneapolis, Minn.). All assays were tested according to the instructions provided by the manufacturer.

Total RNA Isolation:

RNA was extracted from confluent postpartum-derived cells and fibroblasts or for IL-8 expression from cells treated as described above. Cells were lysed with 350 microliters buffer RLT containing beta-mercaptoethanol (Sigma, St. Louis, Mo.) according to the manufacturer's instructions (RNeasy® Mini Kit; Qiagen, Valencia, Calif.). RNA was extracted according to the manufacturer's instructions (RNeasy® Mini Kit; Qiagen, Valencia, Calif.) and subjected to DNase treatment (2.7 U/sample) (Sigma St. Louis, Mo.). RNA was eluted with 50 microliters DEPC-treated water and stored at −80° C.

Reverse Transcription:

RNA was also extracted from human placenta and umbilicus. Tissue (30 milligram) was suspended in 700 microliters of buffer RLT containing 2-mercaptoethanol. Samples were mechanically homogenized and the RNA extraction proceeded according to manufacturer's specification. RNA was extracted with 50 microliters of DEPC-treated water and stored at −80° C. RNA was reversed transcribed using random hexamers with the TaqMan® reverse transcription reagents (Applied Biosystems, Foster City, Calif.) at 25° C. for 10 minutes, 37° C. for 60 minutes, and 95° C. for 10 minutes. Samples were stored at −20° C.

Genes identified by cDNA microarray as uniquely regulated in postpartum cells (signature genes—including oxidized LDL receptor, interleukin-8, rennin and reticulon), were further investigated using real-time and conventional PCR.

Real-Time PCR:

PCR was performed on cDNA samples using Assays-on-Demand® gene expression products: oxidized LDL receptor (Hs00234028); rennin (Hs00166915); reticulon (Hs003825 15); CXC ligand 3 (Hs00171061); GCP-2 (Hs00605742); IL-8 (Hs00174103); and GAPDH (Applied Biosystems, Foster City, Calif.) were mixed with cDNA and TaqMan® Universal PCR master mix according to the manufacturer's instructions (Applied Biosystems, Foster City, Calif.) using a 7000 sequence detection system with ABI Prism 7000 SDS software (Applied Biosystems, Foster City, Calif.). Thermal cycle conditions were initially 50° C. for 2 min and 95° C. for 10 min, followed by 40 cycles of 95° C. for 15 sec and 60° C. for 1 min. PCR data was analyzed according to manufacturer's specifications (User Bulletin #2 from Applied Biosystems for ABI Prism 7700 Sequence Detection System).

Conventional PCR:

Conventional PCR was performed using an ABI PRISM 7700 (Perkin Elmer Applied Biosystems, Boston, Mass., USA) to confirm the results from real-time PCR. PCR was performed using 2 microliters of cDNA solution, 1× AmpliTaq Gold universal mix PCR reaction buffer (Applied Biosystems, Foster City, Calif.) and initial denaturation at 94° C. for 5 minutes. Amplification was optimized for each primer set. For IL-8, CXC ligand 3, and reticulon (94° C. for 15 seconds, 55° C. for 15 seconds and 72° C. for 30 seconds for 30 cycles); for rennin (94° C. for 15 seconds, 53° C. for 15 seconds and 72° C. for 30 seconds for 38 cycles); for oxidized LDL receptor and GAPDH (94° C. for 15 seconds, 55° C. for 15 seconds and 72° C. for 30 seconds for 33 cycles). Primers used for amplification are listed in Table 11-1. Primer concentration in the final PCR reaction was 1 micromolar except for GAPDH, which was 0.5 micromolar. GAPDH primers were the same as real-time PCR, except that the manufacturer's TaqMan® probe was not added to the final PCR reaction. Samples were run on 2% (w/v) agarose gel and stained with ethidium bromide (Sigma, St. Louis, Mo.). Images were captured using a 667 Universal Twinpack film (VWR International, South Plainfield, N.J.) using a focal length Polaroid camera (VWR International, South Plainfield, N.J.).

TABLE 11-1
Primers used

| Primer name | Primers |
|---|---|
| Oxidized LDL | S: 5′-GAGAAATCCAAAGAGCAAATGG-3 |
| receptor | (SEQ ID NO: 1) |
| | A: 5′-AGAATGGAAAACTGGAATAGG-3′ |
| | (SEQ ID NO: 2) |
| Renin | S: 5′-TCTTCGATGCTTCGGATTCC-3′ |
| | (SEQ ID NO: 3) |
| | A: 5′-GAATTCTCGGAATCTCTGTTG-3′ |
| | (SEQ ID NO: 4) |
| Reticulon | S: 5′-TTACAAGCAGTGCAGAAAACC-3′ |
| | (SEQ ID NO: 5) |
| | A: 5′-AGTAAACATTGAAACCACAGCC-3′ |
| | (SEQ ID NO: 6) |
| Interleukin-8 | S: 5′-TCTGCAGCTCTGTGTGAAGG-3′ |
| | (SEQ ID NO: 7) |
| | A: 5′-CTTCAAAAACTTCTCCACAACC-3′ |
| | (SEQ ID NO: 8) |
| Chemokine (CXC) | S: 5′-CCCACGCCACGCTCTCC-3′ |
| ligand 3 | (SEQ ID NO: 9) |
| | A: 5′-TCCTGTCAGTTGGTGCTCC-3′ |
| | (SEQ ID NO: 10) |

Immunofluorescence:

PPDCs were fixed with cold 4% (w/v) paraformaldehyde (Sigma-Aldrich, St. Louis, Mo.) for 10 minutes at room temperature. One isolate each of umbilicus- and placenta-derived cells at passage 0 (P0) (directly after isolation) and passage 11 (P 11) (two isolates of placenta-derived, two isolates of umbilicus-derived cells) and fibroblasts (P 11) were used Immunocytochemistry was performed using antibodies directed against the following epitopes: vimentin (1:500, Sigma, St. Louis, Mo.), desmin (1:150; Sigma—raised against rabbit; or 1:300; Chemicon, Temecula, Calif.—raised against mouse), alpha-smooth muscle actin (SMA; 1:400; Sigma), cytokeratin 18 (CK18; 1:400; Sigma), von Willebrand Factor (vWF; 1:200; Sigma), and CD34 (human CD34 Class III; 1:100; DAKOCytomation, Carpinteria, Calif.). In addition, the following markers were tested on passage 11 postpartum cells: anti-human GRO alpha—PE (1:100; Becton Dickinson, Franklin Lakes, N.J.), anti-human GCP-2 (1:100; Santa Cruz Biotech, Santa Cruz, Calif.), anti-human oxidized LDL receptor 1 (ox-LDL R1; 1:100; Santa Cruz Biotech), and anti-human NOGA-A (1: 100; Santa Cruz, Biotech).

Cultures were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma, St. Louis, Mo.) for 30 minutes to access intracellular antigens. Where the epitope of interest was located on the cell surface (CD34, ox-LDL R1), Triton X-100 was omitted in all steps of the procedure in order to prevent epitope loss. Furthermore, in instances where the primary antibody was raised against goat (GCP-2, ox-LDL R1, NOGO-A), 3% (v/v) donkey serum was used in place of goat serum throughout. Primary antibodies, diluted in blocking solution, were then applied to the cultures for a period of 1 hour at room temperature. The primary antibody solutions were removed and the cultures were washed with PBS prior to application of secondary antibody solutions (1 hour at room temperature) containing block along with goat anti-mouse IgG-Texas Red (1:250; Molecular Probes, Eugene, Oreg.) and/or goat anti-rabbit IgG-Alexa 488 (1:250; Molecular Probes) or donkey anti-goat IgG-FITC (1:150, Santa Cruz Biotech). Cultures were then washed and 10 micromolar DAPI (Molecular Probes) applied for 10 minutes to visualize cell nuclei.

Following immunostaining, fluorescence was visualized using an appropriate fluorescence filter on an Olympus® inverted epi-fluorescent microscope (Olympus, Melville, N.Y.). In all cases, positive staining represented fluorescence signal above control staining where the entire procedure outlined above was followed with the exception of application of a primary antibody solution. Representative images were captured using a digital color video camera and ImagePro® software (Media Cybernetics, Carlsbad, Calif.). For triple-stained samples, each image was taken using only one emission filter at a time. Layered montages were then prepared using Adobe Photoshop® software (Adobe, San Jose, Calif.).

Preparation of Cells for FACS Analysis:

Adherent cells in flasks were washed in phosphate buffered saline (PBS) (Gibco, Carlsbad, Calif.) and detached with Trypsin/EDTA (Gibco, Carlsbad, Calif.). Cells were harvested, centrifuged, and re-suspended 3% (v/v) FBS in PBS at a cell concentration of 1×10 7 per milliliter. One hundred microliter aliquots were delivered to conical tubes. Cells stained for intracellular antigens were permeabilized with Perm/Wash buffer (BD Pharmingen, San Diego, Calif.). Antibody was added to aliquots as per manufactures specifications and the cells were incubated for in the dark for 30 minutes at 4° C. After incubation, cells were washed with PBS and centrifuged to remove excess antibody. Cells requiring a secondary antibody were resuspended in 100 microliters of 3% FBS. Secondary antibody was added as per manufactures specification and the cells were incubated in the dark for 30 minutes at 4° C. After incubation, cells were washed with PBS and centrifuged to remove excess secondary antibody. Washed cells were resuspended in 0.5 milliliters PBS and analyzed by flow cytometry. The following antibodies were used: oxidized LDL receptor 1 (sc-5813; Santa Cruz, Biotech), GROa (555042; BD Pharmingen, Bedford, Mass.), Mouse IgG1 kappa, (P-4685 and M-5284; Sigma), Donkey against Goat IgG (sc-3743; Santa Cruz, Biotech.). Flow cytometry analysis was performed with FACScalibur™ (Becton Dickinson San Jose, Calif.).

### Results

Results of real-time PCR for selected “signature” genes performed on cDNA from cells derived from human placentae, adult and neonatal fibroblasts and Mesenchymal Stem Cells (MSCs) indicate that both oxidized LDL receptor and rennin were expressed at higher level in the placenta-derived cells as compared to other cells. The data obtained from real-time PCR were analyzed by the AACT method and expressed on a logarithmic scale. Levels of reticulon and oxidized LDL receptor expression were higher in umbilicus-derived cells as compared to other cells. No significant difference in the expression levels of CXC ligand 3 and GCP-2 were found between postpartum-derived cells and controls. The results of real-time PCR were confirmed by conventional PCR. Sequencing of PCR products further validated these observations. No significant difference in the expression level of CXC ligand 3 was found between postpartum-derived cells and controls using conventional PCR CXC ligand 3 primers listed above in Table 11-1.

The production of the cytokine, IL-8 in postpartum was elevated in both Growth Medium-cultured and serum-starved postpartum-derived cells. All real-time PCR data was validated with conventional PCR and by sequencing PCR products.

When supernatants of cells grown in serum-free medium were examined for the presence of IL-8, the highest amounts were detected in media derived from umbilical cells and some isolates of placenta cells (Table 11-2). No IL-8 was detected in medium derived from human dermal fibroblasts.

TABLE 11-2
IL-8 protein expression measured by ELISA

| Cell type | IL-8 |
|---|---|
| Human fibroblasts | ND |
| Placenta Isolate 1 | ND |
| UMBC Isolate 1 | 2058.42 ± 144.67 |
| Placenta Isolate 2 | ND |
| UMBC Isolate 2 | 2368.86 ± 22.73 |
| Placenta Isolate3 (normal O₂) | 17.27 ± 8.63 |
| Placenta Isolate 3 (low O₂, W/O | 264.92 ± 9.88 |
| BME) | |

Results of the ELISA assay for interleukin-8 (IL-8) performed on placenta- and umbilical cord-derived cells as well as human skin fibroblasts. Values are presented here are picogram/million cells, n = 2, sem.
ND: Not Detected

Placenta-derived cells were also examined for the production of oxidized LDL receptor, GCP-2 and GROalpha by FACS analysis. Cells tested positive for GCP-2. Oxidized LDL receptor and GRO were not detected by this method.

Placenta-derived cells were also tested for the production of selected proteins by immunocytochemical analysis Immediately after isolation (passage 0), cells derived from the human placenta were fixed with 4% paraformaldehyde and exposed to antibodies for six proteins: von Willebrand Factor, CD34, cytokeratin 18, desmin, alpha-smooth muscle actin, and vimentin. Cells stained positive for both alpha-smooth muscle actin and vimentin. This pattern was preserved through passage 11. Only a few cells (<5%) at passage 0 stained positive for cytokeratin 18.

Cells derived from the human umbilical cord at passage 0 were probed for the production of selected proteins by immunocytochemical analysis Immediately after isolation (passage 0), cells were fixed with 4% paraformaldehyde and exposed to antibodies for six proteins: von Willebrand Factor, CD34, cytokeratin 18, desmin, alpha-smooth muscle actin, and vimentin. Umbilicus-derived cells were positive for alpha-smooth muscle actin and vimentin, with the staining pattern consistent through passage 11.

Summary:

Concordance between gene expression levels measured by microarray and PCR (both real-time and conventional) has been established for four genes: oxidized LDL receptor 1, rennin, reticulon, and IL-8. The expression of these genes was differentially regulated at the mRNA level in PPDCs, with IL-8 also differentially regulated at the protein level. The presence of oxidized LDL receptor was not detected at the protein level by FACS analysis in cells derived from the placenta. Differential expression of GCP-2 and CXC ligand 3 was not confirmed at the mRNA level, however GCP-2 was detected at the protein level by FACS analysis in the placenta-derived cells. Although this result is not reflected by data originally obtained from the micro array experiment, this may be due to a difference in the sensitivity of the methodologies.

Immediately after isolation (passage 0), cells derived from the human placenta stained positive for both alpha-smooth muscle actin and vimentin. This pattern was also observed in cells at passage 11. Vimentin and alpha-smooth muscle actin expression may be preserved in cells with passaging, in the Growth Medium and under the conditions utilized in these procedures. Cells derived from the human umbilical cord at passage 0 were probed for the expression of alpha-smooth muscle actin and vimentin, and were positive for both. The staining pattern was preserved through passage 11.

### Example 12

### In Vitro Immunological Evaluation of Postpartum-Derived Cells

Postpartum-derived cells (PPDCs) were evaluated in vitro for their immunological characteristics in an effort to predict the immunological response, if any, these cells would elicit upon in vivo transplantation. PPDCs were assayed by flow cytometry for the presence of HLA-DR, HLA-DP, HLA-DQ, CD80, CD86, and B7-H2. These proteins are expressed by antigen-presenting cells (APe) and are required for the direct stimulation of naïve CD4+T cells (Abbas & Lichtman, CELLULAR AND MOLECULAR IMMUNOLOGY, 5th Ed. (2003) Saunders, Philadelphia, p. 171). The cell lines were also analyzed by flow cytometry for the expression of HLA-G (Abbas & Lichtman, 2003, supra), CD 178 (Coumans, et al., (1999) Journal of Immunological Methods 224, 185-196), and PD-L2 (Abbas & Lichtman, 2003, supra; Brown, et. al. (2003) The Journal of Immunology, 170:1257-1266). The expression of these proteins by cells residing in placental tissues is thought to mediate the immuno-privileged status of placental tissues in utero. To predict the extent to which placenta- and umbilicus-derived cell lines elicit an immune response in vivo, the cell lines were tested in a one-way mixed lymphocyte reaction (MLR).

### Methods & Materials

Cell Culture:

Cells were cultured to confluence in Growth Medium containing penicillin/streptomycin in T75 flasks (Corning Inc., Corning, N.Y.) coated with 2% gelatin (Sigma, St. Louis, Mo.).

Antibody Staining:

Cells were washed in phosphate buffered saline (PBS) (Gibco, Carlsbad, Calif.) and detached with Trypsin/EDTA (Gibco, Carlsbad, Mo.). Cells were harvested, centrifuged, and re-suspended in 3% (v/v) FBS in PBS at a cell concentration of 1×10⁷ per milliliter. Antibody (Table 12-1) was added to one hundred microliters of cell suspension as per manufacturer's specifications and incubated in the dark for 30 minutes at 4° C. After incubation, cells were washed with PBS and centrifuged to remove unbound antibody. Cells were re-suspended in five hundred microliters of PBS and analyzed by flow cytometry using a FACSCalibur™ instrument (Becton Dickinson, San Jose, Calif.).

TABLE 12-1
Antibodies

| Antibody | Manufacturer | Catalog Number |
|---|---|---|
| HLA-DRDPDQ | BD Pharmingen | 555558 |
| | (San Diego, CA) | |
| CD80 | BD Pharmingen | 557227 |
| | (San Diego, CA) | |
| CD86 | BD Pharmingen | 555665 |
| | (San Diego, CA) | |
| B7-H2 | BD Pharmingen | 552502 |
| | (San Diego, CA) | |
| HLA-G | Abcam | ab 7904-100 |
| | (Cambridgeshire, UK) | |
| CD 178 | Santa Cruz | sc-19681 |
| | (San Cruz, CA) | |
| PD-L2 | BD Pharmingen | 557846 |
| | (San Diego, CA) | |
| Mouse IgG2a | Sigma | F-6522 |
| | (St. Louis, MO) | |
| Mouse IgG1kappa | Sigma | P-4685 |
| | (St. Louis, MO) | |

Mixed Lymphocyte Reaction:

Cryopreserved vials of passage 10 umbilicus-derived cells labeled as cell line A and passage 11 placenta-derived cells labeled as cell line B were sent on dry ice to CTBR (Senneville, Quebec) to conduct a mixed lymphocyte reaction using CTBR SOP No. CAC-031. Peripheral blood mononuclear cells (PBMCs) were collected from multiple male and female volunteer donors. Stimulator (donor) allogeneic PBMC, autologous PBMC, and postpartum cell lines were treated with mitomycin C. Autologous and mitomycin C-treated stimulator cells were added to responder (recipient) PBMCs and cultured for 4 days. After incubation, [³H]-thymidine was added to each sample and cultured for 18 hours. Following harvest of the cells, radiolabeled DNA was extracted, and [³H]-thymidine incorporation was measured using a scintillation counter.

The stimulation index for the allogeneic donor (SIAD) was calculated as the mean proliferation of the receiver plus mitomycin C-treated allogeneic donor divided by the baseline proliferation of the receiver. The stimulation index of the PPDCs was calculated as the mean proliferation of the receiver plus mitomycin C-treated postpartum cell line divided by the baseline proliferation of the receiver.

### Results

Mixed Lymphocyte Reaction—Placenta-Derived Cells:

Seven Human Volunteer Blood donors were screened to identify a single allogeneic donor that would exhibit a robust proliferation response in a mixed lymphocyte reaction with the other six blood donors. This donor was selected as the allogeneic positive control donor. The remaining six blood donors were selected as recipients. The allogeneic positive control donor and placenta-derived cell lines were treated with mitomycin C and cultured in a mixed lymphocyte reaction with the six individual allogeneic receivers. Reactions were performed in triplicate using two cell culture plates with three receivers per plate (Table 12-2). The average stimulation index ranged from 1.3 (plate 2) to 3 (plate 1) and the allogeneic donor positive controls ranged from 46.25 (plate 2) to 279 (plate 1) (Table 12-3).

TABLE 12-2
Mixed Lymphocyte Reaction Data - Cell Line B (Placenta)
DPM for Proliferation Assay

| Analytical | Culture | Replicates | | | | | |
|---|---|---|---|---|---|---|---|
| number | System | 1 | 2 | 3 | Mean | SD | CV |

Plate ID: Plate1

| | | | | | | | |
|---|---|---|---|---|---|---|---|
| IM03-7769 | Proliferation baseline of receiver | 79 | 119 | 138 | 112.0 | 30.12 | 26.9 |
| | Control of autostimulation (Mitomycin C treated autologous cells) | 241 | 272 | 175 | 229.3 | 49.54 | 21.6 |
| | MLR allogenic donor IM03-7768 (Mitomycin C treated) | 23971 | 22352 | 20921 | 22414.7 | 1525.97 | 6.8 |
| | MLR with cell line (Mitomycin C treated cell type B) | 664 | 559 | 1090 | 771.0 | 281.21 | 36.5 |
| SI (donor) | | | | | 200 | | |
| SI (cell line) | | | | | 7 | | |
| IM03-7770 | Proliferation baseline of receiver | 206 | 134 | 262 | 200.7 | 64.17 | 32.0 |
| | Control of autostimulation (Mitomycin C treated autologous cells) | 1091 | 602 | 524 | 739.0 | 307.33 | 41.6 |
| | MLR allogenic donor IM03-7768 (Mitomycin C treated) | 45005 | 43729 | 44071 | 44268.3 | 660.49 | 1.5 |
| | MLR with cell line (Mitomycin C treated cell type B) | 533 | 2582 | 2376 | 1830.3 | 1128.24 | 61.6 |
| SI (donor) | | | | | 221 | | |
| SI (cell line) | | | | | 9 | | |
| IM03-7771 | Proliferation baseline of receiver | 157 | 87 | 128 | 124.0 | 35.17 | 28.4 |
| | Control of autostimulation (Mitomycin C treated autologous cells) | 293 | 138 | 508 | 313.0 | 185.81 | 59.4 |
| | MLR allogenic donor IM03-7768 (Mitomycin C treated) | 24497 | 34348 | 31388 | 30077.7 | 5054.53 | 16.8 |
| | MLR with cell line (Mitomycin C treated cell type B) | 601 | 643 | a | 622.0 | 29.70 | 4.8 |
| SI (donor) | | | | | 243 | | |
| SI (cell line) | | | | | 5 | | |
| IM03-7772 | Proliferation baseline of receiver | 56 | 98 | 51 | 68.3 | 25.81 | 37.8 |
| | Control of autostimulation (Mitomycin C treated autologous cells) | 133 | 120 | 213 | 155.3 | 50.36 | 32.4 |
| | MLR allogenic donor IM03-7768 (Mitomycin C treated) | 14222 | 20076 | 22168 | 18822.0 | 4118.75 | 21.9 |
| | MLR with cell line (Mitomycin C treated cell type B) | a | a | a | a | a | a |
| SI (donor) | | | | | 275 | | |
| SI (cell line) | | | | | a | | |
| IM03-7768 | Proliferation baseline of receiver | 84 | 242 | 208 | 178.0 | 83.16 | 46.7 |
| (allogenic donor) | Control of autostimulation (Mitomycin treated autologous cells) | 361 | 617 | 304 | 427.3 | 166.71 | 39.0 |
| Cell line type B | Proliferation baseline of receiver | 126 | 124 | 143 | 131.0 | 10.44 | 8.0 |
| | Control of autostimulation (Mitomycin treated autologous cells) | 822 | 1075 | 487 | 794.7 | 294.95 | 37.1 |

Plate ID: Plate 2

| | | | | | | | |
|---|---|---|---|---|---|---|---|
| IM03-7773 | Proliferation baseline of receiver | 908 | 181 | 330 | 473.0 | 384.02 | 81.2 |
| | Control of autostimulation (Mitomycin C treated autologous cells) | 269 | 405 | 572 | 415.3 | 151.76 | 36.5 |
| | MLR allogenic donor IM03-7768 (Mitomycin C treated) | 29151 | 28691 | 28315 | 28719.0 | 418.70 | 1.5 |
| | MLR with cell line (Mitomycin C treated cell type B) | 567 | 732 | 905 | 734.7 | 169.02 | 23.0 |
| SI (donor) | | | | | 61 | | |
| SI (cell line) | | | | | 2 | | |
| IM03-7774 | Proliferation baseline of receiver | 893 | 1376 | 185 | 818.0 | 599.03 | 73.2 |
| | Control of autostimulation (Mitomycin C treated autologous cells) | 261 | 381 | 568 | 403.3 | 154.71 | 38.4 |
| | MLR allogenic donor IM03-7768 (Mitomycin C treated) | 53101 | 42839 | 48283 | 48074.3 | 5134.18 | 10.7 |
| | MLR with cell line (Mitomycin C treated cell type B) | 515 | 789 | 294 | 532.7 | 247.97 | 46.6 |
| SI (donor) | | | | | 59 | | |
| SI (cell line) | | | | | 1 | | |
| IM03-7775 | Proliferation baseline of receiver | 1272 | 300 | 544 | 705.3 | 505.69 | 71.7 |
| | Control of autostimulation (Mitomycin C treated autologous cells) | 232 | 199 | 484 | 305.0 | 155.89 | 51.1 |
| | MLR allogenic donor IM03-7768 (Mitomycin C treated) | 23554 | 10523 | 28965 | 21014.0 | 9479.74 | 45.1 |
| | MLR with cell line (Mitomycin C treated cell type B) | 768 | 924 | 563 | 751.7 | 181.05 | 24.1 |
| SI (donor) | | | | | 30 | | |
| SI (cell line) | | | | | 1 | | |
| IM03-7776 | Proliferation baseline of receiver | 1530 | 137 | 1046 | 904.3 | 707.22 | 78.2 |
| | Control of autostimulation (Mitomycin C treated autologous cells) | 420 | 218 | 394 | 344.0 | 109.89 | 31.9 |
| | MLR allogenic donor IM03-7768 (Mitomycin C treated) | 28893 | 32493 | 34746 | 32044.0 | 2952.22 | 9.2 |
| | MLR with cell line (Mitomycin C treated cell type B) | a | a | a | a | a | a |
| SI (donor) | | | | | 35 | | |
| SI (cell line) | | | | | a | | |

TABLE 12-3
Average stimulation index of placenta cells and an
allogeneic donor in a mixed lymphocyte
reaction with six individual allogeneic receivers.

| | Recipient | Placenta |
|---|---|---|
| Plate 1 (receivers 1-3) | 279 | 3 |
| Plate 2 (receivers 4-6) | 46.25 | 1.3 |

Mixed Lymphocyte Reaction—Umbilicus-Derived Cells:

Six human volunteer blood donors were screened to identify a single allogeneic donor that will exhibit a robust proliferation response in a mixed lymphocyte reaction with the other five blood donors. This donor was selected as the allogeneic positive control donor. The remaining five blood donors were selected as recipients. The allogeneic positive control donor and placenta cell lines were mitomycin C-treated and cultured in a mixed lymphocyte reaction with the five individual allogeneic receivers. Reactions were performed in triplicate using two cell culture plates with three receivers per plate (Table 12-4). The average stimulation index ranged from 6.5 (plate 1) to 9 (plate 2) and the allogeneic donor positive controls ranged from 42.75 (plate 1) to 70 (plate 2) (Table 12-5).

TABLE 12-4
Mixed Lymphocyte Reaction Data - Cell Line A (Umbilical cord)
DPM for Proliferation Assay

| Analytical | Culture | Replicates | | | | | |
|---|---|---|---|---|---|---|---|
| number | System | 1 | 2 | 3 | Mean | SD | CV |

Plate ID: Plate1

| | | | | | | | |
|---|---|---|---|---|---|---|---|
| IM04-2478 | Proliferation baseline of receiver | 1074 | 406 | 391 | 623.7 | 390.07 | 62.5 |
| | Control of autostimulation (Mitomycin C treated autologous cells) | 672 | 510 | 1402 | 861.3 | 475.19 | 55.2 |
| | MLR allogenic donor IM04-2477 (Mitomycin C treated) | 43777 | 48391 | 38231 | 43466.3 | 5087.12 | 11.7 |
| | MLR with cell line (Mitomycin C treated cell type A) | 2914 | 5622 | 6109 | 4881.7 | 1721.36 | 35.3 |
| SI (donor) | | | | | 70 | | |
| SI (cell line) | | | | | 8 | | |
| IM04-2479 | Proliferation baseline of receiver | 530 | 508 | 527 | 521.7 | 11.93 | 2.3 |
| | Control of autostimulation (Mitomycin C treated autologous cells) | 701 | 567 | 1111 | 793.0 | 283.43 | 35.7 |
| | MLR allogenic donor IM04-2477 (Mitomycin C treated) | 25593 | 24732 | 22707 | 24344.0 | 1481.61 | 6.1 |
| | MLR with cell line (Mitomycin C treated cell type A) | 5086 | 3932 | 1497 | 3505.0 | 1832.21 | 52.3 |
| SI (donor) | | | | | 47 | | |
| SI (cell line) | | | | | 7 | | |
| IM04-2480 | Proliferation baseline of receiver | 1192 | 854 | 1330 | 1125.3 | 244.90 | 21.8 |
| | Control of autostimulation (Mitomycin C treated autologous cells) | 2963 | 993 | 2197 | 2051.0 | 993.08 | 48.4 |
| | MLR allogenic donor IM04-2477 (Mitomycin C treated) | 25416 | 29721 | 23757 | 26298.0 | 3078.27 | 11.7 |
| | MLR with cell line (Mitomycin C treated cell type A) | 2596 | 5076 | 3426 | 3699.3 | 1262.39 | 34.1 |
| SI (donor) | | | | | 23 | | |
| SI (cell line) | | | | | 3 | | |
| IM04-2481 | Proliferation baseline of receiver | 695 | 451 | 555 | 567.0 | 122.44 | 21.6 |
| | Control of autostimulation (Mitomycin C treated autologous cells) | 738 | 1252 | 464 | 818.0 | 400.04 | 48.9 |
| | MLR allogenic donor IM04-2477 (Mitomycin C treated) | 13177 | 24885 | 15444 | 17835.3 | 6209.52 | 34.8 |
| | MLR with cell line (Mitomycin C treated cell type A) | 4495 | 3671 | 4674 | 4280.0 | 534.95 | 12.5 |
| SI (donor) | | | | | 31 | | |
| SI (cell line) | | | | | 8 | | |

Plate ID: Plate 2

| | | | | | | | |
|---|---|---|---|---|---|---|---|
| IM04-2482 | Proliferation baseline of receiver | 432 | 533 | 274 | 413.0 | 130.54 | 31.6 |
| | Control of autostimulation (Mitomycin C treated autologous cells) | 1459 | 633 | 598 | 896.7 | 487.31 | 54.3 |
| | MLR allogenic donor IM04-2477 (Mitomycin C treated) | 24286 | 30823 | 31346 | 28818.3 | 3933.82 | 13.7 |
| | MLR with cell line (Mitomycin C treated cell type A) | 2762 | 1502 | 6723 | 3662.3 | 2724.46 | 74.4 |
| SI (donor) | | | | | 70 | | |
| SI (cell line) | | | | | 9 | | |
| IM04-2477 | Proliferation baseline of receiver | 312 | 419 | 349 | 360.0 | 54.34 | 15.1 |
| (allogenic donor) | Control of autostimulation (Mitomycin treated autologous cells) | 567 | 604 | 374 | 515.0 | 123.50 | 24.0 |
| Cell line type A | Proliferation baseline of receiver | 5101 | 3735 | 2973 | 3936.3 | 1078.19 | 27.4 |
| | Control of autostimulation (Mitomycin treated autologous cells) | 1924 | 4570 | 2153 | 2882.3 | 1466.04 | 50.9 |

TABLE 12-5
Average stimulation index of umbilical cord-derived cells and an
allogeneic donor in a mixed lymphocyte reaction with five individual
allogeneic receivers.

| | | Umbilical |
|---|---|---|
| | Recipient | Cord |
| Plate 1 (receivers 1-4) | 42.75 | 6.5 |
| Plate 2 (receiver 5) | 70 | 9 |

Antigen Presenting Cell Markers—Placenta-Derived Cells:

Histograms of placenta-derived cells analyzed by flow cytometry show negative expression of HLA-DR, DP, DQ, CD80, CD86, and B7-H2, as noted by fluorescence value consistent with the IgG control, indicating that placental cell lines lack the cell surface molecules required to directly stimulate CD4+T cells.

Immunomodulating Markers—Placenta-Derived Cells:

Histograms of placenta-derived cells analyzed by flow cytometry show positive expression of PD-L2, as noted by the increased value of fluorescence relative to the IgG control, and negative expression of CD178 and HLA-G, as noted by fluorescence value consistent with the IgG control.

Antigen Presenting Cell Markers—Umbilicus-Derived Cells:

Histograms of umbilicus-derived cells analyzed by flow cytometry show negative expression of HLA-DR, DP, DQ, CD80, CD86, and B7-H2, as noted by fluorescence value consistent with the IgG control, indicating that umbilical cell lines lack the cell surface molecules required to directly stimulate CD4+T cells.

Immunomodulating Cell Markers—Umbilicus-Derived Cells:

Histograms of umbilicus-derived cells analyzed by flow cytometry show positive expression of PD-L2, as noted by the increased value of fluorescence relative to the IgG control, and negative expression of CD178 and HLA-G, as noted by fluorescence value consistent with the IgG control.

Summary:

In the mixed lymphocyte reactions conducted with placenta-derived cell lines, the average stimulation index ranged from 1.3 to 3, and that of the allogeneic positive controls ranged from 46.25 to 279. In the mixed lymphocyte reactions conducted with umbilicus-derived cell lines the average stimulation index ranged from 6.5 to 9, and that of the allogeneic positive controls ranged from 42.75 to 70. Placenta- and umbilicus-derived cell lines were negative for the expression of the stimulating proteins HLA-DR, HLA-DP, HLA-DQ, CD80, CD86, and B7-H2, as measured by flow cytometry. Placenta- and umbilicus-derived cell lines were negative for the expression of immuno-modulating proteins HLA-G and CD178 and positive for the expression of PD-L2, as measured by flow cytometry. Allogeneic donor PBMCs contain antigen-presenting cells expressing HLA-DR, DQ, CD8, CD86, and B 7-H2, thereby allowing for the stimulation of naïve CD4+T cells. The absence of antigen-presenting cell surface molecules on placenta- and umbilicus-derived cells required for the direct stimulation of naïve CD4+T cells and the presence of PD-L2, an immunomodulating protein, may account for the low stimulation index exhibited by these cells in a MLR as compared to allogeneic controls.

### Example 13

### Secretion of Trophic Factors by Postpartum-Derived Cells

The secretion of selected trophic factors from placenta- and umbilicus-derived cells was measured. Factors selected for detection included: (1) those known to have angiogenic activity, such as hepatocyte growth factor (HGF) (Rosen et al. (1997) Ciba Found. Symp. 212:215-26), monocyte chemotactic protein 1 (MCP-1) (Salcedo et al. (2000) Blood 96; 34-40), interleukin-8 (IL-8) (Li et al. (2003) J. Immunol. 170:3369-76), keratinocyte growth factor (KGF), basic fibroblast growth factor (bFGF), vascular endothelial growth factor (VEGF) (Hughes et al. (2004) Ann. Thorac. Surg. 77:812-8), matrix metalloproteinase 1 (TIMP1), angiopoietin 2 (ANG2), platelet derived growth factor (PDGF-bb), thrombopoietin (TPO), heparin-binding epidermal growth factor (HB-EGF), stromal-derived factor 1alpha (SDF-1alpha); (2) those known to have neurotrophic/neuroprotective activity, such as brain-derived neurotrophic factor (BDNF) (Cheng et al. (2003) Dev. Biol. 258; 319-33), interleukin-6 (IL-6), granulocyte chemotactic protein-2 (GCP-2), transforming growth factor beta2 (TGFbeta2); and (3) those known to have chemokine activity, such as macrophage inflammatory protein 1 alpha (MIP1a), macrophage inflammatory protein 1 beta (MIP1b), monocyte chemoattractant-1 (MCP-1), Rantes (regulated on activation, normal T cell expressed and secreted), 1309, thymus and activation-regulated chemokine (TARe), Eotaxin, macrophage-derived chemokine (MDC), IL-8).

### Methods & Materials

Cell Culture:

PPDCs from placenta and umbilicus as well as human fibroblasts derived from human neonatal foreskin were cultured in Growth Medium with penicillin/streptomycin on gelatin-coated T75 flasks. Cells were cryopreserved at passage 11 and stored in liquid nitrogen. After thawing of the cells, Growth Medium was added to the cells followed by transfer to a 15 milliliter centrifuge tube and centrifugation of the cells at 150×g for 5 minutes. The supernatant was discarded. The cell pellet was resuspended in 4 milliliters Growth Medium, and cells were counted. Cells were seeded at 375,000 cells/75 cm² flask containing 15 milliliters of Growth Medium and cultured for 24 hours. The medium was changed to a serum-free medium (DMEM-low glucose (Gibco), 0.1% (w/v) bovine serum albumin (Sigma), penicillin/streptomycin (Gibco)) for 8 hours. Conditioned serum-free medium was collected at the end of incubation by centrifugation at 14,000×g for 5 minutes and stored at −20° C.

To estimate the number of cells in each flask, cells were washed with PBS and detached using 2 milliliters trypsin/EDTA. Trypsin activity was inhibited by addition of 8 milliliters Growth Medium. Cells were centrifuged at 150×g for 5 minutes. Supernatant was removed, and cells were resuspended in 1 milliliter Growth Medium. Cell number was estimated using a hemocytometer.

ELISA Assay:

Cells were grown at 37° C. in 5% carbon dioxide and atmospheric oxygen. Placenta-derived cells (batch 101503) also were grown in 5% oxygen or beta-mercaptoethanol (BME). The amount of MCP-1, IL-6, VEGF, SDF-1alpha, GCP-2, IL-8, and TGF-beta 2 produced by each cell sample was measured by an ELISA assay (R&D Systems, Minneapolis, Minn.). All assays were performed according to the manufacturer's instructions.

SearchLight™ Multiplexed ELISA Assay:

Chemokines (MIP1a, MIP1b, MCP-1, Rantes, 1309, TARC, Eotaxin, MDC, IL8), BDNF, and angiogenic factors (HGF, KGF, bFGF, VEGF, TIMP1, ANG2, PDGF-bb, TPO, HB-EGF were measured using SearchLight™ Proteome Arrays (Pierce Biotechnology Inc.). The Proteome Arrays are multiplexed sandwich ELISAs for the quantitative measurement of two to 16 proteins per well. The arrays are produced by spotting a 2×2, 3×3, or 4×4 pattern of four to 16 different capture antibodies into each well of a 96-well plate. Following a sandwich ELISA procedure, the entire plate is imaged to capture chemiluminescent signal generated at each spot within each well of the plate. The amount of signal generated in each spot is proportional to the amount of target protein in the original standard or sample.

### Results

ELISA Assay:

MCP-1 and IL-6 were secreted by placenta- and umbilicus-derived cells and dermal fibroblasts (Table 13-1). SDF-1alpha was secreted by placenta-derived cells cultured in 5% 0 2 and by fibroblasts. GCP-2 and IL-8 were secreted by umbilicus-derived cells and by placenta-derived cells cultured in the presence of BME or 5% 02. GCP-2 also was secreted by human fibroblasts. TGF-beta2 was not detectable by ELISA assay.

TABLE 13-1
ELISA Results: Detection of Trophic Factors

| | | | | | | | TGF- |
|---|---|---|---|---|---|---|---|
| | MCP-1 | IL-6 | VEGF | SDF-1α | GCP-2 | IL-8 | β2 |
| Fibroblast | 17 ± 1 | 61 ± 3 | 29 ± 2 | 19 ± 1 | 21 ± 1 | ND | ND |
| Placenta (042303) | 60 ± 3 | 41 ± 2 | ND | ND | ND | ND | ND |
| Umbilical (022803) | 1150 ± 74 | 4234 ± 289 | ND | ND | 160 ± 11 | 2058 ± 145 | ND |
| Placenta (071003) | 125 ± 16 | 10 ± 1 | ND | ND | ND | ND | ND |
| Umbilical (071003) | 2794 ± 84 | 1356 ± 43 | ND | ND | 2184 ± 98 | 2369 ± 23 | ND |
| Placenta (101503) BME | 21 ± 10 | 67 ± 3 | ND | ND | 44 ± 9 | 17 ± 9 | ND |
| Placenta (101503) 5% O₂, | 77 ± 16 | 339 ± 21 | ND | 1149 ± 137 | 54 ± 2 | 265 ± 10 | ND |
| W/O BME | | | | | | | |

Key:
ND: Not Detected.,
=/− sem

SearchLight™ Multiplexed ELISA Assay:

TIMP1, TPO, KGF, HGF, FGF, HBEGF, BDNF, MIP1b, MCP1, RANTES, 1309, TARC, MDC, and IL-8 were secreted from umbilicus-derived cells (Tables 13-2 and 13-3). TIMP1, TPO, KGF, HGF, HBEGF, BDNF, MIP1a, MCP-1, RANTES, TARC, Eotaxin, and IL-8 were secreted from placenta-derived cells (Tables 13-2 and 13-3). No Ang2, VEGF, or PDGF-bb were detected.

TABLE 13-2
SEARCHLIGHT Multiplexed ELISA assay results

| | TIMP1 | ANG2 | PDGFbb | TPO | KGF | HGF | FGF | VEGF | HBEGF | BDNF |
|---|---|---|---|---|---|---|---|---|---|---|
| hFB | 19306.3 | ND | ND | 230.5 | 5.0 | ND | ND | 27.9 | 1.3 | ND |
| P1 | 24299.5 | ND | ND | 546.6 | 8.8 | 16.4 | ND | ND | 3.81.3 | ND |
| U1 | 57718.4 | ND | ND | 1240.0 | 5.8 | 559.3 | 148.7 | ND | 9.3 | 165.7 |
| P3 | 14176.8 | ND | ND | 568.7 | 5.2 | 10.2 | ND | ND | 1.9 | 33.6 |
| U3 | 21850.0 | ND | ND | 1134.5 | 9.0 | 195.6 | 30.8 | ND | 5.4 | 388.6 |

Key:
hFB (human fibroblasts),
P1 (placenta-derived cells (042303)),
U1 (umbilicus-derived cells (022803)),
P3 (placenta-derived cells (071003)),
U3 (umbilicus-derived cells (071003)).
ND: Not Detected.

TABLE 13-3
SEARCHLIGHT Multiplexed ELISA assay results

| | MIP1a | MIP1b | MCP1 | RANTES | I309 | TARC | Eotaxin | MDC | IL8 |
|---|---|---|---|---|---|---|---|---|---|
| hFB | ND | ND | 39.6 | ND | ND | 0.1 | ND | ND | 204.9 |
| P1 | 79.5 | ND | 228.4 | 4.1 | ND | 3.8 | 12.2 | ND | 413.5 |
| U1 | ND | 8.0 | 1694.2 | ND | 22.4 | 37.6 | ND | 18.9 | 51930.1 |
| P3 | ND | ND | 102.7 | ND | ND | 0.4 | ND | ND | 63.8 |
| U3 | ND | 5.2 | 2018.7 | 41.5 | 11.6 | 21.4 | ND | 4.8 | 10515.9 |

Key:
hFB (human fibroblasts),
P1 (placenta-derived PPDC (042303)),
U1 (umbilicus-derived PPDC (022803)),
P3 (placenta-derived PPDC (071003)),
U3 (umbilicus-derived PPDC (071003)).
ND: Not Detected.

### Example 14

### Short-Term Neural Differentiation of Postpartum-Derived Cells

The ability of placenta- and umbilicus-derived cells (collectively postpartum-derived cells or PPDCs) to differentiate into neural lineage cells was examined.

### Methods & Materials

Isolation and Expansion of Postpartum Cells:

PPDCs from placental and umbilical tissues were isolated and expanded as described in Example 6.

Modified Woodbury-Black Protocol (A):

This assay was adapted from an assay originally performed to test the neural induction potential of bone marrow stromal cells (1). Umbilicus-derived cells (022803) P4 and placenta-derived cells (042203) P3 were thawed and culture expanded in Growth Media at 5,000 cells/cm² until sub-confluence (75%) was reached. Cells were then trypsinized and seeded at 6,000 cells per well of a Titretek II glass slide (VWR International, Bristol, Conn.). As controls, mesenchymal stem cells (P3; 1F2155; Cambrex, Walkersville, Md.), osteoblasts (P5; CC2538; Cambrex), adipose-derived cells (Artecel, U.S. Pat. No. 6,555,374 B1) (P6; Donor 2) and neonatal human dermal fibroblasts (P6; CC2509; Cambrex) were also seeded under the same conditions.

All cells were initially expanded for 4 days in DMEM/F12 medium (Invitrogen, Carlsbad, Calif.) containing 15% (v/v) fetal bovine serum (FBS; Hyclone, Logan, Utah), basic fibroblast growth factor (bFGF; 20 nanograms/milliliter; Peprotech, Rocky Hill, N.J.), epidermal growth factor (EGF; 20 nanograms/milliliter; Peprotech) and penicillin/streptomycin (Invitrogen). After four days, cells were rinsed in phosphate-buffered saline (PBS; Invitrogen) and were subsequently cultured in DMEM/F12 medium+20% (v/v) FBS+penicillin/streptomycin for 24 hours. After 24 hours, cells were rinsed with PBS. Cells were then cultured for 1-6 hours in an induction medium which was comprised of DMEM/FI2 (serum-free) containing 200 mM butylated hydroxyanisole, 10 μM potassium chloride, 5 milligram/milliliter insulin, 10 μM forskolin, 4 μM valproic acid, and 2 μM hydrocortisone (all chemicals from Sigma, St. Louis, Mo.). Cells were then fixed in 100% ice-cold methanol and immunocytochemistry was performed (see methods below) to assess human nestin protein expression.

Modified Woodbury-Black Protocol (B):

PPDCs (umbilicus (022803) P11; placenta (042203) P11 and adult human dermal fibroblasts (1F1853, P11) were thawed and culture expanded in Growth Medium at 5,000 cells/cm² until sub-confluence (75%) was reached. Cells were then trypsinized and seeded at similar density as in (A), but onto (1) 24 well tissue culture-treated plates (TCP, Falcon brand, VWR International), (2) TCP wells+2% (w/v) gelatin adsorbed for 1 hour at room temperature, or (3) TCP wells+20 pg/milliliter adsorbed mouse laminin (adsorbed for a minimum of 2 hours at 37° C.; Invitrogen).

Exactly as in (A), cells were initially expanded and media switched at the aforementioned timeframes. One set of cultures was fixed, as before, at 5 days and 6 hours, this time with ice-cold 4% (w/v) paraformaldehyde (Sigma) for 10 minutes at room temperature. In the second set of cultures, medium was removed and switched to Neural Progenitor Expansion medium (NPE) consisting of Neurobasal-A medium (Invitrogen) containing B27 (B27 supplement; Invitrogen), L-glutamine (4 mM), and penicillin/streptomycin (Invitrogen). NPE medium was further supplemented with retinoic acid (RA; 1 μM; Sigma). This medium was removed 4 days later and cultures were fixed with ice-cold 4% (w/v) paraformaldehyde (Sigma) for 10 minutes at room temperature, and stained for nestin, GFAP, and TuJ1 protein expression (see Table 14-1).

TABLE 14-1
Summary of Primary Antibodies Used

| Antibody | Concentration | Vendor |
|---|---|---|
| Rat 401 (nestin) | 1:200 | Chemicon, Temecula, CA |
| Human Nestin | 1:100 | Chemicon |
| TuJ1 (BIII Tubulin) | 1:500 | Sigma, St. Louis, MO |
| GFAP | 1:2000 | DakoCytomation, |
| | | Carpinteria, CA |
| Tyrosine hydroxylase (TH) | 1:1000 | Chemicon |
| GABA | 1:400 | Chemicon |
| Desmin (mouse) | 1:300 | Chemicon |
| alpha - alpha-smooth muscle | 1:400 | Sigma |
| actin | | |
| Human nuclear protein (hNuc) | 1:150 | Chemicon |

Two Stage Differentiation Protocol:

PPDCs (umbilicus (042203) P11, placenta (022803) P11), adult human dermal fibroblasts (P11; 1F1853; Cambrex) were thawed and culture expanded in Growth Medium at 5,000 cells/cm² until sub-confluence (75%) was reached. Cells were then trypsinized and seeded at 2,000 cells/cm², but onto 24 well plates coated with laminin (BD Biosciences, Franklin Lakes, N.J.) in the presence of NPE media supplemented with bFGF (20 nanograms/milliliter; Peprotech, Rocky Hill, N.J.) and EGF (20 nanograms/milliliter; Peprotech) [whole media composition further referred to as NPE+F+E]. At the same time, adult rat neural progenitors isolated from hippocampus (P4; (062603) were also plated onto 24 welliaminin-coated plates in NPE+F+E media. All cultures were maintained in such conditions for a period of 6 days (cells were fed once during that time) at which time media was switched to the differentiation conditions listed in Table 14-2 for an additional period of 7 days. Cultures were fixed with ice-cold 4% (w/v) paraformaldehyde (Sigma) for 10 minutes at room temperature, and stained for human or rat nestin, GF AP, and TuJ1 protein expression.

TABLE 14-2
Summary of Conditions for Two-Stage Differentiation Protocol

| | A | B |
|---|---|---|
| COND. # | PRE-DIFFERENTIATION | 2^{nd} STAGE DIFF |
| 1 | NPE + F (20 ng/ml) + E (20 ng/ml) | NPE + SHH (200 ng/ml) + F8 (100 ng/ml) |
| 2 | NPE + F (20 ng/ml) + E (20 ng/ml) | NPE + SHH (200 ng/ml) + F8 (100 |
| | | ng/ml) + RA (1 μM) |
| 3 | NPE + F (20 ng/ml) + E (20 ng/ml) | NPE + RA (1 μM) |
| 4 | NPE + F (20 ng/ml) + E (20 ng/ml) | NPE + F (20 ng/ml) + E (20 ng/ml) |
| 5 | NPE + F (20 ng/ml) + E (20 ng/ml) | Growth Medium |
| 6 | NPE + F (20 ng/ml) + E (20 ng/ml) | Condition 1B + MP52 (20 ng/ml) |
| 7 | NPE + F (20 ng/ml) + E (20 ng/ml) | Condition 1B + BMP7 (20 ng/ml) |
| 8 | NPE + F (20 ng/ml) + E (20 ng/ml) | Condition 1B + GDNF (20 ng/ml) |
| 9 | NPE + F (20 ng/ml) + E (20 ng/ml) | Condition 2B + MP52 (20 ng/ml) |
| 10 | NPE + F (20 ng/ml) + E (20 ng/ml) | Condition 2B + BMP7 (20 ng/ml) |
| 11 | NPE + F (20 ng/ml) + E (20 ng/ml) | Condition 2B + GDNF (20 ng/ml) |
| 12 | NPE + F (20 ng/ml) + E (20 ng/ml) | Condition 3B + MP52 (20 ng/ml) |
| 13 | NPE + F (20 ng/ml) + E (20 ng/ml) | Condition 3B + BMP7 (20 ng/ml) |
| 14 | NPE + F (20 ng/ml) + E (20 ng/ml) | Condition 3B + GDNF (20 ng/ml) |
| 15 | NPE + F (20 ng/ml) + E (20 ng/ml) | NPE + MP52 (20 ng/ml) |
| 16 | NPE + F (20 ng/ml) + E (20 ng/ml) | NPE + BMP7 (20 ng/ml) |
| 17 | NPE + F (20 ng/ml) + E (20 ng/ml) | NPE + GDNF (20 ng/ml) |

Multiple Growth Factor Protocol:

Umbilicus-derived cells (P11; (042203)) were thawed and culture expanded in Growth Medium at 5,000 cells/cm² until sub-confluence (75%) was reached. Cells were then trypsinized and seeded at 2,000 cells/cm², onto 24 welliaminin-coated plates (BD Biosciences) in the presence of NPE+F (20 nanograms/milliliter)+E (20 nanograms/milliliter). In addition, some wells contained NPE+F+E+2% FBS or 10% FBS. After four days of “pre-differentiation” conditions, all media were removed and samples were switched to NPE medium supplemented with sonic hedgehog (SHH; 200 nanograms/milliliter; Sigma, St. Louis, Mo.), FGF8 (100 nanograms/milliliter; Peprotech), BDNF (40 nanograms/milliliter; Sigma), GDNF (20 nanograms/milliliter; Sigma), and retinoic acid (1 μM; Sigma). Seven days post medium change, cultures were fixed with ice-cold 4% (w/v) paraformaldehyde (Sigma) for 10 minutes at room temperature, and stained for human nestin, GFAP, TuJ1, desmin, and alpha-smooth muscle actin expression.

Neural Progenitor Co-Culture Protocol:

Adult rat hippocampal progenitors (062603) were plated as neurospheres or single cells (10,000 cells/well) onto laminin-coated 24 well dishes (BD Biosciences) in NPE+F (20 nanograms/milliliter)+E (20 nanograms/milliliter).

Separately, umbilicus-derived cells (042203) P11 and placenta-derived cells (022803) P11 were thawed and culture expanded in NPE+F (20 nanograms/milliliter)+E (20 nanograms/milliliter) at 5,000 cells/cm′ for a period of 48 hours. Cells were then trypsinized and seeded at 2,500 cells/well onto existing cultures of neural progenitors. At that time, existing medium was exchanged for fresh medium. Four days later, cultures were fixed with ice-cold 4% (w/v) paraformaldehyde (Sigma) for 10 minutes at room temperature, and stained for human nuclear protein (hNuc; Chemicon) (Table 14-1 above) to identify PPDCs.

Immunocytochemistry:

Immunocytochemistry was performed using the antibodies listed in Table 14-1. Cultures were washed with phosphate-buffered saline (PBS) and exposed to a protein blocking solution containing PBS, 4% (v/v) goat serum (Chemicon, Temecula, Calif.), and 0.3% (v/v) Triton (Triton X-100; Sigma) for 30 minutes to access intracellular antigens. Primary antibodies, diluted in blocking solution, were then applied to the cultures for a period of 1 hour at room temperature. Next, primary antibodies solutions were removed and cultures washed with PBS prior to application of secondary antibody solutions (1 hour at room temperature) containing blocking solution along with goat anti-mouse IgG-Texas Red (1:250; Molecular Probes, Eugene, Oreg.) and goat anti-rabbit IgG-Alexa 488 (1:250; Molecular Probes). Cultures were then washed and 10 micromolar DAPI (Molecular Probes) applied for 10 minutes to visualize cell nuclei.

Following immunostaining, fluorescence was visualized using the appropriate fluorescence filter on an Olympus inverted epi-fluorescent microscope (Olympus, Melville, N.Y.). In all cases, positive staining represented fluorescence signal above control staining where the entire procedure outlined above was followed with the exception of application of a primary antibody solution. Representative images were captured using a digital color video camera and ImagePro software (Media Cybernetics, Carlsbad, Calif.). For triple-stained samples, each image was taken using only one emission filter at a time. Layered montages were then prepared using Adobe Photoshop software (Adobe, San Jose, Calif.).

### Results

Modified Woodbury-Black Protocol (A):

Upon incubation in this neural induction composition, all cell types transformed into cells with bipolar morphologies and extended processes. Other larger non-bipolar morphologies were also observed. Furthermore, the induced cell populations stained positively for nestin, a marker of multipotent neural stem and progenitor cells.

Modified Woodbury-Black Protocol (B):

When repeated on tissue culture plastic (TCP) dishes, nestin expression was not observed unless laminin was pre-adsorbed to the culture surface. To further assess whether nestin-expressing cells could then go on to generate mature neurons, PPDCs and fibroblasts were exposed to NPE+RA (1 μM), a media composition known to induce the differentiation of neural stem and progenitor cells into such cells (2, 3, 4). Cells were stained for TuJ1, a marker for immature and mature neurons, GFAP, a marker of astrocytes, and nestin. Under no conditions was TuJ1 detected, nor were cells with neuronal morphology observed. Furthermore, nestin and GF AP were no longer expressed by PPDCs, as determined by immunocytochemistry.

Two-Stage Differentiation:

Umbilicus and placenta PPDC isolates (as well as human fibroblasts and rodent neural progenitors as negative and positive control cell types, respectively) were plated on laminin (neural promoting)-coated dishes and exposed to 13 different growth conditions (and two control conditions) known to promote differentiation of neural progenitors into neurons and astrocytes. In addition, two conditions were added to examine the influence of GDF5, and BMP7 on PPDC differentiation. Generally, a two-step differentiation approach was taken, where the cells were first placed in neural progenitor expansion conditions for a period of 6 days, followed by full differentiation conditions for 7 days. Morphologically, both umbilicus- and placenta-derived cells exhibited fundamental changes in cell morphology throughout the time-course of this procedure. However, neuronal or astrocytic-shaped cells were not observed except for in control, neural progenitor-plated conditions Immunocytochemistry, negative for human nestin, TuJ1, and GFAP confirmed the morphological observations.

Multiple Growth Factors:

Following one week's exposure to a variety of neural differentiation agents, cells were stained for markers indicative of neural progenitors (human nestin), neurons (TuJ1), and astrocytes (GFAP). Cells grown in the first stage in non-serum containing media had different morphologies than those cells in serum containing (2% or 10%) media, indicating potential neural differentiation. Specifically, following a two step procedure of exposing umbilicus-derived cells to EGF and bFGF, followed by SHH, FGF8, GDNF, BDNF, and retinoic acid, cells showed long extended processes similar to the morphology of cultured astrocytes. When 2% FBS or 10% FBS was included in the first stage of differentiation, cell number was increased and cell morphology was unchanged from control cultures at high density. Potential neural differentiation was not evidenced by immunocytochemical analysis for human nestin, TuJ1, or GFAP.

Neural Progenitor and PPDC Co-Culture:

PPDCs were plated onto cultures of rat neural progenitors seeded two days earlier in neural expansion conditions (NPE+F+E). While visual confirmation of plated PPDCs proved that these cells were plated as single cells, human-specific nuclear staining (hNuc) 4 days post-plating (6 days total) showed that they tended to ball up and avoid contact with the neural progenitors. Furthermore, where PPDCs attached, these cells spread out and appeared to be innervated by differentiated neurons that were of rat origin, suggesting that the PPDCs may have differentiated into muscle cells. This observation was based upon morphology under phase contrast microscopy. Another observation was that typically large cell bodies (larger than neural progenitors) possessed morphologies resembling neural progenitors, with thin processes spanning out in multiple directions. hNuc staining (found in one half of the cell's nucleus) showed that in some cases these human cells may have fused with rat progenitors and assumed their phenotype. Control wells containing only neural progenitors had fewer total progenitors and apparent differentiated cells than did co-culture wells containing umbilicus or placenta PPDCs, further indicating that both umbilicus- and placenta-derived cells influenced the differentiation and behavior of neural progenitors, either by release of chemokines and cytokines, or by contact-mediated effects.

Summary:

Multiple protocols were conducted to determine the short term potential of PPDCs to differentiate into neural lineage cells. These included phase contrast imaging of morphology in combination with immunocytochemistry for nestin, TuJ1, and GFAP, proteins associated with multipotent neural stem and progenitor cells, immature and mature neurons, and astrocytes, respectively.

### Example 15

### Long-Term Neural Differentiation of Postpartum-Derived Cells

The ability of umbilicus and placenta-derived cells (collectively postpartum-derived cells or PPDCs) to undergo long-term differentiation into neural lineage cells was evaluated.

### Methods & Materials

Isolation and Expansion of PPDCs:

PPDCs were isolated and expanded as described in previous Examples.

PPDC Cell Thaw and Plating:

Frozen aliquots of PPDCs (umbilicus (022803) P11; (042203) P11; (071003) P12; placenta (101503) P7) previously grown in Growth Medium were thawed and plated at 5,000 cells/cm 2 in T-75 flasks coated with laminin (BD, Franklin Lakes, N.J.) in Neurobasal-A medium (Invitrogen, Carlsbad, Calif.) containing B27 (B27 supplement, Invitrogen), L-glutamine (4 mM), and Penicillin/Streptomycin (10 milliliters), the combination of which is herein referred to as Neural Progenitor Expansion (NPE) media. NPE media was further supplemented with bFGF (20 nanograms/milliliter, Peprotech, Rocky Hill, N. J.) and EGF (20 nanograms/milliliter, Peprotech, Rocky Hill, N. J.), herein referred to as NPE+bFGF+EGF.

Control Cell Plating:

In addition, adult human dermal fibroblasts (P11, Cambrex, Walkersville, Md.) and mesenchymal stem cells (P5, Cambrex) were thawed and plated at the same cell seeding density on laminin-coated T-75 flasks in NPE+bFGF+EGF. As a further control, fibroblasts, umbilicus, and placenta PPDCs were grown in Growth Medium for the period specified for all cultures.

Cell Expansion:

Media from all cultures were replaced with fresh media once a week and cells observed for expansion. In general, each culture was passaged one time over a period of one month because of limited growth in NPE+bFGF+EGF.

Immunocytochemistry:

After a period of one month, all flasks were fixed with cold 4% (w/v) paraformaldehyde (Sigma) for 10 minutes at room temperature. Immunocytochemistry was performed using antibodies directed against TuJ1 (BIII Tubulin; 1:500; Sigma, St. Louis, Mo.) and GFAP (glial fibrillary acidic protein; 1:2000; DakoCytomation, Carpinteria, Calif.). Briefly, cultures were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma) for 30 minutes to access intracellular antigens. Primary antibodies, diluted in blocking solution, were then applied to the cultures for a period of 1 hour at room temperature. Next, primary antibodies solutions were removed and cultures washed with PBS prior to application of secondary antibody solutions (1 hour at room temperature) containing block along with goat anti-mouse IgG-Texas Red (1:250; Molecular Probes, Eugene, Oreg.) and goat anti-rabbit IgG-Alexa 488 (1:250; Molecular Probes). Cultures were then washed and 10 micromolar DAPI (Molecular Probes) applied for 10 minutes to visualize cell nuclei.

Following immunostaining, fluorescence was visualized using the appropriate fluorescence filter on an Olympus inverted epi-fluorescent microscope (Olympus, Melville, N.Y.). In all cases, positive staining represented fluorescence signal above control staining where the entire procedure outlined above was followed with the exception of application of a primary antibody solution. Representative images were captured using a digital color video camera and ImagePro software (Media Cybernetics, Carlsbad, Calif.). For triple-stained samples, each image was taken using only one emission filter at a time. Layered montages were then prepared using Adobe Photoshop software (Adobe, San Jose, Calif.).

TABLE 15-1
Summary of Primary Antibodies Used

| Antibody | Concentration | Vendor |
|---|---|---|
| TuJ1 (BIII Tubulin) | 1:500 | Sigma, St. Louis, MO |
| GFAP | 1:2000 | DakoCytomation, |
| | | Carpinteria, CA |

### Results

NPE+bFGF+EGF media slows proliferation of PPDCs and alters their morphology Immediately following plating, a subset of PPDCs attached to the culture flasks coated with laminin. This may have been due to cell death as a function of the freeze/thaw process or because of the new growth conditions. Cells that did attach adopted morphologies different from those observed in Growth Media.

Clones of Umbilicus-Derived Cells Express Neuronal Proteins:

Cultures were fixed at one month post-thawing/plating and stained for the neuronal protein TuJ1 and GFAP, an intermediate filament found in astrocytes. While all control cultures grown in Growth Medium and human fibroblasts and MSCs grown in NPE+bFGF+EGF medium were found to be TuJ1−/GFAP−, TuJ1 was detected in the umbilicus and placenta PPDCs. Expression was observed in cells with and without neuronal-like morphologies. No expression of GFAP was observed in either culture. The percentage of cells expressing TuJ1 with neuronal-like morphologies was less than or equal to 1% of the total population (n=3 umbilicus-derived cell isolates tested). While not quantified, the percentage of TuJ1+cells without neuronal morphologies was higher in umbilicus-derived cell cultures than placenta-derived cell cultures. These results appeared specific as age-matched controls in Growth Medium did not express TuJ1.

Summary:

Methods for generating differentiated neurons (based on TuJ1 expression and neuronal morphology) from umbilicus-derived cells were developed. While expression for TuJ1 was not examined earlier than one month in vitro, it is clear that at least a small population of umbilicus-derived cells can give rise to neurons either through default differentiation or through long-term induction following one month of exposure to a minimal media supplemented with L-glutamine, basic FGF, and EGF.

### Example 16

### PPDC Trophic Factors for Neural Progenitor Support

The influence of umbilicus- and placenta-derived cells (collectively postpartum-derived cells or PPDCs) on adult neural stem and progenitor cell survival and differentiation through non-contact dependent (trophic) mechanisms was examined.

### Methods & Materials

Adult Neural Stem and Progenitor Cell Isolation:

Fisher 344 adult rats were sacrificed by CO2 asphyxiation followed by cervical dislocation. Whole brains were removed intact using bone rongeurs and hippocampus tissue dissected based on coronal incisions posterior to the motor and somatosensory regions of the brain (Paxinos, G. & Watson, C. 1997. The Rat Brain in Stereotaxic Coordinates). Tissue was washed in Neurobasal-A medium (Invitrogen, Carlsbad, Calif.) containing B27 (B27 supplement; Invitrogen), L-glutamine (4 mM; Invitrogen), and penicillin/streptomycin (Invitrogen), the combination of which is herein referred to as Neural Progenitor Expansion (NPE) medium. NPE medium was further supplemented with bFGF (20 nanograms/milliliter, Peprotech, Rocky Hill, N.J.) and EGF (20 nanograms/milliliter, Peprotech, Rocky Hill, N.J.), herein referred to as NPE+bFGF+EGF.

Following wash, the overlying meninges were removed, and the tissue minced with a scalpel. Minced tissue was collected and trypsin/EDTA (Invitrogen) added as 75% of the total volume. DNase (100 microliters per 8 milliliters total volume, Sigma, St. Louis, Mo.) was also added. Next, the tissue/media was sequentially passed through an 18 gauge needle, 20 gauge needle, and finally a 25 gauge needle one time each (all needles from Becton Dickinson, Franklin Lakes, N.J.). The mixture was centrifuged for 3 minutes at 250 g. Supernatant was removed, fresh NPE+bFGF+EGF was added and the pellet resuspended. The resultant cell suspension was passed through a 40 micrometer cell strainer (Becton Dickinson), plated on laminin-coated T-75 flasks (Becton Dickinson) or low cluster 24-well plates (Becton Dickinson), and grown in NPE+bFGF+EGF media until sufficient cell numbers were obtained for the studies outlined.

PPDC Plating:

Postpartum-derived cells (umbilicus (022803) P12, (042103) P12, (071003) P12; placenta (042203) P12) previously grown in Growth Medium were plated at 5,000 cells/transwell insert (sized for 24 well plate) and grown for a period of one week in Growth Medium in inserts to achieve confluence.

Adult Neural Progenitor Plating:

Neural progenitors, grown as neurospheres or as single cells, were seeded onto laminin-coated 24 well plates at an approximate density of 2,000 cells/well in NPE+bFGF+EGF for a period of one day to promote cellular attachment. One day later, transwell inserts containing postpartum cells were added according to the following scheme:
a. Transwell (umbilicus-derived cells in Growth Media, 200 microliters)+ neural progenitors (NPE+bFGF+EGF, 1 milliliter)
b. Transwell (placenta-derived cells in Growth Media, 200 microliters)+ neural progenitors (NPE+bFGF+EGF, 1 milliliter)
c. Transwell (adult human dermal fibroblasts [1 F 1853; Cambrex, Walkersville, Md.] P12 in Growth Media, 200 microliters)+neural progenitors (NPE+bFGF+EGF, 1 milliliter)
d. Control: neural progenitors alone (NPE+bFGF+EGF, 1 milliliter)
e. Control: neural progenitors alone (NPE only, 1 milliliter)

Immunocytochemistry:

After 7 days in co-culture, all conditions were fixed with cold 4% (w/v) paraformaldehyde (Sigma) for a period of 10 minutes at room temperature. Immunocytochemistry was performed using antibodies directed against the epitopes listed in Table 14-1. Briefly, cultures were washed with phosphate-buffered saline (PBS) and exposed to a protein blocking solution containing PBS, 4% (v/v) goat serum (Chemic on, Temecula, Calif.), and 0.3% (v/v) Triton (Triton X-100; Sigma) for 30 minutes to access intracellular antigens. Primary antibodies, diluted in blocking solution, were then applied to the cultures for a period of 1 hour at room temperature. Next, primary antibodies solutions were removed and cultures washed with PBS prior to application of secondary antibody solutions (1 hour at room temperature) containing blocking solution along with goat anti-mouse IgG-Texas Red (1:250; Molecular Probes, Eugene, Oreg.) and goat anti-rabbit IgG-Alexa 488 (1:250; Molecular Probes). Cultures were then washed and 10 micromolar DAPI (Molecular Probes) applied for 10 minutes to visualize cell nuclei.

Following immunostaining, fluorescence was visualized using the appropriate fluorescence filter on an Olympus inverted epi-fluorescent microscope (Olympus, Melville, N.Y.). In all cases, positive staining represented fluorescence signal above control staining where the entire procedure outlined above was followed with the exception of application of a primary antibody solution. Representative images were captured using a digital color video camera and ImagePro software (Media Cybernetics, Carlsbad, Calif.). For triple-stained samples, each image was taken using only one emission filter at a time. Layered montages were then prepared using Adobe Photoshop software (Adobe, San Jose, Calif.).

TABLE 16-1
Summary of Primary Antibodies Used

| Antibody | Concentration | Vendor |
|---|---|---|
| Rat 401 (nestin) | 1:200 | Chemicon, Temecula, CA |
| TuJ1 (BIII Tubulin) | 1:500 | Sigma, St. Louis, MO |
| Tyrosine hydroxylase (TH) | 1:1000 | Chemicon |
| GABA | 1:400 | Chemicon |
| GFAP | 1:2000 | DakoCytomation, |
| | | Carpinteria, CA |
| Myelin Basic Protein (MBP) | 1:400 | Chemicon |

Quantitative Analysis of Neural Progenitor Differentiation:

Quantification of hippocampal neural progenitor differentiation was examined. A minimum of 1000 cells were counted per condition or if less, the total number of cells observed in that condition. The percentage of cells positive for a given stain was assessed by dividing the number of positive cells by the total number of cells as determined by DAPI (nuclear) staining.

Mass Spectrometry Analysis & 2D Gel Electrophoresis:

In order to identify unique, secreted factors as a result of co-culture, conditioned media samples taken prior to culture fixation were frozen down at −80° C. overnight. Samples were then applied to ultrafiltration spin devices (MW cutoff 30 kD). Retentate was applied to immunoaffinity chromatography (anti-Hu-albumin; IgY) (immunoaffinity did not remove albumin from the samples). Filtrate was analyzed by MALDI. The pass through was applied to Cibachron Blue affinity chromatography. Samples were analyzed by SDS-PAGE and 2D gel electrophoresis.

### Results

PPDC Co-Culture Stimulates Adult Neural Progenitor Differentiation:

Following culture with umbilicus- or placenta-derived cells, co-cultured neural progenitor cells derived from adult rat hippocampus exhibited significant differentiation along all three major lineages in the central nervous system. This effect was clearly observed after five days in co-culture, with numerous cells elaborating complex processes and losing their phase bright features characteristic of dividing progenitor cells. Conversely, neural progenitors grown alone in the absence of bFGF and EGF appeared unhealthy and survival was limited.

After completion of the procedure, cultures were stained for markers indicative of undifferentiated stem and progenitor cells (nestin), immature and mature neurons (TuJ1), astrocytes (GFAP), and mature oligodendrocytes (MBP). Differentiation along all three lineages was confirmed while control conditions did not exhibit significant differentiation as evidenced by retention of nestin-positive staining amongst the majority of cells. While both umbilicus- and placenta-derived cells induced cell differentiation, the degree of differentiation for all three lineages was less in co-cultures with placenta-derived cells than in co-cultures with umbilicus-derived cells.

The percentage of differentiated neural progenitors following co-culture with umbilicus-derived cells was quantified (Table 16-2). Umbilicus-derived cells significantly enhanced the number of mature oligodendrocytes (MBP) (24.0% vs. 0% in both control conditions). Furthermore, co-culture enhanced the number of GFAP+astrocytes and TuJ1+neurons in culture (47.2% and 8.7% respectively). These results were confirmed by nestin staining indicating that progenitor status was lost following co-culture (13.4% vs. 71.4% in control condition 4).

Though differentiation also appeared to be influenced by adult human fibroblasts, such cells were not able to promote the differentiation of mature oligodendrocytes nor were they able to generate an appreciable quantity of neurons. Though not quantified, fibroblasts did however, appear to enhance the survival of neural progenitors.

TABLE 16-2
Quantification of progenitor differentiation in control vs transwell co-
culture with umbilical-derived cells (E = EGF, F = bFGF)

| | F + E/Umb | F + E/F + E | F + E/removed |
|---|---|---|---|
| Antibody | [Cond.1] | [Cond. 4] | [Cond. 5] |
| TuJ1 | 8.7% | 2.3% | 3.6% |
| GFAP | 47.2% | 30.2% | 10.9% |
| MBP | 23.0% | 0% | 0% |
| Nestin | 13.4% | 71.4% | 39.4% |

Identification of Unique Compounds:

Conditioned media from umbilicus- and placenta-derived co-cultures, along with the appropriate controls (NPE media±I.7% serum, media from co-culture with fibroblasts), were examined for differences. Potentially unique compounds were identified and excised from their respective 2D gels.

Summary:

Co-culture of adult neural progenitor cells with umbilicus or placenta PPDCs results in differentiation of those cells. Results presented in this example indicate that the differentiation of adult neural progenitor cells following co-culture with umbilicus-derived cells is particularly profound. Specifically, a significant percentage of mature oligodendrocytes was generated in co-cultures of umbilicus-derived cells.

### Example 17

### Transplantation of Postpartum-Derived Cells

Cells derived from the postpartum umbilicus and placenta are useful for regenerative therapies. The tissue produced by postpartum-derived cells (PPDCs) transplanted into SCID mice with a biodegradable material was evaluated. The materials evaluated were Vicryl non-woven, 35/65 PCL/PGA foam, and RAD 16 self-assembling peptide hydrogel.

### Methods & Material

Cell Culture:

Placenta- and umbilicus-derived cells were grown in Growth Medium (DMEM-Iow glucose (Gibco, Carlsbad Calif.), 15% (v/v) fetal bovine serum (Cat. #SH30070.03; Hyclone, Logan, Utah), 0.001% (v/v) betamercaptoethanol (Sigma, St. Louis, Mo.), penicillin/streptomycin (Gibco)) in a gelatin-coated flasks.

Sample Preparation:

One million viable cells were seeded in 15 microliters Growth Medium onto 5 mm diameter, 2.25 mm thick Vicryl non-woven scaffolds (64.33 milligrams/cc; Lot#3547-47-1) or 5 mm diameter 35/65 PCL/PGA foam (Lot#3415-53). Cells were allowed to attach for two hours before adding more Growth Medium to cover the scaffolds. Cells were grown on scaffolds overnight. Scaffolds without cells were also incubated in medium.

RAD16 self-assembling peptides (3D Matrix, Cambridge, Mass.) was obtained as a sterile 1% (w/v) solution in water, which was mixed 1:1 with 1×10⁶ cells in 10% (w/v) sucrose (Sigma, St Louis, Mo.), 10 mM HEPES in Dulbecco's modified medium (DMEM; Gibco) immediately before use. The final concentration of cells in RAD 16 hydrogel was 1×10⁶ cells/100 microliters.

Test Material (N=4/Rx)
a. Vicryl non-woven+1×10⁶ umbilicus-derived cells
b. 35/65 PCL/PGA foam+1×10⁶ umbilicus-derived cells
c. RAD 16 self-assembling peptide+1×10⁶ umbilicus-derived cells
d. Vicryl non-woven+1×10⁶ placenta-derived cells
e. 35/65 PCL/PGA foam+1×10⁶ placenta-derived cells
f. RAD 16 self-assembling peptide+1×10⁶ placenta-derived cells
g. 35/65 PCL/PGA foam
h. Vicryl non-woven

Animal Preparation:

The animals were handled and maintained in accordance with the current requirements of the Animal Welfare Act. Compliance with the above Public Laws were accomplished by adhering to the Animal Welfare regulations (9 CFR) and conforming to the current standards promulgated in the Guide for the Care and Use of Laboratory Animals, 7th edition.

Mice (Mus Musculus)/Fox Chase SCID/Male (Harlan Sprague Dawley, Inc., Indianapolis, Ind.), 5 Weeks of Age:

All handling of the SCID mice took place under a hood. The mice were individually weighed and anesthetized with an intraperitoneal injection of a mixture of 60 milligrams/kg KETASET (ketamine hydrochloride, Aveco Co., Inc., Fort Dodge, Iowa) and 10 milligrams/kg ROMPUN (xylazine, Mobay Corp., Shawnee, Kans.) and saline. After induction of anesthesia, the entire back of the animal from the dorsal cervical area to the dorsal lumbosacral area was clipped free of hair using electric animal clippers. The area was then scrubbed with chlorhexidine diacetate, rinsed with alcohol, dried, and painted with an aqueous iodophor solution of 1% available iodine. Ophthalmic ointment was applied to the eyes to prevent drying of the tissue during the anesthetic period.

Subcutaneous Implantation Technique:

Four skin incisions, each approximately 1.0 cm in length, were made on the dorsum of the mice. Two cranial sites were located transversely over the dorsal lateral thoracic region, about 5-mm caudal to the palpated inferior edge of the scapula, with one to the left and one to the right of the vertebral column. Another two were placed transversely over the gluteal muscle area at the caudal sacro-Iumbar level, about 5-mm caudal to the palpated iliac crest, with one on either side of the midline. Implants were randomly placed in these sites in accordance with the experimental design. The skin was separated from the underlying connective tissue to make a small pocket and the implant placed (or injected for RAD16) about 1-cm caudal to the incision. The appropriate test material was implanted into the subcutaneous space. The skin incision was closed with metal clips.

Animal Housing:

Mice were individually housed in micro isolator cages throughout the course of the study within a temperature range of 64° F.-79° F. and relative humidity of 30% to 70%, and maintained on an approximate 12 hour light/12 hour dark cycle. The temperature and relative humidity were maintained within the stated ranges to the greatest extent possible. Diet consisted of Irradiated Pico Mouse Chow 5058 (Purina Co.) and water fed ad libitum.

Mice were euthanized at their designated intervals by carbon dioxide inhalation. The subcutaneous implantation sites with their overlying skin were excised and frozen for histology.

Histology:

Excised skin with implant was fixed with 10% neutral buffered formalin (Richard-Allan Kalamazoo, Mich.). Samples with overlying and adjacent tissue were centrally bisected, paraffin-processed, and embedded on cut surface using routine methods. Five-micron tissue sections were obtained by microtome and stained with hematoxylin and eosin (Poly Scientific Bay Shore, N.Y.) using routine methods.

### Results

There was minimal ingrowth of tissue into foams (without cells) implanted subcutaneously in SCID mice after 30 days. In contrast there was extensive tissue fill in foams implanted with umbilical-derived cells or placenta-derived cells. Some tissue ingrowth was observed in Vicryl non-woven scaffolds. Non-woven scaffolds seeded with umbilicus- or placenta-derived cells showed increased matrix deposition and mature blood vessels.

Summary:

Synthetic absorbable non-woven/foam discs (5.0 mm diameter×1.0 mm thick) or self-assembling peptide hydrogel were seeded with either cells derived from human umbilicus or placenta and implanted subcutaneously bilaterally in the dorsal spine region of SCID mice. The results demonstrated that postpartum-derived cells could dramatically increase good quality tissue formation in biodegradable scaffolds.

### Example 18

### Telomerase Expression in Umbilical Tissue-Derived Cells

Telomerase functions to synthesize telomere repeats that serve to protect the integrity of chromosomes and to prolong the replicative life span of cells (Liu, K, et al., PNAS, 1999; 96:5147-5152). Telomerase consists of two components, telomerase RNA template (hTER) and telomerase reverse transcriptase (hTERT). Regulation of telomerase is determined by transcription of hTERT but not hTER. Real-time polymerase chain reaction (PCR) for hTERT mRNA thus is an accepted method for determining telomerase activity of cells.

Cell Isolation.

Real-time PCR experiments were performed to determine telomerase production of human umbilical cord tissue-derived cells. Human umbilical cord tissue-derived cells were prepared in accordance the examples set forth above. Generally, umbilical cords obtained from National Disease Research Interchange (Philadelphia, Pa.) following a normal delivery were washed to remove blood and debris and mechanically dissociated. The tissue was then incubated with digestion enzymes including collagenase, dispase and hyaluronidase in culture medium at 37° C. Human umbilical cord tissue-derived cells were cultured according to the methods set forth in the examples above. Mesenchymal stem cells and normal dermal skin fibroblasts (cc-2509 lot #9F0844) were obtained from Cambrex, Walkersville, Md. A pluripotent human testicular embryonal carcinoma (teratoma) cell line nTera-2 cells (NTERA-2 c1.D1), (see, Plaia et al., Stem Cells, 2006; 24(3):531-546) was purchased from ATCC (Manassas, Va.) and was cultured according to the methods set forth above.

Total RNA Isolation.

RNA was extracted from the cells using RNeasy® kit (Qiagen, Valencia, Ca.). RNA was eluted with 50 microliters DEPC-treated water and stored at −80° C. RNA was reverse transcribed using random hexamers with the TaqMan® reverse transcription reagents (Applied Biosystems, Foster City, Ca.) at 25° C. for 10 minutes, 37° C. for 60 minutes and 95° C. for 10 minutes. Samples were stored at −20° C.

Real-Time PCR.

PCR was performed on cDNA samples using the Applied Biosystems Assays-On-Demand™ (also known as TaqMan® Gene Expression Assays) according to the manufacturer's specifications (Applied Biosystems). This commercial kit is widely used to assay for telomerase in human cells. Briefly, hTert (human telomerase gene) (Hs00162669) and human GAPDH (an internal control) were mixed with cDNA and TaqMan® Universal PCR master mix using a 7000 sequence detection system with ABI prism 7000 SDS software (Applied Biosystems). Thermal cycle conditions were initially 50° C. for 2 minutes and 95° C. for 10 minutes followed by 40 cycles of 95° C. for 15 seconds and 60° C. for 1 minute. PCR data was analyzed according to the manufacturer's specifications.

Human umbilical cord tissue-derived cells (ATCC Accession No. PTA-6067), fibroblasts, and mesenchymal stem cells were assayed for hTert and 18S RNA. As shown in Table 18-1, hTert, and hence telomerase, was not detected in human umbilical cord tissue-derived cells.

TABLE 18-1

| | hTert | 18S RNA |
|---|---|---|
| Umbilical cells (022803) | ND | + |
| Fibroblasts | ND | + |

ND—not detected;
+ signal detected

Human umbilical cord tissue-derived cells (isolate 022803, ATCC Accession No. PTA-6067) and nTera-2 cells were assayed and the results showed no expression of the telomerase in two lots of human umbilical cord tissue-derived cells while the teratoma cell line revealed high level of expression (Table 18-2).

TABLE 18-2

| | hTert | | GAPDH | | |
|---|---|---|---|---|---|
| Cell type | Exp. 1 | Exp. 2 | Exp. 1 | Exp. 2 | hTert norm |
| nTera2 | 25.85 | 27.31 | 16.41 | 16.31 | 0.61 |
| 022803 | — | — | 22.97 | 22.79 | — |

Therefore, it can be concluded that the human umbilical tissue-derived cells of the present invention do not express telomerase.

Various patents and other publications are referred to throughout the specification. Each of these publications is incorporated by reference herein, in its entirety.

Although the various aspects of the invention have been illustrated above by reference to examples and preferred embodiments, it will be appreciated that the scope of the invention is defined not by the foregoing description but by the following claims properly construed under principles of patent law.

We claim:

1. A method of treating retinal degeneration comprising administering to the eye of a subject a population of postpartum-derived cells, wherein the cell population secretes bridge molecules, and wherein the bridge molecules are selected from MFG-E8, Gas6, TSP-1 and TSP-2.

2. A method of administering a population of postpartum-derived cells to the eye of a subject, wherein the cell population secretes bridge molecules, and wherein the bridge molecules are selected from MFG-E8, Gas6, TSP-1 and TSP-2.

3. The method of claim 1, wherein the population of postpartum-derived cells comprises human umbilical cord tissue-derived cells isolated from human umbilical cord tissue substantially free of blood.

4. The method of claim 3, wherein the cell population isolated from human umbilical cord tissue substantially free of blood is capable of expansion in culture, has the potential to differentiate into cells of at least a neural phenotype, maintains a normal karyotype upon passaging, and has the following characteristics:
a) potential for 40 population doublings in culture;
b) production of CD10, CD13, CD44, CD73, and CD90;
c) lack of production of CD31, CD34, CD45, CD117, and CD141, and
d) increased expression of genes encoding interleukin 8 and reticulon 1 relative to a human cell that is a fibroblast, a mesenchymal stem cell, or an iliac crest bone marrow cell.

5. The method of claim 1, wherein the cell population secretes receptor tyrosine kinase (RTK) trophic factors.

6. The method of claim 5, wherein the trophic factors are BDNF, NT3, HGF, PDGF-CC, PDGF-DD, and GDNF.

7. The method of claim 2, wherein the cell population secretes receptor tyrosine kinase (RTK) trophic factors.

8. The method of claim 7, wherein the trophic factors are BDNF, NT3, HGF, PDGF-CC, PDGF-DD, and GDNF.

9. The method of claim 4, wherein the cell population secretes receptor tyrosine kinase (RTK) trophic factors selected from the group consisting of BDNF, NT3, HGF, PDGF-CC, PDGF-DD, and GDNF.

10. A method of reducing or protecting retinal cells or tissue from oxidative damage comprising administering to the eye of a subject a population of postpartum-derived cells, wherein the postpartum-derived cells are isolated from human umbilical cord tissue substantially free of blood.

11. A method of rescuing retinal pigment epithelial (RPE) cell dysfunction in retinal degeneration, the method comprising administering to the eye of a subject a population of postpartum-derived cells, wherein the postpartum-derived cells are isolated from human umbilical cord tissue substantially free of blood, wherein the cell population secretes bridge molecules, and wherein the bridge molecules are selected from MFG-E8, Gas6, TSP-1 and TSP-2.

12. A method for reducing the loss of photoreceptor cells in retinal degeneration, the method comprising administering to the eye of a subject a population of postpartum-derived cells in an amount effective to reduce the loss of photoreceptor cells, wherein the postpartum-derived cells are isolated from human umbilical cord tissue substantially free of blood, wherein the population of postpartum-derived cells secretes bridge molecules, and wherein the bridge molecules are selected from MFG-E8, Gas6, TSP-1 and TSP-2.

13. The method of claim 11, wherein the population of postpartum-derived cells secretes receptor tyrosine kinase trophic factors selected from the group consisting of BDNF, NT3, HGF, PDGF-CC, PDGF-DD, and GDNF.

14. The method of claim 12, wherein the population of postpartum-derived cells secretes receptor tyrosine kinase trophic factors selected from the group consisting of BDNF, NT3, HGF, PDGF-CC, PDGF-DD, and GDNF.

15. The method of claim 10, wherein the population of postpartum-derived cells is administered with at least one other agent.

16. A method for reducing the loss of photoreceptor cells in retinal degeneration comprising administering to the eye of a subject a composition comprising a population of postpartum-derived cells, wherein the composition is administered in an amount effective to reduce the loss of photoreceptor cells, wherein the postpartum-derived cells are isolated from human umbilical cord tissue substantially free of blood, wherein postpartum-derived cells secrete bridge molecules, and wherein the bridge molecules are selected from MFG-E8, Gash, TSP-1 and TSP-2.

17. The method of claim 16, wherein the population of postpartum-derived cells secretes receptor tyrosine kinase trophic factors selected from the group consisting of BDNF, NT3, HGF, PDGF-CC, PDGF-DD, and GDNF.

18. The method of claim 17, wherein the trophic factors are BDNF, NT3, HGF, PDGF-CC, PDGF-DD, and GDNF.

19. The method of claim 16, wherein the composition is a pharmaceutical composition.

20. The method of claim 19, wherein the pharmaceutical composition comprises a pharmaceutically acceptable carrier.

21. The method of claim 1, wherein the retinal degeneration is age-related macular degeneration.

22. The method of claim 21, wherein the age-related macular degeneration is dry age-related macular degeneration.

23. The method of claim 11, wherein the cell population isolated from human umbilical cord tissue substantially free of blood is capable of expansion in culture, has the potential to differentiate into cells of at least a neural phenotype, maintains a normal karyotype upon passaging, and has the following characteristics:
a) potential for 40 population doublings in culture;
b) production of CD10, CD13, CD44, CD73, and CD90;
c) lack of production of CD31, CD34, CD45, CD117, and CD141, and
d) increased expression of genes encoding interleukin 8 and reticulon 1 relative to a human cell that is a fibroblast, a mesenchymal stem cell, or an iliac crest bone marrow cell.

24. The method of claim 4, wherein the cell population is positive for HLA-A,B,C, and negative for HLA-DR,DP,DQ.

25. The method of claim 23, wherein the cell population is positive for HLA-A,B,C, and negative for HLA-DR,DP,DQ.

26. The method of claim 1, wherein administration to the eye is selected from administration to the interior of an eye or administration behind the eye.
